(19) United States
(12) Patent Application Publication
(10) Pub. No.: US 2013/0196410 A1
(43) Pub. Date: Aug. 1, 2013

(54) COMPOSITIONS AND METHODS FOR MODIFYING THE GLYCOSYLATION PATTERN OF A POLYPEPTIDE

(71) Applicant: Anthony Rossomando, Cambridge, MA (US)

(72) Inventor: Anthony Rossomando, Cambridge, MA (US)

(73) Assignee: ALNYLAM PHARMACEUTICALS, INC, CAMBRIDGE, MA (US)

(21) Appl. No.: 13/582,098

(22) PCT Filed: Mar. 3, 2011
(86) PCT No.: PCT/US11/26994

§ 371 Date: Mar. 11, 2013

Related U.S. Application Data
(60) Provisional application No. 61/310,889, filed on Mar. 5, 2010.

(51) Int. Cl. C12N 9/24

(52) U.S. Cl. CPC C12N 9/2402
USPC 435/196; 435/209; 435/358; 536/24.5; 435/208; 435/200

(57) ABSTRACT

Provided herein are methods and compositions for expressing a modified polypeptide in a host cell, wherein the modified polypeptide comprises a terminal mannose at an N-linked glycosylation site of the polypeptide. The methods and compositions used herein involve the use of RNA effector molecules (e.g., siRNA, dsRNA etc) administered to a host cell to modify the expression of target genes involved in protein glycosylation (e.g., Mgat1, Mgat4, SLC35A1, SLC35A2 or GNE).

### CROSS REFERENCE TO RELATED APPLICATIONS

This application claims benefit under 35 U.S.C. §119(e) of the U.S. Provisional Application No. 61/310,889 filed on Mar. 5, 2010, the content of which is incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on Apr. 5, 2011, is named ABIO003PCT20021007PCTSequenceListing.txt and is 455,395 bytes in size.

### FIELD OF THE INVENTION

The field of the invention relates to production of a polypeptide having a modified glycosylation pattern in a host cell.

### BACKGROUND

The lysosomal storage diseases are a group of inherited metabolic disorders that result from a lysosomal enzyme defect that causes accumulation of a metabolic substrate of the enzyme. For example, Gaucher's disease is caused by a reduction of glucocerebrosidase activity resulting in the accumulation of glucocerebrosides primarily in mononuclear cells. Symptoms from Gaucher's disease can range from mild to severe and can include enlarged spleen and liver, neurologic complications, lymph node swelling, anemia, skeletal disorders and bone lesions.

Enzyme replacement therapy has been used successfully to manage symptoms of Gaucher's disease and other lysosomal storage diseases such as e.g., Pompe disease and Fabry's disease. Although enzyme replacement therapy is not a cure, such treatments can effectively manage the disorder when administered on a regular basis. In the case of Gaucher's disease, intravenous recombinant glucocerebrosidase administered to patients decreases liver and spleen size, reduces skeletal abnormalities, and reverses other manifestations.

### SUMMARY OF THE INVENTION

The methods and compositions described herein are based, in part, on the discovery that the mannosylation pattern of an expressed polypeptide can be modified in a host cell during production of the polypeptide using RNA effector molecules. For example, the methods and compositions provided herein permit modification of a glycosylation chain at an N-linked glycosylation site of a polypeptide, such that the polypeptide comprises at least one terminal mannose.

Provided herein are methods for producing a polypeptide with a modified glycosylation pattern at an N-linked glycosylation site, comprising the steps of (a) culturing a cell comprising a polypeptide to be modified in the presence of at least one RNA effector molecule that inhibits expression of a gene product involved in protein glycosylation such that at least one polypeptide N-linked glycosylation site is modified to have a terminal mannose, and the cell is cultured under conditions permitting glycosylation and for a sufficient time to allow expression of the polypeptide to be modified; and (b) isolating the polypeptide, wherein the polypeptide produced comprises a terminal mannose in at least one N-linked glycosylation site, thereby producing a polypeptide with a modified glycosylation pattern. The method can be further modified to inhibit expression of the mannose 6 phosphate receptor, which prevents accumulation of the polypeptide product in lysosomes and in one embodiment permits secretion of the polypeptide from the cell during the production process.

Alternatively, provided herein are methods for producing a polypeptide with a modified glycosylation pattern at an N-linked glycosylation site, comprising the steps of culturing a cell comprising a polypeptide to be modified in the presence of at least one RNA effector molecule that inhibits expression of a gene product involved in protein glycosylation such that at least one polypeptide N-linked glycosylation site is modified to have a terminal mannose, and the cell is cultured under conditions permitting glycosylation and for a sufficient time to allow expression of the polypeptide to be modified, wherein the polypeptide comprises a terminal mannose at the at least one N-linked glycosylation site.

In some embodiments, a plurality of N-linked glycosylation sites on a polypeptide produced by this method are modified (e.g., 2, 3, 4, 5, 6, 7, 8, 9). For example, glucocerebrosidase comprises 4 N-linked glycosylation sites and 1, 2, 3, or 4 of the sites can be modified to comprise a terminal mannose.

In one embodiment, the modified N-linked glycosylation site comprises an oligomannosyl structure, such as e.g., Man₂GlcNAc₂, Man₃GlcNAc₂, Man₄GlcNAc₂, Man₅GlcNAc₂, Man₆GlcNAc₂, Man₇GlcNAc₂, Man₈GlcNAc₂, and Man₉GlcNAc₂.

In some embodiments, the polypeptide comprises 2, 3, 4, 5, 6, 7, 8, or 9 terminal mannoses in the at least one N-linked glycosylation site.

The methods and compositions described herein permit inhibition of expression of a gene product involved in glycosylation from a target gene such as e.g., Mgat1, Mgat4 (e.g., Mgat4A and Mgat 4B), SLC35A1, SLC35A2, and GNE by an RNA effector molecule (e.g., an siRNA). Exemplary RNA effector molecules targeting these genes are found herein in Tables 2-24. In some embodiments, two or more RNA effector molecules are cultured with the host cell. In one embodiment, the RNA effector molecule is added to the culture medium of the host cell. In another embodiment the RNA effector molecule is administered in a composition comprising a reagent that facilitates RNA effector molecule uptake into the host cell.

In one embodiment, the RNA effector molecule is administered by means of a continuous infusion into the culture medium. Alternatively, the RNA effector molecule is administered to the culture medium in a discrete dose. Such doses can be given once or repeated throughout the production of the polypeptide (e.g., at a frequency of 6 h, 12 h, 24 h, 36 h, 48 h, 72 h, 84 h, 96 h, or 108 h). In one embodiment, the RNA effector molecule administration is repeated at least three times. While the dosage of a particular RNA effector molecule can be determined by one of skill in the art, an RNA effector molecule will typically be added at a concentration of approximately 0.1 nM, 0.5 nM, 0.75 nM, 1 nM, 2 nM, 5 nM, 10 nM, 20 nM, 30 nM, 40 nM, 50 nM, 60 nM, or any integer therebetween. Alternatively, the RNA effector molecule is added at an amount of 50 molecules per cell, 100 molecules per cell, 200 molecules per cell, 300 molecules per cell, 400 molecules per cell, 500 molecules per cell, 600 molecules per cell, 700 molecules per cell, 800 molecules per cell, 900 molecules per cell, 1000 molecules per cell, 2000 molecules per cell, or 5000 molecules per cell. In another embodiment, the RNA effector molecule is added at a concentration selected from the group consisting of: 0.01 fmol/10⁶ cells, 0.1 fmol/10⁶ cells, 0.5 fmol/10⁶ cells, 0.75 fmol/10⁶ cells, 1 fmol/10⁶ cells, 2 fmol/10⁶ cells, 5 fmol/10⁶ cells, 10 fmol/10⁶ cells, 20 fmol/10⁶ cells, 30 fmol/10⁶ cells, 40 fmol/10⁶ cells, 50 fmol/10⁶ cells, 60 fmol/10⁶ cells, 100 fmol/10⁶ cells, 200 fmol/10⁶ cells, 300 fmol/10⁶ cells, 400 fmol/10⁶ cells, 500 fmol/10⁶ cells, 700 fmol/10⁶ cells, 800 fmol/10⁶ cells, 900 fmol/10⁶ cells, and 1 pmol/10⁶ cells.

In one embodiment, the methods and compositions described herein permit the production of a polypeptide capable of binding a mannose receptor present on macrophages.

In one embodiment, the polypeptide is further modified enzymatically to remove remaining or unwanted glycosylation groups. In another embodiment, the polypeptide is not modified enzymatically to contain the terminal mannose.

In some embodiments, the polypeptide is used in treatment of a lysosomal storage disease (e.g., glucocerebrosidase, idursulfase, alglucosidase alfa, galsulfase, agalsidase beta, and laronidase). In one embodiment, the polypeptide comprises a mutation. For example, a commonly used glucocerebrosidase mutant comprises an arginine to histidine mutation at amino acid 495, which enhances the uptake of glucocerebrosidase by mononuclear cells.

Also provided herein are isolated polypeptide compositions comprising a modified mannosylation pattern produced by a method comprising the steps of (a) culturing a cell comprising a polypeptide to be modified in the presence of at least one RNA effector molecule that inhibits expression of a gene product involved in protein glycosylation such that at least one polypeptide N-linked glycosylation site is modified to have a terminal mannose, and the cell is cultured under conditions permitting glycosylation and for a sufficient time to allow expression of the polypeptide to be modified; and (b) isolating the polypeptide, wherein the polypeptide comprises a terminal mannose at the at least one N-linked glycosylation site.

In some embodiments, the polypeptide lacks a mannose phosphate group and/or has a reduced affinity for the mannose 6 phosphate receptor.

In one embodiment, the polypeptide is glucocerebrosidase. In one embodiment, the glucocerebrosidase polypeptide comprises an arginine to histidine mutation at amino acid 495.

Provided herein are isolated mammalian host cells, in which the mRNA expression of a target gene selected from the group consisting of: Mgat1, Mgat4, SLC35A1, SLC35A2, and GNE is inhibited by RNA interference, wherein when a gene encoding a polypeptide is introduced into the host cell and expressed, the host cell produces a polypeptide comprising the encoded polypeptide molecule which contains a terminal mannose in at least one glycosylation chain (e.g., N-linked glycosylation chain), said polypeptide having increased affinity for the mannose receptor when compared with the polypeptide produced in the presence of Mgat1, Mgat4, SLC35A1, SLC35A2, or GNE expression, thereby producing a polypeptide with increased macrophage internalization.

In one embodiment, the cell is a CHO cell or a CHO cell derivative (e.g., CHO-DG44 cells). In some embodiments, the host cell(s) are cultured in suspension or in a bioreactor. In some embodiments, the cell is cultured in a volume selected from the group consisting of 0.1 L, 0.5 L, 1 L, 5 L, 40 L, 500 L, 5000 L, and 50,000 L.

In one embodiment, the mRNA expression of the target gene is transiently inhibited (e.g., by contacting the cell with at least one RNA effector molecule in a composition comprising a reagent that facilitates RNA effector molecule uptake into the cell). In one embodiment, a plurality of RNA effector molecules are cultured with the cell (e.g., two or more).

In one embodiment, mRNA expression of the target gene is inhibited in the host cell by continuous infusion of at least one RNA effector molecule into a culture medium used for maintaining the cell to produce the polypeptide.

In one embodiment, the continuous infusion is administered at a rate to achieve a desired average percent inhibition for the at least one target gene. In one embodiment, the RNA effector molecule is continuously infused as an admixture comprising a reagent that facilitates RNA effector molecule uptake into the cells (e.g., an emulsion, a liposome, a cationic lipid, a non-cationic lipid, an anionic lipid, a charged lipid, a penetration enhancer, or a transfection reagent).

In another embodiment, the addition of the RNA effector molecule is repeated throughout the production of the polypeptide.

In another embodiment, the addition of the RNA effector molecule is repeated at a frequency selected from the group consisting of: 6 h, 12 h, 24 h, 36 h, 48 h, 72 h, 84 h, 96 h, and 108 h.

In another embodiment, the addition of the RNA effector molecule is repeated at least three times.

In another embodiment, the at least one RNA effector molecule is added at a concentration selected from the group consisting of 0.1 nM, 0.5 nM, 0.75 nM, 1 nM, 2 nM, 5 nM, 10 nM, 20 nM, 30 nM, 40 nM, 50 nM, and 60 nM.

In another embodiment, the at least one RNA effector molecule is added at an amount of 50 molecules per cell, 100 molecules per cell, 200 molecules per cell, 300 molecules per cell, 400 molecules per cell, 500 molecules per cell, 600 molecules per cell, 700 molecules per cell, 800 molecules per cell, 900 molecules per cell, 1000 molecules per cell, 2000 molecules per cell, or 5000 molecules per cell.

In another embodiment, the at least one RNA effector molecule is added at a concentration selected from the group consisting of: 0.01 fmol/10⁶ cells, 0.1 fmol/10⁶ cells, 0.5 fmol/10⁶ cells, 0.75 fmol/10⁶ cells, 1 fmol/10⁶ cells, 2 fmol/10⁶ cells, 5 fmol/10⁶ cells, 10 fmol/10⁶ cells, 20 fmol/10⁶ cells, 30 fmol/10⁶ cells, 40 fmol/10⁶ cells, 50 fmol/10⁶ cells, 60 fmol/10⁶ cells, 100 fmol/10⁶ cells, 200 fmol/10⁶ cells, 300 fmol/10⁶ cells, 400 fmol/10⁶ cells, 500 fmol/10⁶ cells, 700 fmol/10⁶ cells, 800 fmol/10⁶ cells, 900 fmol/10⁶ cells, and 1 pmol/10⁶ cells.

Also described herein are composition(s) comprising at least one RNA effector molecule comprising a nucleic acid sequence complementary to at least one target gene of a host cell, wherein the RNA effector molecule is capable of modulating mannosylation patterns at an N-linked glycosylation site of a polypeptide produced in the host cell, and wherein the target gene is selected from the group consisting of: Mgat1, Mgat4, SLC35A1, SLC35A2, and GNE.

In some embodiments, the RNA effector molecule comprises a duplex region. In other embodiments, the RNA effector molecules are 15-30 or 17-28 nucleotides in length. The RNA effector molecule can further comprise a modified nucleotide, if so desired. In one embodiment, the RNA effector molecule comprises a sequence selected from the group consisting of sequences provided herein in Tables 2-24.

Also provided herein are kits for producing a polypeptide comprising at least one terminal mannose at an N-linked glycosylation site, the kit comprising: (a) at least one RNA effector molecule that inhibits a gene product involved in protein glycosylation in an admixture with a host cell; and (b) instructions and packaging materials therefor.

In some embodiments, the kit provides RNA effector molecules that target hamster Mgat1, Mgat4, SLC35A1, SLC35A2, or GNE, such as those found in Tables 2-6 herein. In other embodiments, the kit provides RNA effector molecules for human Mgat1, Mgat4, SLC35A1, SLC35A2, or GNE, such as those found in Tables 7-10, 20, and 23 herein. In other embodiments, the kit provides RNA effector molecules for mouse Mgat1, Mgat4, SLC35A1, SLC35A2, or GNE, such as those found in Tables 11-14, 21, and 24 herein. In other embodiments, the kit provides RNA effector molecules for rat Mgat1, Mgat4, SLC35A1, SLC35A2, or GNE, such as those found in Tables 15-19, and 22, herein.

The kit can further comprise a cell medium for culturing the host cell or a variety of expression vectors useful for expressing a polypeptide in the host cell (e.g., mammalian cell).

In one embodiment, the RNA effector molecule is provided as a composition comprising an RNA effector molecule and a reagent that facilitates RNA effector molecule uptake into a cell.

The kit can further comprise RNA effector molecules that activate expression of the target gene (i.e., RNA activation or RNAa).

In one embodiment, the kit further comprises an agent that facilitates RNA effector uptake into a cell.

Another aspect described herein relates to an isolated polypeptide that comprises a terminal mannose in at least one N-linked glycosylation site, wherein the glycosylation pattern of the isolated polypeptide has not been modified enzymatically to contain the terminal mannose. In one embodiment, the polypeptide is glucocerebrosidase.

Also described herein are compositions comprising a dsRNA for inhibiting expression of at least one hamster target gene selected from the group consisting of: Mgat1, Mgat4, SLC35A1, SLC35A2, and GNE, the dsRNA comprising (a) a sense strand comprising a sequence selected from the group consisting of: SEQ ID NO. 1-33, SEQ ID NO. 67-94, SEQ ID NO. 123-154, SEQ ID NO. 187-221, and SEQ ID NO. 257-282; and (b) a complementary anti-sense strand comprising a sequence selected from the group consisting of SEQ ID NO. 34-66, SEQ ID NO. 95-122, SEQ ID NO. 155-186, SEQ ID NO. 222-256 and SEQ ID NO. 283-308. In one embodiment, such compositions further comprise a reagent that facilitates uptake of a dsRNA into a cell, for example, an emulsion, a liposome, a cationic or non-cationic lipid, an anionic lipid, a penetration enhancer, a transfection reagent, or a chemical linkage that attaches a ligand, peptide group, a lipophillic group, a targeting moiety etc. Such reagents that facilitate uptake of an RNA effector molecule into a cell are described herein throughout the detailed description.

Also described herein are compositions comprising a dsRNA for inhibiting expression of at least one human target gene selected from the group consisting of: Mgat1, Mgat4A, Mgat4B, SLC35A1, SLC35A2, and GNE, the dsRNA comprising (a) a sense strand comprising a sequence selected from the group consisting of: SEQ ID NO. 310-336, SEQ ID NO. 365-385, SEQ ID NO. 408-435, SEQ ID NO. 465-489, SEQ ID NO. 969-994 and SEQ ID NO. 1116-1141; and (b) a complementary anti-sense strand comprising a sequence selected from the group consisting of SEQ ID NO. 337-363, SEQ ID NO. 386-406, SEQ ID NO. 436-463, SEQ ID NO. 490-514, SEQ ID NO. 995-1020 and SEQ ID NO. 1142-1167. In one embodiment, such compositions further comprise a reagent that facilitates uptake of a dsRNA into a cell, for example, an emulsion, a cationic lipid, a non-cationic lipid, a charged lipid, a liposome, an anionic lipid, a penetration enhancer, a transfection reagent or a modification to the RNA effector molecule to attach e.g., a ligand, a targeting moiety, a peptide, a lipophillic group etc.

Also described herein are compositions comprising a dsRNA for inhibiting expression of at least one mouse target gene selected from the group consisting of: Mgat1, Mgat4A, Mgat4B, SLC35A1, SLC35A2, and GNE, the dsRNA comprising (a) a sense strand comprising a sequence selected from the group consisting of: SEQ ID NO. 516-541, SEQ ID NO. 569-595, SEQ ID NO. 624-644, SEQ ID NO. 667-695, SEQ ID NO. 1022-1042 and SEQ ID NO. 1169-1196; and (b) a complementary anti-sense strand comprising a sequence selected from the group consisting of SEQ ID NO. 542-567, SEQ ID NO. 596-622, SEQ ID NO. 645-665, SEQ ID NO. 696-724, SEQ ID NO. 1043-1063, and SEQ ID NO. 1197-1224. In one embodiment, such compositions further comprise a reagent that facilitates uptake of a dsRNA into a cell, for example, an emulsion, a cationic lipid, a non-cationic lipid, a charged lipid, a liposome, an anionic lipid, a penetration enhancer, a transfection reagent or a modification to the RNA effector molecule to attach e.g., a ligand, a targeting moiety, a peptide, a lipophillic group etc.

Also described herein are compositions comprising a dsRNA for inhibiting expression of at least one rat target gene selected from the group consisting of: Mgat1, Mgat4A, Mgat4B, SLC35A1, SLC35A2, and GNE, the dsRNA comprising (a) a sense strand comprising a sequence selected from the group consisting of: SEQ ID NO. 726-751, SEQ ID NO. 779-802, SEQ ID NO. 828-849, SEQ ID NO. 873-894, SEQ ID NO. 918-942, and SEQ ID NO. 1065-1089; and (b) a complementary anti-sense strand comprising a sequence selected from the group consisting of SEQ ID NO. 752-777, SEQ ID NO. 803-826, SEQ ID NO. 850-871, SEQ ID NO. 895-916, SEQ ID NO. 943-967, and SEQ ID NO. 1090-1114. In one embodiment, such compositions further comprise a reagent that facilitates uptake of a dsRNA into a cell, for example, an emulsion, a cationic lipid, a non-cationic lipid, a charged lipid, a liposome, an anionic lipid, a penetration enhancer, a transfection reagent or a modification to the RNA effector molecule to attach e.g., a ligand, a targeting moiety, a peptide, a lipophillic group etc.

### DEFINITIONS

For convenience, the meaning of certain terms and phrases used in the specification, examples, and appended claims, are provided below. If there is an apparent discrepancy between the usage of a term in other parts of this specification and its definition provided in this section, the definition in this section shall prevail.

“G,” “C,” “A,” “T” and “U” each generally stand for a nucleotide that contains guanine, cytosine, adenine, thymine and uracil as a base, respectively. However, it will be understood that the term “deoxyribonucleotide,” “ribonucleotide,” or “nucleotide” can also refer to a modified nucleotide, as further detailed below, or a surrogate replacement moiety. The skilled person is well aware that a ribonucleotide comprising a thymine base is also referred to as 5-methyl uridine and a deoxyribonucleotide comprising a uracil base is also referred to as deoxy-Uridine in the art. The skilled person is also well aware that guanine, cytosine, adenine, thymine and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

As used herein the term “lysosomal storage disease” refers to a metabolic disorder that results from a defect in lysosomal function. Some non-limiting examples of lyosomal storage diseases include Gaucher's disease, Tay Sachs disease, Fabry's disease, Pompe disease, Sandhoff disease, Wolman disease, Salla disease, Alpha-N-acetylgalactosaminidase deficiency, Neuronal Ceroid Lipofuscinoses, Niemann-Pick Disease, Mucopolysaccharidoses disorders, Krabbe disease, and Farber disease. In one embodiment, the lysosomal storage disease is Gaucher's disease, which results from a hereditary defect in the glucocerebrosidase enzyme.

As used herein, the term “cell comprising a polypeptide to be modified” encompasses a cell that expresses a polypeptide to be modified either endogenously (e.g., a polypeptide native to the cell) or exogenously (e.g., a polypeptide expressed in the cell). In its simplest form, the “cell comprising a polypeptide to be modified” refers to a cell that is capable of producing the polypeptide in the absence of a transgene. Alternatively, the cell can be engineered to express a polypeptide to be modified, using methods and expression systems known to those of skill in the art. In one embodiment, the cell is engineered by administration of a transgene that expresses the polypeptide to be modified. A transgene can be administered by any means known in the art including e.g., vectors, plasmids, viral vectors, incorporation of a transgene into the genome of the host cell. The transgene can be under the control of an inducible promoter. If desired, the cell is treated to enhance the expression of a polypeptide (e.g., native or exogenously expressed) using e.g., means of RNA activation such as RNA duplexes targeting the promoter region of the polypeptide.

As used herein the term “polypeptide to be modified” refers to an endogenous polypeptide of the host cell or an exogenous polypeptide expressed in the host cell. In some embodiments, the polypeptide is mutated compared to the wildtype polypeptide endogenous to the host cell. In some embodiments, the polypeptide is a lysosomally targeted polypeptide (i.e., a polypeptide ordinarily targeted to the lysosome) and the host cell is a mammalian cell. In one embodiment, the polypeptide is a polypeptide modified to be taken up by macrophages. In one embodiment, the polypeptide is useful for treating a lysosomal storage disease. Non-limiting examples of polypeptides that can be produced according to methods provided herein include glucocerebrosidase, idursulfase, alglucosidase alfa, galsulfase, agalsidase beta, and laronidase. As used herein, the term polypeptide encompasses glycoproteins or other polypeptides which have undergone post-translational modification, such as deamidation, glycation, and the like. In one embodiment, post-translational modification of the polypeptide is modified using the methods and compositions described herein. In one embodiment, the polypeptide is modified to include a terminal mannose in at least one glycosylation chain. In one embodiment, the polypeptide is enzymatically active (e.g., glucocerebrosidase hydrolyzes a glucocerebroside).

As used herein, the term “modified glycosylation pattern” refers to the presence of a different glycan chain at an N-linked glycosylation site of a protein when the polypeptide is produced in a cell cultured in the presence of an RNA effector molecule (as described herein) as compared to the glycan chain at the same N-glycosylation site on the polypeptide produced in a cell cultured in the absence of such an RNA effector molecule.

As used herein, the term “terminal mannose” refers to a mannose at the terminus of a branch of a glycosylation chain at an N-glycosylation site of a polypeptide. A single N-linked glycosylation site can comprise a glycosylation chain having several different branch points (e.g., 1, 2, 3, 4, 5, 6, 7, 8, 9, 10, 11, 12, etc.) resulting in a plurality of “branches”. The terminus of each branch comprises a terminal group (e.g., mannose, galactose, N-acetylglucosamine, sialic acid etc). Thus, the term “terminal mannose” refers to a mannose at the terminus of a single branch. However, a single glycosylation chain can comprise a plurality of terminal mannoses at the end of a plurality of glycosylation branches. As used herein, the term “plurality of terminal mannoses” refers to at least two terminal mannoses at an N-linked glycosylation site e.g., 3, 4, 5, 6, 7, 8, 9, 10, 11, 12, or more terminal mannoses. The term “terminal mannose” also encompasses the presence of other terminal groups (e.g., galactose, N-acetylglucosamine, sialic acid etc) at the termini of the other glycosylation branches and is referred to herein as a “hybrid oligosaccharide.” In one embodiment, all of the branches of a glycosylation chain at an N-linked glycosylation site have a terminal mannose and is also referred to herein as an “oligomannosyl structure,” “high mannose structure” or “oligomannose.” The oligomannosyl structure can have 2, 3, 4, 5, 6, 7, 8, 9, or more mannose residues in the glycan chain (e.g., not all mannoses in the chain are terminal mannoses). In one embodiment, the “terminal mannose” is exposed (e.g., the mannose residue is positioned such that it is able to bind to a mannose receptor). One of skill in the art can determine the presence of an exposed terminal mannose by treating the polypeptide with a mannosidase to remove the mannose groups and comparing to a glycosylated form of the polypeptide e.g., that lacks a terminal mannose group. Alternatively, one can determine if at least one terminal mannose is exposed by detecting binding of the modified polypeptide to a mannose receptor using e.g., radioligand binding assays.

As used herein, the term “modified to have a terminal mannose” refers to the modification of a polypeptide to comprise a terminal mannose at an N-linked glycosylation site when the polypeptide is produced in a cell cultured in the presence of an RNA effector molecule as compared to the glycan chain (lacking a terminal mannose) at the same N-linked glycosylation site on the polypeptide produced in the cell cultured in the absence of such an RNA effector molecule.

As used herein the term “N-linked glycosylation site” or “N-glycan site” are used interchangeably to refer to a site comprising a sequon (e.g., amino acid consensus sequence) that permits the addition of an N-linked glycan to the nitrogen group of an asparagine amino acid residue of a polypeptide. In one embodiment, the sequon comprises Asn-X-Ser, wherein X is any amino acid except proline. In another embodiment, the sequon comprises Asn-X-Thr, wherein X is any amino acid except proline.

As used herein, the term “at least one N-linked glycosylation site” refers to at least one, at least two, at least three, at least four, at least five, at least 6, at least 7, at least 8, at least 9, at least 10, at least 11, at least 12, or more N-linked glycosylation sites on a polypeptide. In one embodiment, the N-linked glycosylation site(s) is/are endogenous to the polypeptide. In one embodiment, the polypeptide is engineered to contain the N-linked glycosylation site(s).

A “host cell,” as used herein, is any eukaryotic cell capable of being grown and maintained in cell culture under conditions allowing for production and recovery of useful quantities of a polypeptide, as defined herein. Host cells can be unmodified cells or cell lines, or cell lines which have been genetically modified (e.g., to facilitate production of a polypeptide or biological product). In some embodiments, the host cell is a cell line that has been modified to allow for growth under desired conditions, such as in serum-free media, in cell suspension culture, or in adherent cell culture. In other embodiments, the host cell can be selected from the group consisting of a plant cell, a fungal cell, an insect cell and a mammalian cell. In some embodiments, the host cell is a mammalian cell.

As used herein, the phrase “conditions permitting glycosylation” refers to cell culture conditions that allow glycosylation of the expressed polypeptide in the absence of an RNA effector molecule as described herein. Typically, mammalian cells produce glycosylated proteins under the same conditions or similar conditions that allow expression of an endogenous polypeptide. One of skill in the art can easily determine appropriate conditions that allow polypeptide expression and glycosylation by modifying e.g., temperature, pH, pO₂, CO₂ level, humidity etc. In general, these conditions will also be used to generate a modified polypeptide having a terminal mannose using an RNA effector molecule as described herein. However, one of skill in the art can modify the conditions to enhance modification of the polypeptide, if so desired.

As used herein, the term “RNA effector molecule” refers to an oligonucleotide capable of modulating the expression of a target gene, as defined herein, within a host cell, or a polynucleotide agent capable of forming an oligonucleotide that can modulate the expression of a target gene upon being introduced into a host cell. As used herein, the phrase “in the presence of at least one RNA effector molecule” encompasses exposure of the cell to an RNA effector molecule expressed within the cell, e.g., shRNA, or exposure by exogenous addition of the RNA effector molecule to the cell, e.g., delivery of the RNA effector molecule to the cell, optionally using an agent that facilitates uptake into the cell. A portion of an RNA effector molecule is substantially complementary to at least a portion of the target gene RNA, such as the coding region, the promoter region and the 3′ untranslated region (3′-UTR) of the target gene RNA.

In the context of this invention, the term “oligonucleotide” refers to a polymer or oligomer of nucleotide or nucleoside monomers comprising naturally occurring bases sugars and intersugar (backbone) linkages. The term “oligonucleotide” also includes polymers or oligomers comprising non-naturally occurring monomers, or portions thereof, which function similarly. Such modified or substituted oligonucleotides are often preferred over native forms because of properties such as, for example, enhanced cellular uptake, increased stability in the presence of nucleases, and the like.

Double-stranded and single-stranded oligonucleotides that are effective in inducing RNA interference are also referred to as siRNA, RNAi agent, or iRNA agent, herein. These RNA interference inducing oligonucleotides associate with a cytoplasmic multi-protein complex known as RNAi-induced silencing complex (RISC). Without wishing to be bound by theory, RNA interference leads to Argonaute-mediated post-transcriptional cleavage of target gene mRNA transcripts. In many embodiments, single-stranded and double-stranded RNAi agents are sufficiently long that they can be cleaved by an endogenous molecule, e.g. by Dicer, to produce smaller oligonucleotides that can enter the RISC machinery and participate in RISC mediated cleavage of a target sequence, e.g. a target mRNA.

As used herein, the term “region” or “portion,” when used in reference to an RNA effector molecule refers to a nucleic acid sequence of at least 3, at least 4, at least 5, at least 6, at least 7, at least 8, at least 9, at least 10 or more nucleotides up to and including the entire nucleic acid sequence of a strand of an RNA effector molecule. In some embodiments, the “region” or “portion” when used in reference to an RNA effector molecule includes nucleic acid sequence one nucleotide shorter than the entire nucleic acid sequence of a strand of an RNA effector molecule. Thus, the term “portion” refers to a region of an RNA effector molecule having a desired length to effect complementary binding to a region of a target gene RNA or a desired length of a duplex region. One of skill in the art can vary the length of the “portion” that is complementary to the target gene or arranged in a duplex, such that an RNA effector molecule having desired characteristics (e.g., inhibition of a target gene or stability) is produced. While not wishing to be bound by theory, RNA effector molecules provided herein can modulate expression of target genes by one or more of a variety of mechanisms, including but not limited to, Argonaute-mediated post-transcriptional cleavage of target gene mRNA transcripts (sometimes referred to in the art as RNAi) and/or other pre-transcriptional and/or pre-translational mechanisms.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

Complementary sequences within an RNA effector molecule, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes described herein.

“Complementary” sequences, as used herein, can also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but are not limited to, G:U Wobble or Hoogstein base pairing.

The terms “complementary,” “fully complementary” and “substantially complementary” herein can be used with respect to the base matching between the sense strand and the antisense strand of a dsRNA, or between the antisense strand of an RNA effector molecule agent and a target sequence, as will be understood from the context of their use.

As used herein, a polynucleotide that is “substantially complementary to at least part of” a target gene refers to a polynucleotide that is substantially complementary to a contiguous portion of a target gene of interest (e.g., an mRNA encoded by a target gene, the target gene's promoter region or 3′ UTR). For example, a polynucleotide is complementary to at least a part of a target mRNA if the sequence is substantially complementary to a non-interrupted portion of an mRNA encoded by a target gene.

In some embodiments, a plurality of RNA effector molecules are used to modulate expression of one or more target genes. As used herein, the term “plurality” refers to at least 2 or more RNA effector molecules e.g., 3, 4, 5, 6, 7, 8, 9, 10, 11, 12, 13, 14, 15, 16, 17, 18, 19, 20, 30, 40, 50, 60, 80, 100 RNA effector molecules or more. The term “plurality” can also refer to at least 2 or more target genes, e.g., 3, 4, 5, 6, 7, 8, 9, 10, 11, 12, 13, 14, 15, 16, 17, 18, 19, 20, 30, 40, 50, 60, 70, 80, 90, 100 target genes or more.

As used herein the term “culturing a cell” or “contacting a cell” refers to the treatment of a cell in culture with an agent e.g., at least one RNA effector molecule, often prepared in a composition comprising a reagent that facilitates uptake of the RNA effector molecule into the cell (e.g., Lipofectamine). The step of contacting a cell with an RNA effector molecule(s) can be repeated more than once (e.g., twice, 3×, 4×, 5×, 6×, 7×, 8×, 9×, 10×, 11×, 12×, 13×, 14×, 15×, 16×, 17×, 18×, 19×, 20×, 30×, 40×, 50×, 60×, 70×, 80×, 90×, 100× or more). In one embodiment, the cell is contacted such that the target gene is modulated only transiently, e.g., by addition of an RNA effector molecule composition to the cell culture medium used for the production of the polypeptide where the presence of the RNA effector molecule dissipates over time, i.e., the RNA effector molecule is not constitutively expressed in the cell.

“Introducing into a cell,” when referring to an RNA effector molecule, means facilitating or effecting uptake or absorption into the cell, as is understood by those skilled in the art. Absorption or uptake of an RNA effector molecule can occur through unaided diffusive or active cellular processes, or by auxiliary agents or devices. In vitro introduction into a cell includes methods known in the art such as electroporation and lipofection. Further approaches are described herein below or known in the art.

As used herein, the phrase “reagent that facilitates RNA effector molecule uptake” refers to any agent that enhances uptake of an RNA effector molecule into a host cell by at least 10%, at least 20%, at least 30%, at least 40%, at least 50%, at least 60%, at least 70%, at least 80%, at least 90%, at least 95%, at least 99%, at least 1-fold, at least 2-fold, at least 5-fold, at least 10-fold, at least 100-fold, at least 1000-fold or more compared to an RNA effector molecule administered in the absence of such a reagent. In one embodiment, a cationic or non-cationic lipid molecule useful for preparing a composition or for co-administration with an RNA effector molecule is used as a reagent that facilitates RNA effector molecule uptake. In other embodiments, the reagent that facilitates RNA effector molecule uptake comprises a chemical linkage to attach e.g., a ligand, a peptide group, a lipophillic group, a targeting moiety etc, as described throughout the application herein. In other embodiments, the reagent that facilitates RNA effector molecule uptake comprises a charged lipid, an emulsion, a liposome, a cationic or non-cationic lipid, an anionic lipid, a transfection reagent or a penetration enhancer as described throughout the application herein. In one embodiment, the reagent that facilitates RNA effector molecule uptake used herein comprises a charged lipid as described in U.S. Ser. No. 61/267,419 filed on Dec. 7, 2009, which is herein incorporated by reference in its entirety.

As used herein, a “target gene” refers to a nucleic acid that encodes an RNA, for example, nucleic acid sequences including, but not limited to, genes encoding a polypeptide and genes encoding non coding RNAs. By “target gene RNA” or “target RNA” is meant RNA encoded by the target gene. The skilled person is well aware that a target gene RNA that encodes a polypeptide is more commonly known as messenger RNA (mRNA). The target gene can be a gene derived from a cell, an endogenous gene, a transgene, or exogenous genes such as genes of a pathogen, for example a virus, which is present in the cell after infection thereof. The cell containing the target gene can be derived from or contained in any organism, for example a plant, animal, protozoan, virus, bacterium, or fungus. In some embodiments, the target gene encodes a protein that affects one or more aspects of the production of peptide glycosylation by a host cell, such that modulating expression of the gene permits production of a polypeptide comprising at least one terminal mannose.

The term “expression” as used herein is intended to mean the transcription to an RNA and/or translation to one or more polypeptides from a target gene coding for the sequence of the RNA and/or the polypeptide.

In some embodiments, the target gene encodes a non-coding RNA (ncRNA) that affects one or more aspects of the production of peptide glycosylation by a host cell, such that modulating expression of the gene permits production of a polypeptide comprising at least one terminal mannose. As used herein, a “non-coding RNA” refers to a target gene RNA that is not translated into a protein. The non-coding RNA is also referred to as non-protein-coding RNA (npcRNA), non-messenger RNA (mRNA), small non-messenger RNA (smRNA), and functional RNA (fRNA) in the art. The target gene from which a non-coding RNA is transcribed as the end product is also referred to as an RNA gene or non-coding RNA gene herein. Non-coding RNA genes include highly abundant and functionally important RNAs such as transfer RNA (tRNA) and ribosomal RNA (rRNA), as well as RNAs such as snoRNAs, microRNAs, siRNAs and piRNAs.

The term “modulates expression of,” and the like, in so far as it refers to a target gene, herein refer to the modulation of expression of a target gene, as manifested by a change (e.g., an increase or a decrease) in the amount of target gene RNA which can be isolated from or detected in a first cell or group of cells in which a target gene is transcribed and which has or have been treated such that the expression of a target gene is modulated, as compared to a second cell or group of cells substantially identical to the first cell or group of cells but which has or have not been so treated (control cells). The degree of modulation can be expressed in terms of

$$\frac{\begin{matrix}( target gene RNA in control cells ) - \\ ( target gene RNA in treated cells )\end{matrix}}{( target gene RNA in control cells )} \cdot 100 \%$$

Alternatively, the degree of modulation can be given in terms of a parameter that is functionally linked to target gene expression, e.g., the amount of protein encoded by a target gene, or the number of cells displaying a certain phenotype, e.g., reduced glycosylation of polypeptides. In principle, target gene modulation can be determined in any host cell expressing the target gene, either constitutively or by genomic engineering, and by any appropriate assay.

As described herein, expression of a target gene is inhibited. In one example, expression of a target gene is inhibited by at least 10%, at least 15%, at least 20%, at least 25%, at least 30%, at least 35%, at least 40%, at least 45%, or at least 50% by administration of an RNA effector molecule provided herein. In some embodiments, expression of a target gene is inhibited by at least 60%, at least 70%, or at least 80% by administration of an RNA effector molecule to a host cell. In some embodiments, expression of a target gene is inhibited by at least 85%, at least 90%, or at least 95% or more by administration of an RNA effector molecule as described herein. In one embodiment, expression of the target gene is inhibited by 99% or even 100% (e.g., below detectable limits).

In other instances, expression of a target gene is activated by at least 10%, at least 20%, at least 25%, at least 30%, at least 40%, at least 50%, at least 60%, at least 70%, at least 80%, at least 90%, at least 1-fold, at least 2-fold, at least 5-fold, at least 10-fold, at least 100-fold, at least 1000-fold, at least 10000-fold or more by administration of an RNA effector molecule provided herein.

A “bioreactor,” as used herein, refers generally to any reaction vessel suitable for growing and maintaining cells such that the cells produce a polypeptide, and for recovering such polypeptide. Bioreactors described herein include cell culture systems of varying sizes, such as small culture flasks, Nunc multilayer cell factories, small high yield bioreactors (e.g., MiniPerm, INTEGRA-CELLine), spinner flasks, hollow fiber-WAVE bags (Wave Biotech, Tagelswangen, Switzerland), and industrial scale bioreactors. In some embodiments, the polypeptide is produced in a bioreactor having a capacity suitable for pharmaceutical or industrial scale production of polypeptides (e.g., a volume of at least 2 liters, at least 5 liters, at least 10 liters, at least 25 liters, at least 50 liters, at least 100 liters, or more) and means of monitoring pH, glucose, lactate, temperature, and/or other bioprocess parameters.

As used herein, an “RNA effector composition” comprises an effective amount of an RNA effector molecule and an acceptable carrier. As used herein, “effective amount” refers to that amount of an RNA effector molecule effective to produce a modulatory effect on a bioprocess for the production of a polypeptide.

As used herein, the term “average percent inhibition” refers to the average degree of inhibition of target gene expression over time that is necessary to produce the desired effect (e.g., modification of protein glycosylation) and which is below the degree of inhibition that produces any unwanted or negative effects. In some embodiments, the desired average percent inhibition is at least 20%, at least 30%, at least 40%, at least 50%, at least 60%, at least 70%, at least 80%, at least 85%, at least 90%, at least 95%, at least 99%, or even 100% (i.e., absent). One of skill in the art can use routine cell death assays to determine the upper limit for desired percent inhibition (e.g., level of inhibition that produces unwanted effects). One of skill in the art can also use methods to detect target gene expression (e.g., RT-PCR) to determine an amount of an RNA effector molecule that produces gene modulation. The percent inhibition is described herein as an average value over time, since the amount of inhibition is dynamic and can fluctuate slightly between doses of the RNA effector molecule.

As used herein, the phrase “reduced affinity for the mannose 6 phosphate receptor” refers to a polypeptide produced in the presence of an RNA effector molecule (as described herein) and having at least a 10% reduced ability to bind the mannose 6 phosphate receptor compared to a polypeptide produced in the absence of the RNA effector molecule. One of skill in the art can determine affinity by using e.g., a receptor binding assay (see e.g., Van Patten et al., Glycobiology 17(5):467-478 (2007)) and/or determining the Kd (i.e., the dissociation constant) for the polypeptide binding to the mannose receptor. In other embodiments, the polypeptide has at least 20%, at least 30%, at least 40%, at least 50%, at least 60%, at least 70%, at least 80%, at least 90%, at least 95%, or at least 99% reduction in mannose 6 phosphate receptor binding compared to the polypeptide expressed in the absence of the RNA effector molecule. In one embodiment, the polypeptide expressed in the presence of the RNA effector molecule does not bind the mannose 6 phosphate receptor within detectable limits of a receptor binding assay.

As used herein, the term “transiently inhibited” refers to inhibition of a target gene following administration of a discrete dose of an RNA effector molecule, such that the inhibition of the target gene decreases as the RNA effector molecule is cleared from the cell. In some cases, inhibition may be completely lost in between repeated administrations of an RNA effector molecule in discrete doses. In other embodiments, there may be only a partial loss of inhibition (e.g., 10%, 20%, 30%, 40%, 50%, 60%, 70%, 80%, 90% etc) as the RNA effector molecule activity is cleared. The length of time that inhibition is maintained following treatment with a single dose of RNA effector molecule will depend on the particular RNA effector molecule and/or the target gene. One of skill in the art can easily determine using e.g., ELISA assays to determine the level of inhibition and/or the loss of inhibition over time to choose an appropriate dosing regime to (1) transiently inhibit the target gene, (2) continuously inhibit the target gene, or (3) maintain at least a partial inhibition of the target gene.

The term “acceptable carrier” refers to a carrier for administration of an RNA effector molecule to cultured eukaryotic host cells. Such carriers include, but are not limited to, saline, buffered saline, dextrose, water, glycerol, ethanol, and combinations thereof. The term specifically excludes cell culture medium.

As used herein the phrase “has not been modified enzymatically to contain the terminal mannose” when used to refer to an isolated polypeptide means that the polypeptide has not been subjected to removal of glycosylation groups (e.g., by neuraminidase, galactosidase and/or β-N acetyl glucosaminidase) to expose a terminal mannose following isolation of the polypeptide (e.g., in a separate step). The polypeptides produced using the methods described herein are secreted from the cell with a terminal mannose and do not require an additional enzymatic modification to remove the glycosylation groups. However, one of skill in the art may desire to further modify the peptide using enzymatic modification to remove any remaining or undesired glycosylation groups and such use of an enzyme for modification is also contemplated herein.

As used herein, the term “SNALP” refers to a stable nucleic acid-lipid particle. A SNALP represents a vesicle of lipids coating a reduced aqueous interior comprising a nucleic acid such as an RNA effector molecule or a plasmid from which an RNA effector molecule is transcribed. SNALPs are described, e.g., in U.S. Patent Application Publication Nos. 2006/0240093, 2007/0135372, and U.S. patent application Ser. Nos. 12/343,342, filed on Dec. 23, 2008 and 12/424,367, filed on Apr. 15, 2009. These applications are hereby incorporated by reference in their entirety.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

### DETAILED DESCRIPTION

Provided herein are methods and compositions for expressing a modified polypeptide in a host cell, wherein the modified polypeptide comprises a terminal mannose at an N-linked glycosylation site of the polypeptide. The methods and compositions used herein involve the use of an RNA effector molecule(s) (e.g., siRNA, dsRNA etc) administered to a host cell to modify the expression of target gene(s) involved in protein glycosylation (e.g., Mgat1, Mgat4 (e.g., Mgat4A or Mgat4B), SLC35A1, SLC35A2 or GNE).

### Industrial Production of Polypeptides

The methods and compositions described herein can be applied to any system for producing a polypeptide in a mammalian cell, including polypeptide production on an industrial scale. The present invention may be combined with any known method or composition to enhance the production of a polypeptide or biological product, such as those disclosed in e.g., U.S. Provisional No. 61/293,980 or described herein.

A non-limiting exemplary process for the industrial-scale production of a heterologous polypeptide (e.g., a polypeptide to be modified) in cell culture (e.g., mammalian cell culture) includes the following steps:
i) inoculating mammalian host cells containing a transgene encoding the heterologous protein (e.g., polypeptide to be modified) into a seed culture vessel containing cell culture medium and propagating the cells to reach a minimum threshold cross-seeding density;
ii) transferring the propagated seed culture cells, or a portion thereof, to a large-scale bioreactor;
iii) propagating the large-scale culture under conditions allowing for rapid growth and cell division until the cells reach a predetermined density;
iv) maintaining the culture under conditions that disfavor continued cell growth and/or cell division and facilitate expression of the heterologous protein.

The cells can be cultured in a stirred tank bioreactor system in a fed batch culture process in which the host cells and culture medium are supplied to the bioreactor initially and additional culture nutrients are fed, continuously or in discrete increments, throughout the cell culture process. The fed batch culture process can be semi-continuous, wherein periodically the entire culture (including cells and medium) is removed and replaced. Alternatively, a simple batch culture process can be used in which all components for cell culturing (including the cells and culture medium) are supplied to the culturing vessel at the start of the process. A continuous perfusion process can also be used, in which the cells are immobilized in the culture, e.g., by filtration, encapsulation, anchoring to microcarriers, or the like, and the supernatant is continuously removed from the culturing vessel and replaced with fresh medium during the process.

Steps i)-iii) of the above method generally comprise a “growth” phase, whereas step iv) generally comprises a “production” phase. In some embodiments, fed batch culture or continuous cell culture conditions are tailored to enhance growth and division of the cultured cells in the growth phase and to disfavor cell growth and/or division and facilitate expression of the heterologous protein during the production phase. For example, in some embodiments, a heterologous protein is expressed at levels of about 1 mg/L, or about 2.5 mg/L, or about 5 mg/L or higher. The rate of cell growth and/or division can be modulated by varying culture conditions, such as temperature, pH, dissolved oxygen (dO₂) and the like. For example, suitable conditions for the growth phase can include a pH of between about 6.5 and 7.5, a temperature between about 30° C. to 38° C., and a dO₂ between about 5-90% saturation. In some embodiments, the expression of a heterologous protein can be enhanced in the production phase by inducing a temperature shift to a lower culture temperature (e.g., from about 37° C. to about 30° C.), increasing the concentration of solutes in the cell culture medium, or adding a toxin (e.g., sodium butyrate) to the cell culture medium. A variety of additional protocols and conditions for enhancing growth during the growth phase and/or protein expression during the production phase are known in the art.

In one embodiment, after the production phase the heterologous protein is recovered from the cell culture medium using various methods known in the art. Recovering a secreted heterologous protein or polypeptide typically involves removal of host cells and debris from the medium, for example, by centrifugation or filtration. In some embodiments, the methods provided herein further comprise inhibition of the mannose 6 phosphate receptor such that the expressed polypeptide does not accumulate in lysosomes. In other embodiments, the polypeptide produced in a host cell does not comprise a mannose 6 phosphate group such that it is preferentially secreted rather than imported into lysosomes by mannose 6 phosphate mediated uptake.

In some cases, particularly if the protein is not secreted, protein recovery can also be performed by lysing the cultured host cells, e.g., by mechanical shear, osmotic shock, or enzymatic treatment, to release the contents of the cells into the homogenate. The polypeptide can then be separated from subcellular fragments, insoluble materials, and the like by differential centrifugation, filtration, affinity chromatography, hydrophobic interaction chromatography, ion-exchange chromatography, size exclusion chromatography, electrophoretic procedures (e.g., preparative isoelectric focusing (IEF)), ammonium sulfate precipitation, and the like. Procedures for recovering and purifying particular types of proteins are known in the art.

Methods and compositions useful for enhancing polypeptide production in cells is provided in e.g., U.S. Provisional Application 61/293,980, which is incorporated herein by reference in its entirety. Such methods are directed at e.g., increasing cell growth, increasing cell viability, decreasing apoptosis, decreasing lactate formation, decreasing reactive oxygen species production, modifying post-translational modifications, and decreasing viral contamination of cells in culture.

### Host Cells

In one embodiment, a mammalian host cell is preferred to produce a polypeptide or recombinant polypeptide, particularly if the polypeptide is a biotherapeutic agent or is otherwise intended for administration to or consumption by humans. In some embodiments, the host cell is a Chinese Hamster Ovary (CHO) cell, which is the predominant cell line used for the expression of many recombinant proteins. Additional mammalian cell lines commonly used for the expression of recombinant proteins include, but are not limited to, 293HEK cells, HeLa cells, COS cells, NIH/3T3 cells, Jurkat Cells, NSO cells and HUVEC cells.

In some embodiments, the host cell is a CHO cell derivative that has been genetically modified to facilitate production of recombinant proteins, polypeptides, or other biological products. For example, various CHO cell strains have been developed which permit stable insertion of recombinant DNA into a specific gene or expression region of the cells, amplification of the inserted DNA, and selection of cells exhibiting high level expression of the recombinant protein. Examples of CHO cell derivatives useful in the methods provided herein include, but are not limited to, CHO-K1 cells, CHO-DUKX, CHO-DUKX B1, CHO-DG44 cells, CHO-ICAM-1 cells, and CHO-h1FNγ cells. Methods for expressing recombinant proteins in CHO cells are known in the art and are described, e.g., in U.S. Pat. Nos. 4,816,567 and 5,981,214, herein incorporated by reference in their entirety.

Examples of human cell lines useful in methods provided herein include, but are not limited to, 293T (embryonic kidney), 786-0 (renal), A498 (renal), A549 (alveolar basal epithelial), ACHN (renal), BT-549 (breast), BxPC-3 (pancreatic), CAKI-1 (renal), Capan-1 (pancreatic), CCRF-CEM (leukemia), COLO 205 (colon), DLD-1 (colon), DMS 114 (small cell lung), DU145 (prostate), EKVX (non-small cell lung), HCC-2998 (colon), HCT-15 (colon), HCT-116 (colon), HT29 (colon), HT-1080 (fibrosarcoma), HEK 293 (embryonic kidney), HeLa (cervical carcinoma), HepG2 (hepatocellular carcinoma), HL-60(TB) (leukemia), HOP-62 (non-small cell lung), HOP-92 (non-small cell lung), HS 578T (breast), HT-29 (colon adenocarcinoma), IGR-OV1 (ovarian), IMR32 (neuroblastoma), Jurkat (T lymphocyte), K-562 (leukemia), KM12 (colon), KM20L2 (colon), LAN5 (neuroblastoma), LNCap.FGC (Caucasian prostate adenocarcinoma), LOX IMVI (melanoma), LXFL 529 (non-small cell lung), M14 (melanoma), M19-MEL (melanoma), MALME-3M (melanoma), MCFlOA (mammary epithelial), MCF7 (mammary), MDA-MB-453 (mammary epithelial), MDA-MB-468 (breast), MDA-MB-231 (breast), MDA-N (breast), MOLT-4 (leukemia), NCI/ADR-RES (ovarian), NCI-H226 (non-small cell lung), NCI-H23 (non-small cell lung), NCI-H322M (non-small cell lung), NCI-H460 (non-small cell lung), NCI-H522 (non-small cell lung), OVCAR-3 (ovarian), OVCAR-4 (ovarian), OVCAR-5 (ovarian), OVCAR-8 (ovarian), P388 (leukemia), P388/ADR (leukemia), PC-3 (prostate), PERC6® (E1-transformed embryonal retina), RPMI-7951 (melanoma), RPMI-8226 (leukemia), RXF 393 (renal), RXF-631 (renal), Saos-2 (bone), SF-268 (CNS), SF-295 (CNS), SF-539 (CNS), SHP-77 (small cell lung), SH-SY5Y (neuroblastoma), SK-BR3 (breast), SK-MEL-2 (melanoma), SK-MEL-5 (melanoma), SK-MEL-28 (melanoma), SK-OV-3 (ovarian), SN12K1 (renal), SN12C (renal), SNB-19 (CNS), SNB-75 (CNS)SNB-78 (CNS), SR (leukemia), SW-620 (colon), T-47D (breast), THP-1 (monocyte-derived macrophages), TK-10 (renal), U87 (glioblastoma), U293 (kidney), U251 (CNS), UACC-257 (melanoma), UACC-62 (melanoma), UO-31 (renal), W138 (lung), and XF 498 (CNS).

Examples of rodent cell lines useful in methods provided herein include, but are not limited to, baby hamster kidney (BHK) cells (e.g., BHK21 cells, BHK TK-cells), mouse Sertoli (TM4) cells, buffalo rat liver (BRL 3A) cells, mouse mammary tumor (MMT) cells, rat hepatoma (HTC) cells, mouse myeloma (NSO) cells, murine hybridoma (Sp2/0) cells, mouse thymoma (EL4) cells, Chinese Hamster Ovary (CHO) cells and CHO cell derivatives, murine embryonic (NIH/3T3, 3T3 L1) cells, rat myocardial (H9c2) cells, mouse myoblast (C2C12) cells, and mouse kidney (miMCD-3) cells.

Examples of non-human primate cell lines useful in methods provided herein include, but are not limited to, monkey kidney (CVI-76) cells, African green monkey kidney (VERO-76) cells, green monkey fibroblast (Cos-1) cells, and monkey kidney (CVI) cells transformed by SV40 (Cos-7). Additional mammalian cell lines are known to those of ordinary skill in the art and are catalogued at the American Type Culture Collection catalog (ATCC®, Mamassas, Va.).

In some embodiments, the host cells are suitable for growth in suspension cultures. Suspension-competent host cells are generally monodisperse or grow in loose aggregates without substantial aggregation. Suspension-competent host cells include cells that are suitable for suspension culture without adaptation or manipulation (e.g., hematopoietic cells, lymphoid cells) and cells that have been made suspension-competent by modification or adaptation of attachment-dependent cells (e.g., epithelial cells, fibroblasts).

In some embodiments, the host cell is an attachment dependent cell which is grown and maintained in adherent culture. Examples of human adherent cell lines useful in methods provided herein include, but are not limited to, human neuroblastoma (SH-SY5Y, IMR32 and LAN5) cells, human cervical carcinoma (HeLa) cells, human breast epithelial (MCFlOA) cells, human embryonic kidney (293T) cells, and human breast carcinoma (SK-BR3) cells.

In some embodiments, the host cell is a multipotent stem cell or progenitor cell. Examples of multipotent cells useful in methods provided herein include, but are not limited to, murine embryonic stem (ES-D3) cells, human umbilical vein endothelial (HuVEC) cells, human umbilical artery smooth muscle (HuASMC) cells, human differentiated stem (HKB-II) cells, and human mesenchymal stem (hMSC) cells.

In some embodiments, the host cell is a plant cell, such as a tobacco plant cell.

In some embodiments, the host cell is a fungal cell, such as a cell from Pichia pastoris, a Rhizopus cell, or a Aspergillus cell.

In some embodiments, the host cell is an insect cell, such as SF9 or SF-21 cells from Spodoptera frugiperda or S2 cells from Drosophila melanogaster.

### Polypeptides

The methods and compositions described herein are useful in modifying an expressed polypeptide to comprise a terminal mannose. In some embodiments, the terminal mannose of the modified polypeptide is exposed such that it is capable of binding to the mannose receptor. These methods and compositions are particularly useful for producing polypeptides that are taken up readily by mononuclear cells. In some embodiments, the polypeptides modified using the methods described herein are useful in the treatment of lysosomal storage diseases such as Gaucher's disease, Fabry's disease or Pompe disease. Exemplary polypeptides contemplated for modification with the methods described herein include glucocerebrosidase, idursulfase, alglucosidase alfa, galsulfase, agalsidase beta, and laronidase. Idursulfase is a recombinant protein corresponding to iduronate 2-sulfatase (IDS) (SEQ ID NO. 1230), while alglucosidase alfa is a recombinant form of acid alpha-glucosidase (GAA) (SEQ ID NO. 1232). Galsulfase is a recombinant form of arylsulfatase B (ARSB) (SEQ ID NO. 1234) and agalsidase beta is a recombinant form of alpha galactosidase A (GLA) (SEQ ID NO. 1236). Laronidase is a recombinant protein corresponding to alpha-L-iduronidase (IDUA) (SEQ ID NO. 1238). Glucocerebrosidase (GBA) (SEQ ID NOs. 1228 and 1229) differs in sequence from Arg495His Glucocerebrosidase (SEQ ID NOs. 1225 and 1226) by an arginine to histidine amino acid mutation at position 495.

In some cases, the polypeptide may comprise a mutation compared to the endogenously expressed version of the polypeptide commonly observed in a standard population of individuals. Mutations can be in the nucleic acid sequence (e.g., genomic or mRNA sequence), or alternatively can comprise an amino acid substitution. Such amino acid substitutions can be conserved mutations or non-conserved mutations. As well-known in the art, a “conservative substitution” of an amino acid or a “conservative substitution variant” of a polypeptide refers to an amino acid substitution which maintains: 1) the structure of the backbone of the polypeptide (e.g. a beta sheet or alpha-helical structure); 2) the charge or hydrophobicity of the amino acid; or 3) the bulkiness of the side chain. More specifically, the well-known terminologies “hydrophilic residues” relate to serine or threonine. “Hydrophobic residues” refer to leucine, isoleucine, phenylalanine, valine or alanine. “Positively charged residues” relate to lysine, arginine or histidine. “Negatively charged residues” refer to aspartic acid or glutamic acid. Residues having “bulky side chains” refer to phenylalanine, tryptophan or tyrosine. To avoid doubt as to nomenclature, the term “D144N” or similar terms specifying other specific amino acid substitutions means that the Asp (D) at position 144 is substituted with Asn (N). A “conservative substitution variant” of D144N would substitute a conservative amino acid variant of Asn (N) that is not D.

The terminology “conservative amino acid substitutions” is well known in the art, which relates to substitution of a particular amino acid by one having a similar characteristic (e.g., similar charge or hydrophobicity, similar bulkiness). Examples include aspartic acid for glutamic acid, or isoleucine for leucine. A list of exemplary conservative amino acid substitutions is given in the table below. A conservative substitution mutant or variant will 1) have only conservative amino acid substitutions relative to the parent sequence, 2) will have at least 90% sequence identity with respect to the parent sequence, preferably at least 95% identity, 96% identity, 97% identity, 98% identity or 99% or greater identity; and 3) will retain polypeptide activity as that term is defined herein.

CONSERVATIVE AMINO ACID REPLACEMENTS

| For Amino Acid | Code | Replace With |
|---|---|---|
| Alanine | A | D-ala, Gly, Aib, β-Ala, Acp, L-Cys, D-Cys |
| Arginine | R | D-Arg, Lys, D-Lys, homo-Arg, D-homo-Arg, Met, |
| | | Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S—Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, Aib, β-Ala, Acp |
| Isoleucine | I | D-Ile, Val, D-Val, AdaA, AdaG, Leu, D-Leu, Met, D-Met |
| Leucine | L | D-Leu, Val, D-Val, AdaA, AdaG, Leu, D-Leu, Met, D-Met |
| Lysine | K | D-Lys, Arg, D-Arg, homo-Arg, D-homo-Arg, Met, |
| | | D-Met, Ile, D-Ile, Orn, D-Orn |
| Methionine | M | D-Met, S—Me-Cys, Ile, D-Ile, Leu, D-Leu, Val, D-Val |
| Phenylalanine | F | D-Phe, Tyr, D-Thr, L-Dopa, His, D-His, Trp, D-Trp, |
| | | Trans-3,4 or 5-phenylproline, AdaA, AdaG, cis-3,4 |
| | | or 5-phenylproline, Bpa, D-Bpa |
| Proline | P | D-Pro, L-I-thioazolidine-4-carboxylic acid, D-or-L-1- |
| | | oxazolidine-4-carboxylic acid (Kauer, U.S. Pat. No. |
| | | (4,511,390) |
| Serine | S | D-Ser, Thr, D-Thr, allo-Thr, Met, D-Met, Met (O), |
| | | D-Met (O), L-Cys, D-Cys |
| Threonine | T | D-Thr, Ser, D-Ser, allo-Thr, Met, D-Met, Met (O), D- |
| | | Met (O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met, AdaA, |
| | | AdaG |

A non-conservative mutation is any other amino acid substitution other than the conservative substitutions noted in the above table.

In one embodiment, a glucocerebrosidase enzyme comprises an arginine to histidine mutation at amino acid 495, which aids in uptake of the enzyme by mononuclear cells.

In some embodiments, the polypeptide is further modified to be secreted into the cell culture medium following production in a host cell. Such modifications can include e.g., removal or inhibition of a mannose 6 phosphate group, which prevents uptake into lysosomes of the host cell via a mannose 6 phosphate receptor mediated mechanism.

In one embodiment, the polypeptide to be modified is glucocerebrosidase. In one embodiment, the glucocerebrosidase is enzymatically active as determined by a glucocerebrosidase activity assay (e.g., measuring enzymatic hydrolysis of 4-methyl-umbelliferyl-B-D glucosidase by glucocerebrosidase to a fluorescent product; see e.g., Methods of Enzymology Vol. L pp: 478-479, 1978). In another embodiment, the modified glucocerebrosidase substantially retains the activity of either the wildtype glucocerebrosidase enzyme (e.g., human placental glucocerebrosidase) or the mutant glucocerebrosidase (e.g., Arg495His mutation), each of which have typical glycosylation patterns of native glucocerebrosidase (e.g., unmodified by the methods described herein). By “substantially retain” is meant that the modified polypeptide comprising a terminal mannose retains at least 60% of the activity of the unmodified polypeptide (e.g., wildtype or mutant glucocerebrosidase activity). In some embodiments, the modified polypeptide retains at least 70%, at least 80%, at least 90%, at least 95%, at least 99%, or even 100% of the activity of the unmodified polypeptide. The term “substantially retains” also encompasses an increase in the activity of the modified polypeptide having a terminal mannose of at least 10% compared to the unmodified polypeptide; in some embodiments the increase in activity of the modified polypeptide is at least 20%, at least 30%, at least 40%, at least 50%, at least 60%, at least 70%, at least 80%, at least 90%, at least 95%, at least 99%, at least 1-fold, at least 2-fold, at least 5-fold, at least 10-fold, at least 50-fold, at least 100-fold, at least 1000-fold or more compared to the unmodified polypeptide.

Typically, human placental glucocerebrosidase has four glycosylation sites that can be modified using the methods described herein. In some embodiments, 1, 2, 3, or 4 of the native glycosylation sites of glucocerebrosidase are modified to contain at least one terminal mannose. In another embodiment, the glucocerebrosidase can be modified to express an additional N-linked glycosylation site(s), which can be further modified to contain at least one terminal mannose. It is contemplated herein that modifications made to the glucocerebrosidase do not result in a substantial loss (e.g., >60%) in glucocerebrosidase activity.

### Gene Products Involved in Glycosylation

Essentially any gene product that is involved in protein glycosylation, such that modification of its expression permits production of a polypeptide having a terminal mannose can be used with the methods and compositions described herein. Some exemplary target genes for mammalian cells include e.g., Mgat1, Mgat4, SLC35A1, SLC35A2 and GNE.

Mgat1 (mannosyl (alpha-1,3-)-glycoprotein beta-1,2-N-acetylglucosaminyltransferase; Gene ID MGAT1) encodes a protein located in the Golgi apparatus, which is responsible for the synthesis of hybrid and complex N-glycans. Similarly, Mgat4a or Mgat4b (mannosyl (alpha-1,3-)-glycoprotein beta-1,4-N-acetylglucosaminyltransferase) encodes a glycosyltransferase protein in the Golgi apparatus responsible for producing tri- and multi-antennary branching structures on polypeptides. SCL35A1 (solute carrier family 35 (CMP-sialic acid transporter), member A1) encodes a protein in the Golgi apparatus that facilitates transport of nucleotide sugars, such as CMP-sialic acid, into the Golgi for glycosylation. Another member of the solute carrier family 35 is SLC35A2 (solute carrier family 35 (UDP-galactose transporter), member A2), which permits transport of solutes, including UDP galactose, into the Golgi apparatus. GNE (glucosamine (UDP-N-acetyl)-2-epimerase/N-acetylmannosamine kinase) encodes a bifunctional enzyme that is rate-limiting in the sialic acid biosynthesis pathway. Inhibition of any one of these gene products, or any combination thereof, permits the production of a polypeptide comprising a terminal mannose.

In one embodiment, the cells are treated with RNA effector molecules that target a single gene product selected from the group consisting of: Mgat1, Mgat4, SCL35A1, SCL35A2, and GNE. In another embodiment, the cells are treated with a combination RNA effector molecules that target at least two gene products selected from the group consisting of: Mgat1, Mgat4, SCL35A1, SCL35A2 and GNE. Some non-limiting examples of inhibition of a combination of gene products include: Mgat1/Mgat4; Mgat1/Gne; Mgat1/SCL35A1; Mgat1/SCL35A2; Mgat4/Gne; Mgat4/SCL35A1; Mgat4/SCL35A2; Gne/SCL35A1; Gne/SCL35A2; SCL35A1/SCL35A2; Mgat1/Mgat4/Gne; Mgat1/Mgat4/SCL35A1; Mgat1/Mgat4/SCL35A2; Mgat1/Gne/SCL35A1; Mgat1/Gne/SCL35A2; Mgat1/SCL35A1/SCL35A2; Mgat1/Mgat4/Gne/SCL35A1; Mgat1/Mgat4/Gne/SCL35A2; Mgat4/Gne/SCL35A1; Mgat4/Gne/SCL35A2; Mgat4/SCL35A1/SCL35A2; Gne/SCL35A1/SCL35A2; Mgat1/Mgat4/Gne/SCL35A1; Mgat1/Mgat4/Gne/SCL35A2; Mgat1/Mgat4/SCL35A1/SCL35A2; Mgat1/Gne/SCL35A1/SCL35A2; Mgat4/Gne/SCL35A1/SCL35A2; or Mgat1/Mgat4/Gne/SCL35A1/SCL35A2.

The particular gene products or isoforms that can be targeted to produce a polypeptide having a terminal mannose may vary slightly among different host cells. RNA effector molecules can be designed using the gene or mRNA sequence of a particular gene product in a desired host cell line. However, it is acknowledged that most mammalian cell lines will have similar mechanisms involved in glycosylation such that the RNA effector molecules described herein in Tables 2-24 will be useful in a variety of mammalian cell lines.

### RNA Effector Molecules

Essentially any RNA effector molecule capable of inhibiting expression of a target gene involved in protein glycosylation in a mammalian cell can be used with the methods described herein. Exemplary RNA effector molecules are provided herein in Tables 2-24. In addition, in certain embodiments, an RNA effector molecule capable of increasing expression of an endogenous polypeptide to be modified can be used (e.g., by targeting the promoter region of a polypeptide to be modified using an RNA activating agent). RNA effector molecules can comprise a single strand or more than one strand. The RNA effector molecule can be single-stranded or double-stranded. A single-stranded RNA effector can have double-stranded regions and a double-stranded RNA effector can have single-stranded regions. Without limitations, RNA effector molecules can include, double stranded RNA (dsRNA), microRNA (miRNA), short interfering RNA (siRNA), antisense RNA, promoter-directed RNA (pdRNA), Piwi-interacting RNA (piRNA), expressed interfering RNA (eiRNA), short hairpin RNA (shRNA), antagomirs, decoy RNA, DNA, plasmids and aptamers.

As used herein, the term “double-stranded” refers to an oligonucleotide having a hybridized duplex region that comprises two anti-parallel and substantially complementary nucleic acid strands. The duplex region can be of any length that permits specific degradation of a desired target RNA through a RISC pathway, but will typically range from 9 to 36 base pairs in length, e.g., 15-30 base pairs in length. Considering a duplex between 9 and 36 base pairs, the duplex can be any length in this range, for example, 9, 10, 11, 12, 13, 14, 15, 16, 17, 18, 19, 20, 21, 22, 23, 24, 25, 26, 27, 28, 29, 30, 31, 32, 33, 34, 35, or 36 and any sub-range there between, including, but not limited to 10-15 base pairs, 10-14 base pairs, 10-13 base pairs, 10-12 base pairs, 10-11 base pairs, 15-30 base pairs, 15-26 base pairs, 15-23 base pairs, 15-22 base pairs, 15-21 base pairs, 15-20 base pairs, 15-19 base pairs, 15-18 base pairs, 15-17 base pairs, 18-30 base pairs, 18-26 base pairs, 18-23 base pairs, 18-22 base pairs, 18-21 base pairs, 18-20 base pairs, 19-30 base pairs, 19-26 base pairs, 19-23 base pairs, 19-22 base pairs, 19-21 base pairs, 19-20 base pairs, 20-30 base pairs, 20-26 base pairs, 20-25 base pairs, 20-24 base pairs, 20-23 base pairs, 20-22 base pairs, 20-21 base pairs, 21-30 base pairs, 21-26 base pairs, 21-25 base pairs, 21-24 base pairs, 21-23 base pairs, or 21-22 base pairs. Double-stranded oligonucleotides, e.g., dsRNAs, generated in the cell by processing with Dicer and similar enzymes are generally in the range of 19-22 base pairs in length. One strand, antisense strand, of the duplex region of a double-stranded oligonucleotide comprises a sequence that is substantially complementary to a region of a target RNA. The two strands forming the duplex structure can be from a single oligonucleotide molecule having at least one self-complementary region, or can be formed from two or more separate oligonucleotide molecules. Where the duplex region is formed from two complementary regions of a single molecule, the molecule can have a duplex region separated by a single stranded chain of nucleotides (herein referred to as a “hairpin loop”) between the 3′-end of one strand and the 5′-end of the respective other strand forming the duplex structure. The hairpin loop can comprise at least one unpaired nucleotide; in some embodiments the hairpin loop can comprise at least 2, at least 3, at least 4, at least 5, at least 6, at least 7, at least 8, at least 9, at least 10, at least 20, at least 23 or more unpaired nucleotides. In some embodiments, the hairpin loop comprises 3, 4, 5, 6, or 7 Where the two substantially complementary strands of a double-stranded oligonucleotide are comprised by separate molecules, those molecules need not, but can be covalently connected. Where the two strands are connected covalently by means other than a hairpin loop, the connecting structure is referred to as a “linker.” The term “siRNA effector molecule” is also used herein to refer to a dsRNA as described above.

In some embodiments, the RNA effector molecule is a promoter-directed RNA (pdRNA) which is substantially complementary to at least a portion of a noncoding region of an mRNA transcript of a target gene. In one embodiment, the pdRNA is substantially complementary to at least a portion of the promoter region of a target gene mRNA at a site located upstream from the transcription start site, e.g., more than 100, more than 200, or more than 1,000 bases upstream from the transcription start site. In another embodiment, the pdRNA is substantially complementary to at least a portion of the 3′-UTR of a target gene mRNA transcript. In one embodiment, the pdRNA comprises dsRNA of 18-28 bases optionally having 3′ di- or tri-nucleotide overhangs on each strand. The dsRNA is substantially complementary to at least a portion of the promoter region or the 3′-UTR region of a target gene mRNA transcript. In another embodiment, the pdRNA comprises a gapmer consisting of a single stranded polynucleotide comprising a DNA sequence which is substantially complementary to at least a portion of the promoter or the 3′-UTR of a target gene mRNA transcript, and flanking the polynucleotide sequences (e.g., comprising the 5 terminal bases at each of the 5′ and 3′ ends of the gapmer) comprising one or more modified nucleotides, such as 2′ MOE, 2′OMe, or Locked Nucleic Acid bases (LNA), which protect the gapmer from cellular nucleases.

pdRNA can be used to selectively increase, decrease, or otherwise modulate expression of a target gene. Without being limited to a particular theory, it is believed that pdRNAs modulate expression of target genes by binding to endogenous antisense RNA transcripts which overlap with noncoding regions of a target gene mRNA transcript, and recruiting Argonaute proteins (in the case of dsRNA) or host cell nucleases (e.g., RNase H) (in the case of gapmers) to selectively degrade the endogenous antisense RNAs. In some embodiments, the endogenous antisense RNA negatively regulates expression of the target gene and the pdRNA effector molecule activates expression of the target gene. Thus, in some embodiments, pdRNAs can be used to selectively activate the expression of a target gene by inhibiting the negative regulation of target gene expression by endogenous antisense RNA. Methods for identifying antisense transcripts encoded by promoter sequences of target genes and for making and using promoter-directed RNAs are described, e.g., in International Publication No. WO 2009/046397, herein incorporated by reference in its entirety.

Expressed interfering RNA (eiRNA) can be used to selectively increase, decrease, or otherwise modulate expression of a target gene. Typically, eiRNA, (e.g., expressed dsRNA) is expressed in the first transfected cell from an expression vector. In such a vector, the sense strand and the antisense strand of the dsRNA may be transcribed from the same nucleic acid sequence using e.g., two convergent promoters at either end of the nucleic acid sequence or separate promoters transcribing either a sense or antisense sequence. Alternatively, two plasmids can be cotransfected, with one of the plasmids designed to transcribe one strand of the dsRNA while the other is designed to transcribe the other strand. Methods for making and using eiRNA effector molecules are described, for example, in International Publication No. WO 2006/033756, and in U.S. Pat. Pub. Nos. 2005/0239728 and 2006/0035344, which are incorporated by reference in their entirety.

In some embodiments, the RNA effector molecule comprises a small single-stranded Piwi-interacting RNA (piRNA effector molecule) which is substantially complementary to at least a portion of a target gene, as defined herein, and which selectively binds to proteins of the Piwi or Aubergine subclasses of Argonaute proteins. Without being limited to a particular theory, it is believed that piRNA effector molecules interact with RNA transcripts of target genes and recruit Piwi and/or Aubergine proteins to form a ribonucleoprotein (RNP) complex that induces transcriptional and/or post-transcriptional gene silencing of target genes. A piRNA effector molecule can be about 25-50 nucleotides in length, about 25-39 nucleotides in length, or about 26-31 nucleotides in length. Methods for making and using piRNA effector molecules are described, e.g., in U.S. Pat. Pub. No. 2009/0062228, herein incorporated by reference in its entirety.

In some embodiments, the RNA effector molecule is an siRNA or shRNA effector molecule introduced into an animal host cell by contacting the cell with an invasive bacterium containing one or more siRNA or shRNA effector molecules or DNA encoding one or more siRNA or shRNA effector molecules (a process sometimes referred to as transkingdom RNAi (tkRNAi)). The invasive bacterium can be an attenuated strain of a bacterium selected from the group consisting of Listeria, Shigella, Salmonella, E. coli, and Bifidobacteriae, or a non-invasive bacterium that has been genetically modified to increase its invasive properties, e.g., by introducing one or more genes that enable invasive bacteria to access the cytoplasm of host cells. Examples of such cytoplasm-targeting genes include listeriolysin O of Listeria and the invasin protein of Yersinia pseudotuberculosis. Methods for delivering RNA effector molecules to animal cells to induce transkingdom RNAi (tkRNAi) are described, e.g., in U.S. Pat. Pub. Nos. 20080311081 to Fruehauf et al. and 20090123426 to Li et al., both of which are herein incorporated by reference in their entirety. In one embodiment, the RNA effector molecule is an siRNA molecule. In one embodiment, the RNA effector molecule is not an shRNA molecule.

In some embodiments, the RNA effector molecule comprises a microRNA (miRNA). MicroRNAs are a highly conserved class of small RNA molecules that are transcribed from DNA in the genomes of plants and animals, but are not translated into protein. Pre-microRNAs are processed into miRNAs. Processed microRNAs are single stranded ˜17-25 nucleotide (nt) RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. MicroRNAs cause post-transcriptional silencing of specific target genes, e.g., by inhibiting translation or initiating degradation of the targeted mRNA. In some embodiments, the miRNA is completely complementary with the target nucleic acid. In other embodiments, the miRNA has a region of noncomplementarity with the target nucleic acid, resulting in a “bulge” at the region of non-complementarity. In some embodiments, the region of noncomplementarity (the bulge) is flanked by regions of sufficient complementarity, e.g., complete complementarity, to allow duplex formation. Preferably, the regions of complementarity are at least 8 to 10 nucleotides long (e.g., 8, 9, or 10 nucleotides long). miRNA can inhibit gene expression by, e.g., repressing translation, such as when the miRNA is not completely complementary to the target nucleic acid, or by causing target RNA degradation, when the miRNA binds its target with perfect or a high degree of complementarity.

In further embodiments, the RNA effector molecule may comprise an oligonucleotide agent which targets an endogenous miRNA or pre-miRNA. For example, the RNA effector may target an endogenous miRNA which negatively regulates expression of a target gene, such that the RNA effector alleviates miRNA-based inhibition of the target gene. The oligonucleotide agent can include naturally occurring nucleobases, sugars, and covalent internucleotide (backbone) linkages and/or oligonucleotides having one or more non-naturally-occurring features that confer desirable properties, such as enhanced cellular uptake, enhanced affinity for the endogenous miRNA target, and/or increased stability in the presence of nucleases. In some embodiments, an oligonucleotide agent designed to bind to a specific endogenous miRNA has substantial complementarity, e.g., at least 70, 80, 90, or 100% complementary, with at least 10, 20, or 25 or more bases of the target miRNA. Exemplary oligonucleotide agents that target miRNAs and pre-miRNAs are described, for example, in U.S. Pat. Pub. Nos.: 20090317907, 20090298174, 20090291907, 20090291906, 20090286969, 20090236225, 20090221685, 20090203893, 20070049547, 20050261218, 20090275729, 20090043082, 20070287179, 20060212950, 20060166910, 20050227934, 20050222067, 20050221490, 20050221293, 20050182005, and 20050059005, contents of all of which are herein incorporated by reference.

An miRNA or pre-miRNA can be 16-100 nucleotides in length, and more preferably from 16-80 nucleotides in length. Mature miRNAs can have a length of 16-30 nucleotides, preferably 21-25 nucleotides, particularly 21, 22, 23, 24, or 25 nucleotides. miRNA precursors can have a length of 70-100 nucleotides and can have a hairpin conformation. In some embodiments, miRNAs are generated in vivo from pre-miRNAs by the enzymes cDicer and Drosha. miRNAs or pre-miRNAs can be synthesized in vivo by a cell-based system or can be chemically synthesized. miRNAs can comprise modifications which impart one or more desired properties, such as improved stability, hybridization thermodynamics with a target nucleic acid, targeting to a particular tissue or cell-type, and/or cell permeability, e.g., by an endocytosis-dependent or -independent mechanism. Modifications can also increase sequence specificity, and consequently decrease off-site targeting.

In some embodiments, the RNA effector molecule comprises a single-stranded oligonucleotide that interacts with and directs the cleavage of RNA transcripts of a target gene. It is particularly preferred that single stranded RNA effector molecules comprise a 5′ modification including one or more phosphate groups or analogs thereof to protect the effector molecule from nuclease degradation.

In some embodiments, the RNA effector molecule comprises an antagomir. Antagomirs are single stranded, double stranded, partially double stranded or hairpin structures that target a microRNA. An antagomir consisting essentially of or comprises at least 12 or more contiguous nucleotides substantially complementary to an endogenous miRNA and more particularly a target sequence of an miRNA or pre-miRNA nucleotide sequence. Antagomirs preferably have a nucleotide sequence sufficiently complementary to a miRNA target sequence of about 12 to 25 nucleotides, preferably about 15 to 23 nucleotides, to allow the antagomir to hybridize to the target sequence. More preferably, the target sequence differs by no more than 1, 2, or 3 nucleotides from the sequence of the antagomir. In some embodiments, the antagomir includes a non-nucleotide moiety, e.g., a cholesterol moiety, which can be attached, e.g., to the 3′ or 5′ end of the oligonucleotide agent.

In some embodiments, antagomirs are stabilized against nucleolytic degradation by the incorporation of a modification, e.g., a nucleotide modification. For example, in some embodiments, antagomirs contain a phosphorothioate comprising at least the first, second, and/or third internucleotide linkages at the 5′ or 3′ end of the nucleotide sequence. In further embodiments, antagomirs include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In some preferred embodiments, antagomirs include at least one 2′-O-methyl-modified nucleotide.

In some embodiments, the RNA effector molecule comprises an aptamer which binds to a non-nucleic acid ligand, such as a small organic molecule or protein, e.g., a transcription or translation factor, and subsequently inhibits activity. An aptamer can fold into a specific structure that directs the recognition of a targeted binding site on the non-nucleic acid ligand. Aptamers can contain any of the modifications described herein.

In some embodiments, the RNA effector molecule is a single-stranded “antisense” nucleic acid having a nucleotide sequence that is complementary to at least a portion of a “sense” nucleic acid of a target gene, e.g., the coding strand of a double-stranded cDNA molecule or an RNA sequence, e.g., a pre-mRNA, mRNA, miRNA, or pre-miRNA. Accordingly, an antisense nucleic acid can form hydrogen bonds with a sense nucleic acid target. In an alternative embodiment, the RNA effector molecule comprises a duplex region of at least 9 nucleotides in length.

Given a coding strand sequence (e.g., the sequence of a sense strand of a cDNA molecule), antisense nucleic acids can be designed according to the rules of Watson and Crick base pairing. The antisense nucleic acid can be complementary to a portion of the coding or noncoding region of an RNA, e.g., the region surrounding the translation start site of a pre-mRNA or mRNA, e.g., the 5′ UTR. An antisense oligonucleotide can be, for example, about 10 to 25 nucleotides in length (e.g., 11, 12, 13, 14, 15, 16, 18, 19, 20, 21, 22, 23, or 24 nucleotides in length). In some embodiments, the antisense oligonucleotide comprises one or more modified nucleotides, e.g., phosphorothioate derivatives and/or acridine substituted nucleotides, designed to increase the biological stability of the molecule and/or the physical stability of the duplexes formed between the antisense and target nucleic acids. Antisense oligonucleotides can comprise ribonucleotides only, deoxyribonucleotides only (e.g., oligodeoxynucleotides), or both deoxyribonucleotides and ribonucleotides. For example, an antisense agent consisting only of ribonucleotides can hybridize to a complementary RNA and prevent access of the translation machinery to the target RNA transcript, thereby preventing protein synthesis. An antisense molecule including only deoxyribonucleotides, or deoxyribonucleotides and ribonucleotides, can hybridize to a complementary RNA and the RNA target can be subsequently cleaved by an enzyme, e.g., RNAse H, to prevent translation. The flanking RNA sequences can include 2′-O-methylated nucleotides, and phosphorothioate linkages, and the internal DNA sequence can include phosphorothioate internucleotide linkages. The internal DNA sequence is preferably at least five nucleotides in length when targeting by RNAseH activity is desired.

The skilled artisan will recognize that the term “oligonucleotide” or “nucleic acid molecule” encompasses not only nucleic acid molecules as expressed or found in nature, but also analogs and derivatives of nucleic acids comprising one or more ribo- or deoxyribo-nucleotide/nucleoside analogs or derivatives as described herein or as known in the art. Strictly speaking, a “nucleoside” includes a nucleoside base and a ribose or a 2′-deoxyribose sugar, and a “nucleotide” is a nucleoside with one, two or three phosphate moieties. However, the terms “nucleoside” and “nucleotide” can be considered to be equivalent as used herein. An oligonucleotide can be modified in the nucleobase structure or in the ribose-phosphate backbone structure, e.g., as described herein below. However, the molecules comprising nucleoside analogs or derivatives must retain the ability to form a duplex. As non-limiting examples, an oligonucleotide can also include at least one modified nucleoside including but not limited to a 2′-O-methyl modified nucleoside, a nucleoside comprising a 5′ phosphorothioate group, a terminal nucleoside linked to a cholesterol derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, morpholino nucleoside, a phosphoramidate or a non-natural base comprising nucleoside, or any combination thereof. Alternatively, an oligonucleotide can comprise at least two modified nucleosides, at least 3, at least 4, at least 5, at least 6, at least 7, at least 8, at least 9, at least 10, at least 15, at least 20 or more, up to the entire length of the oligonucleotide. The modifications need not be the same for each of such a plurality of modified nucleosides in an oligonucleotide. When RNA effector molecule is double stranded, each strand can be independently modified as to number, type and/or location of the modified nucleosides. In one embodiment, modified oligonucleotides contemplated for use in methods and compositions described herein are peptide nucleic acids (PNAs) that have the ability to form the required duplex structure and that permit or mediate the specific degradation of a target RNA via a RISC pathway.

A double-stranded oligonucleotide can include one or more single-stranded nucleotide overhangs. As used herein, the term “nucleotide overhang” refers to at least one unpaired nucleotide that protrudes from the duplex structure of a double-stranded oligonucleotide, e.g., a dsRNA. For example, when a 3′-end of one strand of double-stranded oligonucleotide extends beyond the 5′-end of the other strand, or vice versa, there is a nucleotide overhang. A double-stranded oligonucleotide can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog. The overhang(s) can be on the sense strand, the antisense strand or any combination thereof. Furthermore, the nucleotide(s) of an overhang can be present on the 5′ end, 3′ end or both ends of either an antisense or sense strand of a dsRNA.

In one embodiment, the antisense strand of a double-stranded oligonucleotide has a 1-10 nucleotide overhang at the 3′ end and/or the 5′ end. In one embodiment, the sense strand of a double-stranded oligonucleotide has a 1-10 nucleotide overhang at the 3′ end and/or the 5′ end. In another embodiment, one or more of the internucleoside linkages in the overhang is replaced with a phosphorothioate. In some embodiments, the overhang comprises one or more deoxyribonucleoside. In some embodiments, overhang comprises the sequence 5′-dTdT-3. In some embodiments, overhang comprises the sequence 5′-dT*dT-3, wherein * is a phosphorothioate internucleoside linkage.

The terms “blunt” or “blunt ended” as used herein in reference to double-stranded oligonucleotide mean that there are no unpaired nucleotides or nucleotide analogs at a given terminal end of a double-stranded oligonucleotide, i.e., no nucleotide overhang. One or both ends of a double-stranded oligonucleotide can be blunt. Where both ends are blunt, the oligonucleotide is said to be double-blunt ended. To be clear, a “double-blunt ended” oligonucleotide is a double-stranded oligonucleotide that is blunt at both ends, i.e., no nucleotide overhang at either end of the molecule. Most often such a molecule will be double-stranded over its entire length. When only one end of is blunt, the oligonucleotide is said to be single-blunt ended. To be clear, a “single-blunt ended” oligonucleotide is a double-stranded oligonucleotide that is blunt at only one end, i.e., no nucleotide overhang at one end of the molecule. Generally, a single-blunt ended oligonucleotide is blunt ended at the 5′-end of sense stand.

The term “antisense strand” or “guide strand” refers to the strand of an RNA effector molecule, e.g., a dsRNA, which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′ and/or 3′ terminus

The term “sense strand,” or “passenger strand” as used herein, refers to the strand of an RNA effector molecule that includes a region that is substantially complementary to a region of the antisense strand as that term is defined herein.

### Plurality of RNA Effector Molecules

In one embodiment, a plurality of different RNA effector molecules are contacted with the cell culture and permit modulation of one or more target genes (e.g., a gene involved in protein glycosylation). In one embodiment, the RNA effector molecules are contacted with the cell culture during production of the polypeptide.

In some embodiments, RNA effector compositions comprise two or more RNA effector molecules, e.g., two, three, four or more RNA effector molecules. In various embodiments, the two or more RNA effector molecules are capable of modulating expression of the same target gene and/or one or more additional target genes. Advantageously, certain compositions comprising multiple RNA effector molecules are more effective in modifying the glycosylation pattern of a polypeptide, or one or more aspects of such production, than separate compositions comprising the individual RNA effector molecules. In some embodiments, the plurality of RNA effector molecules are selected from those provided in Tables 2-24 herein.

In one embodiment, when a plurality of different RNA effector molecules are used to modulate expression of one or more target genes the plurality of RNA effector molecules are contacted with the culture simultaneously or separately. In addition, each RNA effector molecule can have its own dosage regime. For example, in one embodiment one may prepare a composition comprising a plurality of RNA effector molecules that is contacted with a cell. Alternatively, one may administer one RNA effector molecule at a time to the cell culture. In this manner, one can easily tailor the average percent inhibition desired for each target gene by altering the frequency of administration of a particular RNA effector molecule. Contacting a cell with each RNA effector molecule separately can also prevent interactions between RNA effector molecules that can reduce efficiency of target gene modulation. For ease of use and to prevent potential contamination it may be preferred to administer a cocktail of different RNA effector molecules, thereby reducing the number of doses required and minimizing the chance of introducing a contaminant to the cell culture.

dsRNA Effector Molecules

In some embodiments, RNA effector molecule is a double-stranded oligonucleotide comprising a sense strand and an antisense strand, wherein the antisense strand has a region of complementarity to at least part of a target gene RNA. The sense strand includes a region that is complementary to the antisense strand, such that the two strands hybridize and form a duplex structure when combined under suitable conditions. Typically, region of complementarity is 30 nucleotides or less in length, generally 10-26 nucleotides in length, preferably 18-25 nucleotides in length, and most preferably 19-24 nucleotides in length. Upon contact with a cell expressing the target gene, the RNA effector molecule inhibits the expression of the target gene by at least 10% as assayed by, for example, a PCR or branched DNA (bDNA)-based method, or by a protein-based method, such as by Western blot. Expression of a target gene in cell culture, such as in COS cells, HeLa cells, CHO cells, or the like, can be assayed by measuring target gene mRNA levels, e.g., by bDNA or TaqMan assay, or by measuring protein levels, e.g., by immunofluorescence analysis.

As the ordinarily skilled person will recognize, the targeted region of an RNA targeted for cleavage will most often be part of a larger RNA molecule, often an mRNA molecule. Where relevant, a “part” of an RNA target is a contiguous sequence of an RNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

One of skill in the art will also recognize that the duplex region is a primary functional portion of a double-stranded oligonucleotide, e.g., a duplex region of 9 to 36, e.g., 15-30 base pairs. Thus, in one embodiment, to the extent that it becomes processed to a functional duplex of e.g., 15-30 base pairs that targets a desired RNA for cleavage, an oligonucleotide having a duplex region greater than 30 base pairs is an RNA effector molecule.

The oligonucleotides can be synthesized by standard methods known in the art as further discussed below, e.g., by use of an automated DNA synthesizer, such as are commercially available from, for example, Biosearch, Applied Biosystems, Inc. In one embodiment, a target gene is a human target gene. In specific embodiments, the first sequence is a sense strand of a double-stranded oligonucleotide that includes a sense sequence from one of Tables 2-24, and the second sequence is selected from the group consisting of the antisense sequences of one of Tables 2-24. Alternative RNA effector molecules that target elsewhere in the target sequence provided in Tables 2-24 can readily be determined using the target sequence and the flanking target sequence.

In one aspect, a double-stranded oligonucleotide will include at least two nucleotide sequences selected from the groups of sequences provided in Tables 2-24. In this aspect, one of the two sequences is complementary to the other of the two sequences, with one of the sequences being substantially complementary to a sequence of a target RNA generated in the expression of a target gene. As such, in this aspect, a double-stranded RNA effector molecule will include two oligonucleotides, where one oligonucleotide is described as the sense strand in Tables 2-24, and the second oligonucleotide is described as the antisense strand in Tables 2-24. As described elsewhere herein and as known in the art, the complementary sequences of a double-stranded RNA effector molecule can also be contained as self-complementary regions of a single nucleic acid molecule, as opposed to being on separate oligonucleotides (e.g., shRNA).

The skilled person is well aware that dsRNAs having a duplex structure of between 20 and 23, but specifically 21, base pairs have been hailed as particularly effective in inducing RNA interference (Elbashir et al., EMBO 2001, 20:6877-6888, herein incorporated by reference in its entirety). However, others have found that shorter or longer RNA duplex structures can be effective as well. In the embodiments described above, by virtue of the nature of the oligonucleotide sequences provided in Tables 2-24, dsRNAs described herein can include at least one strand of a length of minimally 21 nt. It can be reasonably expected that shorter duplexes having one of the sequences of Tables 2-24 minus only a few nucleotides on one or both ends may be similarly effective as compared to the dsRNAs described above. Hence, dsRNAs having a partial sequence of at least 15, 16, 17, 18, 19, 20, or more contiguous nucleotides from one of the sequences of Tables 2-24, and differing in their ability to inhibit the expression of a target gene by not more than 5, 10, 15, 20, 25, or 30% inhibition from a dsRNA comprising the full sequence, are contemplated according to the invention.

While a target sequence is generally 15-30 nucleotides in length, there is wide variation in the suitability of particular sequences in this range for directing cleavage of any given target RNA. Various software packages and the guidelines set out herein provide guidance for the identification of optimal target sequences for any given gene target, but an empirical approach can also be taken in which a “window” or “mask” of a given size (as a non-limiting example, 21 nucleotides) is literally or figuratively (including, e.g., in silico) placed on the target RNA sequence to identify sequences in the size range that can serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis and testing of the identified sequences (using assays as described herein or as known in the art) to identify those sequences that perform optimally can identify those RNA sequences that, when targeted with an RNA effector molecule agent, mediate the best inhibition of target gene expression. Thus, while the sequences identified, for example, in Tables 2-24 represent effective target sequences, it is contemplated that further optimization of inhibition efficiency can be achieved by progressively “walking the window” one nucleotide upstream or downstream of the given sequences to identify sequences with equal or better inhibition characteristics.

Further, it is contemplated that for any sequence identified, e.g., in Tables 2-24, further optimization could be achieved by systematically either adding or removing nucleotides to generate longer or shorter sequences and testing those sequences generated by walking a window of the longer or shorter size up or down the target RNA from that point. Again, coupling this approach to generating new candidate targets with testing for effectiveness of RNA effector molecules based on those target sequences in an inhibition assay as known in the art or as described herein can lead to further improvements in the efficiency of inhibition. Further still, such optimized sequences can be adjusted by, e.g., the introduction of modified nucleotides as described herein or as known in the art, addition or changes in overhang, or other modifications as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes, etc.) as an expression inhibitor.

An RNA effector molecule as described herein can contain one or more mismatches to the target sequence. In one embodiment, an RNA effector molecule as described herein contains no more than 3 mismatches. If the antisense strand of the RNA effector molecule contains mismatches to a target sequence, it is preferable that the area of mismatch not be located in the center of the region of complementarity. If the antisense strand of the RNA effector molecule contains mismatches to the target sequence, it is preferable that the mismatch be restricted to be within the last 5 nucleotides from either the 5′ or 3′ end of the region of complementarity. For example, for a 23 nucleotide RNA effector molecule agent RNA strand which is complementary to a region of a target gene, the RNA strand generally does not contain any mismatch within the central 13 nucleotides. The methods described herein or methods known in the art can be used to determine whether an RNA effector molecule containing a mismatch to a target sequence is effective in inhibiting the expression of a target gene. Consideration of the efficacy of RNA effector molecules with mismatches in inhibiting expression of a target gene is important, especially if the particular region of complementarity in a target gene is known to have polymorphic sequence variation within the population.

In yet another embodiment, an oligonucleotide is chemically modified to enhance stability or other beneficial characteristics. Oligonucleotides can be modified to prevent rapid degradation of the oligonucleotides by endo- and exo-nucleases and avoid undesirable off-target effects. The nucleic acids featured in the invention can be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference in its entirety. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) internucleoside linkage modifications, including modification or replacement of the phosphodiester linkages. Specific examples of oligonucleotide compounds useful in this invention include, but are not limited to oligonucleotides containing modified or non-natural internucleoside linkages. Oligonucleotides having modified internucleoside linkages include, among others, those that do not have a phosphorus atom in the internucleoside linkage. For the purposes of this specification, and as sometimes referenced in the art, modified oligonucleotides that do not have a phosphorus atom in their internucleoside linkage(s) can also be considered to be oligonucleosides. In particular embodiments, the modified oligonucleotides will have a phosphorus atom in its internucleoside linkage(s).

Modified internucleoside linkages include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. No. RE39464, each of which is herein incorporated by reference in its entirety.

Modified oligonucleotide internucleoside linkages that do not include a phosphorus atom therein have internucleoside linkages that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, each of which is herein incorporated by reference in its entirety.

In other modified oligonucleotides suitable or contemplated for use in RNA effector molecules, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference in its entirety. Further teaching of PNA compounds can be found, for example, in Nielsen et al., Science, 1991, 254, 1497-1500, herein incorporated by reference in its entirety.

Some embodiments featured in the invention include oligonucleotides with phosphorothioate internucleoside linkages and oligonucleosides with heteroatom internucleoside linkage, and in particular —CH₂—NH—CH₂—, —CH₂—N(CH₃)—O—CH₂— [known as a methylene (methylimino) or MMI], —CH₂—O—N(CH₃)—CH₂—, —CH₂—N(CH₃)—N(CH₃)—CH₂— and —N(CH₃)—CH₂—CH₂— [wherein the native phosphodiester internucleoside linkage is represented as —O—P—O—CH₂—] of the above-referenced U.S. Pat. No. 5,489,677, and the amide backbones of the above-referenced U.S. Pat. No. 5,602,240, both of which are herein incorporated by reference in their entirety. In some embodiments, the oligonucleotides featured herein have morpholino backbone structures of the above-referenced U.S. Pat. No. 5,034,506, herein incorporated by reference in its entirety.

Modified oligonucleotides can also contain one or more substituted sugar moieties. The oligonucleotides featured herein can include one of the following at the 2′ position: H (deoxyribose); OH (ribose); F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙ]ₘCH₃, O(CH₂).ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In some embodiments, oligonucleotides include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples herein below.

Other modifications include 2′-methoxy (2′-OCH₃), 2′-aminopropoxy (2′-OCH₂CH₂CH₂NH₂) and 2′-fluoro (2′-F). Similar modifications can also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotide can also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference in its entirety.

An oligonucleotide can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil, 4-(thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenzyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Modified nucleobases also include natural bases that comprise conjugated moieties, e.g. a ligand described herein.

Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in Int. Appl. No. PCT/US09/038,425, filed Mar. 26, 2009; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compositions featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278, herein incorporated by reference in its entirety) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,457,191; 5,459,255; 5,484,908; 5,502,177; 5,525,711; 5,552,540; 5,587,469; 5,594,121, 5,596,091; 5,614,617; 5,681,941; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference in its entirety, and U.S. Pat. No. 5,750,692, also herein incorporated by reference in its entirety.

The oligonucleotides can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to oligonucleotides has been shown to increase oligonucleotide stability in serum, and to reduce off-target effects (see e.g., Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193, each of which is herein incorporated by reference in its entirety).

Representative U.S. patents that teach the preparation of locked nucleic acid nucleotides include, but are not limited to, the following: U.S. Pat. Nos. 6,268,490; 6,670,461; 6,794,499; 6,998,484; 7,053,207; 7,084,125; and 7,399,845, each of which is herein incorporated by reference in its entirety.

Another modification of the oligonucleotides featured in the invention involves chemically linking to the oligonucleotide one or more ligands, moieties or conjugates that enhance the activity, cellular distribution or cellular uptake of the oligonucleotide. Such moieties include but are not limited to lipid moieties such as a cholesterol moiety (Letsinger et al., Proc. Natl. Acid. Sci. USA, 1989, 86: 6553-6556, herein incorporated by reference in its entirety), cholic acid (Manoharan et al., Biorg. Med. Chem. Let., 1994, 4:1053-1060, herein incorporated by reference in its entirety), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770, each of which is herein incorporated by reference in its entirety), a thiocholesterol (Oberhauser et al., Nucl. Acids Res., 1992, 20:533-538, herein incorporated by reference in its entirety), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54, each of which is herein incorporated by reference in its entirety), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783, each of which is herein incorporated by reference in its entirety), a polyamine or a polyethylene glycol chain (Manoharan et al., Nucleosides & Nucleotides, 1995, 14:969-973, herein incorporated by reference in its entirety), or adamantane acetic acid (Manoharan et al., Tetrahedron Lett., 1995, 36:3651-3654, herein incorporated by reference in its entirety), a palmityl moiety (Mishra et al., Biochim. Biophys. Acta, 1995, 1264:229-237, herein incorporated by reference in its entirety), or an octadecylamine or hexylamino-carbonyloxycholesterol moiety (Crooke et al., J. Pharmacol. Exp. Ther., 1996, 277:923-937, herein incorporated by reference in its entirety).

In one embodiment, a ligand alters the cellular uptake, intracellular targeting or half-life of an RNA effector molecule agent into which it is incorporated. In preferred embodiments a ligand provides an enhanced affinity for a selected target, e.g., molecule, cell or cell type, intracellular compartment, e.g., mitochondria, cytoplasm, peroxisome, lysosome, as, e.g., compared to a composition absent such a ligand. Preferred ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell targeting agent, (e.g., a lectin, glycoprotein, lipid or protein), or an antibody, that binds to a specified cell type such as a CHO cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a CHO cell, or other cell useful in the production of polypeptides. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-κB.

The ligand can be a substance, e.g, a drug, which can increase the uptake of the RNA effector molecule agent into the cell, for example, by disrupting the cell's cytoskeleton, e.g., by disrupting the cell's microtubules, microfilaments, and/or intermediate filaments. The drug can be, for example, taxol, vincristine, vinblastine, cytochalasin, nocodazole, japlakinolide, latrunculin A, phalloidin, swinholide A, indanocine, or myoservin.

One exemplary ligand is a lipid or lipid-based molecule. A lipid or lipid-based ligand can (a) increase resistance to degradation of the conjugate, and/or (b) increase targeting or transport into a target cell or cell membrane. A lipid based ligand can be used to modulate, e.g., binding of the RNA effector molecule composition to a target cell.

In some embodiments, the ligand is a lipid or lipid-based molecule that preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, Naproxen or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid based ligand can be used to modulate, e.g., control the binding of the conjugate to a target tissue. For example, a lipid or lipid-based ligand that binds to HSA more strongly will be less likely to be targeted to the kidney and therefore less likely to be cleared from the embryo. A lipid or lipid-based ligand that binds to HSA less strongly can be used to target the conjugate to the kidney. For example, the lipid based ligand binds HSA, or it binds HSA with a sufficient affinity such that the conjugate will be distributed to a non-kidney tissue but also be reversible. Alternatively, the lipid-based ligand binds HSA weakly or not at all, such that the conjugate will be distributed to the kidney. Other moieties that target to kidney cells can also be used in place of or in addition to the lipid-based ligand.

In another aspect, the ligand is a moiety, e.g., a vitamin, which is taken up by a host cell. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HSA and low density lipoprotein (LDL).

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotides can affect pharmacokinetic distribution of the oligonucleotide, such as by enhancing cellular recognition and uptake. The peptide or peptidomimetic moiety can be about 5-50 amino acids long, e.g., about 5, 10, 15, 20, 25, 30, 35, 40, 45, or 50 amino acids long (see Table 1, for example).

TABLE 1
Exemplary Cell Permeation Peptides

| Cell | | |
|---|---|---|
| Permeation | | |
| Peptide | Amino acid Sequence | Reference |
| Penetratin | RQIKIWFQNRRMKWKK (SEQ ID NO: 1248) | Derossi et al., J. |
| | | Biol. Chem. |
| | | 269: 10444, 1994 |
| Tat fragment | GRKKRRQRRRPPQC (SEQ ID NO: 1249) | Vives et al., J. Biol. |
| (48-60) | | Chem., 272: 16010, |
| | | 1997 |
| Signal | GALFLGWLGAAGSTMGAWSQPKKKRKV | Chaloin et al., |
| Sequence- | (SEQ ID NO: 1250) | Biochem. Biophys. |
| based peptide | | Res. Commun., |
| | | 243: 601, 1998 |
| PVEC | LLIILRRRIRKQAHAHSK | Elmquist et al., |
| | (SEQ ID NO: 1251) | Exp. Cell Res., |
| | | 269: 237, 2001 |
| Transportan | GWTLNSAGYLLKINLKALAALAKKIL | Pooga et al., |
| | (SEQ ID NO: 1252) | FASEB J., 12:67, |
| | | 1998 |
| Amphiphilic | KLALKLALKALKAALKLA | Oehlke et al., Mol. |
| model peptide | (SEQ ID NO: 1253) | Ther., 2: 339, 2000 |
| Arg₉ | RRRRRRRRR (SEQ ID NO: 1254) | Mitchell et al., J. |
| | | Pept. Res., 56: 318, |
| | | 2000 |
| Bacterial | KFFKFFKFFK (SEQ ID NO: 1255) | |
| cell wall | | |
| permeating | | |
| LL-37 | LLGDFFRKSKEKIGKEFKRIVQRIKDFLRNL | |
| | VPRTES (SEQ ID NO: 1256) | |
| Cecropin P1 | SWLSKTAKKLENSAKKRISEGIAIAIQGGPR | |
| | (SEQ ID NO: 1257) | |
| α-defensin | ACYCRIPACIAGERRYGTCIYQGRLWAFCC | |
| | (SEQ ID NO: 1258) | |
| b-defensin | DHYNCVSSGGQCLYSACPIFTKIQGTCYRG | |
| | KAKCCK (SEQ ID NO: 1259) | |
| Bactenecin | RKCRIVVIRVCR (SEQ ID NO: 1260) | |
| PR-39 | RRRPRPPYLPRPRPPPFFPPRLPPRIPPGFPPR | |
| | FPPRFPGKR-NH2 (SEQ ID NO: 1261) | |
| Indolicidin | ILPWKWPWWPWRR-NH2 (SEQ ID NO: | |
| | 1262) | |

A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO: 1263). An RFGF analogue (e.g., amino acid sequence AALLPVLLAAP (SEQ ID NO:1264)) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ (SEQ ID NO:1265)) and the Drosophila Antennapedia protein (RQIKIWFQNRRMKWKK (SEQ ID NO:1266)) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Preferably the peptide or peptidomimetic tethered to a dsRNA agent via an incorporated monomer unit is a cell targeting peptide such as an arginine-glycine-aspartic acid (RGD)-peptide, or RGD mimic A peptide moiety can range in length from about 5 amino acids to about 40 amino acids. The peptide moieties can have a structural modification, such as to increase stability or direct conformational properties. Any of the structural modifications described below can be utilized.

An RGD peptide moiety can be used to target a host cell derived from a tumorous cell e.g., an endothelial tumor cell or a breast cancer tumor cell (Zitzmann et al., Cancer Res., 62:5139-43, 2002). The RGD peptide can be linear or cyclic, and can be modified, e.g., glycosylated or methylated to facilitate targeting to specific tissues. For example, a glycosylated RGD peptide can deliver a RNA effector molecule composition to a cell expressing αᵥβ₃ (Haubner et al., Jour. Nucl. Med., 42:326-336, 2001).

A “cell permeation peptide” is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

Representative U.S. patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; each of which is herein incorporated by reference.

It is not necessary for all positions in a given oligonucleotide to be uniformly modified, and in fact more than one of the aforementioned modifications can be incorporated in a single oligonucleotide or even at a single nucleoside within an oligonucleotide. The present invention also includes oligonucleotides which are chimeric compounds. “Chimeric” oligonucleotides or “chimeras,” in the context of this invention, are oligonucleotides, preferably double-stranded oligonucleotides, which contain two or more chemically distinct regions, each made up of at least one monomer unit, i.e., a nucleotide. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide can serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of RNA effector molecule inhibition of gene expression. Consequently, comparable results can often be obtained with shorter oligonucleotides when chimeric oligonucleotides are used, compared to phosphorothioate deoxydsRNAs hybridizing to the same target region. Cleavage of the RNA target can be routinely detected by gel electrophoresis and, if necessary, associated nucleic acid hybridization techniques known in the art.

In certain instances, the oligonucleotide can be modified by a non-ligand group. A number of non-ligand molecules have been conjugated to oligonucleotides in order to enhance the activity, cellular distribution or cellular uptake of the oligonucleotide, and procedures for performing such conjugations are available in the scientific literature. Such non-ligand moieties have included lipid moieties, such as cholesterol (Kubo, T. et al., Biochem. Biophys. Res. Comm., 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-5-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such oligonucleotide conjugates have been listed above. Typical conjugation protocols involve the synthesis of an oligonucleotide bearing an aminolinker at one or more positions of the sequence. The amino group is then reacted with the molecule being conjugated using appropriate coupling or activating reagents. The conjugation reaction can be performed either with the oligonucleotide still bound to the solid support or following cleavage of the oligonucleotide, in solution phase. Purification of the oligonucleotide conjugate by HPLC typically affords the pure conjugate.

### RNA Activation (RNAa)

In one embodiment, an RNA effector molecule is used herein to activate expression of a polypeptide to be modified in the host cell. An RNA effector molecule can be designed to target the promoter region of the gene that expresses the polypeptide to be modified. Induction of polypeptide expression by targeting promoters induces a potent transcriptional activation of associated genes (see e.g., Li, L C et al., Proc. Natl. Acad. Sci. U.S.A. 103 (46): 17337-42 (2006); Janowski, B A et al., Nat. Chem. Biol. 3 (3): 166-73 (2007); Li, L C et al., Caister Academic Press (2008); Check, E. et al., Nature 448 (7156): 855-8 (2007); Huang V et al., PLoS One 5 (1): e8848 (2010)). RNA activation can be performed in human cells using synthetic dsRNAs termed small activating RNAs (saRNAs) or miRNAs. RNAa can also be used in several mammalian species other than human including non-human primates, mouse and rat, as it appears that RNAa is a general gene regulation mechanism conserved at least in mammals.

In one embodiment, an RNA effector molecule is used to enhance expression of a polypeptide for at least 1 day, at least 2 days, at least 3 days, at least 4 days, at least 5-days, at least 6-days, at least 7 days, at least 8 days, at least 9 days, at least 10 days, at least 11 days, at least 12 days, at least 13 days, at least 14 days, at least 15 days, at least 20 days, at least 25 days, at least 30 days or more. Induction of gene expression using RNAa has been observed to last for over ten days, and without wishing to be bound by theory, may be attributed to epigenetic changes at dsRNA target sites.

In some embodiments, an RNA effector molecule (e.g., RNAa molecule) is used to enhance the expression of e.g., glucocerebrosidase, iduronate 2-sulfatase (e.g., idursulfase), acid alpha glucosidase (e.g., alglucosidase alfa), arylsulfatase B (e.g., galsuflase), alpha galactosidase A (e.g., agalsidase beta), and/or alpha-L-iduronidase (e.g., laronidase). Sequences for the promoter regions for each of these polypeptides to be modified are provided herein (SEQ ID NOs. 1240-1247). One of skill in the art can easily design RNA effector molecules homologous and complementary to the promoter sequence of the desired polypeptide (e.g., SEQ ID NOs. 1240-1247) in order to activate expression of the polypeptide. Exemplary RNAa molecules useful with the methods described herein include, but are not limited to, 18-mers, 19-mers, 20-mers, 21-mers, 22-mers, 23-mers or 24-mers of contiguous base pairs from each of SEQ ID NOs. 1240-1247, wherein U is substituted for T. One of skill in the art can use a “scanning” approach to design RNAa molecules of desired length by beginning at the first nucleotide in the promoter sequence (e.g., the first nucleotide of SEQ ID NO. 1240, SEQ ID NO. 1241, SEQ ID NO. 1242, SEQ ID NO. 1243, SEQ ID NO. 1244, SEQ ID NO. 1245, SEQ ID NO. 1246, or SEQ ID NO. 1247) and counting the desired number of nucleotides along the promoter sequence to produce one possible RNAa molecule. The skilled artisan then moves one nucleotide further into the promoter sequence (e.g., “walks” along the promoter sequence) and again counts the desired number of nucleotides along the promoter sequence to produce a second RNAa molecule. This approach can be used iteratively throughout the sequence to identify various possible RNAa molecules to effect enhanced expression of the target gene.

The following example illustrates the “scanning” or “walking” approach of designing RNAa molecules for use with the methods described herein. For example, in one embodiment, the RNAa molecule is a nucleic acid sequence comprising 20 contiguous nucleic acids (e.g., 20-mer) at positions 1-21, 2-22, 3-23, 4-24, 5-25, 6-26, 7-27, 8-28, 9-29, 10-30, 11-31, 12-32, 13-33, 14-34, 15-35, 16-36, 17-37, 18-38, 19-39, 20-40, 21-41, 22-42, 23-43, 24-44, 25-45, 26-46, 27-47, 28-48, 29-49, 30-50, 31-51, 32-52, 33-53, 34-54, 35-55, 36-56, 37-57, 38-58, 39-59, 40-60, 41-61, 42-62, 43-63, 44-64, 45-65, 46-66, 47-67, 48-68, 49-69, 50-70, 51-71, 52-72, 53-73, 54-74, 55-75, 56-76, 57-77, 58-78, 59-79, 60-80, 61-81, 62-82, 63-83, 64-84, 65-85, 66-86, 67-87, 68-88, 69-89, 70-90, 71-91, 72-92, 73-93, 74-94, 75-95, 76-96, 77-97, 78-98, 79-99, 80-100, 81-101, 82-102, 83-103, 84-104, 85-105, 86-106, 87-107, 88-108, 89-109, 90-110, 91-111, 92-112, 93-113, 94-114, 95-115, 96-116, 97-117, 98-118, 99-119, 100-120, 101-121, 102-122, 103-123, 104-124, 105-125, 106-126, 107-127, 108-128, 109-129, 110-130, 111-131, 112-132, 113-133, 114-134, 115-135, 116-136, 117-137, 118-138, 119-139, 120-140, 121-141, 122-142, 123-143, 124-144, 125-145, 126-146, 127-147, 128-148, 129-149, 130-150, 131-151, 132-152, 133-153, 134-154, 135-155, 136-156, 137-157, 138-158, 139-159, 140-160, 141-161, 142-162, 143-163, 144-164, 145-165, 146-166, 147-167, 148-168, 149-169, 150-170, 151-171, 152-172, 153-173, 154-174, 155-175, 156-176, 157-177, 158-178, 159-179, 160-180, 161-181, 162-182, 163-183, 164-184, 165-185, 166-186, 167-187, 168-188, 169-189, 170-190, 171-191, 172-192, 173-193, 174-194, 175-195, 176-196, 177-197, 178-198, 179-199, 180-200, 181-201, 182-202, 183-203, 184-204, 185-205, 186-206, 187-207, 188-208, 189-209, 190-210, 191-211, 192-212, 193-213, 194-214, 195-215, 196-216, 197-217, 198-218, 199-219, 200-220, 201-221, 202-222, 203-223, 204-224, 205-225, 206-226, 207-227, 208-228, 209-229, 210-230, 211-231, 212-232, 213-233, 214-234, 215-235, 216-236, 217-237, 218-238, 219-239, 220-240, 221-241, 222-242, 223-243, 224-244, 225-245, 226-246, 227-247, 228-248, 229-249, 230-250, 231-251, 232-252, 233-253, 234-254, 235-255, 236-256, 237-257, 238-258, 239-259, 240-260, 241-261, 242-262, 243-263, 244-264, 245-265, 246-266, 247-267, 248-268, 249-269, 250-270, 251-271, 252-272, 253-273, 254-274, 255-275, 256-276, 257-277, 258-278, 259-279, 260-280, 261-281, 262-282, 263-283, 264-284, 265-285, 266-286, 267-287, 268-288, 269-289, 270-290, 271-291, 272-292, 273-293, 274-294, 275-295, 276-296, 277-297, 278-298, 279-299, 280-300, 281-301, 282-302, 283-303, 284-304, 285-305, 286-306, 287-307, 288-308, 289-309, 290-310, 291-311, 292-312, 293-313, 294-314, 295-315, 296-316, 297-317, 298-318, 299-319, 300-320, 301-321, 302-322, 303-323, 304-324, 305-325, 306-326, 307-327, 308-328, 309-329, 310-330, 311-331, 312-332, 313-333, 314-334, 315-335, 316-336, 317-337, 318-338, 319-339, 320-340, 321-341, 322-342, 323-343, 324-344, 325-345, 326-346, 327-347, 328-348, 329-349, or 330-350 of a promoter sequence selected from the group consisting of SEQ ID NOs: 1240-1247, wherein U is substituted for T.

Similarly, one can easily design contiguous base pair 21-mers from a promoter sequence selected from the group consisting of SEQ ID NOs. 1240-1247 (e.g., positions 1-22, 2-23, 3-24 . . . 329-350) or an RNAa molecule comprising contiguous nucleic acid sequences from the desired promoter sequence of any desired length (e.g., 18-mer, 19-mer, 22-mer, 23-mer etc).

In one embodiment, two deoxythymine (dT) molecules are added to one end of an RNAa molecule (e.g., 3′ end). Accordingly, if one desires an RNAa molecule of 21 nucleotides in length (i.e., 21-mer), then 19 contiguous nucleotides from one of the SEQ ID NOs. 1240-1247 are selected using the scanning method illustrated above and the dTdT are added to one end of the RNAa molecule (e.g., 3′ end) to result in a 21 nucleotide molecule in length.

The efficiency of an RNAa molecule for enhancing expression can be assessed by means well known to those of skill in the art, e.g., monitoring expression levels of the desired polypeptide by e.g., RT-PCT, Western Blotting, immunoblotting, etc.

### Delivery of an RNA Effector Molecule to a Host Cell

The delivery of an RNA effector molecule to cells according to methods provided herein can be achieved in a number of different ways. Delivery can be performed directly by administering a composition comprising an RNA effector molecule, e.g. a dsRNA, to the cell culture media. Alternatively, delivery can be performed indirectly by administering one or more vectors that encode and direct the expression of the RNA effector molecule. These alternatives are discussed further below.

### Direct Delivery

RNA effector molecules can be modified by chemical conjugation to lipophilic groups such as cholesterol to enhance cellular uptake and prevent degradation. In an alternative embodiment, RNA effector molecules can be delivered using a drug delivery system such as a nanoparticle, a dendrimer, a polymer, a liposome, or a cationic delivery system. Positively charged cationic delivery systems facilitate binding of an RNA effector molecule (negatively charged oligonucleotide) and also enhance interactions at the negatively charged cell membrane to permit efficient cellular uptake. Cationic lipids, dendrimers, or polymers can either be bound to RNA effector molecules, or induced to form a vesicle or micelle (see e.g., Kim S H., et al (2008) Journal of Controlled Release 129(2):107-116) that encases the RNA effector molecule. Methods for making and using cationic-RNA effector molecule complexes are well within the abilities of those skilled in the art (see e.g., Sorensen, D R., et al (2003) J. Mol. Biol. 327:761-766; Verma, U N., et al (2003) Clin. Cancer Res. 9:1291-1300; Arnold, A S et al (2007) J. Hypertens. 25:197-205, which are incorporated herein by reference in their entirety). Exemplary reagents that facilitate RNA effector molecule uptake into a cell comprising charged lipids are described in e.g., U.S. Ser. No. 61/267,419 (filed Dec. 7, 2009), which is herein incorporated by reference in its entirety.

### Separate and Temporal Administration

Where the RNA effector molecule is a double-stranded molecule, such as a small interfering RNA (siRNA), comprising a sense strand and an antisense strand, the sense strand and antisense strand can be separately and temporally exposed to a cell, cell lysate or cell culture. The phrase “separately and temporally” refers to the introduction of each strand of a double-stranded RNA effector molecule to a cell, cell lysate or cell culture in a single-stranded form, e.g., in the form of a non-annealed mixture of both strands or as separate, i.e., unmixed, preparations of each strand. In some embodiments, there is a time interval between the introduction of each strand which can range from seconds to several minutes to about an hour or more, e.g., 12, 24, 48, 72, 84, 96, or 108 hours or more. Separate and temporal administration can be performed with independently modified or unmodified sense and antisense strands.

It is also contemplated herein that a plurality of RNA effector molecules are administered in a separate and temporal manner. Thus, each of a plurality of RNA effector molecules can be administered at a separate time or at a different frequency interval to achieve the desired average percent inhibition for the target gene. In one embodiment, the RNA effector molecules are added at a concentration from approximately 0.01 nM to 200 nM. In another embodiment, the RNA effector molecules are added at an amount of approximately 50 molecules per cell up to and including 500,000 molecules per cell. In another embodiment, the RNA effector molecules are added at a concentration from about 0.1 fmol/10⁶ cells to about 1 pmol/10⁶ cells.

### Transient Inhibition of a Gene Product

In one embodiment, the RNA effector molecule is delivered to the cell such that expression of the gene product is modulated only transiently, e.g., by addition of an RNA effector molecule composition to the cell culture medium used for the production of the polypeptide where the presence of the RNA effector molecules dissipates over time, i.e., the RNA effector molecule is not constitutively expressed in the cell. This can be achieved by altering the timing between delivery of discrete doses of the RNA effector molecule to e.g., the cell culture medium. One of skill in the art can choose an appropriate dosing regime that permits (1) transient inhibition of the gene product, (2) constitutive inhibition of the gene product, or (3) maintenance of a partial inhibition of the gene product (e.g., 50% inhibition, 60%, 70%, 80%, 20%, 30%, 40% etc) as desired by determining the level of inhibition using e.g., ELISA assays to test for expression of the gene product.

Vector Encoded dsRNAs

In another aspect, an RNA effector molecule for modulating expression of a target gene can be expressed from transcription units inserted into DNA or RNA vectors (see, e.g., Couture, A, et al., TIG. (1996), 12:5-10; Skillern, A., et al., International PCT Publication No. WO 00/22113, Conrad, International PCT Publication No. WO 00/22114, and Conrad, U.S. Pat. No. 6,054,299). Such vectors are also useful for expressing a polypeptide to be modified in the host cell. Expression can be transient (on the order of hours to weeks) or sustained (weeks to months or longer), depending upon the specific construct used and the target cell type. These transgenes can be introduced as a linear construct, a circular plasmid, or a viral vector, which can be an integrating or non-integrating vector. The transgene can also be constructed to permit it to be inherited as an extra chromosomal plasmid (Gassmann, et al., Proc. Natl. Acad. Sci. USA (1995) 92:1292).

The individual strand or strands of an RNA effector molecule can be transcribed from a promoter on an expression vector. Where two separate strands are to be expressed to generate, for example a dsRNA, two separate expression vectors can be co-introduced (e.g., by transfection or infection) into a target cell. Alternatively each individual strand of a dsRNA can be transcribed by promoters, both of which are located on the same expression plasmid. In one embodiment, a dsRNA is expressed as an inverted repeat joined by a linker polynucleotide sequence such that the dsRNA has a stem and loop structure.

RNA effector molecule expression vectors are generally DNA plasmids or viral vectors. Expression vectors compatible with eukaryotic cells, preferably those compatible with vertebrate cells, can be used to produce recombinant constructs for the expression of an RNA effector molecule as described herein. Eukaryotic cell expression vectors are well known in the art and are available from a number of commercial sources. Typically, such vectors are provided containing convenient restriction sites for insertion of the desired nucleic acid segment. RNA effector molecule expressing vectors can be delivered directly to target cells using standard transfection and transduction methods.

RNA effector molecule expression plasmids can be transfected into target cells as a complex with cationic lipid carriers (e.g., Oligofectamine) or non-cationic lipid-based carriers (e.g., Transit-TKO™, Minis Bio LLC, Madison, Wis.). Multiple lipid transfections for RNA effector molecule-mediated knockdowns targeting different regions of a target RNA over a period of a week or more are also contemplated by the invention. Successful introduction of vectors into host cells can be monitored using various known methods. For example, transient transfection can be signaled with a reporter, such as a fluorescent marker, such as Green Fluorescent Protein (GFP). Stable transfection of cells ex vivo can be ensured using markers that provide the transfected cell with resistance to specific environmental factors (e.g., antibiotics and drugs), such as hygromycin B resistance.

Viral vector systems which can be utilized to express an RNA effector molecule and/or a polypeptide to be modified include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g EPV and EBV vectors. Constructs for the recombinant expression of an RNA effector molecule will generally require regulatory elements, e.g., promoters, enhancers, etc., to ensure the expression of the RNA effector molecule in target cells. Other aspects to consider for vectors and constructs are further described below.

Vectors useful for the delivery of an RNA effector molecule and/or a polypeptide to be modified will include regulatory elements (promoter, enhancer, etc.) sufficient for expression of the RNA effector molecule or polypeptide in the desired target cell. The regulatory elements can be chosen to provide either constitutive or regulated/inducible expression.

Expression of the RNA effector molecule and/or polypeptide can be precisely regulated, for example, by using an inducible regulatory sequence that is sensitive to certain physiological regulators, e.g., glucose levels (Docherty et al., 1994, FASEB J. 8:20-24). Such inducible expression systems, suitable for the control of dsRNA expression in cells include, for example, regulation by ecdysone, estrogen, progesterone, doxycycline, tetracycline, chemical inducers of dimerization, and isopropyl-beta-D1-thiogalactopyranoside (IPTG). A person skilled in the art would be able to choose the appropriate regulatory/promoter sequence based on the intended use of the transgene.

In a specific embodiment, viral vectors that contain nucleic acid sequences encoding an RNA effector molecule or polypeptide to be modified can be used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding an RNA effector molecule are cloned into one or more vectors, which facilitates delivery of the nucleic acid into a patient. More detail about retroviral vectors can be found, for example, in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993). Lentiviral vectors contemplated for use include, for example, the HIV based vectors described in U.S. Pat. Nos. 6,143,520; 5,665,557; and 5,981,276, each of which is herein incorporated by reference in its entirety.

Adenoviruses are also contemplated for use in delivery of RNA effector molecules and/or polypeptides to be modified. A suitable AV vector for expressing an RNA effector molecule featured in the invention, a method for constructing the recombinant AV vector, and a method for delivering the vector into target cells, are described in Xia H et al. (2002), Nat. Biotech. 20: 1006-1010.

Use of Adeno-associated virus (AAV) vectors is also contemplated (Walsh et al., Proc. Soc. Exp. Biol. Med. 204:289-300 (1993); U.S. Pat. No. 5,436,146, herein incorporated by reference in its entirety). In one embodiment, the RNA effector molecule can be expressed as two separate, complementary single-stranded RNA molecules from a recombinant AAV vector having, for example, either the U6 or H1 RNA promoters, or the cytomegalovirus (CMV) promoter. Suitable AAV vectors for expressing the dsRNA featured in the invention, methods for constructing the recombinant AV vector, and methods for delivering the vectors into target cells are described in Samulski R et al. (1987), J. Virol. 61: 3096-3101; Fisher K J et al. (1996), J. Virol, 70: 520-532; Samulski R et al. (1989), J. Virol. 63: 3822-3826; U.S. Pat. No. 5,252,479; U.S. Pat. No. 5,139,941; International Patent Application No. WO 94/13788; and International Patent Application No. WO 93/24641, the entire disclosures of which are herein incorporated by reference.

Another preferred viral vector is a pox virus such as a vaccinia virus, for example an attenuated vaccinia such as Modified Virus Ankara (MVA) or NYVAC, an avipox such as fowl pox or canary pox.

The tropism of viral vectors can be modified by pseudotyping the vectors with envelope proteins or other surface antigens from other viruses, or by substituting different viral capsid proteins, as appropriate. For example, lentiviral vectors can be pseudotyped with surface proteins from vesicular stomatitis virus (VSV), rabies, Ebola, Mokola, and the like. AAV vectors can be made to target different cells by engineering the vectors to express different capsid protein serotypes; see, e.g., Rabinowitz J E et al. (2002), J Virol 76:791-801, the entire disclosure of which is herein incorporated by reference.

The pharmaceutical preparation of a vector can include the vector in an acceptable diluent, or can include a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

### Administration to Cells

Compositions described herein can be administered to cells in culture in a variety of methods known to those of skill in the art.

In one embodiment, the composition is administered to the cell using continuous infusion of at least one RNA effector molecule into a culture medium used for maintaining the cell to produce the polypeptide. In one embodiment, the continuous infusion is administered at a rate to achieve a desired average percent inhibition for the at least one target gene. In another embodiment, the addition of the RNA effector molecule is repeated throughout the production of the polypeptide. In another embodiment, addition of the RNA effector molecule is repeated at a frequency selected from the group consisting of: 6 h, 12 h, 24 h, 36 h, 48 h, 72 h, 84 h, 96 h, and 108 h. Alternatively, in one embodiment, the addition of the RNA effector molecule is repeated at least three times.

An appropriate concentration of an RNA effector molecule composition useful to achieve the methods described herein can be determined by one of skill in the art. In one embodiment, the at least one RNA effector molecule is added at a concentration selected from the group consisting of 0.1 nM, 0.5 nM, 0.75 nM, 1 nM, 2 nM, 5 nM, 10 nM, 20 nM, 30 nM, 40 nM, 50 nM, and 60 nM.

In another embodiment, the at least RNA effector molecule is added at an amount of 50 molecules per cell, 100 molecules per cell, 200 molecules per cell, 300 molecules per cell, 400 molecules per cell, 500 molecules per cell, 600 molecules per cell, 700 molecules per cell, 800 molecules per cell, 900 molecules per cell, 1000 molecules per cell, 2000 molecules per cell, or 5000 molecules per cell.

In another embodiment, the at least one RNA effector molecule is added at a concentration selected from the group consisting of: 0.01 fmol/10⁶ cells, 0.1 fmol/10⁶ cells, 0.5 fmol/10⁶ cells, 0.75 fmol/10⁶ cells, 1 fmol/10⁶ cells, 2 fmol/10⁶ cells, 5 fmol/10⁶ cells, 10 fmol/10⁶ cells, 20 fmol/10⁶ cells, 30 fmol/10⁶ cells, 40 fmol/10⁶ cells, 50 fmol/10⁶ cells, 60 fmol/10⁶ cells, 100 fmol/10⁶ cells, 200 fmol/10⁶ cells, 300 fmol/10⁶ cells, 400 fmol/10⁶ cells, 500 fmol/10⁶ cells, 700 fmol/10⁶ cells, 800 fmol/10⁶ cells, 900 fmol/10⁶ cells, and 1 pmol/10⁶ cells.

### Detecting Glycosylation Patterns of a Polypeptide

In mammalian cells, glycans are added to N-linked glycosylation sites as high mannose structures in the endoplasmic reticulum. Glycosidases in the ER and Golgi complex trim these structures back to Man₅GlcNAc₂ prior to the addition of N-acetylglucosamine (GlcNAc), galactose and sialic acid to form complex carbohydrate structures. The methods and compositions herein permit modification of a polypeptide expressed in a mammalian cell to contain a terminal mannose.

The glycosylation pattern of a polypeptide can be determined using any method known to those of skill in the art (see e.g., Glycoproteins (1995) J. Montreuil, H. Schacter and JFG Vliegenthart (eds) Elsevier Science, pg 13-28). For example, electrospray-mass spectrometry (ES-MS) can be used to determine post-translational modifications of a polypeptide including compounds larger than 100 kDa. ES-MS can be further combined with capillary electrophoresis as a separation technique with ES-MS as a detector. For glycoproteins with a molecular mass up to 20 kDa, NMR spectroscopy can be used to analyze post-translational modifications. In addition, the application of gradient-enhanced natural abundance ¹H-¹³C HSQC and HSQC-TOCSY spectroscopy has been shown to be effective for the assignment of the NMR resonances of the carbohydrate chains of an intact glycoprotein.

Fractionation of partial structures can be achieved using e.g., gel permeation chromatography for size fractionation, lectin affinity chromatography, HPLC on anion exchange materials, high pH anion exchange chromatography (HPAEC) and high performance capillary electrophoresis.

To define the structure of glycans completely, several parameters are required: (i) type and number of constituent monosaccharides, including absolute configuration, ring size and anomeric configuration, (ii) monosaccharide sequence (including positions of glycosidic linkages), and (iii) type, number and location of non-carbohydrate substituents. Analysis of monosaccharide composition can be achieved by subjecting samples to methonolysis followed by re-N-Acetylation, trimethylsilylation and GLC or by HPAEC-PAD. Linkage analysis is carried out on partially methylated monosaccharide alditols obtained by permethylation of the sample. Exoglycosides can be employed to gain information on the non-reducing-end monosaccharides with regard to identity and absolute and anomeric configuration. Sequential enzymic degradation with exoglycosidases (e.g., mannosidases) can provide insight into structure, however exoglycosidases are not very specific as to ring size, linkage position, and branching point. Alternatively endoglycosidases can provide additional information.

For oligosaccharides and glycopeptides, advanced mass spectrometric techniques including e.g., FAB-MS, ES-MS, MALD-MS, MALD-TOF-MS and MS-MS can provide structural information as to branching pattern, number and length of branches and sequence. A significant advantage of mass spectrometry is that only low amounts of material are required for analysis.

High resolution ¹H-NMR spectroscopy at 500 or 600 MHz is a powerful method for the identification of N- and O-type carbohydrate chains. Other methods that can be employed include 2-dimensional NMR spectra such as e.g., COSY, HOHAHA, NOESY, HMQC and HMBC.

In one embodiment, oligosaccharides are released using PNGase F, labeled with a fluorescent tag and analyzed by high-performance liquid chromatography-mass spectrometry as described in e.g., Van Patten et al., Glycobiology 17(5):467-478 (2007).

### Determining Binding to Mannose Receptor

In one embodiment provided herein, the modified polypeptide binds to a mannose receptor. Such binding can be determined by a number of methods including e.g., in vitro receptor binding assays known in the art or as described in e.g., Van Patten et al., Glycobiology 17(5):467-478 (2007).

Alternatively, the polypeptide can be evaluated for its ability to be taken up by macrophages using a mannose receptor-mediated uptake mechanism. For example, the NR8383 rat alveolar macrophage cell line, which exhibits reproducible mannose receptor-mediated uptake of mannosylated proteins, can be used to determine the presence of an exposed terminal mannose on a modified polypeptide. Briefly, cells are treated with varying doses of a candidate polypeptide for e.g., 2 h, then washed and lysed. The activity of the polypeptide in the cellular lysate is determined and compared to cells incubated in the absence of the candidate polypeptide.

In vivo methods for determining mannose receptor binding include the use of animal models e.g., mouse models. For example, uptake of a modified glucocerebrosidase into macrophages can be determined using the D409V Gaucher mouse model (see e.g., Xu Y H., et al., Am J Pathol 163:2093-2101). This model is viable; however tissue macrophages still accumulate glucosylceramide over time. Fluorescently labeled polypeptide (e.g., glucocerebrosidase) is administered to D409V mice and macrophages are isolated by FACS analysis to determine the amount of cells that have internalized the peptide (see e.g., Van Patten et al., Glycobiology 17(5):467-478 (2007)).

### Compositions Containing RNA Effector Molecule

In one embodiment, the invention provides compositions containing an RNA effector molecule, as described herein, and an acceptable carrier. In one embodiment, the acceptable carrier is a “reagent that facilitates RNA effector molecule uptake” as that term is used herein. The composition containing the RNA effector molecule is useful for modifying the glycosylation pattern of a polypeptide produced in a host cell. Such compositions are formulated based on the mode of delivery. Provided herein are exemplary RNA effector molecules useful in modifying the glycosylation pattern of an expressed polypeptide. In another embodiment, the methods described herein further comprise treating a cell with a composition that inhibits the mannose 6 phosphate receptor to prevent lysosomal uptake of the produced polypeptide. In one embodiment, the RNA effector molecule is an siRNA. In another embodiment, the RNA effector molecule is not an shRNA.

In another embodiment, a composition is provided herein comprising an RNA effector molecule that inhibits the gene expression of e.g., Mgat1, Mgat4, SLC35A1, SLC35A2, and GNE (e.g., Tables 2-24). This composition can optionally be combined (or administered) with at least one additional RNA effector molecule targeting a gene selected from the group consisting of Mgat1, Mgat4, SLC35A1, SLC35A2, and GNE, such as those provided herein in Tables 2-24. The compositions can also be optionally combined or administered with an agent that enhances production of the polypeptide (see e.g., U.S. Provisional No. 61/293,980).

In one embodiment, the composition further comprises a reagent that facilitates RNA effector uptake into a cell, such as an emulsion, a liposome, a cationic lipid, a non-cationic lipid, an anionic lipid, a charged lipid, a penetration enhancer or alternatively, a modification to the RNA effector molecule to attach e.g., a ligand, peptide, lipophillic group, or targeting moiety.

In one embodiment, the compositions described herein comprise a plurality of RNA effector molecules. In one embodiment of this aspect, each of the plurality of RNA effector molecules is provided at a different concentration. In another embodiment of this aspect, each of the plurality of RNA effector molecules is provided at the same concentration. In another embodiment of this aspect, at least two of the plurality of RNA effector molecules are provided at the same concentration, while at least one other RNA effector molecule in the plurality is provided at a different concentration. It is appreciated by one of skill in the art that a variety of combinations of RNA effector molecules and concentrations can be provided to a cell in culture to produce the desired effects described herein.

The compositions featured herein are administered in amounts sufficient to inhibit expression of target genes. In general, a suitable dose of RNA effector molecule will be in the range of 0.001 to 200.0 milligrams per unit volume or cell density per day. In another embodiment, the RNA effector molecule is provided in the range of 0.001 nM to 200 mM per day, generally in the range of 0.1 nM to 500 nM. For example, the dsRNA can be administered at 0.01 nM, 0.05 nM, 0.1 nM, 0.5 nM, 0.75 nM, 1 nM, 1.5 nM, 2 nM, 3 nM, 10 nM, 20 nM, 30 nM, 40 nM, 50 nM, 100 nM, 200 nM, 400 nM, or 500 nM per single dose.

The composition can be administered once daily, or the RNA effector molecule can be administered as two, three, or more sub-doses at appropriate intervals throughout the day or even using continuous infusion or delivery through a controlled release formulation. In that case, the RNA effector molecule contained in each sub dose must be correspondingly smaller in order to achieve the total daily dosage. The dosage unit can also be compounded for delivery over several days, e.g., using a conventional sustained release formulation, which provides sustained release of the RNA effector molecule e.g., over a several day period. Sustained release formulations are well known in the art and are particularly useful for delivery of agents to a cell culture, such as could be used with the compositions of the present invention. In one embodiment, an RNA effector molecule is contacted with the cells in culture at a final concentration of 1 nM. It should be noted that when administering a plurality of RNA effector molecules that one should consider that the total dose of RNA effector molecules will be higher than when each is administered alone. For example, administration of three RNA effector molecules each at 1 nM (e.g., for effective inhibition of target gene expression) will necessarily result in a total dose of 3 nM to the cell culture. One of skill in the art can modify the necessary amount of each RNA effector molecule to produce effective inhibition of each target gene while preventing any unwanted toxic effects to the cell culture resulting from high concentrations of either the RNA effector molecules or delivery agent.

The effect of a single dose on target gene transcript levels can be long-lasting, such that subsequent doses are administered at not more than 3, 4, or 5 day intervals, or at not more than 1, 2, 3, or 4 week intervals.

It is also noted that, in certain embodiments, it can be beneficial to contact the cells in culture with an RNA effector molecule such that a constant number (or at least a minimum number) of RNA effector molecules per each cell is maintained. Maintaining the levels of the RNA effector molecule as such can ensure that modulation of target gene expression is maintained even at high cell densities.

Alternatively, the amount of an RNA effector molecule can be administered according to the cell density. In such embodiments, the RNA effector molecule(s) is added at a concentration of at least 0.01 fmol/10⁶ cells, at least 0.1 fmol/10⁶ cells, at least 0.5 fmol/10⁶ cells, at least 0.75 fmol/10⁶ cells, at least 1 fmol/10⁶ cells, at least 2 fmol/10⁶ cells, at least 5 fmol/10⁶ cells, at least 10 fmol/10⁶ cells, at least 20 fmol/10⁶ cells, at least 30 fmol/10⁶ cells, at least 40 fmol/10⁶ cells, at least 50 fmol/10⁶ cells, at least 60 fmol/10⁶ cells, at least 100 fmol/10⁶ cells, at least 200 fmol/10⁶ cells, at least 300 fmol/10⁶ cells, at least 400 fmol/10⁶ cells, at least 500 fmol/10⁶ cells, at least 700 fmol/10⁶ cells, at least 800 fmol/10⁶ cells, at least 900 fmol/10⁶ cells, or at least 1 pmol/10⁶ cells, or more.

In an alternate embodiment, the RNA effector molecule is administered at a dose of at least 10 molecules per cell, at least 20 molecules per cell, at least 30 molecules per cell, at least 40 molecules per cell, at least 50 molecules per cell, at least 60 molecules per cell, at least 70 molecules per cell, at least 80 molecules per cell, at least 90 molecules per cell at least 100 molecules per cell, at least 200 molecules per cell, at least 300 molecules per cell, at least 400 molecules per cell, at least 500 molecules per cell, at least 600 molecules per cell, at least 700 molecules per cell, at least 800 molecules per cell, at least 900 molecules per cell, at least 1000 molecules per cell, at least 2000 molecules per cell, at least 5000 molecules per cell or more. In some embodiments, the RNA effector molecule is administered at a dose within the range of 10-100 molecules/cell, 10-90 molecules/cell, 10-80 molecules/cell, 10-70 molecules/cell, 10-60 molecules/cell, 10-50 molecules/cell, 10-40 molecules/cell, 10-30 molecules/cell, 10-20 molecules/cell, 90-100 molecules/cell, 80-100 molecules/cell, 70-100 molecules/cell, 60-100 molecules/cell, 50-100 molecules/cell, 40-100 molecules/cell, 30-100 molecules/cell, 20-100 molecules/cell, 30-60 molecules/cell, 30-50 molecules/cell, 40-50 molecules/cell, 40-60 molecules/cell, or any range therebetween.

In one embodiment of the methods described herein, the RNA effector molecule is provided to the cells in a continuous infusion. The continuous infusion can be initiated at day zero (e.g., the first day of cell culture or day of inoculation with an RNA effector molecule) or can be initiated at any time period during the polypeptide production process. Similarly, the continuous infusion can be stopped at any time point during the polypeptide production process. Thus, the infusion of an RNA effector molecule or composition can be provided and/or removed at a particular phase of cell growth, a window of time in the production process, or at any other desired time point. The continuous infusion can also be provided to achieve an “average percent inhibition” for a target gene, as that term is used herein. In one embodiment, a continuous infusion can be used following an initial bolus administration of an RNA effector molecule to a cell culture. In this embodiment, the continuous infusion maintains the concentration of RNA effector molecule above a minimum level over a desired period of time. The continuous infusion can be delivered at a rate of 0.03-3 pmol/liter of culture/h, for example, at 0.03 pmol/l/h, 0.05 pmol/l/h, 0.08 pmol/l/h, 0.1 pmol/l/h, 0.2 pmol/l/h, 0.3 pmol/l/h, 0.5 pmol/l/h, 1.0 pmol/l/h, 2 pmol/l/h, or 3 pmol/l/h, or any value therebetween. In one embodiment, the RNA effector molecule is administered as a sterile aqueous solution. In another embodiment, the RNA effector molecule is formulated in a cationic or non-cationic lipid formulation. In still another embodiment, the RNA effector molecule is formulated in a cell medium suitable for culturing a host cell (e.g., a serum-free medium). In one embodiment, an initial concentration of RNA effector molecule(s) is supplemented with a continuous infusion of the RNA effector molecule to maintain modulation of expression of a target gene. In another embodiment, the RNA effector molecule is applied to cells in culture at a particular stage of cell growth (e.g., early log phase) in a bolus dosage to achieve a certain concentration (e.g., 1 nM), and provided with a continuous infusion of the RNA effector molecule.

The RNA effector molecule(s) can be administered once daily, or the RNA effector molecule treatment can be repeated (e.g., two, three, or more doses) by adding the composition to the culture medium at appropriate intervals/frequencies throughout the production of the biological product. As used herein the term “frequency” refers to the interval at which transfection or infection of the cell culture occurs and can be optimized by one of skill in the art to maintain the desired level of inhibition for each target gene. In one embodiment, RNA effector molecules are contacted with cells in culture at a frequency of every 48 hours. In other embodiments, the RNA effector molecules are administered at a frequency of e.g., every 4 h, every 6 h, every 12 h, every 18 h, every 24 h, every 36 h, every 72 h, every 84 h, every 96 h, every 5 days, every 7 days, every 10 days, every 14 days, every 3 weeks, or more during the production of the biological product. The frequency can also vary, such that the interval between each dose is different (e.g., first interval 36 h, second interval 48 h, third interval 72 h etc).

The term “frequency” can be similarly applied to nutrient feeding of a cell culture during the production of a polypeptide. The frequency of treatment with RNA effector molecule(s) and nutrient feeding need not be the same. To be clear, nutrients can be added at the time of RNA effector treatment or at an alternate time. The frequency of nutrient feeding can be a shorter interval or a longer interval than RNA effector molecule treatment. As but one example, the dose of RNA effector molecule can be applied at a 48 h interval while nutrient feeding may be applied at a 24 h interval. During the entire length of the interval for producing the biological product (e.g., 3 weeks) there can be more doses of nutrients than RNA effector molecules or less doses of nutrients than RNA effector molecules. Alternatively, the amount (e.g., number) of treatments with RNA effector molecule(s) is equal to that of nutrient feedings.

The frequency of RNA effector molecule treatment can be optimized to maintain an “average percent inhibition” of a particular target gene. As used herein, the term “average percent inhibition” refers to the average degree of inhibition of target gene expression over time that is necessary to produce the desired effect and which is below the degree of inhibition that produces any unwanted or negative effects. In some embodiments, the desired average percent inhibition is at least 20%, at least 30%, at least 40%, at least 50%, at least 60%, at least 70%, at least 80%, at least 85%, at least 90%, at least 95%, at least 99%, or even 100% (i.e., absent). One of skill in the art can use routine cell death assays to determine the upper limit for desired percent inhibition (e.g., level of inhibition that produces unwanted effects). One of skill in the art can also use methods to detect target gene expression (e.g., RT-PCR) to determine an amount of an RNA effector molecule that produces gene modulation. The percent inhibition is described herein as an average value over time, since the amount of inhibition is dynamic and can fluctuate slightly between doses of the RNA effector molecule.

In one embodiment of the methods described herein, the RNA effector molecule is added to the culture medium of the cells in culture. The methods described herein can be applied to any size of cell culture flask and/or bioreactor. For example, the methods can be applied in bioreactors or cell cultures of 1 L, 3 L, 5 L, 10 L, 15 L, 40 L, 100 L, 500 L, 1000 L, 2000 L, 3000 L, 4000 L, 5000 L or larger. In some embodiments, the cell culture size can range from 0.01 L to 5000 L, from 0.1 L to 5000 L, from 1 L to 5000 L, from 5 L to 5000 L, from 40 L to 5000 L, from 100 L-5000 L, from 500 L to 5000 L, from 1000-5000 L, from 2000-5000 L, from 3000-5000 L, from 4000-5000 L, from 4500-5000 L, from 0.01 L to 1000 L, from 0.01-500 L, from 0.01-100 L, from 0.01-40 L, from 15-2000 L, from 40-1000 L, from 100-500 L, from 200-400 L, or any integer therebetween.

The RNA effector molecule(s) can be added during any phase of cell growth including, but not limited to, lag phase, stationary phase, early log phase, mid-log phase, late-log phase, exponential phase, or death phase. It is preferred that the cells are contacted with the RNA effector molecules prior to their entry into the death phase. In some embodiments, it may be desired to contact the cell in an earlier growth phase such as the lag phase, early log phase, mid-log phase or late-log phase. In other embodiments, it may be desired or acceptable to inhibit target gene expression at a later phase in the cell growth cycle (e.g., late-log phase or stationary phase).

RNA effector molecules featured in the invention can be encapsulated within liposomes or can form complexes thereto, in particular to cationic liposomes. Alternatively, RNA effector molecules can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₂₀ alkyl ester (e.g., isopropylmyristate IPM), monoglyceride, diglyceride or acceptable salt thereof.

In one embodiment, an RNA effector molecule featured in the invention is fully encapsulated in the lipid formulation (e.g., to form a SPLP, pSPLP, SNALP, or other nucleic acid-lipid particle). As used herein, the term “SNALP” refers to a stable nucleic acid-lipid particle, including SPLP. As used herein, the term “SPLP” refers to a nucleic acid-lipid particle comprising plasmid DNA encapsulated within a lipid vesicle. SNALPs and SPLPs typically contain a cationic lipid, a non-cationic lipid, and a lipid that prevents aggregation of the particle (e.g., a PEG-lipid conjugate). SPLPs include “pSPLP,” which include an encapsulated condensing agent-nucleic acid complex as set forth in e.g., PCT Publication No. WO 00/03683. The particles in this embodiment typically have a mean diameter of about 50 nm to about 150 nm, more typically about 60 nm to about 130 nm, more typically about 70 nm to about 110 nm, most typically about 70 to about 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; and PCT Publication No. WO 96/40964.

In one embodiment, the lipid to drug ratio (mass/mass ratio) (e.g., lipid to dsRNA ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1.

The cationic lipid of the formulation preferably comprises at least one protonatable group having a pKa of from 4 to 15. The cationic lipid can be, for example, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.C1), 1,2-Dilinoleoyl-3-trimethylaminopropane chloride salt (DLin-TAP.C1), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA), 2,2-Dilinoleyl-4-dimethylaminoethyl-[1,3]-dioxolane, or a mixture thereof. The cationic lipid can comprise from about 20 mol % to about 70 mol % or about 40 mol % to about 60 mol % of the total lipid present in the particle. In one embodiment, cationic lipid can be further conjugated to a ligand.

The non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid may be from about 5 mol % to about 90 mol %, about 10 mol %, or about 58 mol % if cholesterol is included, of the total lipid present in the particle.

The lipid that inhibits aggregation of particles may be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA may be, for example, a PEG-dilauryloxypropyl (Ci₂), a PEG-dimyristyloxypropyl (C₁₄), a PEG-dipalmityloxypropyl (C₁₆), or a PEG-distearyloxypropyl (C₁₈). The lipid that prevents aggregation of particles may be from 0 mol % to about 20 mol % or about 2 mol % of the total lipid present in the particle. In one embodiment, PEG lipid can be further conjugated to a ligand.

In some embodiments, the nucleic acid-lipid particle further includes a steroid such as, cholesterol at, e.g., about 10 mol % to about 60 mol % or about 48 mol % of the total lipid present in the particle.

In one embodiment, the lipid particle comprises a steroid, a PEG lipid and a cationic lipid of formula (I):

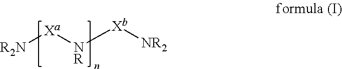

wherein
each X^{a} and X^{b}, for each occurrence, is independently C₁₋₆ alkylene;
n is 0, 1, 2, 3, 4, or 5; each R is independently H,

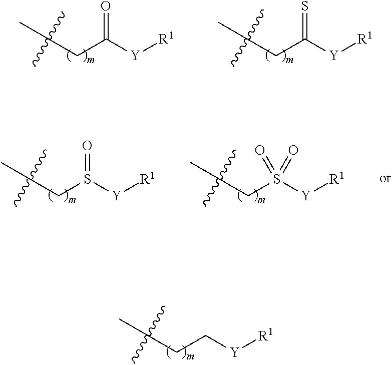

m is 0, 1, 2, 3 or 4; Y is absent, O, NR², or S;
R¹ is alkyl alkenyl or alkynyl; each of which is optionally substituted with one or more substituents; and
R² is H, alkyl alkenyl or alkynyl; each of which is optionally substituted each of which is optionally substituted with one or more substituents. In one example, the lipidoid ND98.4HCl (MW 1487) (Formula 1), Cholesterol (Sigma-Aldrich), and PEG-Ceramide C16 (Avanti Polar Lipids) can be used to prepare lipid RNA effector molecule nanoparticles (e.g., LNP01 particles). Stock solutions of each in ethanol can be prepared as follows: ND98, 133 mg/mL; Cholesterol, 25 mg/mL, PEG-Ceramide C16, 100 mg/mL. The ND98, Cholesterol, and PEG-Ceramide C16 stock solutions can then be combined in a, e.g., 42:48:10 molar ratio. The combined lipid solution can be mixed with aqueous RNA effector molecule (e.g., in sodium acetate pH 5) such that the final ethanol concentration is about 35-45% and the final sodium acetate concentration is about 100-300 mM. Lipid RNA effector molecule nanoparticles typically form spontaneously upon mixing. Depending on the desired particle size distribution, the resultant nanoparticle mixture can be extruded through a polycarbonate membrane (e.g., 100 nm cut-off) using, for example, a thermobarrel extruder, such as Lipex Extruder (Northern Lipids, Inc). In some cases, the extrusion step can be omitted. Ethanol removal and simultaneous buffer exchange can be accomplished by, for example, dialysis or tangential flow filtration. Buffer can be exchanged with, for example, phosphate buffered saline (PBS) at about pH 7, e.g., about pH 6.9, about pH 7.0, about pH 7.1, about pH 7.2, about pH 7.3, or about pH 7.4.

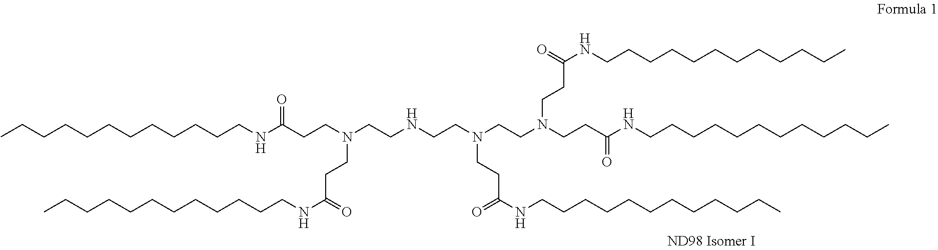 [SMILES: CCCCCCCCCCCCNC(=O)CCN(CCNCCN(CCC(=O)NCCCCCCCCCCCC)CCN(CCC(=O)NCCCCCCCCCCCC)CCC(=O)NCCCCCCCCCCCC)CCC(=O)NCCCCCCCCCCCC]

LNP01 formulations are described, e.g., in International Application Publication No. WO 2008/042973, which is hereby incorporated by reference.

Additional exemplary lipid-dsRNA formulations are as follows:

| | | cationic lipid/non-cationic | |
|---|---|---|---|
| | | lipid/cholesterol/PEG-lipid | |
| | | conjugate | |
| | Cationic Lipid | Lipid:siRNA ratio | Process |
| SNALP | 1,2-Dilinolenyloxy-N,N- | DLinDMA/DPPC/Cholesterol/PEG- | |
| | dimethylaminopropane | cDMA | |
| | (DLinDMA) | (57.1/7.1/34.4/1.4) | |
| | | lipid:siRNA ~ 7:1 | |
| SNALP- | 2,2-Dilinoleyl-4- | XTC/DPPC/Cholesterol/PEG- | |
| XTC | dimethylaminoethyl-[1,3]- | cDMA | |
| | dioxolane (XTC) | 57.1/7.1/34.4/1.4 | |
| | | lipid:siRNA ~ 7:1 | |
| LNP05 | 2,2-Dilinoleyl-4- | XTC/DSPC/Cholesterol/PEG-DMG | Extrusion |
| | dimethylaminoethyl-[1,3]- | 57.5/7.5/31.5/3.5 | |
| | dioxolane (XTC) | lipid:siRNA ~ 6:1 | |
| LNP06 | 2,2-Dilinoleyl-4- | XTC/DSPC/Cholesterol/PEG-DMG | Extrusion |
| | dimethylaminoethyl-[1,3]- | 57.5/7.5/31.5/3.5 | |
| | dioxolane (XTC) | lipid:siRNA ~ 11:1 | |
| LNP07 | 2,2-Dilinoleyl-4- | XTC/DSPC/Cholesterol/PEG-DMG | In-line |
| | dimethylaminoethyl-[1,3]- | 60/7.5/31/1.5, | mixing |
| | dioxolane (XTC) | lipid:siRNA ~ 6:1 | |
| LNP08 | 2,2-Dilinoleyl-4- | XTC/DSPC/Cholesterol/PEG-DMG | In-line |
| | dimethylaminoethyl-[1,3]- | 60/7.5/31/1.5, | mixing |
| | dioxolane (XTC) | lipid:siRNA ~ 11:1 | |
| LNP09 | 2,2-Dilinoleyl-4- | XTC/DSPC/Cholesterol/PEG-DMG | In-line |
| | dimethylaminoethyl-[1,3]- | 50/10/38.5/1.5 | mixing |
| | dioxolane (XTC) | Lipid:siRNA 10:1 | |
| LNP10 | (3aR,5s,6aS)-N,N-dimethyl- | ALN100/DSPC/Cholesterol/PEG- | In-line |
| | 2,2-di((9Z,12Z)-octadeca-9,12- | DMG | mixing |
| | dienyl)tetrahydro-3aH- | 50/10/38.5/1.5 | |
| | cyclopenta[d][1,3]dioxol-5- | Lipid:siRNA 10:1 | |
| | amine (ALN100) | | |
| LNP11 | (6Z,9Z,28Z,31Z)- | MC-3/DSPC/Cholesterol/PEG- | In-line |
| | heptatriaconta-6,9,28,31- | DMG | mixing |
| | tetraen-19-yl 4- | 50/10/38.5/1.5 | |
| | (dimethylamino)butanoate | Lipid:siRNA 10:1 | |
| | (MC3) | | |
| LNP12 | 1,1′-(2-(4-(2-((2-(bis(2- | Tech G1/DSPC/Cholesterol/PEG- | In-line |
| | hydroxydodecyl)amino)ethyl)(2- | DMG | mixing |
| | hydroxydodecyl)amino)ethyl)pi | 50/10/38.5/1.5 | |
| | perazin-1- | Lipid:siRNA 10:1 | |
| | yl)ethylazanediyl)didodecan-2- | | |
| | ol (Tech G1) | | |

LNP09 formulations and XTC comprising formulations are described, e.g., in U.S. Provisional Ser. No. 61/239,686, filed Sep. 3, 2009, which is hereby incorporated by reference. LNP11 formulations and MC3 comprising formulations are described, e.g., in U.S. Provisional Ser. No. 61/244,834, filed Sep. 22, 2009, which is hereby incorporated by reference.

In one embodiment, the lipid particle comprises a charged lipid having the formula:

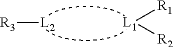

wherein:

R₁ and R₂ are each independently for each occurrence optionally substituted C₁₀-C₃₀ alkyl, optionally substituted C₁₀-C₃₀ alkoxy, optionally substituted C₁₀-C₃₀ alkenyl, optionally substituted C₁₀-C₃₀ alkenyloxy, optionally substituted C₁₀-C₃₀ alkynyl, optionally substituted C₁₀-C₃₀ alkynyloxy, or optionally substituted C₁₀-C₃₀ acyl;

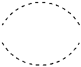

represents a connection between L₂ and L₁ which is:

(1) a single bond between one atom of L₂ and one atom of L₁, wherein
L₁ is C(Rₓ), O, S or N(Q);
L₂ is —CR₅R₆—, —O—, —S—, —N(Q)-,

### ═C(R₅)—, —C(O)N(Q)-, —C(O)O—, —N(Q)C(O)—, —OC(O)—, or —C(O)—;

(2) a double bond between one atom of L₂ and one atom of L₁; wherein
L₁ is C;
L₂ is —CR₅═, —N(Q)═, —N—, —O—N═, —N(O)—N═, or —C(O)N(O)—N═;

(3) a single bond between a first atom of L₂ and a first atom of L₁, and a single bond between a second atom of L₂ and the first atom of L₁, wherein
L₁ is C;
L₂ has the formula

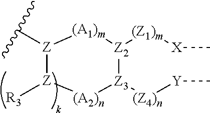

wherein
X is the first atom of L₂, Y is the second atom of L₂, - - - represents a single bond to the first atom of L₁, and X and Y are each, independently, selected from the group consisting of —O—, —S—,
   alkylene, —N(Q)-, —C(O)—, —O(CO)—, —OC(O)N(Q)-, —N(W)C(O)O—, —C(O), —OC(O)O—, —OS(O) (Q₂)O—, and —OP(O)(Q₂)O—;
Z₁ and Z₄ are each, independently, —O—, —S—, —CH₂—, —CHR⁵—, or —CR⁵R⁵—;
Z₂ is CH or N;
Z₃ is CH or N;
or Z₂ and Z₃, taken together, are a single C atom;
A₁ and A₂ are each, independently, —O—, —S—, —CH₂—, —CHR⁵—, or —CR⁵R⁵—;
each Z is N, C(R₅), or C(R₃);
k is 0, 1, or 2;
each m, independently, is 0 to 5;
each n, independently, is 0 to 5;
where m and n taken together result in a 3, 4, 5, 6, 7 or 8 member ring;

(4) a single bond between a first atom of L₂ and a first atom of L₁, and a single bond between the first atom of L₂ and a second atom of L₁, wherein
(A) L₁ has the formula:

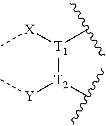

wherein
X is the first atom of L₁, Y is the second atom of L₁, - - - represents a single bond to the first atom of L₂, and X and Y are each, independently, selected from the group consisting of —O—, —S—,
   alkylene, —N(Q)-, —C(O)—, —O(CO)—, —OC(O)N(Q)-, —N(Q)C(O)O—, —C(O)O, —OC(O)O—, —OS(O) (Q₂)O—, and —OP(O)(Q₂)O—;
T₁ is CH or N;
T₂ is CH or N;
or T₁ and T₂ taken together are C═C;
L₂ is CR₅; or
(B) L₁ has the formula:

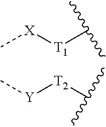

wherein
X is the first atom of L₁, Y is the second atom of L₁, - - - represents a single bond to the first atom of L₂, and X and Y are each, independently, selected from the group consisting of —O—, —S—,
   alkylene, —N(Q)-, —C(O)—, —O(CO)—, —OC(O)N(Q)-, —N(Q)C(O)O—, —C(O)O, —OC(O)O—, —OS(O)(Q₂)O—, and —OP(O)(Q₂)O—;
T₁ is —CR₅R₅—, —N(Q)-, —O—, or —S—;
T₂ is —CR₅R₅—, —N(Q)-, —O—, or —S—;
L₂ is CR₅ or N;

R₃ has the formula:

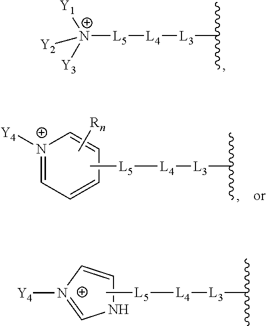

wherein

each of Y₁, Y₂, Y₃, and Y₄, independently, is alkyl, cycloalkyl, aryl, aralkyl, or alkynyl; or

any two of Y₁, Y₂, and Y₃ are taken together with the N atom to which they are attached to form a 3- to 8-member heterocycle; or

Y₁, Y₂, and Y₃ are all be taken together with the N atom to which they are attached to form a bicyclic 5- to 12-member heterocycle;

each Rₙ, independently, is H, halo, cyano, hydroxy, amino, alkyl, alkoxy, cycloalkyl, aryl, heteroaryl, or heterocyclyl;

L₃ is a bond, —N(O)—, —O—, —S—, —(CR₅R₆)ₐ—, —C(O)—, or a combination of any two of these;

L₄ is a bond, —N(O)—, —O—, —S—, —(CR₅R₆)ₐ—, —C(O)—, or a combination of any two of these;

L₅ is a bond, —N(O)—, —O—, —S—, —(CR₅R₆)ₐ—, —C(O)—, or a combination of any two of these;

each occurrence of R₅ and R₆ is, independently, H, halo, cyano, hydroxy, amino, alkyl, alkoxy, cycloalkyl, aryl, heteroaryl, or heterocyclyl; or two R₅ groups on adjacent carbon atoms are taken together to form a double bond between their respective carbon atoms; or two R₅ groups on adjacent carbon atoms and two R₆ groups on the same adjacent carbon atoms are taken together to form a triple bond between their respective carbon atoms;

each a, independently, is 0, 1, 2, or 3;

wherein
an R₅ or R₆ substituent from any of L₃, L₄, or L₅ is optionally taken with an R₅ or R₆ substituent from any of L₃, L₄, or L₅ to form a 3- to 8-member cycloalkyl, heterocyclyl, aryl, or heteroaryl group; and
any one of Y₁, Y₂, or Y₃, is optionally taken together with an R₅ or R₆ group from any of L₃, L₄, and L₅, and atoms to which they are attached, to form a 3- to 8-member heterocyclyl group;

each Q, independently, is H, alkyl, acyl, cycloalkyl, alkenyl, alkynyl, aryl, heteroaryl or heterocyclyl; and

each Q₂, independently, is O, S, N(Q)(Q), alkyl or alkoxy.

In some embodiments,

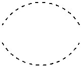

represents a connection between L₂ and L₁ which is a single bond between one atom of L₂ and one atom of L₁, wherein L₁ is C(Rₓ), O, S or N(Q); and L₂ is —CR₅R₆—, —O—, —S—, —N(Q)-, ═C(R₅)—, —C(O)N(Q)-, —C(O)O—, —N(Q)C(O)—, —OC(O)—, or —C(O)—.

In another aspect, a compound having formula I, XIII, XV, XVII, XXXIII, or XXXV:

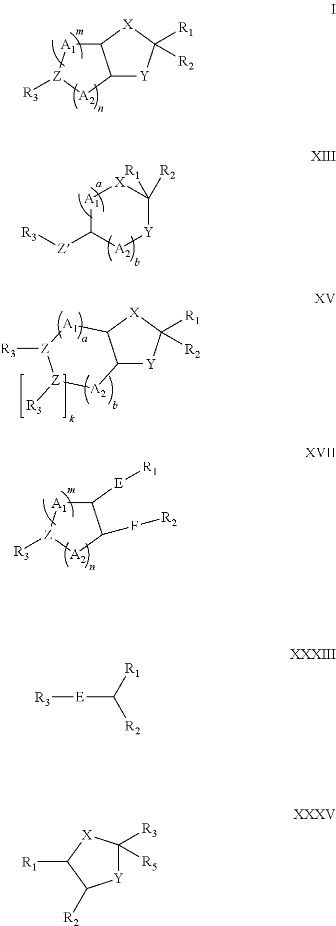

wherein:

R₁ and R₂ are each independently for each occurrence optionally substituted C₁₀-C₃₀ alkyl, optionally substituted C₁₀-C₃₀ alkoxy, optionally substituted C₁₀-C₃₀ alkenyl, optionally substituted C₁₀-C₃₀ alkenyloxy, optionally substituted C₁₀-C₃₀ alkynyl, optionally substituted C₁₀-C₃₀ alkynyloxy, or optionally substituted C₁₀-C₃₀ acyl;

R₃ is independently for each occurrence H, optionally substituted C₁-C₁₀ alkyl, optionally substituted C₂-C₁₀ alkenyl, optionally substituted C₂-C₁₀ alkynyl, optionally substituted alkylheterocycle, optionally substituted heterocyclealkyl, optionally substituted alkylphosphate, optionally substituted phosphoalkyl, optionally substituted alkylphosphorothioate, optionally substituted phosphorothioalkyl, optionally substituted alkylphosphorodithioate, optionally substituted phosphorodithioalkyl, optionally substituted alkylphosphonate, optionally substituted phosphonoalkyl, optionally substituted amino, optionally substituted alkylamino, optionally substituted di(alkyl)amino, optionally substituted aminoalkyl, optionally substituted alkylaminoalkyl, optionally substituted di(alkyl)aminoalkyl, optionally substituted hydroxyalkyl, optionally substituted polyethylene glycol (PEG, mw 100-40K), optionally substituted mPEG (mw 120-40K), optionally substituted heteroaryl, or optionally substituted heterocycle;

at least one R₃ includes a quaternary amine;

X and Y are each independently —O—, —S—,

alkylene, —N(Q)-, —C(O)—, —O(CO)—, —OC(O)N(Q)-, —N(Q)C(O)O—, —C(O)O, —OC(O)O—, —OS(O) (Q₂)O—, or —OP(O)(Q₂)O—;

Q is H, alkyl, ω-aminoalkyl, ω-(substituted)aminoalkyl, ω-phosphoalkyl, or ω-thiophosphoalkyl;

Q₂ is independently for each occurrence O, S, N(Q)(Q), alkyl or alkoxy;

A₁, A₂, A₃, A₄, A₅ and A₆ are each independently —O—, —S—, —CH₂—, —CHR⁵—, —CR⁵R⁵—;

A₈ is independently for each occurrence —CH₂—, —CHR⁵—, —CR⁵R⁵—;

E and F are each independently for each

occurrence —CH₂—, —O—, —S—, —SS—, —CO—, —C(O)O—, —C(O)N(R′)—, —OC(O)N(R′)—, —N(R′)C(O) N(R″)—, —C(O)—N(R′)—N═C(R′″)—; —N(R′)—N═C(R″)—, —O—N═C(R″)—, —C(S)O—, —C(S)N(R′)—, —O C(S)N(R′)—, —N(R′)C(S)N(R″)—, —C(S)—N(R′)—N═C(R′″); —S—N═C(R″); —C(O)S—, —SC(O)N(R′)—, —OC(O)—, —N(R′)C(O)—, —N(R′)C(O)O—, —C(R′″)═N—N(R′)—; —C(R′″)═N—N(R′)—C(O)—, —C(R′″) ═N—O—, —OC(S)—, —SC(O)—, —N(R′)C(S)—, —N(R′)C(S)O—, —N(R′)C(O)S—, —C(R′″)═N—N(R′)—C(S)—, —C(R′″)═N—S—, C[═N(R′)]O, C[═N(R′)]N(R″), —OC[═N(R′)]—, —N(R″)C[═N(R′)]N(R′″)—, —N(R″)C[═N(R′)]—,

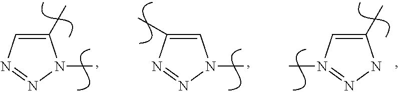

arylene, heteroarylene, cycloalkylene, or heterocyclylene;

Z is N or C(R₃);

Z′ is —O—, —S—, —N(O)—, or alkylene;

each R′, R″, and R′″, independently, is H, alkyl, alkyl, heteroalkyl, aralkyl, cyclic alkyl, or heterocyclyl;

R⁵ is H, halo, cyano, hydroxy, amino, optionally substituted alkyl, optionally substituted alkoxy, or optionally substituted cycloalkyl;

i and j are each independently 0-10; and

a and b are each independently 0-2.

In another aspect, a compound can be selected from the group consisting of:

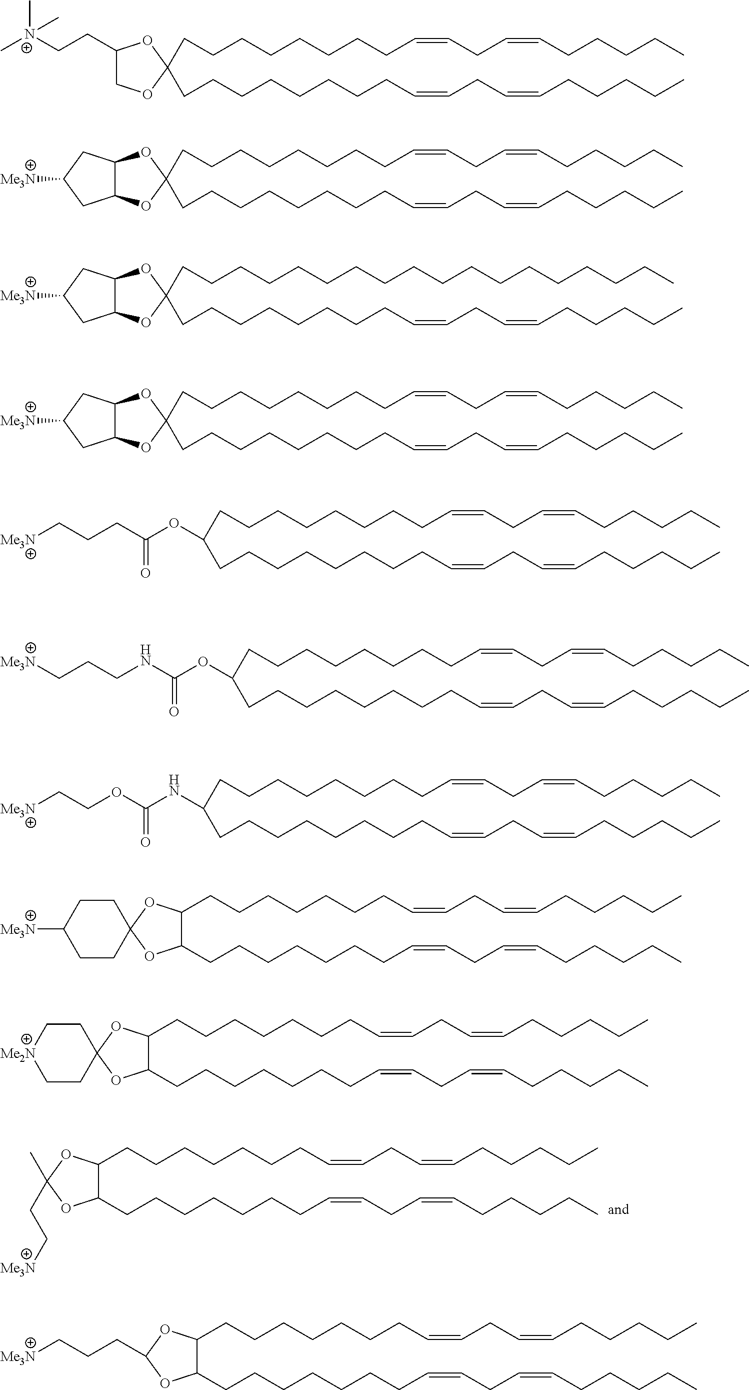

In one embodiment, the lipid particle further comprises a neutral lipid and a sterol. Neutral lipids, when present in the lipid particle, can be any of a number of lipid species which exist either in an uncharged or neutral zwitterionic form at physiological pH. Such lipids include, for example diacylphosphatidylcholine, diacylphosphatidylethanolamine, ceramide, sphingomyelin, dihydrosphingomyelin, cephalin, and cerebrosides. The selection of neutral lipids for use in the particles described herein is generally guided by consideration of, e.g., liposome size and stability of the liposomes in the bloodstream. Preferably, the neutral lipid component is a lipid having two acyl groups, (i.e., diacylphosphatidylcholine and diacylphosphatidylethanolamine) Lipids having a variety of acyl chain groups of varying chain length and degree of saturation are available or may be isolated or synthesized by well-known techniques. In one group of embodiments, lipids containing saturated fatty acids with carbon chain lengths in the range of C₁₀ to C₂₀ are preferred. In another group of embodiments, lipids with mono or diunsaturated fatty acids with carbon chain lengths in the range of C₁₀ to C₂₀ are used. Additionally, lipids having mixtures of saturated and unsaturated fatty acid chains can be used. Preferably, the neutral lipids used in the present invention are DOPE, DSPC, POPC, DPPC or any related phosphatidylcholine. The neutral lipids useful in the present invention may also be composed of sphingomyelin, dihydrosphingomyeline, or phospholipids with other head groups, such as serine and inositol.

The sterol component of the lipid mixture, when present, can be any of those sterols conventionally used in the field of liposome, lipid vesicle or lipid particle preparation. A preferred sterol is cholesterol.

Other protonatable lipids, which carry a net positive charge at about physiological pH, in addition to those specifically described above, may also be included in lipid particles of the present invention. Such protonatable lipids include, but are not limited to, N,N-dioleyl-N,N-dimethylammonium chloride (“DODAC”); N-(2,3-dioleyloxy)propyl-N,N—N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 1,2-Dioleyloxy-3-trimethylaminopropane chloride salt (“DOTAP.C1”); 3β-(N—(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethylammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), N,N-dimethyl-2,3-dioleyloxy)propylamine (“DODMA”), and N-(1,2-dimyristyloxyprop-3-yl)-N,N-dimethyl-N-hydroxyethyl ammonium bromide (“DMRIE”). Additionally, a number of commercial preparations of lipids can be used, such as, e.g., LIPOFECTIN (including DOTMA and DOPE, available from GIBCO/BRL), and LIPOFECTAMINE (comprising DOSPA and DOPE, available from GIBCO/BRL).

Anionic lipids suitable for use in lipid particles of the present invention include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

Additional components that may be present in a lipid particle as described herein include bilayer stabilizing components such as polyamide oligomers (see, e.g., U.S. Pat. No. 6,320,017), peptides, proteins, detergents, lipid-derivatives, such as PEG coupled to phosphatidylethanolamine and PEG conjugated to ceramides (see, U.S. Pat. No. 5,885,613).

The lipid particles described herein may further comprise one or more additional lipids and/or other components such as cholesterol.

As used herein, the term “charged lipid” is meant to include those lipids having one or two fatty acyl or fatty alkyl chains and a quaternary amino head group. The quaternary amine carries a permanent positive charge. The head group can optionally include a ionizable group, such as a primary, secondary, or tertiary amine that may be protonated at physiological pH. The presence of the quaternary amine can alter the pKa of the ionizable group relative to the pKa of the group in a structurally similar compound that lacks the quaternary amine (e.g., the quaternary amine is replaced by a tertiary amine) In some embodiments, a charged lipid is referred to as an “amino lipid.”

Other charged lipids would include those having alternative fatty acid groups and other quaternary groups, including those in which the alkyl substituents are different (e.g., N-ethyl-N-methylamino-, N-propyl-N-ethylamino- and the like). For those embodiments in which R₁ and R₂ are both long chain alkyl or acyl groups, they can be the same or different. In general, lipids (e.g., a charged lipid) having less saturated acyl chains are more easily sized, particularly when the complexes are sized below about 0.3 microns, for purposes of filter sterilization. Charged lipids containing unsaturated fatty acids with carbon chain lengths in the range of C₁₀ to C₂₀ are typical. Other scaffolds can also be used to separate the amino group (e.g., the amino group of the charged lipid) and the fatty acid or fatty alkyl portion of the charged lipid. Suitable scaffolds are known to those of skill in the art.

In certain embodiments, charged lipids of the present invention have at least one protonatable or deprotonatable group, such that the lipid is positively charged at a pH at or below physiological pH (e.g. pH 7.4), and neutral at a second pH, preferably at or above physiological pH. Such lipids are also referred to as charged lipids. It will, of course, be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwiterrionic, are not excluded from use in the invention.

In certain embodiments, protonatable lipids (i.e., charged lipids) according to the invention have a pKa of the protonatable group in the range of about 4 to about 11. Typically, lipids will have a pKa of about 4 to about 7, e.g., between about 5 and 7, such as between about 5.5 and 6.8, when incorporated into lipid particles. Such lipids will be cationic at a lower pH formulation stage, while particles will be largely (though not completely) surface neutralized at physiological pH around pH 7.4. One of the benefits of a pKa in the range of between about 4 and 7 is that at least some nucleic acid associated with the outside surface of the particle will lose its electrostatic interaction at physiological pH and be removed by simple dialysis; thus greatly reducing the particle's susceptibility to clearance. pKa measurements of lipids within lipid particles can be performed, for example, by using the fluorescent probe 2-(p-toluidino)-6-napthalene sulfonic acid (TNS), using methods described in Cullis et al., (1986) Chem Phys Lipids 40, 127-144.

Charged lipids can be prepared for use in transfection by forming into liposomes and mixing with the RNA effector molecules to be introduced into the cell. Methods of forming liposomes are well known in the art and include, but are not limited to, sonication, extrusion, extended vortexing, reverse evaporation, and homogenization, which includes microfluidization.

The reagent that facilitates uptake of an RNA effector molecule into the cell encompasses both single-layered liposomes, which are referred to as unilamellar, and multi-layered liposomes, which are referred to as multilamellar. Lipoplexes are composed of charged lipid bilayers sandwiched between nucleic acid layers, as described, e.g., in Felgner, Scientific American.

LNP01 formulations are described, e.g., in International Application Publication No. WO 2008/042973, which is hereby incorporated by reference in its entirety.

Formulations prepared by either the standard or extrusion-free method can be characterized in similar manners. For example, formulations are typically characterized by visual inspection. They should be whitish translucent solutions free from aggregates or sediment. Particle size and particle size distribution of lipid-nanoparticles can be measured by light scattering using, for example, a Malvern Zetasizer Nano ZS (Malvern, USA). Particles should be about 20-300 nm, such as e.g., 40-100 nm in size. The particle size distribution should be unimodal. The total siRNA effector molecule concentration in the formulation, as well as the entrapped fraction, is estimated using a dye exclusion assay. A sample of the formulated RNA effector molecule can be incubated with an RNA-binding dye, such as Ribogreen (Molecular Probes) in the presence or absence of a formulation disrupting surfactant, e.g., 0.5% Triton-X100. The total RNA effector molecule in the formulation can be determined by the signal from the sample containing the surfactant, relative to a standard curve. The entrapped fraction is determined by subtracting the “free” RNA effector molecule content (as measured by the signal in the absence of surfactant) from the total RNA effector molecule content. Percent entrapped RNA effector molecule is typically >85%. For lipid nanoparticle formulation, the particle size is at least 30 nm, at least 40 nm, at least 50 nm, at least 60 nm, at least 70 nm, at least 80 nm, at least 90 nm, at least 100 nm, at least 110 nm, and at least 120 nm. The suitable range is typically about at least 50 nm to about at least 110 nm, about at least 60 nm to about at least 100 nm, or about at least 80 nm to about at least 90 nm.

In some embodiments, RNA effector molecules featured in the invention are formulated in conjunction with one or more penetration enhancers, surfactants and/or chelators. Suitable surfactants include fatty acids and/or esters or salts thereof, bile acids and/or salts thereof. Suitable bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether.

The compositions of the present invention may be formulated into any of many possible administration forms, including a sustained release form (e.g., tablets, capsules, gel capsules, liquid syrups, and soft gels). The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

### Emulsions

The compositions of the present invention may be prepared and formulated as emulsions. Emulsions are typically heterogeneous systems of one liquid dispersed in another in the form of droplets usually exceeding 0.1 μm in diameter (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions may be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions may contain additional components in addition to the dispersed phases, and the active drug which may be present as a solution in either the aqueous phase, oily phase or itself as a separate phase. Pharmaceutical excipients such as emulsifiers, stabilizers, dyes, and anti-oxidants may also be present in emulsions as needed. Emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y. Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

In one embodiment, the compositions of RNA effector molecules and nucleic acids are formulated as microemulsions. A microemulsion may be defined as a system of water, oil and amphiphile which is a single optically isotropic and thermodynamically stable liquid solution (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (M0310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions afford advantages of improved agent solubilization, protection from enzymatic hydrolysis, possible enhancement of cellular uptake due to surfactant-induced alterations in membrane fluidity and permeability, ease of preparation, and decreased toxicity (see e.g., U.S. Pat. Nos. 6,191,105; 7,063,860; 7,070,802; 7,157,099; Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile compositions, peptides or RNA effector molecules.

Microemulsions of the present invention may also contain additional components and additives such as sorbitan monostearate (Grill 3), Labrasol, and penetration enhancers to improve the properties of the formulation and to enhance the absorption of the RNA effector molecules and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

### Liposomes

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. In some embodiments, it is desirable to use a liposome which is highly deformable and able to pass through fine pores in a cell membrane or between cells grown in culture.

Further advantages of liposomes include: liposomes obtained from natural phospholipids are biocompatible and biodegradable; liposomes can incorporate a wide range of water and lipid soluble drugs; and liposomes can protect encapsulated RNA effector molecules in their internal compartments from metabolism and degradation (see e.g., Wang, B et al., Drug delivery: principles and applications, 2005, John Wiley and Sons, Hoboken, N.J.; Rosoff, in Pharmaceutical Dosage Forms, Lieberman, Rieger and Banker (Eds.), 1988, Marcel Dekker, Inc., New York, N.Y., volume 1, p. 245) in the cell culture medium. Important considerations in the preparation of liposome formulations are the lipid surface charge, vesicle size and the aqueous volume of the liposomes.

Liposomes are useful for the transfer and delivery of active ingredients to the site of action in the cell. Because the liposomal membrane is structurally similar to biological membranes, when liposomes are applied to a cell in culture, the liposomes start to merge with the cellular membranes and as the merging of the liposome and cell progresses, the liposomal contents are emptied into the cell where the RNA effector molecule acts.

Liposomal formulations have been the focus of extensive investigation as the mode of delivery for many compositions. Liposomes fall into two broad classes. Cationic liposomes are positively charged liposomes which interact with the negatively charged polynucleotide molecules to form a stable complex. The positively charged polynucleotide/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun, 1987, 147, 980-985).

Liposomes which are pH-sensitive or negatively-charged, entrap polynucleotide rather than complex with it. Since both the polynucleotide and the lipid are similarly charged, repulsion rather than complex formation occurs. Nevertheless, some polynucleotide is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C₁₂₁₅G, that contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). In addition, antibodies can be conjugated to a polyakylene derivatized liposome (see e.g., PCT Application US 2008/0014255). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al). U.S. Pat. No. 5,540,935 (Miyazaki et al.) and U.S. Pat. No. 5,556,948 (Tagawa et al.) describe PEG-containing liposomes that can be further derivatized with functional moieties on their surfaces. Methods and compositions relating to liposomes comprising PEG can be found in e.g., U.S. Pat. Nos. 6,049,094; 6,224,903; 6,270,806; 6,471,326; and 6,958,241.

As noted above, liposomes may optionally be prepared to contain surface groups, such as antibodies or antibody fragments, small effector molecules for interacting with cell-surface receptors, antigens, and other like compounds, and these groups can facilitate delivery of liposomes and their contents to specific cell populations. Such ligands can be included in the liposomes by including in the liposomal lipids a lipid derivatized with the targeting molecule, or a lipid having a polar-head chemical group that can be derivatized with the targeting molecule in preformed liposomes. Alternatively, a targeting moiety can be inserted into preformed liposomes by incubating the preformed liposomes with a ligand-polymer-lipid conjugate.

Also suitable for inclusion in the lipid particles of the present invention are programmable fusion lipids. Such lipid particles have little tendency to fuse with cell membranes and deliver their payload until a given signal event occurs. This allows the lipid particle to distribute more evenly after injection into an organism or disease site before it starts fusing with cells. The signal event can be, for example, a change in pH, temperature, ionic environment, or time. In the latter case, a fusion delaying or “cloaking” component, such as an ATTA-lipid conjugate or a PEG-lipid conjugate, can simply exchange out of the lipid particle membrane over time. By the time the lipid particle is suitably distributed in the body, it has lost sufficient cloaking agent so as to be fusogenic. With other signal events, it is desirable to choose a signal that is associated with the disease site or target cell, such as increased temperature at a site of inflammation.

In certain embodiments, it is desirable to target the lipid particles of this invention using targeting moieties that are specific to a cell type or tissue. Targeting of lipid particles using a variety of targeting moieties, such as ligands, cell surface receptors, glycoproteins, vitamins (e.g., riboflavin) and monoclonal antibodies, have been previously described (see, e.g., U.S. Pat. Nos. 4,957,773 and 4,603,044). The targeting moieties can comprise the entire protein or fragments thereof. Targeting mechanisms generally require that the targeting agents be positioned on the surface of the lipid particle in such a manner that the target moiety is available for interaction with the target, for example, a cell surface receptor. A variety of different targeting agents and methods are known and available in the art, including those described, e.g., in Sapra, P. and Allen, T M, Prog. Lipid Res. 42(5):439-62 (2003); and Abra, R M et al., J Liposome Res. 12:1-3, (2002).

The use of lipid particles, i.e., liposomes, with a surface coating of hydrophilic polymer chains, such as polyethylene glycol (PEG) chains, for targeting has been proposed (Allen, et al., Biochimica et Biophysica Acta 1237: 99-108 (1995); DeFrees, et al., Journal of the American Chemistry Society 118: 6101-6104 (1996); Blume, et al., Biochimica et Biophysica Acta 1149: 180-184 (1993); Klibanov, et al., Journal of Liposome Research 2: 321-334 (1992); U.S. Pat. No. 5,013,556; Zalipsky, Bioconjugate Chemistry 4: 296-299 (1993); Zalipsky, FEBS Letters 353: 71-74 (1994); Zalipsky, in Stealth Liposomes Chapter 9 (Lasic and Martin, Eds) CRC Press, Boca Raton Fla. (1995). In one approach, a ligand, such as an antibody, for targeting the lipid particle is linked to the polar head group of lipids forming the lipid particle. In another approach, the targeting ligand is attached to the distal ends of the PEG chains forming the hydrophilic polymer coating (Klibanov, et al., Journal of Liposome Research 2: 321-334 (1992); Kirpotin et al., FEBS Letters 388: 115-118 (1996)).

Standard methods for coupling the target agents can be used. For example, phosphatidylethanolamine, which can be activated for attachment of target agents, or derivatized lipophilic compounds, such as lipid-derivatized bleomycin, can be used. Antibody-targeted liposomes can be constructed using, for instance, liposomes that incorporate protein A (see, Renneisen, et al., J. Bio. Chem., 265:16337-16342 (1990) and Leonetti, et al., Proc. Natl. Acad. Sci. (USA), 87:2448-2451 (1990). Other examples of antibody conjugation are disclosed in U.S. Pat. No. 6,027,726, the teachings of which are incorporated herein by reference. Examples of targeting moieties can also include other proteins, specific to cellular components, including antigens associated with neoplasms or tumors. Proteins used as targeting moieties can be attached to the liposomes via covalent bonds (see, Heath, Covalent Attachment of Proteins to Liposomes, 149 Methods in Enzymology 111-119 (Academic Press, Inc. 1987)). Other targeting methods include the biotin-avidin system.

In one exemplary embodiment, the lipid particle comprises a mixture of a charged lipid of the present invention, one or more different neutral lipids, and a sterol (e.g., cholesterol). In certain embodiments, the lipid mixture consists of or consists essentially of a charged lipid as described herein, a neutral lipid, and cholesterol. In further preferred embodiments, the lipid particle consists of or consists essentially of the above lipid mixture in molar ratios of about 50-90% charged lipid, 0-50% neutral lipid, and 0-10% cholesterol. In certain embodiments, the lipid particle can further include a PEG-modified lipid (e.g., a PEG-DMG or PEG-DMA).

In one embodiment, the lipid particle consists of a charged lipid (e.g., a quaternary nitrogen containing lipid) and a protonatable lipid, a neutral lipid or a steroid, or a combination thereof. The particles can be formulated with a nucleic acid therapeutic agent so as to attain a desired N/P ratio. The N/P ratio is the ratio of number of molar equivalent of cationic nitrogen (N) atoms present in the lipid particle to the number of molar equivalent of anionic phosphate (P) of the nucleic acid backbone. For example, the N/P ratio can be in the range of about 1 to about 50. In one example, the range is about 1 to about 20, about 1 to about 10, about 1 to about 5.

In particular embodiments, the lipid particle consists of or consists essentially of a charged lipid described in paragraph [00246] herein, DOPE, and cholesterol. In particular embodiments, the particle includes lipids in the following mole percentages: charged lipid, 45-63 mol %; DOPE, 35-55 mol %; and cholesterol, 0-10 mol %. The particles can be formulated with a nucleic acid therapeutic agent so as to attain a desired N/P ratio. The N/P ratio is the ratio of number of moles cationic nitrogen (N) atoms (i.e., charged lipids) to the number of molar equivalents of anionic phosphate (P) backbone groups of the nucleic acid. For example, the N to P ratio can be in the range of about 5:1 to about 1:1. In certain embodiments, the charged lipid is chosen from those described in paragraph [00215] herein.

In another group of embodiments, the neutral lipid, DOPE, in these compositions is replaced with POPC, DPPC, DPSC or SM.

A number of liposomes comprising nucleic acids are known in the art. WO 96/40062 (Thierry et al.) discloses methods for encapsulating high molecular weight nucleic acids in liposomes. U.S. Pat. No. 5,264,221 (Tagawa et al.) discloses protein-bonded liposomes and asserts that the contents of such liposomes may include a dsRNA. U.S. Pat. No. 5,665,710 (Rahman et al.) describes certain methods of encapsulating oligodeoxynucleotides in liposomes. WO 97/04787 (Love et al.) discloses liposomes comprising dsRNAs targeted to the raf gene. In addition, methods for preparing a liposome composition comprising a nucleic acid can be found in e.g., U.S. Pat. Nos. 6,011,020; 6,074,667; 6,110,490; 6,147,204; 6, 271, 206; 6,312,956; 6,465,188; 6,506,564; 6,750,016; and 7,112,337.

Transfersomes are yet another type of liposome, and are highly deformable lipid aggregates which are attractive candidates for RNA delivery vehicles. Transfersomes may be described as lipid droplets which are so highly deformable that they are easily able to penetrate through pores which are smaller than the droplet. Transfersomes are adaptable to the environment in which they are used, e.g., they are self-optimizing, self-repairing, frequently reach their targets without fragmenting, and often self-loading. To make transfersomes it is possible to add surface edge-activators, usually surfactants, to a standard liposomal composition.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (see e.g., Malmsten, M. Surfactants and polymers in drug delivery, Informa Health Care, New York, N.Y., 2002; Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (see e.g., Malmsten, M. Surfactants and polymers in drug delivery, Informa Health Care, New York, N.Y., 2002; Rieger, in Pharmaceutical Dosage Forms, Marcel Dekker, Inc., New York, N.Y., 1988, p. 285).

### Penetration Enhancers

In one embodiment, the present invention employs various penetration enhancers to effect the efficient delivery of nucleic acids, particularly RNA effector molecules, to the cell in culture. Typically, only lipid soluble or lipophilic compositions readily cross cell membranes. It has been discovered that even non-lipophilic compositions may cross cell membranes if the membrane to be crossed is treated with a penetration enhancer. In addition to aiding the diffusion of non-lipophilic compositions across cell membranes, penetration enhancers also enhance the permeability of lipophilic compositions.

Agents that enhance uptake of RNA effector molecules at the cellular level may also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of dsRNAs. Examples of commercially available transfection reagents include, for example Lipofectamine™ (Invitrogen; Carlsbad, Calif.), Lipofectamine 2000™ (Invitrogen; Carlsbad, Calif.), 293Fectin™ (Invitrogen; Carlsbad, Calif.), Cellfectin™ (Invitrogen; Carlsbad, Calif.), DMRIE-CTM (Invitrogen; Carlsbad, Calif.), FreeStyle™ MAX (Invitrogen; Carlsbad, Calif.), Lipofectamine™ 2000 CD (Invitrogen; Carlsbad, Calif.), Lipofectamine™ (Invitrogen; Carlsbad, Calif.), RNAiMAX (Invitrogen; Carlsbad, Calif.), Oligofectamine™ (Invitrogen; Carlsbad, Calif.), Optifect™ (Invitrogen; Carlsbad, Calif.), X-tremeGENE Q2 Transfection Reagent (Roche; Grenzacherstrasse, Switzerland), DOTAP Liposomal Transfection Reagent (Grenzacherstrasse, Switzerland), DOSPER Liposomal Transfection Reagent (Grenzacherstrasse, Switzerland), or Fugene (Grenzacherstrasse, Switzerland), Transfectam® Reagent (Promega; Madison, Wis.), TransFast™ Transfection Reagent (Promega; Madison, Wis.), Tfx™-20 Reagent (Promega; Madison, Wis.), Tfx™-50 Reagent (Promega; Madison, Wis.), DreamFect™ (OZ Biosciences; Marseille, France), EcoTransfect (OZ Biosciences; Marseille, France), TransPassa D1 Transfection Reagent (New England Biolabs; Ipswich, Mass., USA), LyoVec™/LipoGen™ (Invitrogen; San Diego, Calif., USA), PerFectin Transfection Reagent (Genlantis; San Diego, Calif., USA), NeuroPORTER Transfection Reagent (Genlantis; San Diego, Calif., USA), GenePORTER Transfection reagent (Genlantis; San Diego, Calif., USA), GenePORTER 2 Transfection reagent (Genlantis; San Diego, Calif., USA), Cytofectin Transfection Reagent (Genlantis; San Diego, Calif., USA), BaculoPORTER Transfection Reagent (Genlantis; San Diego, Calif., USA), TroganPORTERT™ transfection Reagent (Genlantis; San Diego, Calif., USA), RiboFect (Bioline; Taunton, Mass., USA), PlasFect (Bioline; Taunton, Mass., USA), UniFECTOR (B-Bridge International; Mountain View, Calif., USA), SureFECTOR (B-Bridge International; Mountain View, Calif., USA), or HiFect™ (B-Bridge International, Mountain View, Calif., USA), among others.

Other agents may be utilized to enhance the penetration of the administered nucleic acids, including glycols such as ethylene glycol and propylene glycol, pyrrols such as 2-pyrrol, azones, and terpenes such as limonene and menthone.

### Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal.

### Other Components

The compositions of the present invention may additionally contain other adjunct components so long as such materials, when added, do not unduly interfere with the biological activities of the components of the compositions of the present invention. The formulations can be sterilized and, if desired, mixed with auxiliary agents which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

Toxicity and therapeutic efficacy of such compounds can be determined by standard cell based assays cell cultures, e.g., cell death assays for determining the level of toxicity or evaluating an LD50 (the dose lethal to 50% of the cells in the population) and the ED50 (the dose therapeutically effective in 50% of the cellular population). The dose ratio between toxic and therapeutic effects is the therapeutic index and it can be expressed as the ratio LD50/ED50. Compounds that exhibit high therapeutic indices are preferred as they are less likely to induce cell toxicity during the production of a modified polypeptide.

The data obtained from cell culture assays can be used in formulating a range of dosages for use in the instant methods. The dosage of compositions featured in the invention lies generally within a range of concentrations that includes the ED50 with little or no toxicity. The dosage may vary within this range depending upon the dosage form employed and the route of administration utilized.

### Methods for Inhibiting Expression of a Gene Product

In yet another aspect, the invention provides a method for inhibiting the expression of a gene product in a host cell. The method includes administering a composition featured in the invention to the host cell such that expression of the gene product is decreased, such as for an extended duration, e.g., at least two, three, four days or more, e.g., one week, two weeks, three weeks, or four weeks or longer. The effect of the decreased expression of the target gene preferably results in a decrease in levels of the protein encoded by the target gene by at least 10%, at least 15%, at least 20%, at least 25%, at least 30%, at least 40%, at least 50%, or at least 60%, or more, as compared to pretreatment levels.

Preferably, the RNA effector molecules useful for the methods and compositions featured in the invention specifically target RNAs (primary or processed) of the target gene. Compositions and methods for inhibiting the expression of these target genes using RNA effector molecules can be prepared and performed as described elsewhere herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the RNA effector molecules and methods featured in the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Kits for Producing a Polypeptide with a Terminal Mannose

In some embodiments, kits are provided for testing the effect of an RNA effector molecule or a series of RNA effector molecules on the production of a polypeptide having a modified glycosylation pattern by a host cell, where the kits comprise a substrate having one or more assay surfaces suitable for culturing host cells under conditions that allow production of the polypeptide. In some embodiments, the exterior of the substrate comprises wells, indentations, demarcations, or the like at positions corresponding to the assay surfaces. In some preferred embodiments, the wells, indentations, demarcations, or the like retain fluid, such as cell culture media, over the assay surfaces.

In some embodiments, the assay surfaces on the substrate are sterile and are suitable for culturing host cells under conditions representative of the cell culture conditions during large-scale (e.g., industrial scale) production of the polypeptide. Advantageously, kits provided herein offer a rapid, cost-effective means for testing a wide-range of agents and/or conditions on the modification of a polypeptide glycosylation pattern, allowing the cell culture conditions to be established prior to full-scale production of the polypeptide.

In some embodiments, one or more assay surfaces of the substrate comprise a concentrated test agent, such as an RNA effector molecule, such that the addition of suitable media to the assay surfaces results in a desired concentration of the RNA effector molecule surrounding the assay surface. In some embodiments, the RNA effector molecules may be printed or ingrained onto the assay surface, or provided in a lyophilized form, e.g., within wells, such that the effector molecules can be reconstituted upon addition of an appropriate amount of media. In some embodiments, the RNA effector molecules are reconstituted by plating cells onto assay surfaces of the substrate.

In some embodiments, kits provided herein further comprise cell culture media suitable for culturing a host cell under conditions allowing for modification of a polypeptide glycosylation pattern. The media can be in a ready to use form or can be concentrated (e.g., as a stock solution), lyophilized, or provided in another reconstitutable form.

In a further embodiment, kits provided herein further comprise one or more reagents suitable for detecting modified polypeptides produced by the host cell. The kit can further comprise reagent(s) suitable for detecting a property of the cell, such as maximum cell density, cell viability, or the like. In some embodiments, reagent(s) suitable for detecting the polypeptide or a property thereof, such as the biological activity, homogeneity, or structure of the polypeptide are provided.

In some embodiments, one or more assay surfaces of the substrate further comprises a reagent that facilitates uptake of RNA effector molecules by host cells. Such reagent carriers for RNA effector molecules are known in the art and/or are described herein. For example, in some embodiments, the carrier is a lipid formulation such as Lipofectamine™ (Invitrogen; Carlsbad, Calif.) or a related formulation. Examples of such carrier formulations are described herein.

In some embodiments, one or more assay surfaces of the substrate comprise an RNA effector molecule or series of RNA effector molecules and a carrier, each in concentrated form, such that plating host cells onto the assay surface(s) results in a concentration of the RNA effector molecule(s) and the carrier effective for facilitating uptake of the RNA effector molecule(s) by the host cells and modulation of the expression of one or more genes targeted by the RNA effector molecules.

In some embodiments, the substrate further comprises a matrix which facilitates three-dimensional cell growth and/or production of the biological product by host cells. In some embodiments, the matrix facilitates anchorage-independent growth of host cells. In further embodiments, the matrix facilitates anchorage-dependent growth of host cells. Non-limiting examples of matrix materials suitable for use with various kits described herein include agar, agarose, methylcellulose, alginate hydrogel (e.g., 5% alginate+5% collagen type I), chitosan, hydroactive hydrocolloid polymer gels, polyvinyl alcohol-hydrogel (PVA-H), polylactide-co-glycolide (PLGA), collagen vitrigel, PHEMA (poly(2-hydroxylmethacrylate)) hydrogels, PVP/PEO hydrogels, BD PuraMatrix™ hydrogels, and copolymers of 2-methacryloyloxyethyl phosphorylcholine (MPC).

In some embodiments, the substrate comprises a microarray plate, a biochip, or the like which allows for the high-throughput, automated testing of a range of test agents, conditions, and/or combinations thereof on the production of a modified polypeptide by cultured host cells. For example, the substrate may comprise a two-dimensional microarray plate or biochip having m columns and n rows of assay surfaces (e.g., residing within wells) which allow for the testing of m×n combinations of test agents and/or conditions (e.g., on a 24, 96 or 384-well microarray plate). The microarray substrates are preferably designed such that all necessary positive and negative controls can be carried out in parallel with testing of the agents and/or conditions.

In some embodiments, kits are provided comprising one or more microarray plates or biochips seeded with a series of RNA effector molecules designed to modify the glycosylation pattern of a polypeptide.

In further embodiments, kits are provided that can further comprise one or more microarray substrates seeded with a set of RNA effector molecules designed to modulate a particular pathway, function, or property of a host cell which affects the production of the biological product. For example, in some embodiments, the RNA effector molecules are directed against target genes comprising a pathway involved in the expression, folding, secretion, or post-translational modification of a recombinant protein product by the host cell.

In another embodiment, the product is a multi-subunit recombinant protein and the RNA effector molecules are directed against target genes involved in post-translation modification of the protein by the host cell, such as methionine oxidation, glycosylation, disulfide bond formation, pyroglutamation and/or protein deamidation.

In some embodiments, kits provided herein allow for the selection or optimization of at least one factor for modifying the glycosylation pattern of a polypeptide. For example, the kits may allow for the selection of an RNA effector molecule from among a series of candidate RNA effector molecules, or for the selection of a concentration or concentration range from a wider range of concentrations of a given RNA effector molecule. In some embodiments, the kits allow for selection of one or more RNA effector molecules from a series of candidate RNA effector molecules directed against a common target gene. In further embodiments, the kits allow for selection of one or more RNA effector molecules from a series of candidate RNA effector molecules directed against two or more functionally related target genes or two or more target genes of a common host cell pathway.

In some embodiments, kits provided herein allow for the selection or optimization of a combination of two or more factors in the production of a modified polypeptide. For example, the kits may allow for the selection of a suitable RNA effector molecule from among a series of candidate RNA effector molecules as well as a concentration of the RNA effector molecule. In further embodiments, kits provided herein allow for the selection of a first RNA effector molecule from a first series of candidate RNA effector molecules and a second RNA effector molecule from a second series of candidate RNA effector molecules. In some embodiments, the first and/or second series of candidate RNA effector molecules are directed against a common target gene. In further embodiments, the first and/or second series of RNA effector molecules are directed against two or more functionally related target genes or two or more target genes of a common host cell pathway.

In one embodiment, a kit for modifying the glycosylation pattern of a polypeptide in a host cell is provided comprising one or more microarray plates seeded with a series of different RNA effector molecules against a common target gene.

In another embodiment, a kit for modifying the glycosylation pattern of a polypeptide in a host cell is provided comprising one or more microarray plates seeded with a range of concentrations of an RNA effector molecule.

In another embodiment, a kit for modifying the glycosylation pattern of a polypeptide in a host cell is provided comprising one or more microarray plates seeded with a series of RNA effector molecules against a plurality of target genes.

In another embodiment, a kit for modifying the glycosylation pattern of a polypeptide in a host cell is provided comprising one or more two-dimensional microarray plates seeded along one dimension (e.g., rows or columns) with a series of RNA effector molecules against a common target gene and along the remaining dimension with a range of concentrations of each RNA effector molecule.

In another embodiment, a kit for modifying the glycosylation pattern of a polypeptide in a host cell is provided comprising one or more two-dimensional microarray plates seeded along one dimension (e.g., rows or columns) with a series of RNA effector molecules against a plurality of target genes and along the remaining dimension with a range of concentrations of each RNA effector molecule of the series.

In another embodiment, a kit for modifying the glycosylation pattern of a polypeptide in a host cell is provided comprising one or more two-dimensional microarray plates seeded along one dimension (e.g., rows or columns) with a first series of RNA effector molecules and along the remaining dimension with a second series of RNA effector molecules, wherein the first series comprises different RNA effector molecules against a first target gene and the second series comprises different RNA effector molecules against a second target gene.

In another embodiment, a kit for modifying the glycosylation pattern of a polypeptide in a host cell is provided comprising one or more two-dimensional microarray plates seeded along one dimension (e.g., rows or columns) with a first series of RNA effector molecules and along the remaining dimension with a second series of RNA effector molecules, wherein the first series comprises different RNA effector molecules against a first target gene and the second series comprises RNA effector molecules against a plurality of additional target genes.

In another embodiment, a kit for modifying the glycosylation pattern of a polypeptide in a host cell is provided comprising one or more two-dimensional microarray plates seeded along one dimension (e.g., rows or columns) with a first series of RNA effector molecules and along the remaining dimension with a second series of RNA effector molecules, wherein the first series comprises RNA effector molecules against a first plurality of target genes and the second series comprises RNA effector molecules against a second plurality of target genes.

Provided herein in one aspect is a kit for producing a polypeptide comprising at least one terminal mannose at an N-linked glycosylation site, the kit comprising: (a) at least one RNA effector molecule that inhibits expression of a gene product involved in protein glycosylation in an admixture with a host cell; and (b) instructions and packaging materials therefor.

In one embodiment of this aspect, the host cell is a CHO cell.

In another embodiment, the gene product involved in protein glycosylation is selected from the group consisting of: Mgat1, Mgat4, SLC35A1, SLC35A2, and GNE. In another embodiment, the at least one RNA effector molecule comprises (a) a sense strand comprising a sequence selected from the group consisting of: SEQ ID NO. 1-33, SEQ ID NO. 67-94, SEQ ID NO. 123-154, SEQ ID NO. 187-221, and SEQ ID NO. 257-282; and (b) a complementary anti-sense strand comprising a sequence selected from the group consisting of SEQ ID NO. 34-66, SEQ ID NO. 95-122, SEQ ID NO. 155-186, SEQ ID NO. 222-256 and SEQ ID NO. 283-308.

In another embodiment, the kit further comprises a cell medium for culturing the host cell. In another embodiment, the kit further comprises an expression vector.

In other embodiments, the RNA effector molecule is provided at a concentration selected from the group consisting of 0.1 nM, 0.5 nM, 0.75 nM, 1 nM, 2 nM, 5 nM, 10 nM, 20 nM, 30 nM, 40 nM, 50 nM, and 60 nM. Alternatively, in other embodiments the RNA effector molecule is provided at an amount of 50 molecules per cell, 100 molecules per cell, 200 molecules per cell, 300 molecules per cell, 400 molecules per cell, 500 molecules per cell, 600 molecules per cell, 700 molecules per cell, 800 molecules per cell, 900 molecules per cell, 1000 molecules per cell, 2000 molecules per cell, or 5000 molecules per cell. In further embodiments, the RNA effector molecule is provided at a concentration selected from the group consisting of: 0.01 fmol/10⁶ cells, 0.1 fmol/10⁶ cells, 0.5 fmol/10⁶ cells, 0.75 fmol/10⁶ cells, 1 fmol/10⁶ cells, 2 fmol/10⁶ cells, 5 fmol/10⁶ cells, 10 fmol/10⁶ cells, 20 fmol/10⁶ cells, 30 fmol/10⁶ cells, 40 fmol/10⁶ cells, 50 fmol/10⁶ cells, 60 fmol/10⁶ cells, 100 fmol/10⁶ cells, 200 fmol/10⁶ cells, 300 fmol/10⁶ cells, 400 fmol/10⁶ cells, 500 fmol/10⁶ cells, 700 fmol/10⁶ cells, 800 fmol/10⁶ cells, 900 fmol/10⁶ cells, and 1 pmol/10⁶ cells.

In another embodiment, the kit further comprises an RNA effector molecule that inhibits expression of the mannose 6 phosphate receptor.

The present invention can be defined in any of the following numbered paragraphs:
1. A method of producing a polypeptide with a modified glycosylation pattern at an N-linked glycosylation site, the method comprising:
   (a) culturing a cell comprising a polypeptide to be modified in the presence of at least one RNA effector molecule that inhibits expression of a gene product involved in protein glycosylation such that at least one polypeptide N-linked glycosylation site is modified to have a terminal mannose, and wherein the cell is cultured under conditions permitting glycosylation and for a sufficient time to allow expression of the polypeptide to be modified; and
   (b) isolating the polypeptide,
   wherein the polypeptide produced by step (a) comprises a terminal mannose in at least one N-linked glycosylation site, thereby producing a polypeptide with a modified glycosylation pattern.
2. The method of paragraph 1, further comprising culturing the cell with an RNA effector molecule that inhibits expression of the mannose 6 phosphate receptor.
3 The method of any of paragraphs 1-2, wherein at least two N-linked glycosylation sites are modified.
4. The method of any of paragraphs 1-3, wherein at least three N-linked glycosylation sites are modified.
5. The method of any of paragraphs 1-4, wherein at least four N-linked glycosylation sites are modified.
6. The method of any of paragraphs 1-5, wherein the modified N-linked glycosylation site comprises an oligomannosyl structure.
7. The method of paragraph 6, wherein the modified N-linked glycosylation site consists of an oligomannosyl structure selected from the group consisting of: Man₂GlcNAc₂, Man₃GlcNAc₂, Man₄GlcNAc₂, Man₅ GlcNAc₂, Man₆GlcNAc₂, Man₇GlcNAc₂, Man₈GlcNAc₂, and Man₉GlcNAc₂.
8. The method of any of paragraphs 1-7, wherein the polypeptide comprises 2, 3, 4, 5, 6, 7, 8, or 9 terminal mannoses in the at least one N-linked glycosylation site.
9. The method of any of paragraphs 1-8, wherein the gene product that is inhibited is selected from the group consisting of: Mgat1, Mgat4, SLC35A1, SLC35A2, and GNE.
10. The method of any of paragraphs 1-9, wherein the polypeptide binds a mannose receptor present on macrophages.
11. The method of any of paragraphs 1-10, wherein the polypeptide is secreted from the cell.
12. The method of any of paragraphs 1-11, wherein the at least one RNA effector molecule is an siRNA.
13. The method of claim any of paragraphs 1-12, wherein the at least one RNA effector molecule comprises
   (a) a sense strand comprising a sequence selected from the group consisting of: SEQ ID NO. 1-33, SEQ ID NO. 67-94, SEQ ID NO. 123-154, SEQ ID NO. 187-221, and SEQ ID NO. 257-282; and
   (b) a complementary anti-sense strand comprising a sequence selected from the group consisting of SEQ ID NO. 34-66, SEQ ID NO. 95-122, SEQ ID NO. 155-186, SEQ ID NO. 222-256 and SEQ ID NO. 283-308.
14. The method of any of paragraphs 1-13, wherein step (a) is performed by adding the RNA effector molecule to a culture medium used to produce the polypeptide.
15. The method of paragraph 14, wherein the RNA effector molecule is added in combination with a reagent that facilitates RNA effector molecule uptake into the cell.
16. The method of any of paragraphs 1-15, wherein the polypeptide is used in treatment of a lysosomal storage disease.
17. The method of paragraph 16, wherein the polypeptide is selected from the group consisting of: glucocerebrosidase, idursulfase, alglucosidase alfa, galsulfase, agalsidase beta, and laronidase.
18. The method of paragraph 17, wherein the polypeptide comprises at least one mutation.
19. The method of any of paragraphs 1-18, wherein the polypeptide is glucocerebrosidase.
20. The method of paragraph 19, wherein the glucocerebrosidase comprises an arginine to histidine mutation at amino acid 495.
21. The method of any of paragraphs 1-20, wherein two or more RNA effector molecules are cultured with the cell.
22. An isolated polypeptide comprising a modified mannosylation pattern produced by the method of paragraph 1, wherein the polypeptide comprises a terminal mannose at at least one N-linked glycosylation site.
23. The polypeptide of paragraph 22, wherein the polypeptide lacks a mannose phosphate group.
24. The polypeptide of any of paragraphs 22-23, wherein the polypeptide has a reduced affinity for the mannose 6 phosphate receptor.
25. The polypeptide any of paragraphs 22-24, wherein at least two N-linked glycosylation sites are modified.
26. The polypeptide of any of paragraphs 22-25, wherein at least three N-linked glycosylation sites are modified.
27. The polypeptide of any of claims 22-26, wherein at least four N-linked glycosylation sites are modified.
28. The polypeptide of any of paragraphs 22-27, wherein the modified N-linked glycosylation site comprises an oligomannosyl structure.
29. The polypeptide of any of paragraphs 22-28, wherein the modified N-linked glycosylation site consists of an oligomannosyl structure selected from the group consisting of: Man₂GlcNAc₂, Man₃GlcNAc₂, Man₄GlcNAc₂, Man₅ GlcNAc₂, Man₆GlcNAc₂, Man₇GlcNAc₂, Man₈GlcNAc₂, and Man₉GlcNAc₂.
30. The polypeptide of any of paragraphs 22-29, wherein the polypeptide comprises 2, 3, 4, 5, 6, 7, 8, or 9 terminal mannoses in the at least one N-linked glycosylation chain.
31. The polypeptide of any of paragraphs 22-30, wherein the polypeptide binds a mannose receptor present on macrophages.
32. The polypeptide of any of paragraphs 22-31, wherein the polypeptide is secreted from the cell.
33. The polypeptide of any of paragraphs 22-32, wherein the polypeptide is used in treatment of lysosomal storage disease.
34. The polypeptide of paragraph 33, wherein the polypeptide is selected from the group consisting of: glucocerebrosidase, idursulfase, alglucosidase alfa, galsulfase, agalsidase beta, and laronidase.
35. The polypeptide of any of paragraphs 22-34, wherein the polypeptide comprises at least one mutation.
36. The polypeptide of any of paragraphs 22-35, wherein the polypeptide is glucocerebrosidase.
37. The polypeptide of paragraph 36, wherein the glucocerebrosidase comprises an arginine to histidine mutation at amino acid 495.
38. An isolated mammalian host cell, in which the mRNA expression of a target gene selected from the group consisting of: Mgat1, Mgat4, SLC35A1, SLC35A2, and GNE is inhibited by RNA interference, wherein when a gene encoding a polypeptide is introduced into the host cell and expressed, the host cell produces a polypeptide comprising the encoded polypeptide molecule which contains a terminal mannose in at least one glycosylation chain, said polypeptide having increased affinity for the mannose receptor when compared with the polypeptide produced in the presence of Mgat1, Mgat4, SLC35A1, SLC35A2, or GNE expression, thereby producing a polypeptide with increased macrophage internalization.
39. The host cell of paragraph 38, wherein the cell is a CHO cell.
40. The host cell of any of paragraphs 38-39, wherein the polypeptide is used to treat a lysosomal storage disease.
41. The host cell of any of paragraphs 38-40, wherein the polypeptide is selected from the group consisting of: glucocerebrosidase, idursulfase, alglucosidase alfa, galsulfase, agalsidase beta, and laronidase.
42. The host cell of paragraph 41, wherein the polypeptide comprises at least one mutation.
43. The host cell of any of paragraphs 38-42, wherein the polypeptide is glucocerebrosidase.
44. The host cell of paragraph 43, wherein the glucocerebrosidase comprises an arginine to histidine mutation at amino acid 495.
45. The host cell of any of paragraphs 38-44, wherein the polypeptide is introduced with an expression vector.
46. The host cell of any of paragraphs 38-45, wherein the cell is cultured in suspension.
47. The host cell of any of paragraphs 38-46, wherein the cell is cultured in a bioreactor.
48. The host cell of paragraphs 46 or 47, wherein the cell is cultured in a volume selected from the group consisting of 0.1 L, 0.5 L, 1 L, 5 L, 40 L, 500 L, 5000 L, and 50,000 L.
49. The host cell of any of paragraphs 38-48, wherein the polypeptide is secreted from the cell.
50. The host cell of any of paragraphs 38-49, wherein at least two N-linked glycosylation sites of the polypeptide are modified.
51. The host cell of any of paragraphs 38-50, wherein at least three N-linked glycosylation sites of the polypeptide are modified.
52. The host cell of any of paragraphs 38-51, wherein at least four N-linked glycosylation sites of the polypeptide are modified.
53. The host cell of any of paragraphs 38-52, wherein the modified N-linked glycosylation site of the polypeptide comprises an oligomannosyl structure.
54. The host cell of any of paragraphs 38-53, wherein the modified N-linked glycosylation site of the peptide comprises a glycosylation chain selected from the group consisting of: Man₂GlcNAc₂, Man₃GlcNAc₂, Man₄GlcNAc₂, Man₅ GlcNAc₂, Man₆GlcNAc₂, Man₇GlcNAc₂, Man₈GlcNAc₂, and Man₉GlcNAc₂.
55. The host cell of any of paragraphs 38-54, wherein the polypeptide comprises 2, 3, 4, 5, 6, 7, 8, or 9 terminal mannoses at the at least one N-linked glycosylation site.
56. The host cell of any of paragraphs 38-55, wherein the polypeptide binds a mannose receptor present on macrophages.
57. The host cell of any of paragraphs 38-56, wherein the mRNA expression of the target gene is transiently inhibited.
58. The host cell of paragraph 57, wherein the mRNA expression is transiently inhibited by contacting the cell with at least one RNA effector molecule.
59. The host cell of any of paragraphs 38-58, further comprising adding a reagent that facilitates RNA effector molecule uptake into the cell.
60. The host cell of any of paragraphs 38-59, wherein the at least one RNA effector molecule comprises an siRNA.
61. The host cell of any of paragraphs 38-60, wherein the at least one RNA effector molecule comprises
   (a) a sense strand comprising a sequence selected from the group consisting of: SEQ ID NO. 1-33, SEQ ID NO. 67-94, SEQ ID NO. 123-154, SEQ ID NO. 187-221, and SEQ ID NO. 257-282; and
   (b) a complementary anti-sense strand comprising a sequence selected from the group consisting of SEQ ID NO. 34-66, SEQ ID NO. 95-122, SEQ ID NO. 155-186, SEQ ID NO. 222-256 and SEQ ID NO. 283-308.
62. The host cell of paragraph 58, wherein two or more RNA effector molecules are cultured with the cell.
63. A composition comprising at least one RNA effector molecule comprising a nucleic acid sequence complementary to at least one target gene of a host cell, wherein the RNA effector molecule is capable of modulating mannosylation patterns at an N-linked glycosylation site of a polypeptide produced in the host cell, and wherein the target gene is selected from the group consisting of: Mgat1, Mgat4, SLC35A1, SLC35A2, and GNE.
64. The composition of paragraph 63, wherein the at least one RNA effector molecule comprises a duplex region.
65. The composition of any of paragraphs 63-64, wherein the at least one RNA effector molecule is 15-30 nucleotides in length.
66. The composition of any of paragraphs 63-65, wherein the at least one RNA effector molecule is 17-28 nucleotides in length.
67. The composition of any of paragraphs 63-66, wherein the at least one RNA effector molecule comprises a modified nucleotide.
68. The composition of any of paragraphs 63-67, wherein the at least one RNA effector molecule comprises
   (a) a sense strand comprising a sequence selected from the group consisting of: SEQ ID NO. 1-33, SEQ ID NO. 67-94, SEQ ID NO. 123-154, SEQ ID NO. 187-221, and SEQ ID NO. 257-282; and
   (b) a complementary anti-sense strand comprising a sequence selected from the group consisting of SEQ ID NO. 34-66, SEQ ID NO. 95-122, SEQ ID NO. 155-186, SEQ ID NO. 222-256 and SEQ ID NO. 283-308.
69. The composition of any of paragraphs 63-68, further comprising an RNA effector molecule that inhibits expression of the mannose 6 phosphate receptor.
70. An isolated polypeptide that comprises a terminal mannose in at least one N-linked glycosylation site, wherein the glycosylation pattern of the isolated polypeptide has not been modified enzymatically to contain the terminal mannose.
71. The isolated polypeptide of paragraph 70, wherein the polypeptide is glucocerebrosidase.
72. A composition comprising a dsRNA for inhibiting expression of a target gene selected from the group consisting of: Mgat1, Mgat4, SLC35A1, SLC35A2, and GNE, the dsRNA comprising
   (a) a sense strand comprising a sequence selected from the group consisting of: SEQ ID NO. 1-33, SEQ ID NO. 67-94, SEQ ID NO. 123-154, SEQ ID NO. 187-221, and SEQ ID NO. 257-282; and
   (b) a complementary anti-sense strand comprising a sequence selected from the group consisting of SEQ ID NO. 34-66, SEQ ID NO. 95-122, SEQ ID NO. 155-186, SEQ ID NO. 222-256 and SEQ ID NO. 283-308.

All patents and other publications identified in the specification are expressly incorporated herein by reference in their entirety for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

To the extent not already indicated, it will be understood by those of ordinary skill in the art that any one of the various embodiments herein described and illustrated can be further modified to incorporate features shown in any of the other embodiments disclosed herein.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Example 1

### Vector Construction

The gene sequence for CEREZYME® was purchased from BLUE HERON BIOTECHNOLOGY®. A Polymerase chain reaction (PCR) was performed using primers containing the 5′ cloning site (NotI) and 3′ cloning site (AscI) using the PHUSION® High Fidelity PCR kit from NEW ENGLAND BIOLABS® using the protocol recommended by the manufacturer. The DNA sequence and its protein translation are listed in Appendix A. Primers specific for the CEREZYME® were designed and synthesized by INTEGRATED DNA TECHNOLOGIES® (IDT). The reaction was examined by agarose gel electrophoresis for the presence of a 1570 bp fragment and purified on the PCR Purification Kit (QIAGEN®). Approximately 4 micrograms of PCR product were digested with the restriction enzymes NotI and AscI for three hours using manufacturers recommended conditions (NEW ENGLAND BIOLABS®). The reaction was subsequently purified using the PCR Purification Kit (QIAGEN®).

The vector GV90 was digested with the enzymes NotI and AscI for 2-3 hours and the treated with alkaline phosphatase for 1 hour. The vector was subsequently purified by phenol extraction followed by ethanol precipitation.

The ligation mixture contained 50 ng of NotI & AscI digested vector and 2 and 4 fold excess (separately) of CEREZYME® insert using the NEW ENGLAND BIOLABS® Quick Ligation kit. DH5 alpha cells were transformed and recombinants were selected by resistance to 100 ug/ml Ampicillin. Individual colonies were screened by restriction digestion for the presence of the insert. A clone with the correct restriction digestion pattern was selected and grown on large scale. DNA was prepared and used in subsequent CHO cell transfections.

### Transfection of CHO Cells

Chinese Hamster Ovary host cell line DG44 was purchased from INVITROGENT™ or directly obtained from Larry Chasin (Columbia University). The cells were transfected with the CEREZYME® expression vector using FUGENE® mediated transfection at a FUGENE®/DNA ratio of 3/1 (v/w). After a thirty minute incubation at room temperature the DNA lipid complexes were added to two million CHO DG44 cells and incubated overnight. On the next day cells were shifted to nucleoside deficient media to select transfected cells. High expressing cells were screened with an anti beta-glucocerebrosidase antibody using the method of Brezinsky et. al.

In order to produce the oligomannose form of the glucocerebrosidase, siRNAs against the following target hamster genes were used: Mgat1, Mgat4B, SLC35A1, SLC35A2, GNE. Exemplary siRNA sequences directed at these target genes is provided herein in Tables 2-6.

TABLE 2
RNA effector molecules sequences targeting GNE (hamster)

| | | SEQ | | SEQ | |
|---|---|---|---|---|---|
| Oligo | Start | ID | Sense Sequence | ID | Antisense Sequence |
| Name | Pos. | NO. | 5′ to 3′ | NO. | 5′ to 3′ |
| CHO1322.1_13-31_s | 13 | 1 | AGCCAUGGUAGAGUCAGUA | 34 | UACUGACUCUACCAUGGCU |
| CHO1322.1_82-100_s | 82 | 2 | CAUCAUGAUUGUUCAUGGA | 35 | UCCAUGAACAAUCAUGAUG |
| CHO1322.1_315-333_s | 315 | 3 | GCCCUUCCUAUGACAAACU | 36 | AGUUUGUCAUAGGAAGGGC |
| CHO1322.1_318-336_s | 318 | 4 | CUUCCUAUGACAAACUGCU | 37 | AGCAGUUUGUCAUAGGAAG |
| CHO1322.1_357-375_s | 357 | 5 | AUAUGAGCAUCAUUCGGAU | 38 | AUCCGAAUGAUGCUCAUAU |
| CHO1322.1_402-420_s | 402 | 6 | AAGAUUACAUUGUUGCACU | 39 | AGUGCAACAAUGUAAUCUU |
| CHO1322.1_477-495_s | 477 | 7 | UGGAUGCACUUAUCUCAUU | 40 | AAUGAGAUAAGUGCAUCCA |
| CHO1322.1_597-615_s | 597 | 8 | UUCGUGCAGUCAAGCAUGU | 41 | ACAUGCUUGACUGCACGAA |
| CHO1322.1_604-622_s | 604 | 9 | AGUCAAGCAUGUCCCAUUU | 42 | AAAUGGGACAUGCUUGACU |
| CHO1322.1_615-633_s | 615 | 10 | UCCCAUUUGACCAGUUUAU | 43 | AUAAACUGGUCAAAUGGGA |
| CHO1322.1_616-634_s | 616 | 11 | CCCAUUUGACCAGUUUAUA | 44 | UAUAAACUGGUCAAAUGGG |
| CHO1322.1_645-663_s | 645 | 12 | CCCAUGCUGGUUGUAUGAU | 45 | AUCAUACAACCAGCAUGGG |
| CHO1322.1_727-745_s | 727 | 13 | AACACGCCAGAUAGGAAGA | 46 | UCUUCCUAUCUGGCGUGUU |
| CHO1322.1_777-795_s | 777 | 14 | AUGCUGACACCCAAGAUAA | 47 | UUAUCUUGGGUGUCAGCAU |
| CHO1322.1_778-796_s | 778 | 15 | UGCUGACACCCAAGAUAAA | 48 | UUUAUCUUGGGUGUCAGCA |
| CHO1322.1_798-816_s | 798 | 16 | UAUUACAAGCGCUCCACCU | 49 | AGGUGGAGCGCUUGUAAUA |
| CHO1322.1_811-829_s | 811 | 17 | CCACCUUCAGUUCGGUAAA | 50 | UUUACCGAACUGAAGGUGG |
| CHO1322.1_816-834_s | 816 | 18 | UUCAGUUCGGUAAACAGUA | 51 | UACUGUUUACCGAACUGAA |
| CHO1322.1_1049-1067_s | 1049 | 19 | AAGGGUGAAAUAGUUAAGA | 52 | UCUUAACUAUUUCACCCUU |
| CHO1322.1_1050-1068_s | 1050 | 20 | AGGGUGAAAUAGUUAAGAA | 53 | UUCUUAACUAUUUCACCCU |
| CHO1322.1_1106-1124_s | 1106 | 21 | AUUAGUUUAAUCCUGCAGA | 54 | UCUGCAGGAUUAAACUAAU |
| CHO1322.1_1169-1187_s | 1169 | 22 | AUUCUGGGAGUAGGCAUUU | 55 | AAAUGCCUACUCCCAGAAU |
| CHO1322.1_1342-1360_s | 1342 | 23 | AGAAAGGAAGUUUGGCCAA | 56 | UUGGCCAAACUUCCUUUCU |
| CHO1322.1_1378-1396_s | 1378 | 24 | CUUUGUGACACUCAUUACA | 57 | UGUAAUGAGUGUCACAAAG |
| CHO1322.1_1382-1400_s | 1382 | 25 | GUGACACUCAUUACAGGCA | 58 | UGCCUGUAAUGAGUGUCAC |
| CHO1322.1_1674-1692_s | 1674 | 26 | ACGUGAAGGCCCAGAAUAU | 59 | AUAUUCUGGGCCUUCAGUC |
| CHO1322.1_1684-1702_s | 1684 | 27 | CCAGAAUAUCCUACGAACA | 60 | UGUUCGUAGGAUAUUCUGG |
| CHO1322.1_1687-1705_s | 1687 | 28 | GAAUAUCCUACGAACAGCU | 61 | AGCUGUUCGUAGGAUAUUC |
| CHO1322.1_1797-1815_s | 1797 | 29 | UCCACAUUGUCAAGGACGU | 62 | ACGUCCUUGACAAUGUGGA |
| CHO1322.1_1864-1882_s | 1846 | 30 | UUCAGACUUGGUUGACCCU | 63 | AGGGUCAACCAAGUCUGAA |
| CHO1322.1_1999-2017_s | 1999 | 31 | UUAGAUCAGUACUUCUUCA | 64 | UGAAGAAGUACUGAUCUAA |
| CHO1322.1_2166-2184_s | 2166 | 32 | CUAGAUUAAAGGUGGAUCU | 65 | AGAUCCACCUUUAAUCUAG |
| CHO1322.12211-2229_s | 2211 | 33 | AAUGGGUCUUUCCUCUUAA | 66 | UUAAGAGGAAAGACCCAUU |

TABLE 3
RNA effector molecules targeting MGAT1 (hamster)

| | | SEQ | | SEQ | |
|---|---|---|---|---|---|
| Oligo | Start | ID | Sense Sequence | ID | Antisense Sequence |
| Name | Pos. | NO. | 5′ to 3′ | NO. | 5′ to 3' |
| CHO5594.1_84-102_s | 84 | 67 | GGUUACUACAAGAUCGCCA | 95 | UGGCGAUCUUGUAGUAACC |
| CHO5594.1_124-142_s | 124 | 68 | GCCAGAUCUUCAACAAGUU | 96 | AACUUGUUGAAGAUCUGGC |
| CHO5594.1_187-205_s | 187 | 69 | GCACCAGACUUCUUUGAGU | 97 | ACUCAAAGAAGUCUGGUGC |
| CHO5594.1_188-206_s | 188 | 70 | CACCAGACUUCUUUGAGUA | 98 | UACUCAAAGAAGUCUGGUG |
| CHO5594.1_190-208_s | 190 | 71 | CCAGACUUCUUUGAGUACU | 99 | AGUACUCAAAGAAGUCUGG |
| CHO5594.1_252-270_s | 252 | 72 | GUGUGUGUCUGCUUGGAAU | 100 | AUUCCAAGCAGACACACAC |
| CHO5594.1_258-276_s | 258 | 73 | GUCUGCUUGGAAUGACAAU | 101 | AUUGUCAUUCCAAGCAGAC |
| CHO5594.1_315-333_s | 315 | 74 | GCUCUAUCGAACAGACUUU | 102 | AAAGUCUGUUCGAUAGAGC |
| CHO5594.1_448-466_s | 448 | 75 | GCCUGUAUUCGUCCAGAAA | 103 | UUUCUGGACGAAUACAGGC |
| CHO5594.1_450-468_s | 450 | 76 | CUGUAUUCGUCCAGAAAUU | 104 | AAUUUCUGGACGAAUACAG |
| CHO5594.1_460-478_s | 460 | 77 | CCAGAAAUUUCAAGAACGA | 105 | UCGUUCUUGAAAUUUCUGG |
| CHO5594.1_461-479_s | 461 | 78 | CAGAAAUUUCAAGAACGAU | 106 | AUCGUUCUUGAAAUUUCUG |
| CHO5594.1_463-481_s | 463 | 79 | GAAAUUUCAAGAACGAUGA | 107 | UCAUCGUUCUUGAAAUUUC |
| CHO5594.1_509-527_s | 509 | 80 | GGCAGUUCUUUGAUCAGCA | 108 | UGCUGAUCAAAGAACUGCC |
| CHO5594.1_520-538_s | 520 | 81 | GAUCAGCAUCUUAAGUUCA | 109 | UGAACUUAAGAUGCUGAUC |
| CHO5594.1_561-579_s | 561 | 82 | GUCUUUCACCCAGUUGGAU | 110 | AUCCAACUGGGUGAAAGAC |
| CHO5594.1_563-581_s | 563 | 83 | CUUUCACCCAGUUGGAUUU | 111 | AAAUCCAACUGGGUGAAAG |
| CHO5594.1_567-585_s | 567 | 84 | CACCCAGUUGGAUUUGUCA | 112 | UGACAAAUCCAACUGGGUG |
| CHO5594.1_571-589_s | 571 | 85 | CAGUUGGAUUUGUCAUACU | 113 | AGUAUGACAAAUCCAACUG |
| CHO5594.1_602-620_s | 602 | 86 | CUUAUGACCGGGAUUUCCU | 114 | AGGAAAUCCCGGUCAUAAG |
| CHO5594.1_665-683_s | 665 | 87 | GGACCAAUGAUCAGAAGGA | 115 | UCCUUCUGAUCAUUGGUCC |
| CHO5594.1_699-717_s | 699 | 88 | GGUACAGUACACUAGCAGA | 116 | UCUGCUAGUGUACUGUACC |
| CHO5594.1_703-721_s | 703 | 89 | CAGUACACUAGCAGAGACA | 117 | UGUCUCUGCUAGUGUACUG |
| CHO5594.1_797-815_s | 797 | 90 | GCGUUGUCACUUUCCAGUU | 118 | AACUGGAAAGUGACAACGC |
| CHO5594.1_901-919_s | 901 | 91 | GCUGGAUCUGCUUGUCAUA | 119 | UAUGACAAGCAGAUCCAGC |
| CHO5594.1_904-922_s | 904 | 92 | GGAUCUGCUUGUCAUAUCA | 120 | UGAUAUGACAAGCAGAUCC |
| CHO5594.1_905-923_s | 905 | 93 | GAUCUGCUUGUCAUAUCAU | 121 | AUGAUAUGACAAGCAGAUC |
| CHO5594.1_914-932_s | 914 | 94 | GUCAUAUCAUGAGCUGAGA | 122 | UCUCAGCUCAUGAUAUGAC |

TABLE 4
RNA effector molecules targeting MGAT4b (hamster)

| | | SEQ | | SEQ | |
|---|---|---|---|---|---|
| Oligo | Start | ID | Sense Sequence | ID | Antisense Sequence |
| Name | Pos. | NO. | 5′ to 3′ | NO. | 5′ to 3′ |
| CHO2254.1_53-71_s | 53 | 123 | AGCGCUCUAAGGAGCUAAA | 155 | UUUAGCUCCUUAGAGCGCU |
| CHO2254.1_56-74_s | 56 | 124 | GCUCUAAGGAGCUAAACCU | 156 | AGGUUUAGCUCCUUAGAGC |
| CHO2254.1_347-365_s | 347 | 125 | CGUACCUGACUGACACAUU | 157 | AAUGUGUCAGUCAGGUACG |
| CHO2254.1_359-377_s | 359 | 126 | ACACAUUGCACUCGCUCAU | 158 | AUGAGCGAGUGCAAUGUGU |
| CHO2254.1_361-379_s | 361 | 127 | ACAUUGCACUCGCUCAUCU | 159 | AGAUGAGCGAGUGCAAUGU |
| CHO2254.1_611-629_s | 611 | 128 | AGAACCUGGAUUACUGCUU | 160 | AAGCAGUAAUCCAGGUUCU |
| CHO2254.1_683-701_s | 683 | 129 | ACAUUGUAGCCAAGCCCAA | 161 | UUGGGCUUGGCUACAAUGU |
| CHO2254.1_694-712_s | 694 | 130 | AAGCCCAACUACUUGAGCA | 162 | UGCUCAAGUAGUUGGGCUU |
| CHO2254.1_824-842_s | 824 | 131 | UGGAAUUCAUCCUUAUGUU | 163 | AACAUAAGGAUGAAUUCCA |
| CHO2254.1_831-849_s | 831 | 132 | CAUCCUUAUGUUCUACCGA | 164 | UCGGUAGAACAUAAGGAUG |
| CHO2254.1_833-851_s | 833 | 133 | UCCUUAUGUUCUACCGAGA | 165 | UCUCGGUAGAACAUAAGGA |
| CHO2254.1_843-861_s | 843 | 134 | CUACCGAGACAAGCCUAUU | 166 | AAUAGGCUUGUCUCGGUAG |
| CHO2254.1_845-863_s | 845 | 135 | ACCGAGACAAGCCUAUUGA | 167 | UCAAUAGGCUUGUCUCGGU |
| CHO2254.1_847-865_s | 847 | 136 | CGAGACAAGCCUAUUGACU | 168 | AGUCAAUAGGCUUGUCUCG |
| CHO2254.1_884-902_s | 884 | 137 | UGUGGGUGAAAGUCUGCAA | 169 | UUGCAGACUUUCACCCACA |
| CHO2254.1_901-919_s | 901 | 138 | AACCCUGAGAAAGAUGCGA | 170 | UCGCAUCUUUCUCAGGGUU |
| CHO2254.1_902-920_s | 902 | 139 | ACCCUGAGAAAGAUGCGAA | 171 | UUCGCAUCUUUCUCAGGGU |
| CHO2254.1_903-921_s | 903 | 140 | CCCUGAGAAAGAUGCGAAA | 172 | UUUCGCAUCUUUCUCAGGG |
| CHO2254.1_1885-1903_s | 1885 | 141 | AUACACUACUUUAUGUGCU | 173 | AGCACAUAAAGUAGUGUAU |
| CHO2254.1_1887-1905_s | 1887 | 142 | ACACUACUUUAUGUGCUGU | 174 | ACAGCACAUAAAGUAGUGU |
| CHO2254.1_1934-1952_s.6 | 1934 | 143 | UUUCACGUUAAGUUCGCAU | 175 | AUGCGAACUUAACGUGAAA |
| CHO2254.1_1935-1953_s2.4 | 1935 | 144 | UUCACGUUAAGUUCGCAUA | 176 | UAUGCGAACUUAACGUGAA |
| CHO2254.1_1936-1954_s | 1936 | 145 | UCACGUUAAGUUCGCAUAU | 177 | AUAUGCGAACUUAACGUGA |
| CHO2254.1_1937-1955_s | 1937 | 146 | CACGUUAAGUUCGCAUAUA | 178 | UAUAUGCGAACUUAACGUG |
| CHO2254.1_1940-1958_s | 1940 | 147 | GUUAAGUUCGCAUAUACUU | 179 | AAGUAUAUGCGAACUUAAC |
| CHO2254.1_1942-1960_s | 1942 | 148 | UAAGUUCGCAUAUACUUCU | 180 | AGAAGUAUAUGCGAACUUA |
| CHO2254.1_1943-1961_s | 1943 | 149 | AAGUUCGCAUAUACUUCUA | 181 | UAGAAGUAUAUGCGAACUU |
| CHO2254.1_1945-1963_s | 1945 | 150 | GUUCGCAUAUACUUCUAUA | 182 | UAUAGAAGUAUAUGCGAAC |
| CHO2254.1_1952-1970_s | 1952 | 151 | UAUACUUCUAUAAGAGCGU | 183 | ACGCUCUUAUAGAAGUAUA |
| CHO2254.1_1957-1975_s | 1957 | 152 | UUCUAUAAGAGCGUGACUU | 184 | AAGUCACGCUCUUAUAGAA |
| CHO2254.1_1964-1982_s | 1964 | 153 | AGAGCGUGACUUGUAAUAA | 185 | UUAUUACAAGUCACGCUCU |
| CHO2254.1_1965-1983_s | 1965 | 154 | GAGCGUGACUUGUAAUAAA | 186 | UUUAUUACAAGUCACGCUC |

TABLE 5
RNA effector molecules targeting SLC35A1 (hamster)

| | | SEQ | | SEQ | |
|---|---|---|---|---|---|
| Oligo | Start | ID | Sense Sequence | ID | Antisense Sequence |
| Name | Pos. | NO. | 5′ to 3′ | NO. | 5′ to 3′ |
| CHO4117 | 148 | 187 | GCAGCUUAUACCGUAG | 222 | AAGCUACGGUAUAAGCU |
| .1_148- | | | CUU | | GC |
| 166_s | | | | | |
| CHO4117 | 149 | 188 | CAGCUUAUACCGUAGC | 223 | AAAGCUACGGUAUAAGC |
| .1_149- | | | UUU | | UG |
| 167_s | | | | | |
| CHO4117 | 159 | 189 | CGUAGCUUUAAGAUAC | 224 | UGUGUAUCUUAAAGCUA |
| .1_159- | | | ACA | | CG |
| 177_s | | | | | |
| CHO4117 | 160 | 190 | GUAGCUUUAAGAUACA | 225 | UUGUGUAUCUUAAAGCU |
| .1_160- | | | CAA | | AC |
| 178_s | | | | | |
| CHO4117 | 223 | 191 | GUCACAGAAGUUAUAA | 226 | ACUUUAUAACUUCUGUG |
| .1_223- | | | AGU | | AC |
| 241_s | | | | | |
| CHO4117 | 240 | 192 | GUUACUGAUAAGUGU | 227 | UCCAACACUUAUCAGUA |
| .1_240- | | | UGGA | | AC |
| 258_s | | | | | |
| CHO4117 | 275 | 193 | CUGGAAGUUUGGGUA | 228 | AAUCUACCCAAACUUCC |
| .1_275- | | | GAUU | | AG |
| 293_s | | | | | |
| CHO4117 | 278 | 194 | GAAGUUUGGGUAGAU | 229 | UUAAAUCUACCCAAACU |
| .1_278- | | | UUAA | | UC |
| 296_s | | | | | |
| CHO4117 | 285 | 195 | GGGUAGAUUUAAGGC | 230 | AGAUGCCUUAAAUCUAC |
| .1_285- | | | AUCU | | CC |
| 303_s | | | | | |
| CHO4117 | 286 | 196 | GGUAGAUUUAAGGCA | 231 | AAGAUGCCUUAAAUCUA |
| .1_286- | | | UCUU | | CC |
| 304_s | | | | | |
| CHO4117 | 387 | 197 | GGCUUUCCUAGCUCUU | 232 | ACUAAGAGCUAGGAAAG |
| .1_387- | | | AGU | | CC |
| 405_s | | | | | |
| CHO4117 | 389 | 198 | CUUUCCUAGCUCUUAG | 233 | AUACUAAGAGCUAGGAA |
| .1_389- | | | UAU | | AG |
| 407_s | | | | | |
| CHO4117 | 414 | 199 | CAGUAUACCAGGUUAC | 234 | UAGGUAACCUGGUAUAC |
| .1_414- | | | CUA | | UG |
| 432_s | | | | | |
| CHO4117 | 489 | 200 | CUCAGCAAAUUACAUG | 235 | ACCCAUGUAAUUUGCUG |
| .1_489- | | | GGU | | AG |
| 507_s | | | | | |
| CHO4117 | 600 | 201 | GCUUUGGUGCAAUAGC | 236 | AUAGCUAUUGCACCAAA |
| .1_600- | | | UAU | | GC |
| 618_s | | | | | |
| CHO4117 | 609 | 202 | CAAUAGCUAUUGCUGU | 237 | AAUACAGCAAUAGCUAU |
| .1_609- | | | AUU | | UG |
| 627_s | | | | | |
| CHO4117 | 620 | 203 | GCUGUAUUGUGCUCUG | 238 | AUCCAGAGCACAAUACA |
| .1_620- | | | GAU | | GC |
| 638_s | | | | | |
| CHO4117 | 635 | 204 | GGAUUUGCAGGAGUU | 239 | AAUAAACUCCUGCAAAU |
| .1_635- | | | UAUU | | CC |
| 653_s | | | | | |
| CHO4117 | 696 | 205 | GGGUGAGAAACAUUCA | 240 | AAGUGAAUGUUUCUCAC |
| .1_696- | | | CUU | | CC |
| 714_s | | | | | |
| CHO4117 | 917 | 206 | GAUACUCCUAUAACGA | 241 | UAGUCGUUAUAGGAGUA |
| .1_917- | | | CUA | | UC |
| 935_s | | | | | |
| CHO4117 | 921 | 207 | CUCCUAUAACGACUAA | 242 | AGUUUAGUCGUUAUAGG |
| .1_921- | | | ACU | | AG |
| 939_s | | | | | |
| CHO4117 | 930 | 208 | CGACUAAACUGUCAAU | 243 | AUUAUUGACAGUUUAGU |
| .1_930- | | | AAU | | CG |
| 948_s | | | | | |
| CHO4117 | 931 | 209 | GACUAAACUGUCAAUA | 244 | UAUUAUUGACAGUUUAG |
| .1_931- | | | AUA | | UC |
| 949_s | | | | | |
| CHO4117 | 971 | 210 | CCAGAUGGUAGCUUAA | 245 | UGUUUAAGCUACCAUCU |
| .1_971- | | | ACA | | GG |
| 989_s | | | | | |
| CHO4117 | 974 | 211 | GAUGGUAGCUUAAACA | 246 | UAUUGUUUAAGCUACCA |
| .1_974- | | | AUA | | UC |
| 992_s | | | | | |
| CHO4117 | 977 | 212 | GGUAGCUUAAACAAUA | 247 | UGAUAUUGUUUAAGCUA |
| .1_977- | | | UCA | | CC |
| 995_s | | | | | |
| CHO4117 | 978 | 213 | GUAGCUUAAACAAUAU | 248 | UUGAUAUUGUUUAAGCU |
| .1_978- | | | CAA | | AC |
| 996_s | | | | | |
| CHO4117 | 106 | 214 | GUGAAACUACAAUAUU | 249 | UUGAAUAUUGUAGUUUC |
| .1_1060- | 0 | | CAA | | AC |
| 1078_s | | | | | |
| CHO4117 | 109 | 215 | GGUAUCUGAAGGUUCA | 250 | ACUUGAACCUUCAGAUA |
| .1_1095- | 5 | | AGU | | CC |
| 1113_s | | | | | |
| CHO4117 | 113 | 216 | CUGUUCUCUCGUUCAG | 251 | UACCUGAACGAGAGAAC |
| .1_1136- | 6 | | GUA | | AG |
| 1154_s | | | | | |
| CHO4117 | 124 | 217 | GUGAAGAAUGAAUAA | 252 | UCUCUUAUUCAUUCUUC |
| .1_1246- | 6 | | GAGA | | AC |
| 1264_s | | | | | |
| CHO4117 | 128 | 218 | CUGACUUCUGUCUGGG | 253 | UUACCCAGACAGAAGUC |
| .1_1286- | 6 | | UAA | | AG |
| 1304_s | | | | | |
| CHO4117 | 136 | 219 | GCUGUUAGCUGAUAUA | 254 | AAGUAUAUCAGCUAACA |
| .1_1363- | 3 | | CUU | | GC |
| 1381_s | | | | | |
| CHO4117 | 144 | 220 | GGCUCUCAAUUUGUGA | 255 | AGUUCACAAAUUGAGAG |
| .1_1443- | 3 | | ACU | | CC |
| 1461_s | | | | | |
| CHO4117 | 144 | 221 | GCUCUCAAUUUGUGAA | 256 | AAGUUCACAAAUUGAGA |
| .1_1444- | 4 | | CUU | | GC |
| 1462_s | | | | | |

TABLE 6
RNA effector molecules targeting SLC35A2 (hamster)

| | | SEQ | | SEQ | |
|---|---|---|---|---|---|
| Oligo | Start | ID | Sense Sequence | ID | Antisense Sequence |
| Name | Pos. | NO. | 5′ to 3′ | NO. | 5′ to 3′ |
| CHO1453 | 165 | 257 | UCAUCCUUAGCAUCCG | 283 | UAUCGGAUGCUAAGGAU |
| .1_165- | | | AUA | | GA |
| 183_s | | | | | |
| CHO1453 | 166 | 258 | CAUCCUUAGCAUCCGA | 284 | AUAUCGGAUGCUAAGGA |
| .1_166- | | | UAU | | UG |
| 184_s | | | | | |
| CHO1453 | 173 | 259 | AGCAUCCGAUAUGCUC | 285 | UACGAGCAUAUCGGAUG |
| .1_173- | | | GUA | | CU |
| 191_s | | | | | |
| CHO1453 | 452 | 260 | CAGCUCAAGAUCCUGA | 286 | UAGUCAGGAUCUUGAGC |
| .1_452- | | | CUA | | UG |
| 470_s | | | | | |
| CHO1453 | 870 | 261 | UGGCUGUUGUAGUCAA | 287 | UACUUGACUACAACAGC |
| .1_870- | | | GUA | | CA |
| 888_s | | | | | |
| CHO1453 | 970 | 262 | UCACCUGGACCCAUUA | 288 | AAAUAAUGGGUCCAGGU |
| .1_970- | | | UUU | | GA |
| 988_s | | | | | |
| CHO1453 | 104 | 263 | AGUGCAGUCAAAGCCA | 289 | UUAUGGCUUUGACUGCA |
| .1_1043- | 3 | | UAA | | CU |
| 1061_s | | | | | |
| CHO1453 | 131 | 264 | CCACACUUCUAGAGGG | 290 | UAUCCCUCUAGAAGUGU |
| .1_1315- | 5 | | AUA | | GG |
| 1333_s | | | | | |
| CHO1453 | 131 | 265 | CACACUUCUAGAGGGA | 291 | AUAUCCCUCUAGAAGUG |
| .1_1316- | 6 | | UAU | | UG |
| 1334_s | | | | | |
| CHO1453 | 142 | 266 | AGGCUAACCUCUUUGG | 292 | UUCCCAAAGAGGUUAGC |
| .1_1425- | 5 | | GAA | | CU |
| 1443_s | | | | | |
| CHO1453 | 156 | 267 | UUCUUCAGUAACGACU | 293 | AUUAGUCGUUACUGAAG |
| .1_1564- | 4 | | AAU | | AA |
| 1582_s | | | | | |
| CHO1453 | 170 | 268 | GAAGAUCGGCCUGUUG | 294 | UUACAACAGGCCGAUCU |
| .1_1709- | 9 | | UAA | | UC |
| 1727_s | | | | | |
| CHO1453 | 179 | 269 | CAAUAAGCACCAUUUA | 295 | AAGUAAAUGGUGCUUAU |
| .1_1792- | 2 | | CUU | | UG |
| 1810_s | | | | | |
| CHO1453 | 181 | 270 | UAUGUCGGGCAUUUGU | 296 | AUCACAAAUGCCCGACA |
| .1_1815- | 5 | | GAU | | UA |
| 1833_s | | | | | |
| CHO1453 | 181 | 271 | AUGUCGGGCAUUUGUG | 297 | UAUCACAAAUGCCCGAC |
| .1_1816- | 6 | | AUA | | AU |
| 1834_s | | | | | |
| CHO1453 | 181 | 272 | UGUCGGGCAUUUGUGA | 298 | AUAUCACAAAUGCCCGA |
| .1_1817- | 7 | | UAU | | CA |
| 1835_s | | | | | |
| CHO1453 | 182 | 273 | GCAUUUGUGAUAUCAG | 299 | AACCUGAUAUCACAAAU |
| .1_1823- | 3 | | GUU | | GC |
| 1841_s | | | | | |
| CHO1453 | 201 | 274 | UCAUGGCGGUGUCAGA | 300 | AAUUCUGACACCGCCAU |
| .1_2012- | 2 | | AUU | | GA |
| 2030_s | | | | | |
| CHO1453 | 201 | 275 | UGGCGGUGUCAGAAUU | 301 | AUCAAUUCUGACACCGC |
| .1_2015- | 5 | | GAU | | CA |
| 2033_s | | | | | |
| CHO1453 | 206 | 276 | AAAGCUUACUAACUCC | 302 | UAAGGAGUUAGUAAGCU |
| .1_2065- | 5 | | UUA | | UU |
| 2083_s | | | | | |
| CHO1453 | 207 | 277 | UACUAACUCCUUAACU | 303 | UACAGUUAAGGAGUUAG |
| .1_2071- | 1 | | GUA | | UA |
| 2089_s | | | | | |
| CHO1453 | 207 | 278 | ACUAACUCCUUAACUG | 304 | AUACAGUUAAGGAGUUA |
| .1_2072- | 2 | | UAU | | GU |
| 2090_s | | | | | |
| CHO1453 | 219 | 279 | AAUGAACAUAUGUCAG | 305 | AUCCUGACAUAUGUUCA |
| .1_2198- | 8 | | GAU | | UU |
| 2216_s | | | | | |
| CHO1453 | 219 | 280 | AUGAACAUAUGUCAGG | 306 | UAUCCUGACAUAUGUUC |
| .1_2199- | 9 | | AUA | | AU |
| 2217_s | | | | | |
| CHO1453 | 221 | 281 | UCAGGAUACCCAAUGC | 307 | UUGGCAUUGGGUAUCCU |
| .1_2210- | 0 | | CAA | | GA |
| 2228_s | | | | | |
| CHO1453 | 221 | 282 | GAUACCCAAUGCCAAA | 308 | UUAUUUGGCAUUGGGUA |
| .1_2214- | 4 | | UAA | | UC |
| 2232_s | | | | | |

TABLE 7
RNA effector molecules targeting GNE (human;
NM_005476.4; SEQ ID NO: 309)

| | | SEQ | | SEQ | |
|---|---|---|---|---|---|
| Oligo | Start | ID | Sense Sequence | ID | Antisense Sequence |
| Name | Pos. | NO. | 5′ to 3′ | NO. | 5′ to 3′ |
| NM_0054 | 88 | 310 | ACUUGUAACCGUGCAG | 337 | AAUCUGCACGGUUACAA |
| 76.4_nols | | | AUU | | GU |
| texon_88- | | | | | |
| 106 | | | | | |
| NM_0054 | 202 | 311 | UAUGGAAAUACAUAUC | 338 | UUCGAUAUGUAUUUCCA |
| 76.4_nols | | | GAA | | UA |
| texon_202- | | | | | |
| 220 | | | | | |
| NM_0054 | 461 | 312 | GGACCAUUGAUGACUC | 339 | AUAGAGUCAUCAAUGGU |
| 76.4_nols | | | UAU | | CC |
| texon_461- | | | | | |
| 479 | | | | | |
| NM_0054 | 645 | 313 | UCGCAUGUGGCUAGGU | 340 | AUCACCUAGCCACAUGC |
| 76.4_nols | | | GAU | | GA |
| texon_645- | | | | | |
| 663 | | | | | |
| NM_0054 | 862 | 314 | CCCAACUUUCGUGCAG | 341 | UAACUGCACGAAAGUUG |
| 76.4_nols | | | UUA | | GG |
| texon_862- | | | | | |
| 880 | | | | | |
| NM_0054 | 974 | 315 | UUGGAACACCUGUGAU | 342 | UUGAUCACAGGUGUUCC |
| 76.4_nols | | | CAA | | AA |
| texon_974- | | | | | |
| 992 | | | | | |
| NM_0054 | 115 | 316 | GUUUCUCAAAUCUAUC | 343 | AUCGAUAGAUUUGAGAA |
| 76.4_nols | 8 | | GAU | | AC |
| texon_115 | | | | | |
| 8-1176 | | | | | |
| NM_0054 | 116 | 317 | UCUCAAAUCUAUCGAU | 344 | AAGAUCGAUAGAUUUGA |
| 76.4_nols | 1 | | CUU | | GA |
| texon_116 | | | | | |
| 1-1179 | | | | | |
| NM_0054 | 126 | 318 | UCUAAGUGCCUUGGCC | 345 | AACGGCCAAGGCACUUA |
| 76.4_nols | 0 | | GUU | | GA |
| texon_126 | | | | | |
| 0-1278 | | | | | |
| NM_0054 | 129 | 319 | CGAACCUCCGAGUUGC | 346 | AUUGCAACUCGGAGGUU |
| 76.4_nols | 2 | | AAU | | CG |
| texon_129 | | | | | |
| 2-1310 | | | | | |
| NM_0054 | 134 | 320 | AUACUCAGUUCAAUCC | 347 | UUAGGAUUGAACUGAGU |
| 76.4_nols | 3 | | UAA | | AU |
| texon_134 | | | | | |
| 3-1361 | | | | | |
| NM_0054 | 157 | 321 | CCCUGUGUGGGUAGAC | 348 | AUUGUCUACCCACACAG |
| 76.4_nols | 2 | | AAU | | GG |
| texon_157 | | | | | |
| 2-1590 | | | | | |
| NM_0054 | 191 | 322 | AUCUCAUCCAAGCUGC | 349 | UUCGCAGCUUGGAUGAG |
| 76.4_nols | 9 | | GAA | | AU |
| texon_191 | | | | | |
| 9-1937 | | | | | |
| NM_0054 | 192 | 323 | UCUCAUCCAAGCUGCG | 350 | UUUCGCAGCUUGGAUGA |
| 76.4_nols | 0 | | AAA | | GA |
| texon_192 | | | | | |
| 0-1938 | | | | | |
| NM_0054 | 193 | 324 | CGAAACUUGGCAAUGC | 351 | UUCGCAUUGCCAAGUUU |
| 76.4_nols | 4 | | GAA | | CG |
| texon_193 | | | | | |
| 4-1952 | | | | | |
| NM_0054 | 218 | 325 | ACUACACAACACGCAG | 352 | AUCCUGCGUGUUGUGUA |
| 76.4_nols | 6 | | GAU | | GU |
| texon_218 | | | | | |
| 6-2204 | | | | | |
| NM_0054 | 218 | 326 | ACACAACACGCAGGAU | 353 | UAGAUCCUGCGUGUUGU |
| 76.4_nols | 9 | | CUA | | GU |
| texon_218 | | | | | |
| 9-2207 | | | | | |
| NM_0054 | 227 | 327 | GUCUUUAGGAUGACCG | 354 | AAACGGUCAUCCUAAAG |
| 76.4_nols | 0 | | UUU | | AC |
| texon_227 | | | | | |
| 0-2288 | | | | | |
| NM_0054 | 227 | 328 | UUAGGAUGACCGUUUC | 355 | UAAGAAACGGUCAUCCU |
| 76.4_nols | 4 | | UUA | | AA |
| texon_227 | | | | | |
| 4-2292 | | | | | |
| NM_0054 | 227 | 329 | UAGGAUGACCGUUUCU | 356 | UUAAGAAACGGUCAUCC |
| 76.4_nols | 5 | | UAA | | UA |
| texon_227 | | | | | |
| 5-2293 | | | | | |
| NM_0054 | 228 | 330 | ACCGUUUCUUAACAAU | 357 | UUGAUUGUUAAGAAACG |
| 76.4_nols | 2 | | CAA | | GU |
| texon_228 | | | | | |
| 2-2300 | | | | | |
| NM_0054 | 371 | 331 | ACCCUAGGGUGUCCAU | 358 | UUAAUGGACACCCUAGG |
| 76.4_nols | 0 | | UAA | | GU |
| texon_371 | | | | | |
| 0-3728 | | | | | |
| NM_0054 | 385 | 332 | ACUAACUGCCACCACU | 359 | AUAAGUGGUGGCAGUUA |
| 76.4_nols | 4 | | UAU | | GU |
| texon_385 | | | | | |
| 4-3872 | | | | | |
| NM_0054 | 393 | 333 | UAUCUCAACAUAUGAG | 360 | UACCUCAUAUGUUGAGA |
| 76.4_nols | 1 | | GUA | | UA |
| texon_393 | | | | | |
| 1-3949 | | | | | |
| NM_0054 | 469 | 334 | CCUAGUGGGUUAGUGU | 361 | UUCACACUAACCCACUA |
| 76.4_nols | 2 | | GAA | | GG |
| texon_469 | | | | | |
| 2-4710 | | | | | |
| NM_0054 | 499 | 335 | ACUCAUGGGAAGGCUA | 362 | UAUUAGCCUUCCCAUGA |
| 76.4_nols | 3 | | AUA | | GU |
| texon_499 | | | | | |
| 3-5011 | | | | | |
| NM_0054 | 499 | 336 | UCAUGGGAAGGCUAAU | 363 | UAUAUUAGCCUUCCCAU |
| 76.4_nols | 5 | | AUA | | GA |
| texon_499 | | | | | |
| 5-5013 | | | | | |

TABLE 8
RNA effector molecules targeting MGAT1 (human;
NM_001114619.1; SEQ ID NO: 364)

| | | SEQ | | SEQ | |
|---|---|---|---|---|---|
| Oligo | Start | ID | Sense Sequence | ID | Antisense Sequence |
| Name | Pos. | NO. | 5′ to 3′ | NO. | 5′ to 3′ |
| NM_0011 | 98 | 365 | GCUGCCUCCUAAUCCC | 386 | UAUGGGAUUAGGAGGCA |
| 14619.1_ | | | AUA | | GC |
| 98-116 | | | | | |
| NM_0011 | 100 | 366 | UGCCUCCUAAUCCCAU | 387 | ACUAUGGGAUUAGGAGG |
| 14619.1_ | | | AGU | | CA |
| 100-118 | | | | | |
| NM_0011 | 242 | 367 | UUGUGCUGUGGGGCGC | 388 | AUAGCGCCCCACAGCAC |
| 14619.1_ | | | UAU | | AA |
| 242-260 | | | | | |
| NM_0011 | 114 | 368 | CUCAAGAACGAUGACC | 389 | AAAGGUCAUCGUUCUUG |
| 14619.1_ | 0 | | UUU | | AG |
| 1140- | | | | | |
| 1158 | | | | | |
| NM_0011 | 114 | 369 | CGAUGACCUUUGGCCG | 390 | UUGCGGCCAAAGGUCAU |
| 14619.1_ | 8 | | CAA | | CG |
| 1148- | | | | | |
| 1166 | | | | | |
| NM_0011 | 158 | 370 | UUGCCACAUCAUGAGC | 391 | UCAGCUCAUGAUGUGGC |
| 14619.1_ | 3 | | UGA | | AA |
| 1583- | | | | | |
| 1601 | | | | | |
| NM_0011 | 171 | 371 | GAUUAUUCUCCCGUUC | 392 | UGAGAACGGGAGAAUAA |
| 14619.1_ | 3 | | UCA | | UC |
| 1713- | | | | | |
| 1731 | | | | | |
| NM_0011 | 174 | 372 | GGGGAACUAUUCUAGG | 393 | UACCCUAGAAUAGUUCC |
| 14619.1_ | 6 | | GUA | | CC |
| 1746- | | | | | |
| 1764 | | | | | |
| NM_0011 | 176 | 373 | GUAUGUUGCGGGGUA | 394 | UUAAUACCCCGCAACAU |
| 14619.1 | 2 | | UUAA | | AC |
| 1762- | | | | | |
| 1780 | | | | | |
| NM_0011 | 176 | 374 | UGCGGGGUAUUAAGCA | 395 | UCCUGCUUAAUACCCCG |
| 14619.1_ | 8 | | GGA | | CA |
| 1768- | | | | | |
| 1786 | | | | | |
| NM_0011 | 176 | 375 | GCGGGGUAUUAAGCAG | 396 | UUCCUGCUUAAUACCCC |
| 14619.1_ | 9 | | GAA | | GC |
| 1769- | | | | | |
| 1787 | | | | | |
| NM_0011 | 186 | 376 | GCAGAGAGUUUGGCAA | 397 | ACGUUGCCAAACUCUCU |
| 14619.1_ | 4 | | CGU | | GC |
| 1864- | | | | | |
| 1882 | | | | | |
| NM_0011 | 186 | 377 | CAGAGAGUUUGGCAAC | 398 | AACGUUGCCAAACUCUC |
| 14619.1_ | 5 | | GUU | | UG |
| 1865- | | | | | |
| 1883 | | | | | |
| NM_0011 | 186 | 378 | GAGUUUGGCAACGUUC | 399 | AGCGAACGUUGCCAAAC |
| 14619.1_ | 9 | | GCU | | UC |
| 1869- | | | | | |
| 1887 | | | | | |
| NM_0011 | 187 | 379 | UUGGCAACGUUCGCUC | 400 | AGAGAGCGAACGUUGCC |
| 14619.1_ | 3 | | UCU | | AA |
| 1873- | | | | | |
| 1891 | | | | | |
| NM_0011 | 187 | 380 | UGGCAACGUUCGCUCU | 401 | AAGAGAGCGAACGUUGC |
| 14619.1_ | 4 | | CUU | | CA |
| 1874- | | | | | |
| 1892 | | | | | |
| NM_0011 | 196 | 381 | CCCAGUGGGGACUGAG | 402 | UAACUCAGUCCCCACUG |
| 14619.1_ | 7 | | UUA | | GG |
| 1967- | | | | | |
| 1985 | | | | | |
| NM_0011 | 196 | 382 | CCAGUGGGGACUGAGU | 403 | AUAACUCAGUCCCCACU |
| 14619.1_ | 8 | | UAU | | GG |
| 1968- | | | | | |
| 1986 | | | | | |
| NM_0011 | 200 | 383 | UGUGGCCAAAAUGAUA | 404 | UAGUAUCAUUUUGGCCA |
| 14619.1_ | 5 | | CUA | | CA |
| 2005- | | | | | |
| 2023 | | | | | |
| NM_0011 | 226 | 384 | UACCUCAGAGAGGGAC | 405 | AUAGUCCCUCUCUGAGG |
| 14619.1_ | 5 | | UAU | | UA |
| 2265- | | | | | |
| 2283 | | | | | |
| NM_0011 | 235 | 385 | GACAGAAUUCGAUCUG | 406 | AGGCAGAUCGAAUUCUG |
| 14619.1_ | 6 | | CCU | | UC |
| 2356- | | | | | |
| 2374 | | | | | |

TABLE 9
RNA effector molecules targeting MGAT4A (human;
NM_012214.2; SEQ ID NO: 407)

| | | SEQ | | SEQ | |
|---|---|---|---|---|---|
| Oligo | Start | ID | Sense Sequence | ID | Antisense Sequence |
| Name | Pos. | NO. | 5′ to 3′ | NO. | 5′ to 3′ |
| NM_0122 | 372 | 408 | ACUUUGUCUUGGUAUA | 436 | UAGUAUACCAAGACAAA |
| 14.2_372- | | | CUA | | GU |
| 390 | | | | | |
| NM_0122 | 548 | 409 | AAGUAAGGAUGCGUU | 437 | AUUCAACGCAUCCUUAC |
| 14.2_548- | | | GAAU | | UU |
| 566 | | | | | |
| NM_0122 | 549 | 410 | AGUAAGGAUGCGUUG | 438 | UAUUCAACGCAUCCUUA |
| 14.2_549- | | | AAUA | | CU |
| 567 | | | | | |
| NM_0122 | 690 | 411 | GUACAGAUUGGCAACG | 439 | UUCCGUUGCCAAUCUGU |
| 14.2_690- | | | GAA | | AC |
| 708 | | | | | |
| NM_0122 | 788 | 412 | CCUUAUUGAUAACCUG | 440 | AUACAGGUUAUCAAUAA |
| 14.2_788- | | | UAU | | GG |
| 806 | | | | | |
| NM_0122 | 196 | 413 | AAAGAUAGUUAAGCA | 441 | UACAUGCUUAACUAUCU |
| 14.2_196 | 7 | | UGUA | | UU |
| 7-1985 | | | | | |
| NM_0122 | 206 | 414 | GGAAAGUGAAUCUCCC | 442 | UAUGGGAGAUUCACUUU |
| 14.2_206 | 0 | | AUA | | CC |
| 0-2078 | | | | | |
| NM_0122 | 206 | 415 | AGUGAAUCUCCCAUAA | 443 | UUAUUAUGGGAGAUUCA |
| 14.2_206 | 4 | | UAA | | CU |
| 4-2082 | | | | | |
| NM_0122 | 399 | 416 | CCAGCUUGGUAUACUA | 444 | AUUUAGUAUACCAAGCU |
| 14.2_399 | 2 | | AAU | | GG |
| 2-4010 | | | | | |
| NM_0122 | 440 | 417 | CUGGAUGUGAGACCAA | 445 | UAAUUGGUCUCACAUCC |
| 14.2_440 | 4 | | UUA | | AG |
| 4-4422 | | | | | |
| NM_0122 | 466 | 418 | CUUGCAUACACAAUCG | 446 | AACCGAUUGUGUAUGCA |
| 14.2_466 | 3 | | GUU | | AG |
| 3-4681 | | | | | |
| NM_0122 | 488 | 419 | AUUGUAGUGGUCGCCC | 447 | UAAGGGCGACCACUACA |
| 14.2_488 | 7 | | UUA | | AU |
| 7-4905 | | | | | |
| NM_0122 | 502 | 420 | CAAAGGUUGGGACUAG | 448 | AAACUAGUCCCAACCUU |
| 14.2_502 | 7 | | UUU | | UG |
| 7-5045 | | | | | |
| NM_0122 | 541 | 421 | UUAUCUUAGUCUCAUG | 449 | AUGCAUGAGACUAAGAU |
| 14.2_541 | 0 | | CAU | | AA |
| 0-5428 | | | | | |
| NM_0122 | 556 | 422 | GAGAUGGUUGAAUACC | 450 | AAGGGUAUUCAACCAUC |
| 14.2_556 | 5 | | CUU | | UC |
| 5-5583 | | | | | |
| NM_0122 | 561 | 423 | GUGGUUGCACUAGUCA | 451 | UAUUGACUAGUGCAACC |
| 14.2_561 | 1 | | AUA | | AC |
| 1-5629 | | | | | |
| NM_0122 | 591 | 424 | UAUCAUGUUAUGUAGC | 452 | UUUGCUACAUAACAUGA |
| 14.2_591 | 4 | | AAA | | UA |
| 4-5932 | | | | | |
| NM_0122 | 643 | 425 | CCUGUAGUAUGCUGGA | 453 | AUAUCCAGCAUACUACA |
| 14.2_643 | 9 | | UAU | | GG |
| 9-6457 | | | | | |
| NM_0122 | 657 | 426 | CAUCUUACUGAGUAGC | 454 | AACGCUACUCAGUAAGA |
| 14.2_657 | 8 | | GUU | | UG |
| 8-6596 | | | | | |
| NM_0122 | 658 | 427 | UACUGAGUAGCGUUUG | 455 | UACCAAACGCUACUCAG |
| 14.2_658 | 3 | | GUA | | UA |
| 3-6601 | | | | | |
| NM_0122 | 658 | 428 | ACUGAGUAGCGUUUGG | 456 | UUACCAAACGCUACUCA |
| 14.2_658 | 4 | | UAA | | GU |
| 4-6602 | | | | | |
| NM_0122 | 679 | 429 | AUCUGUGUAUCGAGGG | 457 | AAUCCCUCGAUACACAG |
| 14.2_679 | 9 | | AUU | | AU |
| 9-6817 | | | | | |
| NM_0122 | 723 | 430 | UAUGCUACGAUAACUA | 458 | UACUAGUUAUCGUAGCA |
| 14.2_723 | 5 | | GUA | | UA |
| 5-7253 | | | | | |
| NM_0122 | 724 | 431 | ACGAUAACUAGUAUGC | 459 | UAAGCAUACUAGUUAUC |
| 14.2_724 | 1 | | UUA | | GU |
| 1-7259 | | | | | |
| NM_0122 | 740 | 432 | GGCACUAACUAGAUCA | 460 | AAAUGAUCUAGUUAGUG |
| 14.2_740 | 4 | | UUU | | CC |
| 4-7422 | | | | | |
| NM_0122 | 752 | 433 | AGCACUUUGCGCGAAG | 461 | UUACUUCGCGCAAAGUG |
| 14.2_752 | 6 | | UAA | | CU |
| 6-7544 | | | | | |
| NM_0122 | 799 | 434 | GUCCCAUUCAGAACAC | 462 | AAAGUGUUCUGAAUGGG |
| 14.2_799 | 3 | | UUU | | AC |
| 3-8011 | | | | | |
| NM_0122 | 815 | 435 | AUUCCUAUCCGUAGUA | 463 | UAUUACUACGGAUAGGA |
| 14.2_815 | 9 | | AUA | | AU |
| 9-8177 | | | | | |

TABLE 10
RNA effector molecules targeting MGAT4B
(human; NM_014275.4; SEQ ID NO: 464)

| | | SEQ | | SEQ | |
|---|---|---|---|---|---|
| Oligo | Start | ID | Sense Sequence | ID | Antisense Sequence |
| Name | Pos. | NO. | 5′ to 3′ | NO. | 5′ to 3′ |
| NM_0142 | 583 | 465 | AAGAGGGCCGUGUCAG | 490 | UUUCUGACACGGCCCUC |
| 75.4_583- | | | AAA | | UU |
| 601 | | | | | |
| NM_0142 | 837 | 466 | CUCGUACCUGACUGAC | 491 | AGUGUCAGUCAGGUACG |
| 75.4_837- | | | ACU | | AG |
| 855 | | | | | |
| NM_0142 | 839 | 467 | CGUACCUGACUGACAC | 492 | AGAGUGUCAGUCAGGUA |
| 75.4_839- | | | UCU | | CG |
| 857 | | | | | |
| NM_0142 | 923 | 468 | CCGAGACUGACUCACA | 493 | UACUGUGAGUCAGUCUC |
| 75.4_923- | | | GUA | | GG |
| 941 | | | | | |
| NM_0142 | 976 | 469 | UUCCCCACGGAGAUCC | 494 | AAUGGAUCUCCGUGGGG |
| 75.4_976- | | | AUU | | AA |
| 994 | | | | | |
| NM_0142 | 109 | 470 | CCAAACAGAACCUCGA | 495 | UAAUCGAGGUUCUGUUU |
| 75.4_109 | 7 | | UUA | | GG |
| 7-1115 | | | | | |
| NM_0142 | 109 | 471 | AAACAGAACCUCGAUU | 496 | AGUAAUCGAGGUUCUGU |
| 75.4_109 | 9 | | ACU | | UU |
| 9-1117 | | | | | |
| NM_0142 | 110 | 472 | CAGAACCUCGAUUACU | 497 | AGCAGUAAUCGAGGUUC |
| 75.4_110 | 2 | | GCU | | UG |
| 2-1120 | | | | | |
| NM_0142 | 110 | 473 | AGAACCUCGAUUACUG | 498 | AAGCAGUAAUCGAGGUU |
| 75.4_110 | 3 | | CUU | | CU |
| 3-1121 | | | | | |
| NM_0142 | 112 | 474 | UCCUCAUGAUGUACGC | 499 | UGCGCGUACAUCAUGAG |
| 75.4_112 | 1 | | GCA | | GA |
| 1-1139 | | | | | |
| NM_0142 | 130 | 475 | GCCUGAUUGUAGAGUU | 500 | AUGAACUCUACAAUCAG |
| 75.4_130 | 7 | | CAU | | GC |
| 7-1325 | | | | | |
| NM_0142 | 138 | 476 | AAAGUCUGCAACCCCG | 501 | UCUCGGGGUUGCAGACU |
| 75.4_138 | 4 | | AGA | | UU |
| 4-1402 | | | | | |
| NM_0142 | 139 | 477 | GAGAAGGAUGCGAAGC | 502 | AGUGCUUCGCAUCCUUC |
| 75.4_139 | 9 | | ACU | | UC |
| 9-1417 | | | | | |
| NM_0142 | 178 | 478 | GACAACCCUCAGUCAG | 503 | UGUCUGACUGAGGGUUG |
| 75.4_178 | 9 | | ACA | | UC |
| 9-1807 | | | | | |
| NM_0142 | 197 | 479 | UGGGUGAUUCUGAGCG | 504 | UCUCGCUCAGAAUCACC |
| 75.4_197 | 5 | | AGA | | CA |
| 5-1993 | | | | | |
| NM_0142 | 228 | 480 | AGGCCGUUUUAGAAGA | 505 | AGCUCUUCUAAAACGGC |
| 75.4_228 | 0 | | GCU | | CU |
| 0-2298 | | | | | |
| NM_0142 | 228 | 481 | GCCGUUUUAGAAGAGC | 506 | AAAGCUCUUCUAAAACG |
| 75.4_228 | 2 | | UUU | | GC |
| 2-2300 | | | | | |
| NM_0142 | 239 | 482 | UUCACGUAAGUCCACA | 507 | AUAUGUGGACUUACGUG |
| 75.4_239 | 2 | | UAU | | AA |
| 2-2410 | | | | | |
| NM_0142 | 239 | 483 | UCACGUAAGUCCACAU | 508 | UAUAUGUGGACUUACGU |
| 75.42_39 | 3 | | AUA | | GA |
| 3-2411 | | | | | |
| NM_0142 | 239 | 484 | CGUAAGUCCACAUAUA | 509 | AAGUAUAUGUGGACUUA |
| 75.4_239 | 6 | | CUU | | CG |
| 6-2414 | | | | | |
| NM_0142 | 240 | 485 | UAUACUUCUAUAAGAG | 510 | ACGCUCUUAUAGAAGUA |
| 75.4_240 | 8 | | CGU | | UA |
| 8-2426 | | | | | |
| NM_0142 | 241 | 486 | UAUAAGAGCGUGACUU | 511 | UACAAGUCACGCUCUUA |
| 75.4_241 | 6 | | GUA | | UA |
| 6-2434 | | | | | |
| NM_0142 | 241 | 487 | UAAGAGCGUGACUUGU | 512 | AUUACAAGUCACGCUCU |
| 75.4_241 | 8 | | AAU | | UA |
| 8-2436 | | | | | |
| NM_0142 | 242 | 488 | AGAGCGUGACUUGUAA | 513 | UUAUUACAAGUCACGCU |
| 75.4_242 | 0 | | UAA | | CU |
| 0-2438 | | | | | |
| NM_0142 | 244 | 489 | GUUAAUGAAGUGUGU | 514 | AGGCACACACUUCAUUA |
| 75.4_244 | 2 | | GCCU | | AC |
| 2-1460 | | | | | |

TABLE 11
RNA effector molecules targeting GNE
(mouse; NM_015828.3; SEQ ID NO: 515)

| | | SEQ | | SEQ | |
|---|---|---|---|---|---|
| Oligo | Start | ID | Sense Sequence | ID | Antisense Sequence |
| Name | Pos. | NO. | 5′ to 3′ | NO. | 5′ to 3′ |
| NM_0158 | 79 | 516 | GAUGGAAACACACGCG | 542 | AUGCGCGUGUGUUUCCA |
| 28.3_79- | | | CAU | | UC |
| 97 | | | | | |
| NM_0158 | 252 | 517 | CGAUCAUGUUCGGCAU | 543 | UUGAUGCCGAACAUGAU |
| 28.3_252- | | | CAA | | CG |
| 270 | | | | | |
| NM_0158 | 315 | 518 | CCCACCUGAUUGACGA | 544 | UAGUCGUCAAUCAGGUG |
| 28.3_315- | | | CUA | | GG |
| 333 | | | | | |
| NM_0158 | 316 | 519 | CCACCUGAUUGACGAC | 545 | AUAGUCGUCAAUCAGGU |
| 28.3_316- | | | UAU | | GG |
| 334 | | | | | |
| NM_0158 | 336 | 520 | GAAACACAUACCGCAU | 546 | AUCAUGCGGUAUGUGUU |
| 28.3_336- | | | GAU | | UC |
| 354 | | | | | |
| NM_0158 | 462 | 521 | ACGUCCUCAAUCGCCU | 547 | UUCAGGCGAUUGAGGAC |
| 28.3_462- | | | GAA | | GU |
| 480 | | | | | |
| NM_0158 | 762 | 522 | AUAUGAGCAUCAUUCG | 548 | AUCCGAAUGAUGCUCAU |
| 28.3_762- | | | GAU | | AU |
| 780 | | | | | |
| NM_0158 | 782 | 523 | UGGCUAGGCGAUGAUG | 549 | UUACAUCAUCGCCUAGC |
| 28.3_782- | | | UAA | | CA |
| 800 | | | | | |
| NM_0158 | 846 | 524 | ACAUUAAGCAUUCCAU | 550 | UUUAUGGAAUGCUUAAU |
| 28.3_846- | | | AAA | | GU |
| 864 | | | | | |
| NM_0158 | 882 | 525 | UGGAUGCCCUGAUCUC | 551 | AACGAGAUCAGGGCAUC |
| 28.3_882- | | | GUU | | CA |
| 900 | | | | | |
| NM_0158 | 888 | 526 | CCCUGAUCUCGUUUAA | 552 | UUGUUAAACGAGAUCAG |
| 28.3_888- | | | CAA | | GG |
| 906 | | | | | |
| NM_0158 | 100 | 527 | AGUCAAGCACGUCCCG | 553 | AAACGGGACGUGCUUGA |
| 28.3_100 | 6 | | UUU | | CU |
| 6-1024 | | | | | |
| NM_0158 | 110 | 528 | UCGGAACACCCGUGAU | 554 | UUGAUCACGGGUGUUCC |
| 28.3_110 | 4 | | CAA | | GA |
| 4-1122 | | | | | |
| NM_0158 | 121 | 529 | UACACCUCCAGUUCGG | 555 | UUGCCGAACUGGAGGUG |
| 28.3_121 | 2 | | CAA | | UA |
| 2-1230 | | | | | |
| NM_0158 | 147 | 530 | ACUCAGUUCAACCCUA | 556 | UUUUAGGGUUGAACUGA |
| 28.3_147 | 5 | | AAA | | GU |
| 5-1493 | | | | | |
| NM_0158 | 213 | 531 | UGAACAUCCUCCACAC | 557 | AUAGUGUGGAGGAUGU |
| 28.3_213 | 3 | | UAU | | UCA |
| 3-2151 | | | | | |
| NM_0158 | 325 | 532 | GGGAAGGCUUAGUUU | 558 | UAGUAAACUAAGCCUUC |
| 28.3_325 | 0 | | ACUA | | CC |
| 0-3268 | | | | | |
| NM_0158 | 325 | 533 | GCUUAGUUUACUAGUC | 559 | AAGGACUAGUAAACUAA |
| 28.3_325 | 6 | | CUU | | GC |
| 6-3274 | | | | | |
| NM_0158 | 353 | 534 | UCCAAGUUAUGACGGC | 560 | AAGGCCGUCAUAACUUG |
| 28.3_353 | 2 | | CUU | | GA |
| 2-3550 | | | | | |
| NM_0158 | 353 | 535 | CCAAGUUAUGACGGCC | 561 | UAAGGCCGUCAUAACUU |
| 28.3_353 | 3 | | UUA | | GG |
| 3-3551 | | | | | |
| NM_0158 | 376 | 536 | UGUCUCUGUAAUCUCG | 562 | AAGCGAGAUUACAGAGA |
| 28.3_376 | 2 | | CUU | | CA |
| 2-3780 | | | | | |
| NM_0158 | 419 | 537 | AGGAGCACGUACCGAC | 563 | UAUGUCGGUACGUGCUC |
| 28.3_419 | 2 | | AUA | | CU |
| 2-4210 | | | | | |
| NM_0158 | 469 | 538 | UUACUAUAGUUCCACG | 564 | AUACGUGGAACUAUAGU |
| 28.3_469 | 8 | | UAU | | AA |
| 8-4716 | | | | | |
| NM_0158 | 485 | 539 | GAUCUUCCGACCCCAC | 565 | UUUGUGGGGUCGGAAGA |
| 28.3_485 | 3 | | AAA | | UC |
| 3-4871 | | | | | |
| NM_0158 | 489 | 540 | GAGAACUGCUGCCGUC | 566 | AUUGACGGCAGCAGUUC |
| 28.3_489 | 2 | | AAU | | UC |
| 2-4910 | | | | | |
| NM_0158 | 535 | 541 | CUAUUAUAAAGGACCA | 567 | UAAUGGUCCUUUAUAAU |
| 28.3_535 | 8 | | UUA | | AG |
| 8-5376 | | | | | |

TABLE 12
RNA effector molecules targeting MGAT1
(mouse; NM_010794.3; SEQ ID NO: 568)

| | | SEQ | | SEQ | |
|---|---|---|---|---|---|
| Oligo | Start | ID | Sense Sequence | ID | Antisense Sequence |
| Name | Pos. | NO. | 5′ to 3′ | NO. | 5′ to 3′ |
| NM_0107 | 918 | 569 | AUCAAGGAGCAUUAUG | 596 | AAGCAUAAUGCUCCUUG |
| 94.3_918- | | | CUU | | AU |
| 936 | | | | | |
| NM_0107 | 919 | 570 | UCAAGGAGCAUUAUGC | 597 | AAAGCAUAAUGCUCCUU |
| 94.3_919- | | | UUU | | GA |
| 937 | | | | | |
| NM_0107 | 105 | 571 | GGCGCUGCUUGGAUAA | 598 | AACUUAUCCAAGCAGCG |
| 94.3_105 | 7 | | GUU | | CC |
| 7-1075 | | | | | |
| NM_0107 | 119 | 572 | ACCUGAGUAACAUUGC | 599 | ACGGCAAUGUUACUCAG |
| 94.3_119 | 8 | | CGU | | GU |
| 8-1216 | | | | | |
| NM_0107 | 144 | 573 | GAGCAGAUGGUAGACU | 600 | UUGAGUCUACCAUCUGC |
| 94.3_144 | 0 | | CAA | | UC |
| 0-1458 | | | | | |
| NM_0107 | 146 | 574 | UGAGCUGCUCUAUCGA | 601 | UGUUCGAUAGAGCAGCU |
| 94.3_146 | 6 | | ACA | | CA |
| 6-1484 | | | | | |
| NM_0107 | 147 | 575 | GCUCUAUCGAACAGAC | 602 | AAAGUCUGUUCGAUAGA |
| 94.3_147 | 2 | | UUU | | GC |
| 2-1490 | | | | | |
| NM_0107 | 159 | 576 | AGGACGGGCUUGUAUU | 603 | ACGAAUACAAGCCCGUC |
| 94.3_159 | 8 | | CGU | | CU |
| 8-1616 | | | | | |
| NM_0107 | 160 | 577 | GGGCUUGUAUUCGUCC | 604 | UCUGGACGAAUACAAGC |
| 94.3_160 | 3 | | AGA | | CC |
| 3-1621 | | | | | |
| NM_0107 | 160 | 578 | GGCUUGUAUUCGUCCA | 605 | UUCUGGACGAAUACAAG |
| 94.3_160 | 4 | | GAA | | CC |
| 4-1622 | | | | | |
| NM_0107 | 163 | 579 | ACUAUGACCUUUGGUC | 606 | UGCGACCAAAGGUCAUA |
| 94.3_163 | 2 | | GCA | | GU |
| 2-1650 | | | | | |
| NM_0107 | 181 | 580 | GUAAGGACCAAUGAUC | 607 | UCUGAUCAUUGGUCCUU |
| 94.3_181 | 8 | | AGA | | AC |
| 8-1836 | | | | | |
| NM_0107 | 185 | 581 | GUGCGGGUACAGUACA | 608 | UAGUGUACUGUACCCGC |
| 94.3_185 | 1 | | CUA | | AC |
| 1-1869 | | | | | |
| NM_0107 | 219 | 582 | UUCUAGUGCACAAAUC | 609 | UAUGAUUUGUGCACUAG |
| 94.3_219 | 9 | | AUA | | AA |
| 9-2217 | | | | | |
| NM_0107 | 221 | 583 | AAUCAUAGGAUGAGA | 610 | UAACUCUCAUCCUAUGA |
| 94.3_221 | 1 | | GUUA | | UU |
| 1-2229 | | | | | |
| NM_0107 | 222 | 584 | UGAGAGUUAUACUCCU | 611 | AACAGGAGUAUAACUCU |
| 94.3_222 | 1 | | GUU | | CA |
| 1-2239 | | | | | |
| NM_0107 | 223 | 585 | CCUGUUGUCAAGGGAG | 612 | AUACUCCCUUGACAACA |
| 94.3_223 | 4 | | UAU | | GG |
| 4-2252 | | | | | |
| NM_0107 | 223 | 586 | CUGUUGUCAAGGGAGU | 613 | AAUACUCCCUUGACAAC |
| 94.3_223 | 5 | | AUU | | AG |
| 5-2253 | | | | | |
| NM_0107 | 225 | 587 | GUGGUAUGUUCGGGGC | 614 | UAUGCCCCGAACAUACC |
| 94.3_225 | 6 | | AUA | | AC |
| 6-2274 | | | | | |
| NM_0107 | 241 | 588 | GCCCAUGAGCCCUCUU | 615 | AUAAAGAGGGCUCAUGG |
| 94.3_241 | 7 | | UAU | | GC |
| 7-2435 | | | | | |
| NM_0107 | 253 | 589 | CUGUCAGAGUUAGCGU | 616 | UCCACGCUAACUCUGAC |
| 94.3_253 | 3 | | GGA | | AG |
| 3-2551 | | | | | |
| NM_0107 | 253 | 590 | UGUCAGAGUUAGCGUG | 617 | AUCCACGCUAACUCUGA |
| 94.3_253 | 4 | | GAU | | CA |
| 4-2552 | | | | | |
| NM_0107 | 253 | 591 | GUCAGAGUUAGCGUGG | 618 | AAUCCACGCUAACUCUG |
| 94.3_253 | 5 | | AUU | | AC |
| 5-2553 | | | | | |
| NM_0107 | 253 | 592 | AGAGUUAGCGUGGAU | 619 | AGCAAUCCACGCUAACU |
| 94.3_253 | 8 | | UGCU | | CU |
| 8-2556 | | | | | |
| NM_0107 | 261 | 593 | AGCAGAGCAGUGCGUU | 620 | AACAACGCACUGCUCUG |
| 94.3_261 | 3 | | GUU | | CU |
| 3-2631 | | | | | |
| NM_0107 | 263 | 594 | AGAUGGAAAGUCUAU | 621 | ACGCAUAGACUUUCCAU |
| 94.3_263 | 5 | | GCGU | | CU |
| 5-2653 | | | | | |
| NM_0107 | 263 | 595 | GGAAAGUCUAUGCGUG | 622 | ACCCACGCAUAGACUUU |
| 94.3_263 | 9 | | GGU | | CC |
| 9-2657 | | | | | |

TABLE 13
RNA effector molecules targeting MGAT4A
(mouse; NM_173870.2; SEQ ID NO: 623)

| | | SEQ | | SEQ | |
|---|---|---|---|---|---|
| Oligo | Start | ID | Sense Sequence | ID | Antisense Sequence |
| Name | Pos. | NO. | 5′ to 3′ | NO. | 5′ to 3′ |
| NM_1738 | 525 | 624 | CGGGAGUUUCAAUAGU | 645 | AUAACUAUUGAAACUCC |
| 70.2_525- | | | UAU | | CG |
| 543 | | | | | |
| NM_1738 | 901 | 625 | GCUUGAAGACGAUAUU | 646 | AAUAAUAUCGUCUUCAA |
| 70.2_901- | | | AUU | | GC |
| 919 | | | | | |
| NM_1738 | 116 | 626 | CAGAAGGCAAACCUAC | 647 | UUCGUAGGUUUGCCUUC |
| 70.2_116 | 3 | | GAA | | UG |
| 3-1181 | | | | | |
| NM_1738 | 165 | 627 | CUUUCGACUUUCCGUU | 648 | AAUAACGGAAAGUCGAA |
| 70.2_165 | 7 | | AUU | | AG |
| 7-1675 | | | | | |
| NM_1738 | 233 | 628 | CAUUAUAGAUCAACGC | 649 | AUGGCGUUGAUCUAUAA |
| 70.2_233 | 7 | | CAU | | UG |
| 7-2355 | | | | | |
| NM_1738 | 250 | 629 | CGAGGGUUAUAUUACU | 650 | UACAGUAAUAUAACCCU |
| 70.2_250 | 3 | | GUA | | CG |
| 3-2521 | | | | | |
| NM_1738 | 290 | 630 | GUCCCAAGUAGCGCUA | 651 | AACUAGCGCUACUUGGG |
| 70.2_290 | 9 | | GUU | | AC |
| 9-2927 | | | | | |
| NM_1738 | 327 | 631 | CACAAAUAAAUCCCAC | 652 | AUAGUGGGAUUUAUUU |
| 70.2_327 | 2 | | UAU | | GUG |
| 2-3290 | | | | | |
| NM_1738 | 400 | 632 | GUGUCAAAUGCAGUAC | 653 | UUAGUACUGCAUUUGAC |
| 70.2_400 | 3 | | UAA | | AC |
| 3-4021 | | | | | |
| NM_1738 | 400 | 633 | GUCAAAUGCAGUACUA | 654 | AAUUAGUACUGCAUUUG |
| 70.2_400 | 5 | | AUU | | AC |
| 5-4023 | | | | | |
| NM_1738 | 434 | 634 | GGUGGGACAGUCAAGA | 655 | UAAUCUUGACUGUCCCA |
| 70.2_434 | 6 | | UUA | | CC |
| 6-4364 | | | | | |
| NM_1738 | 435 | 635 | CAAGAUUACCCGGCUA | 656 | AUGUAGCCGGGUAAUCU |
| 70.2_435 | 7 | | CAU | | UG |
| 7-4375 | | | | | |
| NM_1738 | 441 | 636 | CUCAAGAGGUACGUUU | 657 | UUCAAACGUACCUCUUG |
| 70.2_441 | 8 | | GAA | | AG |
| 8-4436 | | | | | |
| NM_1738 | 453 | 637 | CAUUGGUUCUUGAAAU | 658 | AUGAUUUCAAGAACCAA |
| 70.2_453 | 1 | | CAU | | UG |
| 1-4549 | | | | | |
| NM_1738 | 470 | 638 | GGCAAAGGUUGUUCUA | 659 | AACUAGAACAACCUUUG |
| 70.2_470 | 2 | | GUU | | CC |
| 2-4720 | | | | | |
| NM_1738 | 523 | 639 | CCUGCGGUAGACUAGU | 660 | AAAACUAGUCUACCGCA |
| 70.2_523 | 0 | | UUU | | GG |
| 0-5248 | | | | | |
| NM_1738 | 549 | 640 | CUAGAUGCUCCUAAAU | 661 | AUAAUUUAGGAGCAUCU |
| 70.2_549 | 8 | | UAU | | AG |
| 8-5516 | | | | | |
| NM_1738 | 591 | 641 | CAAGCUAUUAACUGCU | 662 | AUUAGCAGUUAAUAGCU |
| 70.2_591 | 5 | | AAU | | UG |
| 5-5933 | | | | | |
| NM_1738 | 607 | 642 | GGGCAUUCCGAGCUAU | 663 | UACAUAGCUCGGAAUGC |
| 70.2_607 | 7 | | GUA | | CC |
| 7-6095 | | | | | |
| NM_1738 | 615 | 643 | CAUAUAUGCUACGCUA | 664 | AAUUAGCGUAGCAUAUA |
| 70.2_615 | 6 | | AUU | | UG |
| 6-6174 | | | | | |
| NM_1738 | 659 | 644 | GCUGGUAAGCGUACCU | 665 | UUCAGGUACGCUUACCA |
| 70.2_659 | 1 | | GAA | | GC |
| 1-6609 | | | | | |

TABLE 14
RNA effector molecules targeting MGAT4B (mouse; NM_145926.2;
SEQ ID NO: 666)

| Oligo | Start | SEQ ID | Sense Sequence | SEQ ID | Antisense Sequence |
|---|---|---|---|---|---|
| Name | Pos. | NO. | 5′ to 3′ | NO. | 5′ to 3′ |
| NM_145926.2_ | 123 | 667 | UUUACCAGCGCGAGUU | 696 | AGGAACUCGCGCUGGUA |
| 123-141 | | | CCU | | AA |
| NM_145926.2_ | 260 | 668 | GGAGAAGGCAAUCGCA | 697 | AAGUGCGAUUGCCUUCU |
| 260-278 | | | CUU | | CC |
| NM_145926.2_ | 435 | 669 | UAUCCGUGGUGAUGGG | 698 | AUGCCCAUCACCACGGA |
| 435-453 | | | CAU | | UA |
| NM_145926.2_ | 436 | 670 | AUCCGUGGUGAUGGGC | 699 | AAUGCCCAUCACCACGG |
| 436-454 | | | AUU | | AU |
| NM_145926.2_ | 470 | 671 | GAGGUGCACUCGUACU | 700 | UCAAGUACGAGUGCACC |
| 470-488 | | | UGA | | UC |
| NM_145926.2_ | 479 | 672 | UCGUACUUGACUGACA | 701 | AUGUGUCAGUCAAGUAC |
| 479-497 | | | CAU | | GA |
| NM_145926.2_ | 580 | 673 | GUACACUUCGGCAGUG | 702 | UGUCACUGCCGAAGUGU |
| 580-598 | | | ACA | | AC |
| NM_145926.2_ | 737 | 674 | ACCAAACAGAACCUCG | 703 | AAUCGAGGUUCUGUUUG |
| 737-755 | | | AUU | | GU |
| NM_145926.2_ | 744 | 675 | AGAACCUCGAUUACUG | 704 | AAGCAGUAAUCGAGGUU |
| 744-762 | | | CUU | | CU |
| NM_145926.2_ | 780 | 676 | AGUCCAAAGGCAUCUA | 705 | UAGUAGAUGCCUUUGGA |
| 780-798 | | | CUA | | CU |
| NM_145926.2_ | 890 | 677 | AUCCUGGAGUUCUCGC | 706 | ACUGCGAGAACUCCAGG |
| 890-908 | | | AGU | | AU |
| NM_145926.2_ | 897 | 678 | AGUUCUCGCAGUUGGG | 707 | AAGCCCAACUGCGAGAA |
| 897-915 | | | CUU | | CU |
| NM_145926.2_ | 1040 | 679 | GAGAAGGAUGCGAAAC | 708 | AAUGUUUCGCAUCCUUC |
| 1040-1058 | | | AUU | | UC |
| NM_145926.2_ | 1042 | 680 | GAAGGAUGCGAAACAU | 709 | ACAAUGUUUCGCAUCCU |
| 1042-1060 | | | UGU | | UC |
| NM_145926.2_ | 1052 | 681 | AAACAUUGUGAUCGGC | 710 | UCUGCCGAUCACAAUGU |
| 1052-1070 | | | AGA | | UU |
| NM_145926.2_ | 1053 | 682 | AACAUUGUGAUCGGCA | 711 | UUCUGCCGAUCACAAUG |
| 1053-1071 | | | GAA | | UU |
| NM_145926.2_ | 1128 | 683 | CAUCACUGGCGGGCAA | 712 | AUUUUGCCCGCCAGUGA |
| 1128-1146 | | | AAU | | UG |
| NM_145926.2_ | 1221 | 684 | GCACAAGCCUCAAGAC | 713 | UACGUCUUGAGGCUUGU |
| 1221-1239 | | | GUA | | GC |
| NM_145926.2_ | 1224 | 685 | CAAGCCUCAAGACGUA | 714 | UGGUACGUCUUGAGGCU |
| 1224-1242 | | | CCA | | UG |
| NM_145926.2_ | 1265 | 686 | UACUUGCGGGAGGAUU | 715 | AGAAAUCCUCCCGCAAG |
| 1265-1283 | | | UCU | | UA |
| NM_145926.2_ | 1305 | 687 | CAGGAGACUUUAUCCG | 716 | AACCGGAUAAAGUCUCC |
| 1305-1323 | | | GUU | | UG |
| NM_145926.2_ | 1376 | 688 | AUCGAGCACCCGGAAG | 717 | UAUCUUCCGGGUGCUCG |
| 1376-1394 | | | AUA | | AU |
| NM_145926.2_ | 1377 | 689 | UCGAGCACCCGGAAGA | 718 | UUAUCUUCCGGGUGCUC |
| 1377-1395 | | | UAA | | GA |
| NM_145926.2_ | 1383 | 690 | ACCCGGAAGAUAAGCU | 719 | AAGAGCUUAUCUUCCGG |
| 1383-1401 | | | CUU | | GU |
| NM_145926.2_ | 1525 | 691 | CUUCUACAAGGGUGUA | 720 | AGCUACACCCUUGUAGA |
| 1525-1543 | | | GCU | | AG |
| NM_145926.2_ | 1574 | 692 | CUGGAAGCACUACGUC | 721 | AGAGACGUAGUGCUUCC |
| 1574-1592 | | | UCU | | AG |
| NM_145926.2_ | 1579 | 693 | AGCACUACGUCUCUCC | 722 | AAUGGAGAGACGUAGUG |
| 1579-1597 | | | AUU | | CU |
| NM_145926.2_ | 1610 | 694 | CCGGUGUGGGUCAUUU | 723 | UCAAAAUGACCCACACC |
| 1610-1628 | | | UGA | | GG |
| NM_145926.2 | 1617 | 695 | GGGUCAUUUUGAGUG | 724 | AUCUCACUCAAAAUGAC |
| 1617-1635 | | | AGAU | | CC |

TABLE 15
RNA effector molecules targeting GNE (rat; NM_053765.2;
SEQ ID NO: 725)

| Oligo | Start | SEQ ID | Sense Sequence | SEQ ID | Antisense Sequence |
|---|---|---|---|---|---|
| Name | Pos. | NO. | 5′ to 3′ | NO. | 5′ to 3′ |
| NM_053765.2_ | 347 | 726 | AGAACGGGAAUAACCG | 752 | UUCCGGUUAUUCCCGUU |
| 347-365 | | | GAA | | CU |
| NM_053765.2_ | 363 | 727 | GAAGCUUCGGGUUUGC | 753 | AACGCAAACCCGAAGCU |
| 363-381 | | | GUU | | UC |
| NM_053765.2_ | 374 | 728 | UUUGCGUUGCCACCUG | 754 | UUGCAGGUGGCAACGCA |
| 374-392 | | | CAA | | AA |
| NM_053765.2_ | 482 | 729 | CUCACCUGAUCGACGA | 755 | UAGUCGUCGAUCAGGUG |
| 482-500 | | | CUA | | AG |
| NM_053765.2_ | 545 | 730 | ACACCAGGCUACACAC | 756 | AUCGUGUGUAGCCUGGU |
| 545-563 | | | GAU | | GU |
| NM_053765.2_ | 549 | 731 | CAGGCUACACACGAUU | 757 | AACAAUCGUGUGUAGCC |
| 549-567 | | | GUU | | UG |
| NM_053765.2_ | 558 | 732 | CACGAUUGUUAGAGGG | 758 | UUCCCCUCUAACAAUCG |
| 558-576 | | | GAA | | UG |
| NM_053765.2_ | 942 | 733 | UCGGAUGUGGCUAGGU | 759 | AUCACCUAGCCACAUCC |
| 942-960 | | | GAU | | GA |
| NM_053765.2_ | 1006 | 734 | ACCACCGACAUUAAGC | 760 | AAUGCUUAAUGUCGGUG |
| 1006-1024 | | | AUU | | GU |
| NM_053765.2_ | 1011 | 735 | CGACAUUAAGCAUUCC | 761 | UAUGGAAUGCUUAAUGU |
| 1011-1029 | | | AUA | | CG |
| NM_053765.2_ | 1160 | 736 | CCAAUUUCCGCGCAGU | 762 | UUGACUGCGCGGAAAUU |
| 1160-1178 | | | CAA | | GG |
| NM_053765.2_ | 1173 | 737 | AGUCAAGCACGUCCCG | 763 | AAACGGGACGUGCUUGA |
| 1173-1191 | | | UUU | | CU |
| NM_053765.2_ | 1379 | 738 | UACACCUCCAGUUCGG | 764 | UUACCGAACUGGAGGUG |
| 1379-1397 | | | UAA | | UA |
| NM_053765.2_ | 1390 | 739 | UUCGGUAAACAGUACC | 765 | AAGGGUACUGUUUACCG |
| 1390-1408 | | | CUU | | AA |
| NM_053765.2_ | 1440 | 740 | UCCAAGGAUUUUAAAG | 766 | AAACUUUAAAAUCCUUG |
| 1440-1458 | | | UUU | | GA |
| NM_053765.2_ | 1589 | 741 | CGAAUCUGAGAGUGGC | 767 | AUCGCCACUCUCAGAUU |
| 1589-1607 | | | GAU | | CG |
| NM_053765.2_ | 1590 | 742 | GAAUCUGAGAGUGGCG | 768 | UAUCGCCACUCUCAGAU |
| 1590-1608 | | | AUA | | UC |
| NM_053765.2_ | 1594 | 743 | CUGAGAGUGGCGAUAG | 769 | UAACUAUCGCCACUCUC |
| 1594-1612 | | | UUA | | AG |
| NM_053765.2_ | 1601 | 744 | UGGCGAUAGUUAGCAU | 770 | UUCAUGCUAACUAUCGC |
| 1601-1619 | | | GAA | | CA |
| NM_053765.2_ | 1610 | 745 | UUAGCAUGAAGGGUG | 771 | AUUUCACCCUUCAUGCU |
| 1610-1628 | | | AAAU | | AA |
| NM_053765.2_ | 1667 | 746 | AGGAAAGGAUUAGUC | 772 | AUUAGACUAAUCCUUUC |
| 1667-1685 | | | UAAU | | CU |
| NM_053765.2_ | 1943 | 747 | AGAACUUUGUGACGCU | 773 | AUGAGCGUCACAAAGUU |
| 1943-1961 | | | CAU | | CU |
| NM_053765.2_ | 2551 | 748 | AGUGGAACCACGCUCU | 774 | AAGAGAGCGUGGUUCCA |
| 2551-2569 | | | CUU | | CU |
| NM_053765.2_ | 2765 | 749 | CUCUCCAGAAUACACG | 775 | UUACGUGUAUUCUGGAG |
| 2765-2783 | | | UAA | | AG |
| NM_053765.2_ | 2766 | 750 | UCUCCAGAAUACACGU | 776 | UUUACGUGUAUUCUGGA |
| 2766-2784 | | | AAA | | GA |
| NM_053765.2_ | 2768 | 751 | UCCAGAAUACACGUAA | 777 | AAUUUACGUGUAUUCUG |
| 2768-2786 | | | AUU | | GA |

TABLE 16
RNA effector molecules targeting MGAT1 (rat; NM_030861.1;
SEQ ID NO: 778)

| Oligo | Start | SEQ ID | Sense Sequence | SEQ ID | Antisense Sequence |
|---|---|---|---|---|---|
| Name | Pos. | NO. | 5′ to 3′ | NO. | 5′ to 3′ |
| NM_030861.1_ | 700 | 779 | CCAGGGUUACUACAAG | 803 | AAUCUUGUAGUAACCCU |
| 700-718 | | | AUU | | GG |
| NM_030861.1_ | 933 | 780 | UGCUCUAUCGAACAGA | 804 | AAGUCUGUUCGAUAGAG |
| 933-951 | | | CUU | | CA |
| NM_030861.1_ | 934 | 781 | GCUCUAUCGAACAGAC | 805 | AAAGUCUGUUCGAUAGA |
| 934-952 | | | UUU | | GC |
| NM_030861.1_ | 1066 | 782 | GGCUUGUAUUCGUCCA | 806 | UUCUGGACGAAUACAAG |
| 1066-1084 | | | GAA | | CC |
| NM_030861.1_ | 1281 | 783 | UGAGGACCAAUGAUCG | 807 | UUCCGAUCAUUGGUCCU |
| 1281-1299 | | | GAA | | CA |
| NM_030861.1_ | 1313 | 784 | GUGCGGGUACAGUACA | 808 | UAGUGUACUGUACCCGC |
| 1313-1331 | | | CUA | | AC |
| NM_030861.1_ | 1335 | 785 | GAGACAGCUUUAAGGC | 809 | AAGGCCUUAAAGCUGUC |
| 1335-1353 | | | CUU | | UC |
| NM_030861.1_ | 1472 | 786 | UGGAAUGGCUAUGAUC | 810 | UAGGAUCAUAGCCAUUC |
| 1472-1490 | | | CUA | | CA |
| NM_030861.1_ | 1594 | 787 | UCUUGGAUCUAUAGAU | 811 | AAGAUCUAUAGAUCCAA |
| 1594-1612 | | | CUU | | GA |
| NM_030861.1_ | 1639 | 788 | GUGGCAUUCUAGUGCA | 812 | UUGUGCACUAGAAUGCC |
| 1639-1657 | | | CAA | | AC |
| NM_030861.1_ | 1640 | 789 | UGGCAUUCUAGUGCAC | 813 | UUUGUGCACUAGAAUGC |
| 1640-1658 | | | AAA | | CA |
| NM_030861.1_ | 1641 | 790 | GGCAUUCUAGUGCACA | 814 | AUUUGUGCACUAGAAUG |
| 1641-1659 | | | AAU | | CC |
| NM_030861.1_ | 1646 | 791 | UCUAGUGCACAAAUCA | 815 | UUAUGAUUUGUGCACUA |
| 1646-1664 | | | UAA | | GA |
| NM_030861.1_ | 1649 | 792 | AGUGCACAAAUCAUAA | 816 | AUCUUAUGAUUUGUGCA |
| 1649-1667 | | | GAU | | CU |
| NM_030861.1_ | 1701 | 793 | UGUGGUAUGUUCGGG | 817 | AUGCCCCGAACAUACCA |
| 1701-1719 | | | GCAU | | CA |
| NM_030861.1_ | 1702 | 794 | GUGGUAUGUUCGGGGC | 818 | UAUGCCCCGAACAUACC |
| 1702-1720 | | | AUA | | AC |
| NM_030861.1_ | 1869 | 795 | UUAGUACAUGAGCCCA | 819 | AAGUGGGCUCAUGUACU |
| 1869-1887 | | | CUU | | AA |
| NM_030861.1_ | 1954 | 796 | UUGUGGCCAACUGAGA | 820 | UAGUCUCAGUUGGCCAC |
| 1954-1972 | | | CUA | | AA |
| NM_030861.1_ | 1979 | 797 | GGGAUUCACUGUCAGA | 821 | AACUCUGACAGUGAAUC |
| 1979-1997 | | | GUU | | CC |
| NM_030861.1_ | 1997 | 798 | UAGAUUGCAUGGCCGG | 822 | AACCCGGCCAUGCAAUC |
| 1997-2015 | | | GUU | | UA |
| NM_030861.1_ | 2209 | 799 | CCACUUCUGAAAGUUA | 823 | AACUAACUUUCAGAAGU |
| 2209-2227 | | | GUU | | GG |
| NM_030861.1_ | 2219 | 800 | AAGUUAGUUCCCUUUG | 824 | AUGCAAAGGGAACUAAC |
| 2219-2237 | | | CAU | | UU |
| NM_030861.1_ | 2389 | 801 | UUCCUAAGCAUUCCCA | 825 | AUGUGGGAAUGCUUAGG |
| 2389-2407 | | | CAU | | AA |
| NM_030861.1_ | 2392 | 802 | CUAAGCAUUCCCACAU | 826 | AAGAUGUGGGAAUGCUU |
| 2392-2410 | | | CUU | | AG |

TABLE 17
RNA effector molecules targeting MGAT4A (rat; NM_001012225.2;
SEQ ID NO: 827)

| Oligo | Start | SEQ ID | Sense Sequence | SEQ ID | Antisense Sequence |
|---|---|---|---|---|---|
| Name | Pos. | NO. | 5′ to 3′ | NO. | 5′ to 3′ |
| NM_001012225.2_ | 2469 | 828 | AUUCUAGAUCAACGCC | 850 | AAUGGCGUUGAUCUAGA |
| 2469-2487 | | | AUU | | AU |
| NM_001012225.2_ | 2470 | 829 | UUCUAGAUCAACGCCA | 851 | AAAUGGCGUUGAUCUAG |
| 2470-2488 | | | UUU | | AA |
| NM_001012225.2_ | 2501 | 830 | CUGCCAUUCCAGAUUG | 852 | AACCAAUCUGGAAUGGC |
| 2501-2519 | | | GUU | | AG |
| NM_001012225.2_ | 2677 | 831 | CACAGAAACCCGUGUG | 853 | AAUCACACGGGUUUCUG |
| 2677-2695 | | | AUU | | UG |
| NM_001012225.2_ | 2901 | 832 | GGAUGAGUCAUAUUG | 854 | UACCCAAUAUGACUCAU |
| 2901-2919 | | | GGUA | | CC |
| NM_001012225.2_ | 2905 | 833 | GAGUCAUAUUGGGUA | 855 | AUUAUACCCAAUAUGAC |
| 2905-2923 | | | UAAU | | UC |
| NM_001012225.2_ | 3727 | 834 | UGGAUGAGUGGAUUA | 856 | UAGUUAAUCCACUCAUC |
| 3727-3745 | | | ACUA | | CA |
| NM_001012225.2_ | 4585 | 835 | UAGCCUAGAUGUGUGU | 857 | AUAACACACAUCUAGGC |
| 4585-4603 | | | UAU | | UA |
| NM_001012225.2_ | 5502 | 836 | UAGCCAGACUUGCGUU | 858 | AAUAACGCAAGUCUGGC |
| 5502-5520 | | | AUU | | UA |
| NM_001012225.2_ | 5503 | 837 | AGCCAGACUUGCGUUA | 859 | AAAUAACGCAAGUCUGG |
| 5503-5521 | | | UUU | | CU |
| NM_001012225.2_ | 5575 | 838 | UCUCCUAGAUACUCCU | 860 | UUUAGGAGUAUCUAGGA |
| 5575-5593 | | | AAA | | GA |
| NM_001012225.2_ | 5576 | 839 | CUCCUAGAUACUCCUA | 861 | AUUUAGGAGUAUCUAGG |
| 5576-5594 | | | AAU | | AG |
| NM_001012225.2_ | 6145 | 840 | GUAGCAACAUUUACGG | 862 | AAGCCGUAAAUGUUGCU |
| 6145-6163 | | | CUU | | AC |
| NM_001012225.2_ | 6148 | 841 | GCAACAUUUACGGCUU | 863 | UACAAGCCGUAAAUGUU |
| 6148-6166 | | | GUA | | GC |
| NM_001012225.2_ | 6258 | 842 | CAUAUGAUGCUACAAA | 864 | AUCUUUGUAGCAUCAUA |
| 6258-6276 | | | GAU | | UG |
| NM_001012225.2_ | 6588 | 843 | ACAGCUAGGUACUUGU | 865 | AUCACAAGUACCUAGCU |
| 6588-6606 | | | GAU | | GU |
| NM_001012225.2_ | 6589 | 844 | CAGCUAGGUACUUGUG | 866 | UAUCACAAGUACCUAGC |
| 6589-6607 | | | AUA | | UG |
| NM_001012225.2_ | 6677 | 845 | CCUUUAGCUUGCGUAG | 867 | AUGCUACGCAAGCUAAA |
| 6677-6695 | | | CAU | | GG |
| NM_001012225.2_ | 6689 | 846 | GUAGCAUUCCACCCUU | 868 | AUAAAGGGUGGAAUGCU |
| 6689-6707 | | | UAU | | AC |
| NM_001012225.2_ | 6969 | 847 | UGAGGUACCUGUUGAA | 869 | AAAUUCAACAGGUACCU |
| 6969-6987 | | | UUU | | CA |
| NM_001012225.2_ | 7019 | 848 | UGCUAUAAUCGAAACC | 870 | UUAGGUUUCGAUUAUAG |
| 7019-7037 | | | UAA | | CA |
| NM_001012225.2_ | 7020 | 849 | GCUAUAAUCGAAACCU | 871 | AUUAGGUUUCGAUUAUA |
| 7020-7038 | | | AAU | | GC |

TABLE 18
RNA effector molecules targeting MGAT4B (rat; NM_001127533.1;
SEQ ID NO: 872)

| Oligo | Start | SEQ ID | Sense Sequence | SEQ ID | Antisense Sequence |
|---|---|---|---|---|---|
| Name | Pos. | NO. | 5′ to 3′ | NO. | 5′ to 3′ |
| NM_001127533.1_ | 395 | 873 | CACUGAGAGACCGUUU | 895 | UGCAAACGGUCUCUCAG |
| 395-413 | | | GCA | | UG |
| NM_001127533.1_ | 734 | 874 | ACUUGACUGACACAUU | 896 | UGCAAUGUGUCAGUCAA |
| 734-752 | | | GCA | | GU |
| NM_001127533.1_ | 814 | 875 | GCCGAGACUGAUACAC | 897 | ACUGUGUAUCAGUCUCG |
| 814-832 | | | AGU | | GC |
| NM_001127533.1_ | 870 | 876 | CCCCACAGAGAUCCAU | 898 | AGAAUGGAUCUCUGUGG |
| 870-888 | | | UCU | | GG |
| NM_001127533.1_ | 988 | 877 | ACCAAACAGAACCUCG | 899 | AAUCGAGGUUCUGUUUG |
| 988-1006 | | | AUU | | GU |
| NM_001127533.1_ | 991 | 878 | AAACAGAACCUCGAUU | 900 | AGUAAUCGAGGUUCUGU |
| 991-1009 | | | ACU | | UU |
| NM_001127533.1_ | 1013 | 879 | UCCUCAUGAUGUAUGC | 901 | UGUGCAUACAUCAUGAG |
| 1013-1031 | | | ACA | | GA |
| NM_001127533.1_ | 1210 | 880 | GAAUUCAUCCUUAUGU | 902 | AGAACAUAAGGAUGAAU |
| 1210-1228 | | | UCU | | UC |
| NM_001127533.1_ | 1287 | 881 | CCCUGAGAAGGAUGCG | 903 | UUUCGCAUCCUUCUCAG |
| 1287-1305 | | | AAA | | GG |
| NM_001127533.1_ | 1611 | 882 | UUUCCGAAGCGGGAAC | 904 | AAUGUUCCCGCUUCGGA |
| 1611-1629 | | | AUU | | AA |
| NM_001127533.1_ | 1613 | 883 | UCCGAAGCGGGAACAU | 905 | UCAAUGUUCCCGCUUCG |
| 1613-1631 | | | UGA | | GA |
| NM_001127533.1_ | 1634 | 884 | ACCCAGAAGAUAAGCU | 906 | AAGAGCUUAUCUUCUGG |
| 1634-1652 | | | CUU | | GU |
| NM_001127533.1_ | 1776 | 885 | CUUCUACAAGGGUGUA | 907 | AGCUACACCCUUGUAGA |
| 1776-1794 | | | GCU | | AG |
| NM_001127533.1_ | 2173 | 886 | GAACUGAACCGAACCG | 908 | AAACGGUUCGGUUCAGU |
| 2173-2191 | | | UUU | | UC |
| NM_001127533.1_ | 2259 | 887 | GCGGAACACUGGAAUG | 909 | AUGCAUUCCAGUGUUCC |
| 2259-2277 | | | CAU | | GC |
| NM_001127533.1_ | 2265 | 888 | CACUGGAAUGCAUACA | 910 | UAGUGUAUGCAUUCCAG |
| 2265-2283 | | | CUA | | UG |
| NM_001127533.1_ | 2324 | 889 | UUUUCACGUAAGUUCG | 911 | AUGCGAACUUACGUGAA |
| 2324-2342 | | | CAU | | AA |
| NM_001127533.1_ | 2329 | 890 | ACGUAAGUUCGCAUAU | 912 | AGUAUAUGCGAACUUAC |
| 2329-2347 | | | ACU | | GU |
| NM_001127533.1_ | 2336 | 891 | UUCGCAUAUACUUCUA | 913 | UUAUAGAAGUAUAUGCG |
| 2336-2354 | | | UAA | | AA |
| NM_001127533.1_ | 2351 | 892 | AUAAGAGCGUGACUUG | 914 | UUACAAGUCACGCUCUU |
| 2351-2369 | | | UAA | | AU |
| NM_001127533.1_ | 2365 | 893 | UGUAAUAAAGGGUUA | 915 | UCAUUAACCCUUUAUUA |
| 2365-2383 | | | AUGA | | CA |
| NM_001127533.1_ | 2366 | 894 | GUAAUAAAGGGUUAA | 916 | UUCAUUAACCCUUUAUU |
| 2366-2384 | | | UGAA | | AC |

TABLE 19
RNA effector molecules targeting SLC35A1 (rat; NM_001107924.1;
SEQ ID NO: 917)

| Oligo | Start | SEQ ID | Sense Sequence | SEQ ID | Antisense Sequence |
|---|---|---|---|---|---|
| Name | Pos. | NO. | 5′ to 3′ | NO. | 5′ to 3′ |
| NM_001107924.1_ | 63 | 918 | GCCGCUUAUACCAUAG | 943 | AAGCUAUGGUAUAAGCG |
| 63-81 | | | CUU | | GC |
| NM_001107924.1_ | 64 | 919 | CCGCUUAUACCAUAGC | 944 | AAAGCUAUGGUAUAAGC |
| 64-82 | | | UUU | | GG |
| NM_001107924.1_ | 70 | 920 | AUACCAUAGCUUUAAG | 945 | UAUCUUAAAGCUAUGGU |
| 70-88 | | | AUA | | AU |
| NM_001107924.1_ | 71 | 921 | UACCAUAGCUUUAAGA | 946 | AUAUCUUAAAGCUAUGG |
| 71-89 | | | UAU | | UA |
| NM_001107924.1_ | 72 | 922 | ACCAUAGCUUUAAGAU | 947 | UAUAUCUUAAAGCUAUG |
| 72-90 | | | AUA | | GU |
| NM_001107924.1_ | 102 | 923 | GCGGAAGGACUCUACU | 948 | AAAAGUAGAGUCCUUCC |
| 102-120 | | | UUU | | GC |
| NM_001107924.1_ | 158 | 924 | ACUGAUAAGUGUCGGC | 949 | AAGGCCGACACUUAUCA |
| 158-176 | | | CUU | | GU |
| NM_001107924.1_ | 166 | 925 | GUGUCGGCCUUCUAGC | 950 | UUAGCUAGAAGGCCGAC |
| 166-184 | | | UAA | | AC |
| NM_001107924.1_ | 191 | 926 | AGGCAGUUUGGGUAG | 951 | AAAUCUACCCAAACUGC |
| 191-209 | | | AUUU | | CU |
| NM_001107924.1_ | 193 | 927 | GCAGUUUGGGUAGAU | 952 | UUAAAUCUACCCAAACU |
| 193-211 | | | UUAA | | GC |
| NM_001107924.1_ | 446 | 928 | UGGUGGGGUCACACUU | 953 | UACAAGUGUGACCCCAC |
| 446-464 | | | GUA | | CA |
| NM_001107924.1_ | 669 | 929 | UUGUCGGAUGGCGCUG | 954 | UUUCAGCGCCAUCCGAC |
| 669-687 | | | AAA | | AA |
| NM_001107924.1_ | 672 | 930 | UCGGAUGGCGCUGAAA | 955 | UAAUUUCAGCGCCAUCC |
| 672-690 | | | UUA | | GA |
| NM_001107924.1_ | 703 | 931 | UUUUCUAUGGCUACAC | 956 | UACGUGUAGCCAUAGAA |
| 703-721 | | | GUA | | AA |
| NM_001107924.1_ | 706 | 932 | UCUAUGGCUACACGUA | 957 | UAAUACGUGUAGCCAUA |
| 706-724 | | | UUA | | GA |
| NM_001107924.1_ | 715 | 933 | ACACGUAUUAUGUCUG | 958 | AACCAGACAUAAUACGU |
| 715-733 | | | GUU | | GU |
| NM_001107924.1_ | 863 | 934 | UGGAUUGCAGAUAACA | 959 | AAGUGUUAUCUGCAAUC |
| 863-881 | | | CUU | | CA |
| NM_001107924.1_ | 1027 | 935 | GGACUAAACUGUUGAU | 960 | AUUAUCAACAGUUUAGU |
| 1027-1045 | | | AAU | | CC |
| NM_001107924.1_ | 1433 | 936 | UAUUCAAGCAACAACG | 961 | AAACGUUGUUGCUUGAA |
| 1433-1451 | | | UUU | | UA |
| NM_001107924.1_ | 1469 | 937 | UUCAAGUGCCAAAGCC | 962 | AACGGCUUUGGCACUUG |
| 1469-1487 | | | GUU | | AA |
| NM_001107924.1_ | 1681 | 938 | UCUAGGUGACGACUGA | 963 | UUCUCAGUCGUCACCUA |
| 1681-1699 | | | GAA | | GA |
| NM_001107924.1_ | 1744 | 939 | UUUGCUGUCAGCUGAU | 964 | UAUAUCAGCUGACAGCA |
| 1744-1762 | | | AUA | | AA |
| NM_001107924.1_ | 1783 | 940 | GCCGCUUUUAUACUUU | 965 | AAGAAAGUAUAAAAGCG |
| 1783-1801 | | | CUU | | GC |
| NM_001107924.1_ | 1784 | 941 | CCGCUUUUAUACUUUC | 966 | AAAGAAAGUAUAAAAGC |
| 1784-1802 | | | UUU | | GG |
| NM_001107924.1_ | 1807 | 942 | UAAAGUAUGGUUACCU | 967 | AACAGGUAACCAUACUU |
| 1807-1825 | | | GUU | | UA |

TABLE 20
RNA effector molecules targeting SLC35A1 (human; NM_006416.4;
SEQ ID NO: 968)

| Oligo | Start | SEQ ID | Sense Sequence | SEQ ID | Antisense Sequence |
|---|---|---|---|---|---|
| Name | Pos. | NO. | 5′ to 3′ | NO. | 5′ to 3′ |
| NM_006416.4_ | 153 | 969 | CAGUCUAUACCAUAGC | 995 | AAAGCUAUGGUAUAGAC |
| 153-171 | | | UUU | | UG |
| NM_006416.4_ | 189 | 970 | CAGACAAAGAACUCUA | 996 | AAGUAGAGUUCUUUGUC |
| 189-207 | | | CUU | | UG |
| NM_006416.4_ | 217 | 971 | AGCCGUGUGUAUCACA | 997 | UUCUGUGAUACACACGG |
| 217-235 | | | GAA | | CU |
| NM_006416.4_ | 283 | 972 | UAGUCUGGGUAGAUUC | 998 | UUUGAAUCUACCCAGAC |
| 283-301 | | | AAA | | UA |
| NM_006416.4_ | 349 | 973 | GUUAAGUGUGCCAUCG | 999 | UAACGAUGGCACACUUA |
| 349-367 | | | UUA | | AC |
| NM_006416.4_ | 535 | 974 | UGCUGGAGUUACGCUU | 1000 | UACAAGCGUAACUCCAG |
| 535-553 | | | GUA | | CA |
| NM_006416.4_ | 544 | 975 | UACGCUUGUACAGUGG | 1001 | UUUCCACUGUACAAGCG |
| 544-562 | | | AAA | | UA |
| NM_006416.4_ | 608 | 976 | GGGUUUGGCGCUAUAG | 1002 | UAGCUAUAGCGCCAAAC |
| 608-626 | | | CUA | | CC |
| NM_006416.4_ | 609 | 977 | GGUUUGGCGCUAUAGC | 1003 | AUAGCUAUAGCGCCAAA |
| 609-627 | | | UAU | | CC |
| NM_006416.4_ | 718 | 978 | GUAUCUAUCAGGGAUU | 1004 | AAUAAUCCCUGAUAGAU |
| 718-736 | | | AUU | | AC |
| NM_006416.4_ | 794 | 979 | UUCUAUGGUUACACAU | 1005 | AAUAUGUGUAACCAUAG |
| 794-812 | | | AUU | | AA |
| NM_006416.4_ | 805 | 980 | CACAUAUUAUGUCUGG | 1006 | AAACCAGACAUAAUAUG |
| 805-823 | | | UUU | | UG |
| NM_006416.4_ | 1134 | 981 | UGCAUUAAACUAGAGC | 1007 | AAGGCUCUAGUUUAAUG |
| 1134-1152 | | | CUU | | CA |
| NM_006416.4_ | 1141 | 982 | AACUAGAGCCUUAAGU | 1008 | UUGACUUAAGGCUCUAG |
| 1141-1159 | | | CAA | | UU |
| NM_006416.4_ | 1164 | 983 | AGAAGGUAGCAUAAAC | 1009 | UUUGUUUAUGCUACCUU |
| 1164-1182 | | | AAA | | CU |
| NM_006416.4_ | 1364 | 984 | GAGAUGAUACGGUGU | 1010 | UUUAACACCGUAUCAUC |
| 1364-1382 | | | UAAA | | UC |
| NM_006416.4_ | 1386 | 985 | AAUCAUGGUAAGGCUA | 1011 | UUGUAGCCUUACCAUGA |
| 1386-1404 | | | CAA | | UU |
| NM_006416.4_ | 1424 | 986 | GGGACAAUGUCUAAGG | 1012 | AACCCUUAGACAUUGUC |
| 1424-1442 | | | GUU | | CC |
| NM_006416.4_ | 1425 | 987 | GGACAAUGUCUAAGGG | 1013 | UAACCCUUAGACAUUGU |
| 1425-1443 | | | UUA | | CC |
| NM_006416.4_ | 1588 | 988 | AAGACAGGCUAGUUCA | 1014 | UUAUGAACUAGCCUGUC |
| 1588-1606 | | | UAA | | UU |
| NM_006416.4_ | 1589 | 989 | AGACAGGCUAGUUCAU | 1015 | UUUAUGAACUAGCCUGU |
| 1589-1607 | | | AAA | | CU |
| NM_006416.4_ | 1637 | 990 | AUUUCAUAACUCGGAC | 1016 | AUUGUCCGAGUUAUGAA |
| 1637-1655 | | | AAU | | AU |
| NM_006416.4_ | 1638 | 991 | UUUCAUAACUCGGACA | 1017 | AAUUGUCCGAGUUAUGA |
| 1638-1656 | | | AUU | | AA |
| NM_006416.4_ | 1639 | 992 | UUCAUAACUCGGACAA | 1018 | AAAUUGUCCGAGUUAUG |
| 1639-1657 | | | UUU | | AA |
| NM_006416.4_ | 1711 | 993 | UGCCAUUCAUUAUCUG | 1019 | AACCAGAUAAUGAAUGG |
| 1711-1729 | | | GUU | | CA |
| NM_006416.4_ | 1816 | 994 | ACUGUUGGGCUCUCAA | 1020 | UUAUUGAGAGCCCAACA |
| 1816-1834 | | | UAA | | GU |

TABLE 21
RNA effector molecules targeting SLC35A1 (mouse; NM_011895.3;
SEQ ID NO: 1021)

| Oligo | Start | SEQ ID | Sense Sequence | SEQ ID | Antisense Sequence |
|---|---|---|---|---|---|
| Name | Pos. | NO. | 5′ to 3′ | NO. | 5′ to 3′ |
| NM_011895.3_ | 253 | 1022 | CCGCUUACACCGUAGC | 1043 | AAAGCUACGGUGUAAGC |
| 253-271 | | | UUU | | GG |
| NM_011895.3_ | 254 | 1023 | CGCUUACACCGUAGCU | 1044 | UAAAGCUACGGUGUAAG |
| 254-272 | | | UUA | | CG |
| NM_011895.3_ | 378 | 1024 | ACUGGCAGUUUGGGUA | 1045 | AUCUACCCAAACUGCCA |
| 378-396 | | | GAU | | GU |
| NM_011895.3_ | 433 | 1025 | CCAAGGAACUGGCGAA | 1046 | AACUUCGCCAGUUCCUU |
| 433-451 | | | GUU | | GG |
| NM_011895.3_ | 455 | 1026 | UGUGCCAUCACUAGUG | 1047 | AUACACUAGUGAUGGCA |
| 455-473 | | | UAU | | CA |
| NM_011895.3_ | 527 | 1027 | GUACCAGGUGACCUAU | 1048 | UUGAUAGGUCACCUGGU |
| 527-545 | | | CAA | | AC |
| NM_011895.3_ | 610 | 1028 | AGUGGAUUUCCGUCUU | 1049 | AUGAAGACGGAAAUCCA |
| 610-628 | | | CAU | | CU |
| NM_011895.3_ | 671 | 1029 | AGCUACAAAAGUCGUG | 1050 | UACCACGACUUUUGUAG |
| 671-689 | | | GUA | | CU |
| NM_011895.3_ | 774 | 1030 | UUAAAGAGUUCCGACA | 1051 | AAGUGUCGGAACUCUUU |
| 774-792 | | | CUU | | AA |
| NM_011895.3_ | 826 | 1031 | CAGGGAUCGUUGUGAC | 1052 | AACGUCACAACGAUCCC |
| 826-844 | | | GUU | | UG |
| NM_011895.3_ | 1052 | 1032 | UGGAUUACAGAUAACA | 1053 | AAGUGUUAUCUGUAAUC |
| 1052-1070 | | | CUU | | CA |
| NM_011895.3_ | 1190 | 1033 | AUUUGAAUCUCAAGAG | 1054 | AAUCUCUUGAGAUUCAA |
| 1190-1208 | | | AUU | | AU |
| NM_011895.3_ | 1254 | 1034 | AACCCCAGAUGGUAGG | 1055 | UAACCUACCAUCUGGGG |
| 1254-1272 | | | UUA | | UU |
| NM_011895.3_ | 1255 | 1035 | ACCCCAGAUGGUAGGU | 1056 | UUAACCUACCAUCUGGG |
| 1255-1273 | | | UAA | | GU |
| NM_011895.3_ | 1256 | 1036 | CCCCAGAUGGUAGGUU | 1057 | UUUAACCUACCAUCUGG |
| 1256-1273 | | | AAA | | GG |
| NM_011895.3_ | 1462 | 1037 | CAUGGAGGUGCCAUGG | 1058 | AUACCAUGGCACCUCCA |
| 1462-1480 | | | UAU | | UG |
| NM_011895.3_ | 1572 | 1038 | AUAUUCAAGCAACGAG | 1059 | AACCUCGUUGCUUGAAU |
| 1572-1590 | | | GUU | | AU |
| NM_011895.3_ | 1573 | 1039 | UAUUCAAGCAACGAGG | 1060 | AAACCUCGUUGCUUGAA |
| 1573-1591 | | | UUU | | UA |
| NM_011895.3_ | 1574 | 1040 | AUUCAAGCAACGAGGU | 1061 | UAAACCUCGUUGCUUGA |
| 1574-1592 | | | UUA | | AU |
| NM_011895.3_ | 1877 | 1041 | CUGUCAGCUGAUAUAC | 1062 | AAAGUAUAUCAGCUGAC |
| 1877-1895 | | | UUU | | AG |
| NM_011895.3_ | 1936 | 1042 | UAAAGUAUGGUUACCU | 1063 | AACAGGUAACCAUACUU |
| 1936-1954 | | | GUU | | UA |

TABLE 22
RNA effector molecules targeting SLC35A2 (rat; NM_001127642.1;
SEQ ID NO: 1064)

| Oligo | Start | SEQ ID | Sense Sequence | SEQ ID | Antisense Sequence |
|---|---|---|---|---|---|
| Name | Pos. | NO. | 5′ to 3′ | NO. | 5′ to 3′ |
| NM_001127642.1_ | 110 | 1065 | ACCGGCGCCUCAAGUA | 1090 | AUAUACUUGAGGCGCCG |
| 110-128 | | | UAU | | GU |
| NM_001127642.1_ | 111 | 1066 | CCGGCGCCUCAAGUAU | 1091 | UAUAUACUUGAGGCGCC |
| 111-129 | | | AUA | | GG |
| NM_001127642.1_ | 112 | 1067 | CGGCGCCUCAAGUAUA | 1092 | AUAUAUACUUGAGGCGC |
| 112-130 | | | UAU | | CG |
| NM_001127642.1_ | 116 | 1068 | GCCUCAAGUAUAUAUC | 1093 | AAGGAUAUAUACUUGAG |
| 116-134 | | | CUU | | GC |
| NM_001127642.1_ | 363 | 1069 | GGUGCCCUCUCUCAUC | 1094 | AUAGAUGAGAGAGGGCA |
| 363-381 | | | UAU | | CC |
| NM_001127642.1_ | 387 | 1070 | GCAGAAUAACCUCCAG | 1095 | AUACUGGAGGUUAUUCU |
| 387-405 | | | UAU | | GC |
| NM_001127642.1_ | 825 | 1071 | UGUCUGGGGUGUCGUA | 1096 | UAGUACGACACCCCAGA |
| 825-843 | | | CUA | | CA |
| NM_001127642.1_ | 826 | 1072 | GUCUGGGGUGUCGUAC | 1097 | UUAGUACGACACCCCAG |
| 826-844 | | | UAA | | AC |
| NM_001127642.1_ | 827 | 1073 | UCUGGGGUGUCGUACU | 1098 | UUUAGUACGACACCCCA |
| 827-845 | | | AAA | | GA |
| NM_001127642.1_ | 831 | 1074 | GGGUGUCGUACUAAAC | 1099 | UUGGUUUAGUACGACAC |
| 831-849 | | | CAA | | CC |
| NM_001127642.1_ | 967 | 1075 | UUCCACCUGGACCCAU | 1100 | AUAAUGGGUCCAGGUGG |
| 967-985 | | | UAU | | AA |
| NM_001127642.1_ | 968 | 1076 | UCCACCUGGACCCAUU | 1101 | AAUAAUGGGUCCAGGUG |
| 968-986 | | | AUU | | GA |
| NM_001127642.1_ | 1356 | 1077 | UGAACGCUUCCUGAUA | 1102 | AUCUAUCAGGAAGCGUU |
| 1356-1374 | | | GAU | | CA |
| NM_001127642.1_ | 1431 | 1078 | CUCUUUGGGAACAGGG | 1103 | AACCCCUGUUCCCAAAG |
| 1431-1449 | | | GUU | | AG |
| NM_001127642.1_ | 1562 | 1079 | AGCUCUUCAGUAACGA | 1104 | UAGUCGUUACUGAAGAG |
| 1562-1580 | | | CUA | | CU |
| NM_001127642.1_ | 1580 | 1080 | AAUGACUACUCGUGGG | 1105 | AACCCCACGAGUAGUCA |
| 1580-1598 | | | GUU | | UU |
| NM_001127642.1_ | 1596 | 1081 | GUUCCAUUUCCUAUUG | 1106 | AUACAAUAGGAAAUGGA |
| 1596-1614 | | | UAU | | AC |
| NM_001127642.1_ | 1636 | 1082 | UCACCCUGGAUCAUGA | 1107 | UUGUCAUGAUCCAGGGU |
| 1636-1654 | | | CAA | | GA |
| NM_001127642.1_ | 1810 | 1083 | GCUGGGACUAAACUCU | 1108 | AUAAGAGUUUAGUCCCA |
| 1810-1828 | | | UAU | | GC |
| NM_001127642.1_ | 1815 | 1084 | GACUAAACUCUUAUCA | 1109 | UACUGAUAAGAGUUUAG |
| 1815-1833 | | | GUA | | UC |
| NM_001127642.1_ | 1816 | 1085 | ACUAAACUCUUAUCAG | 1110 | AUACUGAUAAGAGUUUA |
| 1816-1834 | | | UAU | | GU |
| NM_001127642.1_ | 1907 | 1086 | GACUGACUAACCUCUG | 1111 | UAACAGAGGUUAGUCAG |
| 1907-1925 | | | UUA | | UC |
| NM_001127642.1_ | 1942 | 1087 | UCCUGCUAUCUUUACA | 1112 | UACUGUAAAGAUAGCAG |
| 1942-1960 | | | GUA | | GA |
| NM_001127642.1_ | 1943 | 1088 | CCUGCUAUCUUUACAG | 1113 | AUACUGUAAAGAUAGCA |
| 1943-1961 | | | UAU | | GG |
| NM_001127642.1_ | 1958 | 1089 | GUAUUUCUUAGGUGA | 1114 | AAACUCACCUAAGAAAU |
| 1958-1976 | | | GUUU | | AC |

TABLE 23
RNA effector molecules targeting SLC35A2 (human;
NM_001032289.1; SEQ ID NO: 1115)

| | | SEQ | | SEQ | |
|---|---|---|---|---|---|
| Oligo | Start | ID | Sense Sequence | ID | Antisense Sequence |
| Name | Pos. | NO. | 5′ to 3′ | NO. | 5′ to 3′ |
| NM_ | 121 | 1116 | GCCUGAAGUACAUAUC | 1142 | AGGGAUAUGUACUUCAG |
| 001032289.1_ | | | CCU | | GC |
| 121-139 | | | | | |
| NM_ | 122 | 1117 | CCUGAAGUACAUAUCC | 1143 | UAGGGAUAUGUACUUCA |
| 001032289.1_ | | | CUA | | GG |
| 122-140 | | | | | |
| NM_ | 125 | 1118 | GAAGUACAUAUCCCUA | 1144 | AGCUAGGGAUAUGUACU |
| 001032289.1_ | | | GCU | | UC |
| 125-143 | | | | | |
| NM_ | 127 | 1119 | AGUACAUAUCCCUAGC | 1145 | ACAGCUAGGGAUAUGUA |
| 001032289.1_ | | | UGU | | CU |
| 127-145 | | | | | |
| NM_ | 130 | 1120 | ACAUAUCCCUAGCUGU | 1146 | AGCACAGCUAGGGAUAU |
| 001032289.1_ | | | GCU | | GU |
| 130-148 | | | | | |
| NM_ | 169 | 1121 | UCAUCCUCAGCAUCCG | 1147 | UAGCGGAUGCUGAGGAU |
| 001032289.1_ | | | CUA | | GA |
| 169-187 | | | | | |
| NM_ | 246 | 1122 | GUGCUCAAAGGUCUCA | 1148 | AGGUGAGACCUUUGAGC |
| 001032289.1_ | | | CCU | | AC |
| 246-264 | | | | | |
| NM_ | 273 | 1123 | CUGCUCUUCGCACAGA | 1149 | UCUUCUGUGCGAAGAGC |
| 001032289.1_ | | | AGA | | AG |
| 273-291 | | | | | |
| NM_ | 279 | 1124 | UUCGCACAGAAGAGGG | 1150 | UACCCCUCUUCUGUGCG |
| 001032289.1_ | | | GUA | | AA |
| 279-297 | | | | | |
| NM_ | 283 | 1125 | CACAGAAGAGGGGUAA | 1151 | ACGUUACCCCUCUUCUG |
| 001032289.1_ | | | CGU | | UG |
| 283-301 | | | | | |
| NM_ | 285 | 1126 | CAGAAGAGGGGUAACG | 1152 | UCACGUUACCCCUCUUC |
| 001032289.1_ | | | UGA | | UG |
| 285-303 | | | | | |
| NM_ | 296 | 1127 | UAACGUGAAGCACCUG | 1153 | AACCAGGUGCUUCACGU |
| 001032289.1_ | | | GUU | | UA |
| 296-314 | | | | | |
| NM_ | 342 | 1128 | CAGUAUGUGGACACGC | 1154 | UGAGCGUGUCCACAUAC |
| 001032289.1_ | | | UCA | | UG |
| 342-360 | | | | | |
| NM_ | 346 | 1129 | AUGUGGACACGCUCAA | 1155 | AGCUUGAGCGUGUCCAC |
| 001032289.1_ | | | GCU | | AU |
| 346-364 | | | | | |
| NM_ | 373 | 1130 | CCUCUCUCAUCUACAC | 1156 | AAGGUGUAGAUGAGAG |
| 001032289.1_ | | | CUU | | AGG |
| 373-391 | | | | | |
| NM_ | 378 | 1131 | CUCAUCUACACCUUGC | 1157 | UCUGCAAGGUGUAGAUG |
| 001032289.1_ | | | AGA | | AG |
| 378-396 | | | | | |
| NM_ | 385 | 1132 | ACACCUUGCAGAAUAA | 1158 | AGGUUAUUCUGCAAGGU |
| 001032289.1_ | | | CCU | | GU |
| 385-403 | | | | | |
| NM_ | 388 | 1133 | CCUUGCAGAAUAACCU | 1159 | UGGAGGUUAUUCUGCAA |
| 001032289.1_ | | | CCA | | GG |
| 388-406 | | | | | |
| NM_ | 392 | 1134 | GCAGAAUAACCUCCAG | 1160 | AUACUGGAGGUUAUUCU |
| 001032289.1_ | | | UAU | | GC |
| 392-410 | | | | | |
| NM_ | 394 | 1135 | AGAAUAACCUCCAGUA | 1161 | ACAUACUGGAGGUUAUU |
| 001032289.1_ | | | UGU | | CU |
| 394-412 | | | | | |
| NM_ | 395 | 1136 | GAAUAACCUCCAGUAU | 1162 | AACAUACUGGAGGUUAU |
| 001032289.1_ | | | GUU | | UC |
| 395-413 | | | | | |
| NM_ | 400 | 1137 | ACCUCCAGUAUGUUGC | 1163 | AUGGCAACAUACUGGAG |
| 001032289.1_ | | | CAU | | GU |
| 400-418 | | | | | |
| NM_ | 413 | 1138 | UGCCAUCUCUAACCUA | 1164 | UGGUAGGUUAGAGAUG |
| 001032289.1_ | | | CCA | | GCA |
| 413-431 | | | | | |
| NM_ | 416 | 1139 | CAUCUCUAACCUACCA | 1165 | AGCUGGUAGGUUAGAGA |
| 001032289.1_ | | | GCU | | UG |
| 416-434 | | | | | |
| NM_ | 424 | 1140 | ACCUACCAGCUGCCAC | 1166 | AAAGUGGCAGCUGGUAG |
| 001032289.1_ | | | UUU | | GU |
| 424-442 | | | | | |
| NM_ | 427 | 1141 | UACCAGCUGCCACUUU | 1167 | UGGAAAGUGGCAGCUGG |
| 001032289.1_ | | | CCA | | UA |
| 427-445 | | | | | |

TABLE 24
RNA effector molecules targeting SLC35A2 (mouse; NM_078484.2;
SEQ ID NO: 1168)

| | | SEQ | | SEQ | |
|---|---|---|---|---|---|
| Oligo | Start | ID | Sense Sequence | ID | Antisense Sequence |
| Name | Pos. | NO. | 5′ to 3′ | NO. | 5′ to 3′ |
| NM_ | 33 | 1169 | GAAACGGACGUGAUGG | 1197 | UAUCCAUCACGUCCGUU |
| 078484.2_ | | | AUA | | UC |
| 33-51 | | | | | |
| NM_ | 166 | 1170 | UUGGAACCUGGGUCCA | 1198 | UAGUGGACCCAGGUUCC |
| 078484.2_ | | | CUA | | AA |
| 166-184 | | | | | |
| NM_ | 200 | 1171 | GC CUCAAGUAUAUAUC | 1199 | AAGGAUAUAUACUUGAG |
| 078484.2_ | | | CUU | | GC |
| 200-218 | | | | | |
| NM_ | 206 | 1172 | AGUAUAUAUCCUUAGC | 1200 | ACAGCUAAGGAUAUAUA |
| 078484.2_ | | | UGU | | CU |
| 206-224 | | | | | |
| NM_ | 325 | 1173 | GUGCUCAAAGGUCUCA | 1201 | AGGUGAGACCUUUGAGC |
| 078484.2_ | | | CCU | | AC |
| 325-343 | | | | | |
| NM_ | 349 | 1174 | CUGCUGCUCUUCGCAC | 1202 | UUUGUGCGAAGAGCAGC |
| 078484.2_ | | | AAA | | AG |
| 349-367 | | | | | |
| NM_ | 350 | 1175 | UGCUGCUCUUCGCACA | 1203 | UUUUGUGCGAAGAGCAG |
| 078484.2_ | | | AAA | | CA |
| 350-368 | | | | | |
| NM_ | 352 | 1176 | CUGCUCUUCGCACAAA | 1204 | UCUUUUGUGCGAAGAGC |
| 078484.2_ | | | AGA | | AG |
| 352-370 | | | | | |
| NM_ | 451 | 1177 | CCCUCUCUCAUCUAUA | 1205 | AGGUAUAGAUGAGAGA |
| 078484.2_ | | | CCU | | GGG |
| 451-469 | | | | | |
| NM_ | 455 | 1178 | CUCUCAUCUAUACCUU | 1206 | UGCAAGGUAUAGAUGAG |
| 078484.2_ | | | GCA | | AG |
| 455-473 | | | | | |
| NM_ | 469 | 1179 | UUGCAGAAUAACCUCC | 1207 | ACUGGAGGUUAUUCUGC |
| 078484.2_ | | | AGU | | AA |
| 469-487 | | | | | |
| NM_ | 542 | 1180 | AGAUCCUGACUACAGC | 1208 | AGCGCUGUAGUCAGGAU |
| 078484.2_ | | | GCU | | CU |
| 542-560 | | | | | |
| NM_ | 785 | 1181 | GUUCUGUGUGGCUUCG | 1209 | UUACGAAGCCACACAGA |
| 078484.2_ | | | UAA | | AC |
| 785-803 | | | | | |
| NM_ | 788 | 1182 | CUGUGUGGCUUCGUAA | 1210 | AGGUUACGAAGCCACAC |
| 078484.2_ | | | CCU | | AG |
| 788-806 | | | | | |
| NM_ | 791 | 1183 | UGUGGCUUCGUAACCU | 1211 | UGUAGGUUACGAAGCCA |
| 078484.2_ | | | ACA | | CA |
| 791-809 | | | | | |
| NM_ | 910 | 1184 | GUCUGGGGUGUAGUAC | 1212 | UUAGUACUACACCCCAG |
| 078484.2_ | | | UAA | | AC |
| 910-928 | | | | | |
| NM_ | 914 | 1185 | GGGGUGUAGUACUAA | 1213 | UGGUUUAGUACUACACC |
| 078484.2_ | | | ACCA | | CC |
| 914-932 | | | | | |
| NM_ | 915 | 1186 | GGGUGUAGUACUAAAC | 1214 | UUGGUUUAGUACUACAC |
| 078484.2_ | | | CAA | | CC |
| 915-933 | | | | | |
| NM_ | 919 | 1187 | GUAGUACUAAACCAAG | 1215 | AGGCUUGGUUUAGUACU |
| 078484.2_ | | | CCU | | AC |
| 919-937 | | | | | |
| NM_ | 920 | 1188 | UAGUACUAAACCAAGC | 1216 | AAGGCUUGGUUUAGUAC |
| 078484.2_ | | | CUU | | UA |
| 920-938 | | | | | |
| NM_ | 1052 | 1189 | UCCACCUGGACCCAUU | 1217 | AAUAAUGGGUCCAGGUG |
| 078484.2_ | | | AUU | | GA |
| 1052-1070 | | | | | |
| NM_ | 1297 | 1190 | AUGCUGGCCUGUCUUC | 1218 | AACGAAGACAGGCCAGC |
| 078484.2_ | | | GUU | | AU |
| 1297-1315 | | | | | |
| NM_ | 1336 | 1191 | AACUGGGACUAAACUC | 1219 | UAAGAGUUUAGUCCCAG |
| 078484.2_ | | | UUA | | UU |
| 1336-1354 | | | | | |
| NM_ | 1341 | 1192 | GGACUAAACUCUUAUC | 1220 | ACUGAUAAGAGUUUAGU |
| 078484.2_ | | | AGU | | CC |
| 1341-1359 | | | | | |
| NM_ | 1344 | 1193 | CUAAACUCUUAUCAGU | 1221 | AAUACUGAUAAGAGUUU |
| 078484.2_ | | | AUU | | AG |
| 1344-1362 | | | | | |
| NM_ | 1351 | 1194 | CUUAUCAGUAUUAGGG | 1222 | UACCCCUAAUACUGAUA |
| 078484.2_ | | | GUA | | AG |
| 1351-1369 | | | | | |
| NM_ | 1422 | 1195 | GGGCUGACAUGACUAA | 1223 | AGGUUAGUCAUGUCAGC |
| 078484.2_ | | | CCU | | CC |
| 1422-1440 | | | | | |
| NM_ | 1445 | 1196 | UAAUGGGCCCACCUCU | 1224 | AGUAGAGGUGGGCCCAU |
| 078484.2_ | | | ACU | | UA |
| 1445-1463 | | | | | |

Arg495His Glucocerebrosidase nucleotide sequence
(SEQ ID NO. 1225)
atggctggcagcctcacaggtttgcttctacttcaggcagtgtcgtgggcatcaggtgcccgcccctgcatccctaaaagcttcggct
acagctcggtggtgtgtgtctgcaatgccacatactgtgactcctttgaccccccgacctttcctgcccttggtaccttcagccgctatg
agagtacacgcagtgggcgacggatggagctgagtatggggcccatccaggctaatcacacgggcacaggcctgctactgaccc
tgcagccagaacagaagttccagaaagtgaagggatttggaggggccatgacagatgctgctgctctcaacatccttgccctgtcac
cccctgcccaaaatttgctacttaaatcgtacttctctgaagaaggaatcggatataacatcatccgggtacccatggccagctgtgac
ttctccatccgcacctacacctatgcagacacccctgatgatttccagttgcacaacttcagcctcccagaggaagataccaagctca
agatacccctgattcaccgagccctgcagttggcccagcgtcccgtttcactccttgccagcccctggacatcacccacttggctcaa
gaccaatggagcggtgaatgggaaggggtcactcaagggacagcccggagacatctaccaccagacctgggccagatactttgt
gaagttcctggatgcctatgctgagcacaagttacagttctgggcagtgacagctgaaaatgagccttctgctgggctgttgagtgga
tacccatccagtgcctgggatcacccctgaacatcagcgagacttcattgcccgtgacctaggtcctaccctcgccaacagtactc
accacaatgtccgcctactcatgctggatgaccaacgcttgctgctgccccactgggcaaaggtggtactgacagacccagaagca
gctaaatatgttcatggcattgctgtacattggtacctggactttctggctccagccaaagccaccctaggggagacacaccgcctgtt
ccccaacaccatgctattgcctcagaggcctgtgtgggctccaagttctgggagcagagtgtgcggctaggctcctgggatcgag
ggatgcagtacagccacagcatcatcacgaacctcctgtaccatgtggtcggctggaccgactggaaccttgccctgaaccccgaa
ggaggacccaattgggtgcgtaactttgtcgacagtcccatcattgtagacatcaccaaggacacgttttacaaacagcccatgttcta
ccaccttggccacttcagcaagttcattcctgagggctcccagagagtggggctggttgccagtcagaagaacgacctggacgcag
tggcactgatgcatcccgatggctctgctgttgtggtcgtgctaaaccgctcctctaaggatgtgcctcttaccatcaaggatcctgctg
tgggctcctggagacaatctcacctggctactccattcacacctacctgtggcatcgccagtga
Arg495His Glucocerebrosidase protein sequence
(SEQ ID NO. 1226)
MAGSLTGLLLLQAVSWASGARPCIPKSFGYSSVVCVCNATYCDSFDPPTFPALGTFS
RYESTRSGRRMELSMGPIQANHTGTGLLLTLQPEQKFQKVKGFGGAMTDAAALNI
LALSPPAQNLLLKSYFSEEGIGYNIIRVPMASCDFSIRTYTYADTPDDFQLHNFSLPEE
DTKLKIPLIHRALQLAQRPVSLLASPWTSPTWLKTNGAVNGKGSLKGQPGDIYHQT
WARYFVKFLDAYAEHKLQFWAVTAENEPSAGLLSGYPFQCLGFTPEHQRDFIARD
LGPTLANSTHHNVRLLMLDDQRLLLPHWAKVVLTDPEAAKYVHGIAVHWYLDFL
APAKATLGETHRLFPNTMLFASEACVGSKFWEQSVRLGSWDRGMQYSHSIITNLLY
HVVGWTDWNLALNPEGGPNWVRNFVDSPIIVDITKDTFYKQPMFYHLGHFSKFIPE
GSQRVGLVASQKNDLDAVALMHPDGSAVVVVLNRSSKDVPLTIKDPAVGFLETISP
GYSIHTYLWHRQ*
GV90 Vector Sequence
(SEQ ID NO. 1227)
CTAGAgcatgcatctcaattagtcagcaaccatagtcccgcccctaactccgcccatcccgcccctaactccgcccagttccgc
ccattctccgccccatggctgactaattttttttatttatgcagaggccgaggccgcctcggcctctgagctattccagaagtagtgagg
aggcttttttggaggcctaggcttttgcaaaaagctttatccccgctgccatcatggttcgaccattgaactgcatcgtcgccgtgtccc
aagatatggggattggcaagaacggagacctaccctggcctccgctcaggaacgagttcaagtacttccaaagaatgaccacaac
ctatcagtggaaggtaaacagaatctggtgattatgggtaggaaaacctggttctccattcctgagaagaatcgacctttaaaggaca
gaattaatatagttctcagtagagaactcaaagaaccaccacgaggagctcattttcttgccaaaagtttggatgatgccttaagactta
ttgaacaaccggaattggcaagtaaagtagacatggtttggatagtcggaggcagttctgtttaccaggaagccatgaatcaaccag
gccacctcagactctttgtgacaaggatcatgcaggaatttgaaagtgacacgtttttcccagaaattgatttggggaaatataaacttct
cccagaatacccaggcgtcctctctgaggtccaggaggaaaaaggcatcaagtataagtttgaagtctacgagaagaaagactaac
aggaagatgattcaagttctctgctcccctcctaaagctatgcatttttataagaccatgggacttttgctggctttagatctttgtgaagg
aaccttacttctgtggtgtgacataattggacaaactacctacagagatttaaagctctaaggtaaatataaaatttttaagtgtataatgt
gttaaactactgattctaattgtttgtgtattttagattccaacctatggaactgatgaatgggagcagtggtggaatgcctttaatgagga
aaacctgttttgctcagaagaaatgccatctagtgatgatgaggctactgctgactctcaacattctactcctccaaaaaagaagagaa
aggtagaagaccccaaggactttccttcagaattgctaagttttttgagtcatgctgtgtttagtaatagaactcttgcttgctttgctattta
caccacaaaggaaaaagctgcactgctatacaagaaaattatggaaaaatattctgtaacctttataagtaggcataacagttataatc
ataacatactgttttttcttactccacacaggcatagagtgtctgctattaataactatgctcaaaaattgtgtacctttagattttaatttgta
aaggggttaataaggaatatttgatgtatagtgccttgactagagatcataatcagccataccacatttgtagaggttttacttgctttaaa
aaacctcccacacctccccctgaacctgaaacataaaatgaatgcaattgttgttgttaacttgtttattgcagcttataatggttacaaat
aaagcaatagcatcacaaattGTCGAcctgcaggcatgcaagcttggcgtaatcatggtcatagctgtttcctgtgtgaaattgtta
tccgctcacaattccacacaacatacgagccggaagcataaagtgtaaagcctggggtgcctaatgagtgagctaactcacattaatt
gcgttgcgctcactgcccgctttccagtcgggaaacctgtcgtgccagctgcattaatgaatcggccaacgcgcggggagaggcg
gtttgcgtattgggcgctcttccgcttcctcgctcactgactcgctgcgctcggtcgttcggctgcggcgagcggtatcagctcactca
aaggcggtaatacggttatccacagaatcaggggataacgcaggaaagaacatgtgagcaaaaggccagcaaaaggccaggaa
ccgtaaaaaggccgcgttgctggcgtttttccataggctccgcccccctgacgagcatcacaaaaatcgacgctcaagtcagaggt
ggcgaaacccgacaggactataaagataccaggcgtttccccctggaagctccctcgtgcgctctcctgttccgaccctgccgctta
ccggatacctgtccgcctttctcccttcgggaagcgtggcgctttctcatagctcacgctgtaggtatctcagttcggtgtaggtcgttc
gctccaagctgggctgtgtgcacgaaccccccgttcagcccgaccgctgcgccttatccggtaactatcgtcttgagtccaacccgg
taagacacgacttatcgccactggcagcagccactggtaacaggattagcagagcgaggtatgtaggcggtgctacagagttcttg
aagtggtggcctaactacggctacactagaagaacagtatttggtatctgcgctctgctgaagccagttaccttcggaaaaagagttg
gtagctcttgatccggcaaacaaaccaccgctggtagcggtggtttttttgtttgcaagcagcagattacgcgcagaaaaaaaggatc
tcaagaagatcctttgatcttttctacggggtctgacgctcagtggaacgaaaactcacgttaagggattttggtcatgagattatcaaa
aaggatcttcacctagatccttttaaattaaaaatgaagttttaaatcaatctaaagtatatatgagtaaacttggtctgacagttaccaatg
cttaatcagtgaggcacctatctcagcgatctgtctatttcgttcatccatagttgcctgactccccgtcgtgtagataactacgatacgg
gagggcttaccatctggccccagtgctgcaatgataccgcgagacccacgctcaccggctccagatttatcagcaataaaccagcc
agccggaagggccgagcgcagaagtggtcctgcaactttatccgcctccatccagtctattaattgttgccgggaagctagagtaag
tagttcgccagttaatagtttgcgcaacgttgttgccattgctacaggcatcgtggtgtcacgctcgtcgtttggtatggcttcattcagct
ccggttcccaacgatcaaggcgagttacatgatcccccatgttgtgcaaaaaagcggttagctccttcggtcctccgatcgttgtcag
aagtaagttggccgcagtgttatcactcatggttatggcagcactgcataattctatactgtcatgccatccgtaagatgctffictgtga
ctggtgagtactcaaccaagtcattctgagaatagtgtatgcggcgaccgagttgctcttgcccggcgtcaatacgggataataccgc
gccacatagcagaactttaaaagtgctcatcattggaaaacgttcttcggggcgaaaactctcaaggatcttaccgctgttgagatcca
gttcgatgtaacccactcgtgcacccaactgatcttcagcatcttttactttcaccagcgtttctgggtgagcaaaaacaggaaggcaa
aatgccgcaaaaaagggaataagggcgacacggaaatgttgaatactcatactatcattttcaatattattgaagcatttatcagggtt
attgtctcatgagcggatacatatttgaatgtatttagaaaaataaacaaataggggttccgcgcacatttccccgaaaagtgccacctg
acgtctaagaaaccattattatcatgacattaacctataaaaataggcgtatcacgaggccctttcgtctcgcgcgtttcggtgatgacg
gtgaaaacctctgacacatgcagctcccggagacggtcacagcttgtctgtaagcggatgccgggagcagacaagcccgtcagg
gcgcgtcagcgggtgttggcgggtgtcggggctggcttaactatgcggcatcagagcagattgtactgagagtgcaccatatgcgg
tgtgaaataccgcacagatgcgtaaggagaaaataccgcatcaggcgccattcgccattcaggctgcgcaactgttgggaagggc
gatcggtgcgggcctcttcgctattacgccagctggcgaaagggggatgtgctgcaaggcgattaagttgggtaacgccagggtttt
cccagtcacgacgttgtaaaacgacggccagtgAATTCTACGGGCCAGATTTACGCGTTGACATTG
ATTATTGACTAGTTATTAATAGTAATCAATTACGGGGTCATTAGTTCATAGCCC
ATATATGGAGTTCCGCGTTACATAACTTACGGTAAATGGCCCGCCTGGCTGACC
GCCCAACGACCCCCGCCCATTGACGTCAATAATGACGTATGTTCCCATAGTAAC
GCCAATAGGGACTTTCCATTGACGTCAATGGGTGGAGTATTTACGGTAAACTGC
CCACTTGGCAGTACATCAAGTGTATCATATGCCAAGTACGCCCCCTATTGACGT
CAATGACGGTAAATGGCCCGCCTGGCATTATGCCCAGTACATGACCTTATGGGA
CTTTCCTACTTGGCAGTACATCTACGTATTAGTCATCGCTATTACCATGGTGATG
CGGTTTTGGCAGTACATCAATGGGCGTGGATAGCGGTTTGACTCACGGGGATTT
CCAAGTCTCCACCCCATTGACGTCAATGGGAGTTTGTTTTGGCACCAAAATCAA
CGGGACTTTCCAAAATGTCGTAACAACTCCGCCCCATTGAAGCAAATGGGCGGT
AGGCGTGTACGGTGGGAGGTCTATATAAGCAGGAGCTCGTCAGATCGCCTGGA
GACGCCATCCACGCTGTTTTGACCTCCATAGAAGACACCGGGACCGATCCAGCC
TCCGCGGCCGGGAACGGTGCATTGGAACGCGGATTCCCCGTGCCAAGAGTGAC
GTAAGTACCGCCTATAGACTCTATAGGCACACCCCTTTGGCTCTTATGCATGCT
ATACTGTTTTTGGCTTGGGGCCTATACACCCCCGCTTCCTTATGCTATAGGTGAT
GGTATAGCTTAGCCTATAGGTGTGGGTTATTGACCATTATTGACCACTCCCCTAT
TGGTGACGATACTTTCCATTACTAATCCATAACATGGCTCTTTGCCACAACTATC
TCTATTGGCTATATGCCAATACTCTGTCCTTCAGAGACTGACACGGACTCTGTAT
TTTTACAGGATGGGGTCCCATTTATTATTTACAAATTCACATATACAACAACGC
CGTCCCCCGTGCCCGCAGTTTTTATTAAACATAGCGTGGGATCTCCACGCGAAT
CTCGGGTACGTGTTCCGGACATGGGCTCTTCTCCGGTAGCGGCGGAGCTTCCAC
ATCCGAGCCCTGGTCCCATGCCTCCAGCGGCTCATGGTCGCTCGGCAGCTCCTT
GCTCCTAACAGTGGAGGCCAGACTTAGGCACAGCACAATGCCCACCACCACCA
GTGTGCCGCACAAGGCCGTGGCGGTAGGGTATGTGTCTGAAAATGAGTCGGAG
ATTGGGCTCGCACCGCTGACGCAGATGGAAGACTTAAGGCAGCGGCAGAAGAA
GATGCAGGCAGCTGAGTTGTTGTATTCTGATAAGAGTCAGAGGTAACTCCCGTT
GCGGTGCTGTTAACGGTGGAGGGCAGTGTAGTCTGAGCAGTACTCGTTGCTGCC
GCGCGCGCCACCAGACATAATAGCTGACAGACTAACAGACTGTTCCTTTCCATG
GGTCTTTTCTGCAGTCACCGTCCTTGCTTGCAATCGCGGCCGCAGGCGCGCCGG
ATCCGCTGATCAGCCTCGACTGTGCCTTCTAGTTGCCAGCCATCTGTTGTTTGCC
CCTCCCCCGTGCCTTCCTTGACCCTGGAAGGTGCCACTCTCACTGTCCTTTCCTA
ATAAAATGAGGAAATTGCATCGCATTGTCTGAGTAGGTGTCATTCTATTCTGGG
GGGTGGGGTGGGGCAGGACAGCAAGGGGGAGGATTGGGAAGACAATAGCAGG
CATGCTGGGGT
Human glucocerebrosidase, variant 1: Genbank Accession No. NM_000157
(SEQ ID NO. 1228)
acatcgggaa gccggaatta cttgcagggc taacctagtg cctatagcta aggcaggtac ctgcatcctt gtttttgttt
agtggatcct ctatccttca gagactctgg aacccctgtg gtcttctctt catctaatga ccctgagggg atggagtttt
caagtccttc cagagaggaa tgtcccaagc ctttgagtag ggtaagcatc atggctggca gcctcacagg attgcttcta
cttcaggcag tgtcgtgggc atcaggtgcc cgcccctgca tccctaaaag cttcggctac agctcggtgg tgtgtgtctg
caatgccaca tactgtgact cctttgaccc cccgaccttt cctgcccttg gtaccttcag ccgctatgag agtacacgca
gtgggcgacg gatggagctg agtatggggc ccatccaggc taatcacacg ggcacaggcc tgctactgac cctgcagcca
gaacagaagt tccagaaagt gaagggattt ggaggggcca tgacagatgc tgctgctctc aacatccttg ccctgtcacc
ccctgcccaa aatttgctac ttaaatcgta cttctctgaa gaaggaatcg gatataacat catccgggta cccatggcca
gctgtgactt ctccatccgc acctacacct atgcagacac ccctgatgat ttccagttgc acaacttcag cctcccagag
gaagatacca agctcaagat acccctgatt caccgagccc tgcagttggc ccagcgtccc gtttcactcc ttgccagccc
ctggacatca cccacttggc tcaagaccaa tggagcggtg aatgggaagg ggtcactcaa gggacagccc ggagacatct
accaccagac ctgggccaga tactttgtga agttcctgga tgcctatgct gagcacaagt tacagttctg ggcagtgaca
gctgaaaatg agccttctgc tgggctgttg agtggatacc ccttccagtg cctgggcttc acccctgaac atcagcgaga
cttcattgcc cgtgacctag gtcctaccct cgccaacagt actcaccaca atgtccgcct actcatgctg gatgaccaac
gcttgctgct gccccactgg gcaaaggtgg tactgacaga cccagaagca gctaaatatg ttcatggcat tgctgtacat
tggtacctgg actttctggc tccagccaaa gccaccctag gggagacaca ccgcctgttc cccaacacca tgctctttgc
ctcagaggcc tgtgtgggct ccaagttctg ggagcagagt gtgcggctag gctcctggga tcgagggatg cagtacagcc
acagcatcat cacgaacctc ctgtaccatg tggtcggctg gaccgactgg aaccttgccc tgaaccccga aggaggaccc
aattgggtgc gtaactttgt cgacagtccc atcattgtag acatcaccaa ggacacgttt tacaaacagc ccatgttcta
ccaccttggc cacttcagca agttcattcc tgagggctcc cagagagtgg ggctggttgc cagtcagaag aacgacctgg
acgcagtggc actgatgcat cccgatggct ctgctgttgt ggtcgtgcta aaccgctcct ctaaggatgt gcctcttacc
atcaaggatc ctgctgtggg cttcctggag acaatctcac ctggctactc cattcacacc tacctgtggc gtcgccagtg
atggagcaga tactcaagga ggcactgggc tcagcctggg cattaaaggg acagagtcag ctcacacgct gtctgtgact
aaagagggca cagcagggcc agtgtgagct tacagcgacg taagcccagg ggcaatggtt tgggtgactc actttcccct
ctaggtggtg ccaggggctg gaggccccta gaaaaagatc agtaagcccc agtgtccccc cagcccccat gcttatgtga
acatgcgctg tgtgctgctt gctttggaaa ctgggcctgg gtccaggcct agggtgagct cactgtccgt acaaacacaa
gatcagggct gagggtaagg aaaagaagag actaggaaag ctgggcccaa aactggagac tgtttgtctt tcctggagat
gcagaactgg gcccgtggag cagcagtgtc agcatcaggg cggaagcctt aaagcagcag cgggtgtgcc
caggcaccca gatgattcct atggcaccag ccaggaaaaa tggcagctct taaaggagaa aatgtttgag cccaaaaaaa
aaaaaaaaaa aaaa
Human glucocerebrosidase, variant 1: Genbank Accession No. NP_000148
(SEQ ID NO. 1229)

| | |
|---|---|
| 1 | mefsspsree cpkplsrvsi magsltglll lqavswasga rpcipksfgy ssvvcvcnat |
| 61 | ycdsfdpptf palgtfsrye strsgrrmel smgpiqanht gtgllltlqp eqkfqkvkgf |
| 121 | ggamtdaaal nilalsppaq nlllksyfse egigyniirv pmascdfsir tytyadtpdd |
| 181 | fqlhnfslpe edtklkipli hralqlaqrp vsllaspwts ptwlktngav ngkgslkgqp |
| 241 | gdiyhqtwar yfvkfldaya ehklqfwavt aenepsagll sgypfqclgf tpehqrdfia |
| 301 | rdlgptlans thhnvrllml ddqrlllphw akvvltdpea akyvhgiavh wyldflapak |
| 361 | atlgethrlf pntmlfasea cvgskfweqs vrlgswdrgm qyshsiitnl lyhvvgwtdw |
| 421 | nlalnpeggp nwvrnfvdsp iivditkdtf ykqpmfyhlg hfskfipegs qrvglvasqk |
| 481 | ndldavalmh pdgsavvvvl nrsskdvplt ikdpavgfle tispgysiht ylwrrq |

Homo sapiens iduronate 2-sulfatase (IDS), transcript variant 1 (Idursulfase)
Genbank Accession No. NM_000202
(SEQ ID NO. 1230)
agaacccgcc ccggagggga gggacgcagg gaagagtcgc acggacgcac tcgcgctgcg gccagcgccc
gggcctgcgg gcccgggcgg cggctgtgtt gcgcagtctt catgggttcc cgacgaggag gtctctgtgg ctgcggcggc
ggctgctaac tgcgccacct gctgcagcct gtccccgccg ctctgaagcg gccgcgtcga agccgaaatg ccgccacccc
ggaccggccg aggccttctc tggctgggtc tggttctgag ctccgtctgc gtcgccctcg gatccgaaac gcaggccaac
tcgaccacag atgctctgaa cgttcttctc atcatcgtgg atgacctgcg cccctccctg ggctgttatg gggataagct
ggtgaggtcc ccaaatattg accaactggc atcccacagc ctcctcttcc agaatgcctt tgcgcagcaa gcagtgtgcg
ccccgagccg cgtttctttc ctcactggca ggagacctga caccacccgc ctgtacgact tcaactccta ctggagggtg
cacgctggaa acttctccac catcccccag tacttcaagg agaatggcta tgtgaccatg tcggtgggaa aagtctttca
ccctgggata tcttctaacc ataccgatga ttctccgtat agctggtctt ttccacctta tcatccttcc tctgagaagt
atgaaaacac taagacatgt cgagggccag atggagaact ccatgccaac ctgctttgcc ctgtggatgt gctggatgtt
cccgagggca ccttgcctga caaacagagc actgagcaag ccatacagtt gttggaaaag atgaaaacgt cagccagtcc
tttcttcctg gccgttgggt atcataagcc acacatcccc ttcagatacc ccaaggaatt tcagaagttg tatcccttgg
agaacatcac cctggccccc gatcccgagg tccctgatgg cctaccccct gtggcctaca acccctggat ggacatcagg
caacgggaag acgtccaagc cttaaacatc agtgtgccgt atggtccaat tcctgtggac tttcagcgga aaatccgcca
gagctacttt gcctctgtgt catatttgga tacacaggtc ggccgcctct tgagtgcttt ggacgatctt cagctggcca
acagcaccat cattgcattt acctcggatc atgggtgggc tctaggtgaa catggagaat gggccaaata cagcaatttt
gatgttgcta cccatgttcc cctgatattc tatgttcctg gaaggacggc ttcacttccg gaggcaggcg agaagctttt
cccttacctc gacccttttg attccgcctc acagttgatg gagccaggca ggcaatccat ggaccttgtg gaacttgtgt
ctctttttcc cacgctggct ggacttgcag gactgcaggt tccacctcgc tgccccgttc cttcatttca cgttgagctg
tgcagagaag gcaagaacct tctgaagcat tttcgattcc gtgacttgga agaggatccg tacctccctg gtaatccccg
tgaactgatt gcctatagcc agtatccccg gccttcagac atccctcagt ggaattctga caagccgagt ttaaaagata
taaagatcat gggctattcc atacgcacca tagactatag gtatactgtg tgggttggct tcaatcctga tgaatttcta
gctaactttt ctgacatcca tgcaggggaa ctgtattttg tggattctga cccattgcag gatcacaata tgtataatga
ttcccaaggt ggagatcttt tccagttgtt gatgccttga gttttgccaa ccatggatgg caaatgtgat gtgctccctt
ccagctggtg agaggaggag ttagagctgg tcgttttgtg attacccata atattggaag cagcctgagg gctagttaat
ccaaacatgc atcaacaatt tggcctgaga atatgtaaca gccaaacctt ttcgtttagt ctttattaaa atttataatt
ggtaattgga ccagtttttt ttttaatttc cctcttttta aaacagttac ggcttattta ctgaataaat acaaagcaaa
caaactcaag ttatgtcata cctttggata cgaagaccat acataataac caaacataac attatacaca aagaatactt
tcattatttg tggaatttag tgcatttcaa aaagtaatca tatatcaaac taggcaccac actaagttcc tgattatttt gtttataatt
taataatata tcttatgagc cctatatatt caaaatatta tgttaacatg taatccatgt ttctttttca aatctaaagt taaaaaaaaa
tagcagaagc cagtgtctta aagtctatct tttgtttcta agaccatggg atttcataat ctcaagataa aatatgtatg
aagtaattaa tgtagaattt ttacaccaaa taataaataa tgcttaataa actagagata tgagatgtgt aggaaatttg
gttaaacttt tttcagatac tttctggccc aaataataat ttgttagcaa ataatatgac ccttgaactc aatggccatc
tattaaaaga ctgttgttca cactggaaaa catttaaaga tgtgactata tccatgggtg gattgaatca ctcaaaatat
attagtatcc ttctttaggg atggttggtt acagacatgt atttattcag gaggcagaaa atattccatt ttaattgctt
attaaagaaa acattaaatt ctaaattatt ttgaggactg tgaagacttt tcattagtgt aatattaggt cattgtcaat
ctcccagaat gtagttctat attctctaaa tatgaaagta tccagaaagg ccagtggtag taaaaagctt agtgtatata
atctcaaaag ggatggaata tttacagctc atatttataa catgttgaat cttctcagtt atcagtagtc atcagaagtg
tcaatagctt tctaaataaa tattaaatat ctactgtcct gtagtgaagg agtaattttt agtaattttc tctttacaaa gtctccagtg
tttccaggta aatatttgtg aaacaaaata cagcaaacta cattgttact tcagtgtatt gttgccaaaa atgacaagat
attatattaa aatcagtaaa ttttagacag attttaaaaa ttaattagcc tacaatagag gttatatggt aacacggtga
tcttctaagc agttaagtga ctgactgttc tggcaacaac gacttctccg tgactgaagg gccctgttca tttcctgatc
ctgaagctcg tctctctttt gagcctccgc ttgctttggt cgatggtttc cctcagcttt ttctttgctg ttcttcatcc tcgttgttgc
tgtcatcatg ttcactgtgg cttttacaat acagcctgta aattccttat gacatagttc agtgcatttg gctttatcgc
ctgctccaca gttctttacc tttacttggc ttagagaaac tgtatctttg ttgcttcata taacctttcc ccaaccccac
taagctggac ataacttatt agtggtcctc ccgtcacttt atttgtagaa atctctcttt cacatgagca ggggttcttt
catgtggttt agctgacagc agaactagtg attctagaca ttttgcatgg ccctcattca gtggctcaca aacatgaggg
agcatcagaa ctacttgagg ggcttgttaa aacccagtgc gttagaagtc ggatgcggtg gctcacacct gtaatcccag
cactttggga ggcccaggca ggcggatcac ttgaggttag gagttcaaga ccagcctggc caacatggtg aaaccccgtc
tctactaaaa atacaaaagt tagccgggtg tggtggtgca tgcctgtaat cccagcttct tgggaggcca aggcacaaga
atcgcttgaa ccaggagacg gaggtttcag tgaatgaaga tcgtgccatt gtattccagc ctcggcaaca cagcaggact
gtgattttct ttggagactc ctagattttc tgtggttttg aactgaattt gttggatgtt ggcaagtgcc tcttatgagc tgtttcttta
tcctgcattt gccccacaaa gacttatctg gaggtgagca aagtatgttt ggtagtgagg tcacaaaggc aatcagcccc
ttcctcccca ctcccattgc catcttctca gtccttctcc ctttctttcc aagtagttta cccacccctc ctctttcctc ccctgtccct
aaaataatcc acgtgtcttc ctaaaatctc tctttgatcc tgtcctttga taacaccgtc agtgcctact actgggtcta
gacagacctc tgttgagcag tcagagtctt ccctgactcc acaatgcccc tttccttggc tgaccagtat gactactggt
ccccaccttt cccttgccta tccctacctc cctcctacta ggttgtccca tccctctctt cacccattca ttcatgacca
tttttcacta ccaagctccc cccctcccga aggaggctga ggtttttgtg actctctaga ctctattgtg ggatggaatg
aacattgcta aagaatcttg tgttcgcttt actttaaaaa ggtatttttt tcctaattat aaaactgatg tgtcagttac ggaaaaatta
gaaatgcagc acaaatacat gaatatttta ccacgaaatt gccatataat atcttgtctt ttttgggggt gtgaattttt
tgcattgttc tgatcatatt ctttatcatg taatttatgt tcttttttac taagtattat gtgtggttat tatagatttt cacaaagata
tattgctggt aatatatttt attgtgtagt cttataattt acttaacctt ctttcaattg ttagaaattt aggctatttc cagattttca
gtattgtaaa taatgctgtg atgaccaatt ttgtgaataa aatgttttta tgtatttcag attattccct taggatagtc tctcagtgcc
aagttgtcaa aaacatctct attttgctta tcttcctgct ctcttgctgc cttagggggt agtaaactga aacataaagt
aaacatgcat acaaataaaa aacataaaac aaaaataagc aacctgatgg taataggtga aagtggtaac ctgttttaac
tttgaattct tgccgggcgc ggtggctcac gcctgtaatc ccagcacttt gggaggctga ggcgggtgga tcacgaggtc
aggagttcaa aaccagcctg gccaagatgg tgaaatcccg tctctactaa aaatacaaaa attagccggg cgtggtggcg
ggcgcctgta atcccagcta cttgggaggc tgaggcagag aattgcttga acccaggagg cggaggttgc agtgagccaa
gatcgcgcca ctgcactcca gcctgggtga cagagcgaga ctccgtctca aataaaaaac aacaaaaaac aaaaaaaact
taaaattctt tgcttgttag tgaccttgat catggttctc tttgtacgat agttgggcat ctgtatttcc acttgtgtga atttgccttt
aaattttggt tatgggtttc accttttaaa ataatcaaac atatttatct tttcctgtgt gataggtttt tttctgtatc ttttcctgtt
aaacacacag acccctcccc aatctggaca ttgaataaat attcattttc ctttgcattg ttaaaaaaaa aaaaaaaaaa
aaaaaa
Homo sapiens iduronate 2-sulfatase (IDS), transcript variant 1 (Idursulfase)
Genbank Accession No. NP_000193
(SEQ ID NO. 1231)

| | |
|---|---|
| 1 | mppprtgrgl lwlglvlssv cvalgsetqa nsttdalnvl liivddlrps lgcygdklvr |
| 61 | spnidqlash sllfqnafaq qavcapsrvs fltgrrpdtt rlydfnsywr vhagnfstip |
| 121 | qyfkengyvt msvgkvfhpg issnhtddsp yswsfppyhp ssekyentkt crgpdgelha |
| 181 | nllcpvdvld vpegtlpdkq steqaiqlle kmktsaspff lavgyhkphi pfrypkefqk |
| 241 | lyplenitla pdpevpdglp pvaynpwmdi rqredvqaln isvpygpipv dfqrkirqsy |
| 301 | fasvsyldtq vgrllsaldd lqlanstiia ftsdhgwalg ehgewakysn fdvathvpli |
| 361 | fyvpgrtasl peageklfpy ldpfdsasql mepgrqsmdl velvslfptl aglaglqvpp |
| 421 | rcpvpsfhve lcregknllk hfrfrdleed pylpgnprel iaysqyprps dipqwnsdkp |
| 481 | slkdikimgy sirtidyryt vwvgfnpdef lanfsdihag elyfvdsdpl qdhnmyndsq |
| 541 | ggdlfqllmp |

Homo sapiens Acid Alpha Glucosidase, variant 1 Genbank Accession No.
NM_000152
(SEQ ID NO: 1232)

| | |
|---|---|
| 1 | acccgcctct gcgcgccccc gggcacgacc ccggagtctc cgcgggcggc cagggcgcgc |
| 61 | gtgcgcggag gtgagccggg ccggggctgc ggggcttccc tgagcgcggg ccgggtcggt |
| 121 | ggggcggtcg gctgcccgcg cggcctctca gttgggaaag ctgaggttgt cgccggggcc |
| 181 | gcgggtggag gtcggggatg aggcagcagg taggacagtg acctcggtga cgcgaaggac |
| 241 | cccggccacc tctaggttct cctcgtccgc ccgttgttca gcgagggagg ctctgcgcgt |
| 301 | gccgcagctg acggggaaac tgaggcacgg agcgggcctg taggagctgt ccaggccatc |
| 361 | tccaaccatg ggagtgaggc acccgccctg ctcccaccgg ctcctggccg tctgcgccct |
| 421 | cgtgtccttg gcaaccgctg cactcctggg gcacatccta ctccatgatt tcctgctggt |
| 481 | tccccgagag ctgagtggct cctccccagt cctggaggag actcacccag ctcaccagca |
| 541 | gggagccagc agaccagggc cccgggatgc ccaggcacac cccggccgtc ccagagcagt |
| 601 | gcccacacag tgcgacgtcc cccccaacag ccgcttcgat tgcgcccctg acaaggccat |
| 661 | cacccaggaa cagtgcgagg cccgcggctg ttgctacatc cctgcaaagc aggggctgca |
| 721 | gggagcccag atggggcagc cctggtgctt cttcccaccc agctacccca gctacaagct |
| 781 | ggagaacctg agctcctctg aaatgggcta cacggccacc ctgacccgta ccacccccac |
| 841 | cttcttcccc aaggacatcc tgaccctgcg gctggacgtg atgatggaga ctgagaaccg |
| 901 | cctccacttc acgatcaaag atccagctaa caggcgctac gaggtgccct tggagacccc |
| 961 | gcatgtccac agccgggcac cgtccccact ctacagcgtg gagttctccg aggagccctt |
| 1021 | cggggtgatc gtgcgccggc agctggacgg ccgcgtgctg ctgaacacga cggtggcgcc |
| 1081 | cctgttcttt gcggaccagt tccttcagct gtccacctcg ctgccctcgc agtatatcac |
| 1141 | aggcctcgcc gagcacctca gtcccctgat gctcagcacc agctggacca ggatcaccct |
| 1201 | gtggaaccgg gaccttgcgc ccacgcccgg tgcgaacctc tacgggtctc accctttcta |
| 1261 | cctggcgctg gaggacggcg ggtcggcaca cggggtgttc ctgctaaaca gcaatgccat |
| 1321 | ggatgtggtc ctgcagccga gccctgccct tagctggagg tcgacaggtg ggatcctgga |
| 1381 | tgtctacatc ttcctgggcc cagagcccaa gagcgtggtg cagcagtacc tggacgttgt |
| 1441 | gggatacccg ttcatgccgc catactgggg cctgggcttc cacctgtgcc gctggggcta |
| 1501 | ctcctccacc gctatcaccc gccaggtggt ggagaacatg accagggccc acttccccct |
| 1561 | ggacgtccag tggaacgacc tggactacat ggactcccgg agggacttca cgttcaacaa |
| 1621 | ggatggcttc cgggacttcc cggccatggt gcaggagctg caccagggcg gccggcgcta |
| 1681 | catgatgatc gtggatcctg ccatcagcag ctcgggccct gccgggagct acaggcccta |
| 1741 | cgacgagggt ctgcggaggg gggttttcat caccaacgag accggccagc cgctgattgg |
| 1801 | gaaggtatgg cccgggtcca ctgccttccc cgacttcacc aaccccacag ccctggcctg |
| 1861 | gtgggaggac atggtggctg agttccatga ccaggtgccc ttcgacggca tgtggattga |
| 1921 | catgaacgag ccttccaact tcatcagggg ctctgaggac ggctgcccca acaatgagct |
| 1981 | ggagaaccca ccctacgtgc ctggggtggt tggggggacc ctccaggcgg ccaccatctg |
| 2041 | tgcctccagc caccagtttc tctccacaca ctacaacctg cacaacctct acggcctgac |
| 2101 | cgaagccatc gcctcccaca gggcgctggt gaaggctcgg gggacacgcc catttgtgat |
| 2161 | ctcccgctcg acctttgctg gccacggccg atacgccggc cactggacgg gggacgtgtg |
| 2221 | gagctcctgg gagcagctcg cctcctccgt gccagaaatc ctgcagttta acctgctggg |
| 2281 | ggtgcctctg gtcggggccg acgtctgcgg cttcctgggc aacacctcag aggagctgtg |
| 2341 | tgtgcgctgg acccagctgg gggccttcta ccccttcatg cggaaccaca acagcctgct |
| 2401 | cagtctgccc caggagccgt acagcttcag cgagccggcc cagcaggcca tgaggaaggc |
| 2461 | cctcaccctg cgctacgcac tcctccccca cctctacaca ctgttccacc aggcccacgt |
| 2521 | cgcgggggag accgtggccc ggcccctctt cctggagttc cccaaggact ctagcacctg |
| 2581 | gactgtggac caccagctcc tgtgggggga ggccctgctc atcaccccag tgctccaggc |
| 2641 | cgggaaggcc gaagtgactg gctacttccc cttgggcaca tggtacgacc tgcagacggt |
| 2701 | gccagtagag gcccttggca gcctcccacc cccacctgca gctccccgtg agccagccat |
| 2761 | ccacagcgag gggcagtggg tgacgctgcc ggcccccctg gacaccatca acgtccacct |
| 2821 | ccgggctggg tacatcatcc ccctgcaggg ccctggcctc acaaccacag agtcccgcca |
| 2881 | gcagcccatg gccctggctg tggccctgac caagggtggg gaggcccgag gggagctgtt |
| 2941 | ctgggacgat ggagagagcc tggaagtgct ggagcgaggg gcctacacac aggtcatctt |
| 3001 | cctggccagg aataacacga tcgtgaatga gctggtacgt gtgaccagtg agggagctgg |
| 3061 | cctgcagctg cagaaggtga ctgtcctggg cgtggccacg gcgccccagc aggtcctctc |
| 3121 | caacggtgtc cctgtctcca acttcaccta cagccccgac accaaggtcc tggacatctg |
| 3181 | tgtctcgctg ttgatgggag agcagtttct cgtcagctgg tgttagccgg gcggagtgtg |
| 3241 | ttagtctctc cagagggagg ctggttcccc agggaagcag agcctgtgtg cgggcagcag |
| 3301 | ctgtgtgcgg gcctgggggt tgcatgtgtc acctggagct gggcactaac cattccaagc |
| 3361 | cgccgcatcg cttgtttcca cctcctgggc cggggctctg gcccccaacg tgtctaggag |
| 3421 | agctttctcc ctagatcgca ctgtgggccg gggccctgga gggctgctct gtgttaataa |
| 3481 | gattgtaagg tttgccctcc tcacctgttg ccggcatgcg ggtagtatta gccacccccc |
| 3541 | tccatctgtt cccagcaccg gagaaggggg tgctcaggtg gaggtgtggg gtatgcacct |
| 3601 | gagctcctgc ttcgcgcctg ctgctctgcc ccaacgcgac cgctgcccgg ctgcccagag |
| 3661 | ggctggatgc ctgccggtcc ccgagcaagc ctgggaactc aggaaaattc acaggacttg |
| 3721 | ggagattcta aatcttaagt gcaattattt ttaataaaag gggcatttgg aatcaaaaaa |
| 3781 | aa |

Homo sapiens Acid Alpha Glucosidase, variant 1 Genbank Accession No.
NP_000143
(SEQ ID NO: 1233)

| | |
|---|---|
| 1 | mgvrhppcsh rllavcalvs lataallghi llhdfllvpr elsgsspvle ethpahqqga |
| 61 | srpgprdaqa hpgrpravpt qcdvppnsrf dcapdkaitq eqceargccy ipakqglqga |
| 121 | qmgqpwcffp psypsyklen lsssemgyta tltrttptff pkdiltlrld vmmetenrlh |
| 181 | ftikdpanrr yevpletphv hsrapsplys vefseepfgv ivrrqldgry llnttvaplf |
| 241 | fadqflqlst slpsqyitgl aehlsplmls tswtritlwn rdlaptpgan lygshpfyla |
| 301 | ledggsahgv fllnsnamdv vlqpspalsw rstggildvy iflgpepksv vqqyldvvgy |
| 361 | pfmppywglg fhlcrwgyss taitrqvven mtrahfpldv qwndldymds rrdftfnkdg |
| 421 | frdfpamvqe lhqggrrymm ivdpaisssg pagsyrpyde glrrgvfitn etgqpligkv |
| 481 | wpgstafpdf tnptalawwe dmvaefhdqv pfdgmwidmn epsnfirgse dgcpnnelen |
| 541 | ppyvpgvvgg tlqaaticas shqflsthyn lhnlygltea iashralvka rgtrpfvisr |
| 601 | stfaghgrya ghwtgdvwss weqlassvpe ilqfnllgvp lvgadvcgfl gntseelcvr |
| 661 | wtqlgafypf mrnhnsllsl pqepysfsep aqqamrkalt lryallphly tlfhqahvag |
| 721 | etvarplfle fpkdsstwtv dhqllwgeal litpvlqagk aevtgyfplg twydlqtvpv |
| 781 | ealgslpppp aaprepaihs egqwvtlpap ldtinvhlra gyiiplqgpg ltttesrqqp |
| 841 | malavaltkg geargelfwd dgeslevler gaytqvifla rnntivnelv rvtsegaglq |
| 901 | lqkvtvlgva tapqqvlsng vpvsnftysp dtkvldicvs llmgeqflvs wc |

Homo sapiens Arylsulfatase B, variant 1 Genbank Accession No. NM_000046
(SEQ ID NO.: 1234)

| | |
|---|---|
| 1 | aaaagtgaat acatgatttt atttaactca ttaataagga aattggtaag gtgttaaaac |
| 61 | caattcaaag gacaatccaa agaacagatc aggaatacta aaataaatat gcaagcggag |
| 121 | gtgaaactgt tttccttggt agtggtggag gggaaggatt gctactccgc tggataaagt |
| 181 | tcatttgtgt atatataaat aagaattatt ttccattgtt atttatctat aacttataaa |
| 241 | gttgtaaaca acttccacgg aatcagactc aacctggaag ggtatggtct ctaggcaatg |
| 301 | caaaaatttt cccctacacc tgttaacaac tataatatct ccagacagag tagacagaaa |
| 361 | gtctggatgg caacgggaat ctactggtca tacggctaac ttcctaattc aataagcacg |
| 421 | tgactaaagg attttttcct tccactcaga tatttcaggc taactagata ctgtgtgctt |
| 481 | cttagtgtca ctgcttagtg ggggagccag ctctgagtgg ggtcatatcc ggacaagcga |
| 541 | atgagctatt tattcaatga ccacgcaaca ctccaaatcc tcccagggca acttgaaagt |
| 601 | aaccgcacct tccaaagggc accgtgcaat cagactgtgt gtttggcctc ctgtttgcta |
| 661 | gtggggagga agcggcttca tgggtgtaca ctacgcataa atgaatgtga aaggctattt |
| 721 | agacctctgc cttttcaccg tcctcccacc tgccacaggc tgggctcttg tgctagaaat |
| 781 | gacttgctag ctagacatca tggttcagga tctgagtcag aggtttaacc atttataagc |
| 841 | ttttttctta tgaaaaattg gcactaatta taatgtctaa ctgtcagagt tgttgcaggc |
| 901 | tttacaggag acgcgggctg tgaagatgct ttgtaaattg tgaagcgtta ttaaagaaca |
| 961 | catctttttt ttttaggaaa ccacagtgca aatttaattg ccggggaaga taacgggcct |
| 1021 | tggtgccctc caagcgtcag ctgagtttcc aagaagccgg gcagcgggcg cccgcgggtt |
| 1081 | cgtctctggc tcctcctccg ccacagcagc cgggggcccg ggtcggaggc ggcgggggcc |
| 1141 | gagcgcccgg cctcgcaagc ccacggcccg ctgggggtgc cgtcccgcgc cggggcggag |
| 1201 | caggccccgg cagcccagtt cctcattcta tcagcggtac aaggggctgg tggcgccaca |
| 1261 | ggcgctggga ccgcgggcgg acaaggatgg gtccgcgcgg cgcggcgagc ttgccccgag |
| 1321 | gccccggacc tcggcggctg ctcctccccg tcgtcctccc gctgctgctg ctgctgttgt |
| 1381 | tggcgccgcc gggctcgggc gccggggcca gccggccgcc ccacctggtc ttcttgctgg |
| 1441 | cagacgacct aggctggaac gacgtcggct tccacggctc ccgcatccgc acgccgcacc |
| 1501 | tggacgcgct ggcggccggc ggggtgctcc tggacaacta ctacacgcag ccgctgtgca |
| 1561 | cgccgtcgcg gagccagctg ctcactggcc gctaccagat ccgtacaggt ttacagcacc |
| 1621 | aaataatctg gccctgtcag cccagctgtg ttcctctgga tgaaaaactc ctgccccagc |
| 1681 | tcctaaaaga agcaggttat actacccata tggtcggaaa atggcacctg ggaatgtacc |
| 1741 | ggaaagaatg ccttccaacc cgccgaggat ttgataccta ctttggatat ctcctgggta |
| 1801 | gtgaagatta ttattcccat gaacgctgta cattaattga cgctctgaat gtcacacgat |
| 1861 | gtgctcttga ttttcgagat ggcgaagaag ttgcaacagg atataaaaat atgtattcaa |
| 1921 | caaacatatt caccaaaagg gctatagccc tcataactaa ccatccacca gagaagcctc |
| 1981 | tgtttctcta ccttgctctc cagtctgtgc atgagcccct tcaggtccct gaggaatact |
| 2041 | tgaagccata tgactttatc caagacaaga acaggcatca ctatgcagga atggtgtccc |
| 2101 | ttatggatga agcagtagga aatgtcactg cagctttaaa aagcagtggg ctctggaaca |
| 2161 | acacggtgtt catcttttct acagataacg gagggcagac tttggcaggg ggtaataact |
| 2221 | ggccccttcg aggaagaaaa tggagcctgt gggaaggagg cgtccgaggg gtgggctttg |
| 2281 | tggcaagccc cttgctgaag cagaagggcg tgaagaaccg ggagctcatc cacatctctg |
| 2341 | actggctgcc aacactcgtg aagctggcca ggggacacac caatggcaca aagcctctgg |
| 2401 | atggcttcga cgtgtggaaa accatcagtg aaggaagccc atcccccaga attgagctgc |
| 2461 | tgcataatat tgacccgaac ttcgtggact cttcaccgtg tcccaggaac agcatggctc |
| 2521 | cagcaaagga tgactcttct cttccagaat attcagcctt taacacatct gtccatgctg |
| 2581 | caattagaca tggaaattgg aaactcctca cgggctaccc aggctgtggt tactggttcc |
| 2641 | ctccaccgtc tcaatacaat gtttctgaga taccctcatc agacccacca accaagaccc |
| 2701 | tctggctctt tgatattgat cgggaccctg aagaaagaca tgacctgtcc agagaatatc |
| 2761 | ctcacatcgt cacaaagctc ctgtcccgcc tacagttcta ccataaacac tcagtccccg |
| 2821 | tgtacttccc tgcacaggac ccccgctgtg atcccaaggc cactggggtg tggggccctt |
| 2881 | ggatgtagga tttcagggag gctagaaaac ctttcaattg gaagttggac ctcaggcctt |
| 2941 | ttctcacgac tcttgtctca tttgttatcc caacctgggt tcacttggcc cttctcttgc |
| 3001 | tcttaaacca caccgaggtg tctaatttca acccctaatg catttaagaa gctgataaaa |
| 3061 | tctgcaacac tcctgctgtt ggctggagca tgtgtctaga ggtgggggtg gctgggttta |
| 3121 | tccccctttc ctaagccttg ggacagctgg gaacttaact tgaaatagga agttctcact |
| 3181 | gaatcctgga ggctggaaca gctggctctt ttagactcac aagtcagacg ttcgattccc |
| 3241 | ctctgccaat agccagtttt attggagtga atcacatttc ttacgcaaat gaagggagca |
| 3301 | gacagtgatt aatggttctg ttggccaagg cttctccctg tcggtgaagg atcatgttca |
| 3361 | ggcactccaa gtgaaccacc cctcttggtt caccccttac tcacttatct catcacagag |
| 3421 | cataaggccc attttgttgt tcaggtcaac agcaaaatgc ctgcaccatg actgtggctt |
| 3481 | ttaaaataaa gaaatgtgtt tttatcgtaa tttatttccc cccagccatt gctcactctg |
| 3541 | tctagacttc ctgccacttc caattcttct gtggcttttc ctgcctttcc ttttgacctc |
| 3601 | agtagtccta tccctgggaa ggccactttg cttctctacc tgagcacccc tgatttctgg |
| 3661 | aacgctgctg agccctgcct tacttttgcc cctagggctg aagctagagg cctccccgta |
| 3721 | ataggcggtg gagttgctct gtgaggatgt tcatggtaga cactaagagg gctgggtggg |
| 3781 | agatgcttgg ctctgtggca tctgttcagc gaggcttttc ctatattgca tggagttagt |
| 3841 | cattgtgatt gtagctttat ttcataatat attaagactt gcactgctat ttactagcag |
| 3901 | tgagaagaaa cctcaggaaa ggatatgaaa aagcaagtgg ccagtgtctg ggatactggg |
| 3961 | ccttggtaaa gcagaggagg gcacacccac agtcctctta ttctctgttt tactgcttgt |
| 4021 | tttgaggttc tggggtctgg caaagaggat gcagtttgac acctgcagcc ctttctcaat |
| 4081 | cccactaatg tcttactaat gtggaacagt ccatattagc tccagagagt gtcaaaccca |
| 4141 | gagaaatgtg tgcaaaaatg atactctttt ctgcattagc cccaccattg tgttcaccaa |
| 4201 | tgcttggaac actgcctgaa ggcactcatt ttttaatttt tattttattt ttaatttttt |
| 4261 | atatctttat gagacgatct cactctgtca ccaggttgga gtacagtggt acaatcacaa |
| 4321 | ctcaccgtag cctcaaactc ctgggctcaa gtgattctcc cacctcaggc acccaaatag |
| 4381 | ctggaactac aggcatatac cgccacaccc agctaatttt attttttgaa aagacaaggt |
| 4441 | tccctatgtt gcccagctgg tcttaaactc ctgggctcca gcaattatcc cagcttgggc |
| 4501 | tccaaaagtg ctgggattac aggcatgagt caccatgcct ggcctcattt tttaaaacaa |
| 4561 | atgaataaat ggacaaatga gtaaatgaga aagtctcaca ccatgaaaga tgctagtcca |
| 4621 | atgagctgaa tacagaggta atataaatgt cttccagctg ttgcttttct gttctcaagc |
| 4681 | tgcccctcct ggggtaggag cataatctac atcactgggc agtcacagga cactctatag |
| 4741 | caaggttgta gcgtcctctc cagtgggggg agaaaaggaa ctgtgcctac caaaggtact |
| 4801 | ctcttgtcag caatttccat ttctatactt tatgggacac tagaaactaa aagcaacaaa |
| 4861 | taatctgata taagtccttg tatagtcatc cttcaattca gtagcaatat tttctggtca |
| 4921 | ctactaacct gtattgtatt aaaatgagac tattggaagg aaatggtgct aaaactaata |
| 4981 | acatctctta ccaaccttta cccaactcct gggttggcaa acagctgacc aaactgccat |
| 5041 | cacctcccac ttggaagtgt atggccgaca gcatgaaata gctgagccca gatgttcctt |
| 5101 | ctgcatcctc cgaatcccag ggctgggtgt aggtagccgt tggaggccat cgctacaggg |
| 5161 | cacctatctg ttatcgctgc tgtcctccca acagctgtct ccagttctag ttccttggtt |
| 5221 | ttcaggcaca gtgggggatg ttctgcaccc agtggacttc aaaagagttt tgaagactta |
| 5281 | attttttgaa aaacaagtac ttgagatttt ggtttatcca taatagaatg tatttcatta |
| 5341 | gattctctga ttctatataa gaatgtgaaa agattgatat attgttgtta gaaataatgt |
| 5401 | tatttctttc caattttttt tttttttttt tttgagatgg agtctcgctc tgtcacccag |
| 5461 | gctggagtgc agtggtgtga tctcggctca ctgcagcctc taactcccag gttcaagcta |
| 5521 | ttctcctgcc tcagcctccc aagtagctgg attacaggca tacaccacca cgcctggcta |
| 5581 | tgttttgtat ttttcgtaga gatagggttt caccatgttg gccaggctgg tctcaaactc |
| 5641 | ctgacctcaa gtgatccacc cacttcagct tcccaaagca ctgggattac aggtgtgagc |
| 5701 | cactgtgccc ggcaaatttt tttaccttta cagaaggttt tgcttattta attgtgagct |
| 5761 | catttttctt tgttactttt gtccccccag atttggggga caaaataaaa ttaatctttt |
| 5821 | aaaatgtgtc agccatatgt atggggcttc catttggggt gaggagaaag ttctggaact |
| 5881 | agatagtggt catggttata caacatcata aatgcaatta ctgccactga attgtatgtt |
| 5941 | ttaaagtggt taaaatgtta agttttatgt tttattacaa tttttaaatg tgtcaaccaa |
| 6001 | ctttatagta cataaattat atctcagtaa agctgttaaa taaataaata tagtaaaaat |
| 6061 | tttagaacta aaaaaa |

Homo sapiens Arylsulfatase B, variant 1 Genbank Accession No. NP_000037
(SEQ ID NO. 1235)

| | |
|---|---|
| 1 | mgprgaaslp rgpgprrlll pvvlplllll llappgsgag asrpphlvfl laddlgwndv |
| 61 | gfhgsrirtp hldalaaggv lldnyytqpl ctpsrsqllt gryqirtglq hqiiwpcqps |
| 121 | cvpldekllp qllkeagytt hmvgkwhlgm yrkeclptrr gfdtyfgyll gsedyysher |
| 181 | ctlidalnvt rcaldfrdge evatgyknmy stniftkrai alitnhppek plflylalqs |
| 241 | vheplqvpee ylkpydfiqd knrhhyagmv slmdeavgnv taalkssglw nntvfifstd |
| 301 | nggqtlaggn nwplrgrkws lweggvrgvg fvaspllkqk gvknrelihi sdwlptivkl |
| 361 | arghtngtkp ldgfdvwkti segspsprie llhnidpnfv dsspcprnsm apakddsslp |
| 421 | eysafntsvh aairhgnwkl ltgypgcgyw fpppsqynvs eipssdpptk tlwlfdidrd |
| 481 | peerhdlsre yphivtklls rlqfyhkhsv pvyfpaqdpr cdpkatgvwg pwm |

Homo sapiens alpha galactosidase A Genbank Accession No. NM_000169
(SEQ ID NO. 1236)

| | |
|---|---|
| 1 | aaacaataac gtcattattt aataagtcat cggtgattgg tccgcccctg aggttaatct |
| 61 | taaaagccca ggttacccgc ggaaatttat gctgtccggt caccgtgaca atgcagctga |
| 121 | ggaacccaga actacatctg ggctgcgcgc ttgcgcttcg cttcctggcc ctcgtttcct |
| 181 | gggacatccc tggggctaga gcactggaca atggattggc aaggacgcct accatgggct |
| 241 | ggctgcactg ggagcgcttc atgtgcaacc ttgactgcca ggaagagcca gattcctgca |
| 301 | tcagtgagaa gctcttcatg gagatggcag agctcatggt ctcagaaggc tggaaggatg |
| 361 | caggttatga gtacctctgc attgatgact gttggatggc tccccaaaga gattcagaag |
| 421 | gcagacttca ggcagaccct cagcgctttc ctcatgggat tcgccagcta gctaattatg |
| 481 | ttcacagcaa aggactgaag ctagggattt atgcagatgt tggaaataaa acctgcgcag |
| 541 | gcttccctgg gagttttgga tactacgaca ttgatgccca gacctttgct gactggggag |
| 601 | tagatctgct aaaatttgat ggttgttact gtgacagttt ggaaaatttg gcagatggtt |
| 661 | ataagcacat gtccttggcc ctgaatagga ctggcagaag cattgtgtac tcctgtgagt |
| 721 | ggcctcttta tatgtggccc tttcaaaagc ccaattatac agaaatccga cagtactgca |
| 781 | atcactggcg aaattttgct gacattgatg attcctggaa aagtataaag agtatcttgg |
| 841 | actggacatc ttttaaccag gagagaattg ttgatgttgc tggaccaggg ggttggaatg |
| 901 | acccagatat gttagtgatt ggcaactttg gcctcagctg gaatcagcaa gtaactcaga |
| 961 | tggccctctg ggctatcatg gctgctcctt tattcatgtc taatgacctc cgacacatca |
| 1021 | gccctcaagc caaagctctc cttcaggata aggacgtaat tgccatcaat caggacccct |
| 1081 | tgggcaagca agggtaccag cttagacagg gagacaactt tgaagtgtgg gaacgacctc |
| 1141 | tctcaggctt agcctgggct gtagctatga taaaccggca ggagattggt ggacctcgct |
| 1201 | cttataccat cgcagttgct tccctgggta aaggagtggc ctgtaatcct gcctgcttca |
| 1261 | tcacacagct cctccctgtg aaaaggaagc tagggttcta tgaatggact tcaaggttaa |
| 1321 | gaagtcacat aaatcccaca ggcactgttt tgcttcagct agaaaataca atgcagatgt |
| 1381 | cattaaaaga cttactttaa aatgtttatt ttattgcc |

Homo sapiens alpha galactosidase A Genbank Accession No. NP_000160
(SEQ ID NO. 1237)

| | |
|---|---|
| 1 | mqlrnpelhl gcalalrfla lvswdipgar aldnglartp tmgwlhwerf mcnldcqeep |
| 61 | dsciseklfm emaelmvseg wkdagyeylc iddcwmapqr dsegrlqadp qrfphgirql |
| 121 | anyvhskglk lgiyadvgnk tcagfpgsfg yydidaqtfa dwgvdllkfd gcycdslenl |
| 181 | adgykhmsla lnrtgrsivy scewplymwp fqkpnyteir qycnhwrnfa diddswksik |
| 241 | sildwtsfnq erivdvagpg gwndpdmlvi gnfglswnqq vtqmalwaim aaplfmsndl |
| 301 | rhispqakal lqdkdviain qdplgkqgyq lrqgdnfevw erplsglawa vaminrqeig |
| 361 | gprsytiava slgkgvacnp acfitqllpv krklgfyewt srlrshinpt gtvllqlent |
| 421 | mqmslkdll |

Homo sapiens alpha-L-iduronidase Genbank Accession No. NM_000203
(SEQ ID NO. 1238)

| | |
|---|---|
| 1 | gtcacatggg gtgcgcgccc agactccgac ccggaggcgg aaccggcagt gcagcccgaa |
| 61 | gccccgcagt ccccgagcac gcgtggccat gcgtcccctg cgcccccgcg ccgcgctgct |
| 121 | ggcgctcctg gcctcgctcc tggccgcgcc cccggtggcc ccggccgagg ccccgcacct |
| 181 | ggtgcatgtg gacgcggccc gcgcgctgtg gcccctgcgg cgcttctgga ggagcacagg |
| 241 | cttctgcccc ccgctgccac acagccaggc tgaccagtac gtcctcagct gggaccagca |
| 301 | gctcaacctc gcctatgtgg gcgccgtccc tcaccgcggc atcaagcagg tccggaccca |
| 361 | ctggctgctg gagcttgtca ccaccagggg gtccactgga cggggcctga gctacaactt |
| 421 | cacccacctg gacgggtacc tggaccttct cagggagaac cagctcctcc cagggtttga |
| 481 | gctgatgggc agcgcctcgg gccacttcac tgactttgag gacaagcagc aggtgtttga |
| 541 | gtggaaggac ttggtctcca gcctggccag gagatacatc ggtaggtacg gactggcgca |
| 601 | tgtttccaag tggaacttcg agacgtggaa tgagccagac caccacgact ttgacaacgt |
| 661 | ctccatgacc atgcaaggct tcctgaacta ctacgatgcc tgctcggagg gtctgcgcgc |
| 721 | cgccagcccc gccctgcggc tgggaggccc cggcgactcc ttccacaccc caccgcgatc |
| 781 | cccgctgagc tggggcctcc tgcgccactg ccacgacggt accaacttct tcactgggga |
| 841 | ggcgggcgtg cggctggact acatctccct ccacaggaag ggtgcgcgca gctccatctc |
| 901 | catcctggag caggagaagg tcgtcgcgca gcagatccgg cagctcttcc ccaagttcgc |
| 961 | ggacaccccc atttacaacg acgaggcgga cccgctggtg ggctggtccc tgccacagcc |
| 1021 | gtggagggcg gacgtgacct acgcggccat ggtggtgaag gtcatcgcgc agcatcagaa |
| 1081 | cctgctactg gccaacacca cctccgcctt cccctacgcg ctcctgagca acgacaatgc |
| 1141 | cttcctgagc taccacccgc accccttcgc gcagcgcacg ctcaccgcgc gcttccaggt |
| 1201 | caacaacacc cgcccgccgc acgtgcagct gttgcgcaag ccggtgctca cggccatggg |
| 1261 | gctgctggcg ctgctggatg aggagcagct ctgggccgaa gtgtcgcagg ccgggaccgt |
| 1321 | cctggacagc aaccacacgg tgggcgtcct ggccagcgcc caccgccccc agggcccggc |
| 1381 | cgacgcctgg cgcgccgcgg tgctgatcta cgcgagcgac gacacccgcg cccaccccaa |
| 1441 | ccgcagcgtc gcggtgaccc tgcggctgcg cggggtgccc cccggcccgg gcctggtcta |
| 1501 | cgtcacgcgc tacctggaca acgggctctg cagccccgac ggcgagtggc ggcgcctggg |
| 1561 | ccggcccgtc ttccccacgg cagagcagtt ccggcgcatg cgcgcggctg aggacccggt |
| 1621 | ggccgcggcg ccccgcccct tacccgccgg cggccgcctg accctgcgcc ccgcgctgcg |
| 1681 | gctgccgtcg cttttgctgg tgcacgtgtg tgcgcgcccc gagaagccgc ccgggcaggt |
| 1741 | cacgcggctc cgcgccctgc ccctgaccca agggcagctg gttctggtct ggtcggatga |
| 1801 | acacgtgggc tccaagtgcc tgtggacata cgagatccag ttctctcagg acggtaaggc |
| 1861 | gtacaccccg gtcagcagga agccatcgac cttcaacctc tttgtgttca gcccagacac |
| 1921 | aggtgctgtc tctggctcct accgagttcg agccctggac tactgggccc gaccaggccc |
| 1981 | cttctcggac cctgtgccgt acctggaggt ccctgtgcca agagggcccc catccccggg |
| 2041 | caatccatga gcctgtgctg agccccagtg ggttgcacct ccaccggcag tcagcgagct |
| 2101 | ggggctgcac tgtgcccatg ctgccctccc atcaccccct ttgcaatata tttttatatt |
| 2161 | ttattatttt cttttatatc ttggtaaaaa aaaaaaaaaa aaa |

Homo sapiens alpha-L-iduronidase Genbank Accession No. NP_000194
(SEQ ID NO. 1239)

| | |
|---|---|
| 1 | mrplrpraal lallasllaa ppvapaeaph lvhvdaaral wplrrfwrst gfcpplphsq |
| 61 | adqyvlswdq qlnlayvgav phrgikqvrt hwllelvttr gstgrglsyn fthldgyldl |
| 121 | lrenqllpgf elmgsasghf tdfedkqqvf ewkdlvssla rryigrygla hvskwnfetw |
| 181 | nepdhhdfdn vsmtmqgfln yydacseglr aaspalrlgg pgdsfhtppr splswgllrh |
| 241 | chdgtnfftg eagvrldyis lhrkgarssi sileqekvva qqirqlfpkf adtpiyndea |
| 301 | dplvgwslpq pwradvtyaa mvvkviaqhq nlllanttsa fpyallsndn aflsyhphpf |
| 361 | aqrtltarfq vnntrpphvq llrkpvltam gllalldeeq lwaevsqagt vldsnhtvgv |
| 421 | lasahrpqgp adawraavli yasddtrahp nrsvavtlrl rgyppgpgly yvtryldngl |
| 481 | cspdgewrrl grpvfptaeq frrmraaedp vaaaprplpa ggrltlrpal rlpslllyhy |
| 541 | carpekppgq vtrlralplt qgqlvlvwsd ehvgskclwt yeiqfsqdgk aytpvsrkps |
| 601 | tfnlfvfspd tgavsgsyrv raldywarpg pfsdpvpyle vpvprgppsp gnp |

### Promoter region sequences for arylsulfataseB (ARSB), acid alpha glucosidase (GAA), glucocerebrosidase (GBA), alpha galactosidase (GLA), iduronate 2-sulfatase (IDS) and alpha-L-iduronidase (IDUA)

Arylsulfatase B Promoter Region, Variant 1 (SEQ ID NO: 1240):

Gene region ARSB var1; Chromosome Accession NC 000005.9; and Chromosome Coords, c78073031-78072682 >ref|NT_{—}006713.15|:28667041-28667390 Homo sapiens chromosome 5 genomic contig, GRCh37 reference primary assembly:

1>> AGGAGCTGAGGCCTCCAGCCAAGAGCCTTGTGAGTGAGTCAGCTTA
AAAGTGGAGCCTGCAGCCCCACTCAAGTCTTCAGATGACCACAGGCCTGC
TTGACAACAATCTCACGAGAGACC
CTGAGCCAGAACCACTGGGCTAAACCACTCCCAGTGTCCTTACCCTCATA
GAATGTGTGAAGTTATGAATGTTTATTGTTTTAAGCTGCTAAGTTTTGGG
GTAATTTGTTACGCAGCAATAGAGAGCTAATACTCTCTTTTACTGTCACT
TTGTTTTCACTCCTACCCTTCCTTGACCTCCCTCTAATACCTTATTTTAT
AGTTTATATTCTTAGGGCAATGATTGACAC<< 350

Arylsulfatase B Promoter Region, Variant 2 (SEQ ID NO: 1241):

Gene region ARSB var2; Chromosome Accession NC 000005.9; and Chromosome Coords, c78073622-78073273->ref|NT_{—}006713.15|:c28667981-28667632 Homo sapiens chromosome 5 genomic contig, GRCh37 reference primary assembly:

1>> GCTCACTGCAGCCTCTAACTCCCAGGTTCAAGCTATTCTCCTGCCT
CAGCCTCCCAAGTAGCTGGATTACAGGCATACACCACCACGCCTGGCTAT
GTTTTGTATTTTTCGTAGAGATAGGGTTTCACCATGTTGGCCAGGCTGGT
CTCAAACTCCTGACCTCAAGTGATCCACCCACTTCAGCTTCCCAAAGCAC
TGGGATTACAGGTGTGAGCCACTGTGCCCGGCAAATTTTTTTACCTTTAC
AGAAGGTTTTGCTTATTTAATTGTGAGCTCATTTTTCTTTGTTACTTTTG
TCCCCCCAGATTTGGGGGACAAAATAAAATTAATCTTTTAAAATGTGTCA
GCCA<< 350

Acid Alpha Glucosidase Promoter Sequence (SEQ ID NO: 1242):

Gene region GAA; Chromosome Accession NC 000017.10; and Chromosome Coords, c78075005-78075354 ref|NT_{—}010783.15|:43349157-43349506 Homo sapiens chromosome 17 genomic contig, GRCh37 reference primary assembly

1>> CTACGAGCTGCGGGGACCCAGGCCGGGGCAGCGGGGGCCACGCCCC
ATCTCCGACCCCACGGGGACCGGGCCGGGACTGCGCCAGCGGGGGCCTCG
CCCCGTCTCTGACCCCAGAGGAACCGGCAGCGGGCAGCACGCGTGGGCCT
CTCCCCGCGGGACGCCGGACGCGCAGCCAGACGCGCTCCCCAGGCCCCCT
CCGAGAGCGAGGACGCGCCCAGGCCCGCTCTGCCGGAGCCGCCACTGGGG
GGCGTAGCGCGGACGCGCACCCTTGCCTCGGGCGCCTGCGCGGGAGGCCG
CGTCACGTGACCCACCGCGGCCCCGCCCCGCGACGAGCTCCCGCCGGTCA
CGTG<< 350

Glucocerebrosidase Promoter Sequence, Alternate Promoter (SEQ ID NO: 1243):

Gene region GBA alt Prom; Chromosome Accession NC_{—}000001.10; and Chromosome Coords, c155207676-155207327 ref|NT_{—}004487.19|:c6696318-6695969 Homo sapiens chromosome 1 genomic contig, GRCh37 reference primary assembly

1>> CTGTCACCCAGGCTGGAGTGCTGTGGCGCCATCTTCACTCACTGTA
ACCTCTGCCTCCTGAGTTCAAGCAATTCTCCTGCCTCAGCCTTCCAAGTA
GCTGGGATTATAGGCGCCTGCCACCAGGCCCAGCTGATTTTTCTATTTTT
AGTAGAGACGGGGTTTCGCCAGGCTGTTCTCGAACTCCTGAACTCAAGTG
ATCCACCTGCCTCGGCTTCCCAAAGTGCTGGGATTACAGGTGTGAGCCAC
CACACCCAGCTGGTCTGGTCCACTTTCTTGGCCGGATCATTCATGACCTT
TCTCTTGCCAGGTTCCTGGATGCCTATGCTGAGCACAAGTTACAGTTCTG
GGCA<< 350

Glucocerebrosidase Promoter Sequence (SEQ ID NO: 1244)

Gene region GBA; Chromosome Accession NC_{—}000001.10; and Chromosome Coords, c155204242-155203893 ref|NT_{—}004487.19|:c6692884-6692535 Homo sapiens chromosome 1 genomic contig, GRCh37 reference primary assembly

1>> GTCAGTGTGAGTGGCTTTATTCTGGGTGGCAGCACCCCGTGTCCGG
CTGTACCAACAACGAGGAGGCACGGGGGCCTCTGGAATGCATGAGAGTAG
AAAAACCAGTCTTGGGAGCGTGAGGACAAATCATTCCTCTTCATCCTCCT
CAGCCATGCCCAGGGTCCGGGTGCCTGGGGCCCGAGCAGGCGTTGCCCGC
TGGATGGAGACAATGCCGCTGAGCAAGGCGTAGCCCACCATGGCTGCCAG
TCCTGCCAGCACAGATAGGATCTGGTTCCGGCGCCGGTATGGCTCCTCCT
CAGTCTCTGGGCCTGCTGGTGTCTGGCGTTGCGGTGGTACCTCAGCTGAG
GGTC<< 350

Acid Alpha Glucosidase Promoter Sequence (SEQ ID NO: 1245)

Gene region GLA; Chromosome Accession NC000023.10; and Chromosome Coords, c100652778-100652429 ref|NT_{—}011651.17|:c23949086-23948737 Homo sapiens chromosome X genomic contig, GRCh37 reference primary assembly

1>> AACTACTACTTCCTGTCCACCTTTTTCTCCATTCACTTTAAAAGCT
CAAGGCTAGGTGGCTCATGCCTGTAATCCCAGCACTTTGGGAGGCTGAGG
CGGGCAGATCACCTGAGGTCGGGACTTTGAGACCCGCCTGGACAACATGG
TGAAACCCCATTTCTAATAAAAATATAAAAATTAGCCAGGTGTGGTGGCG
CACGCCTGTGGTCCCAGCTACTCTGGGGGCTGAGGCATGAGAATCGCTTG
AACCCGGGAGGTGGAGGTTGCATTGAGCTGAGATCATGCCACCTCACTCC
AGCCTGGGCAACAAAGATTCCATCTCAAAAAAAAAAAAAAAAGCCAGGCA
CAGT<< 350

Iduronate 2-Sulfatase Promoter Sequence (SEQ ID NO: 1246)

Gene regionIDS; Chromosome Accession NC000023.10; and Chromosome Coords, c148559948-148560297 ref|NT_{—}011681.16|:5002624-5002973 Homo sapiens chromosome X genomic contig, GRCh37 reference primary assembly

1>> AAAAAAAAAAATTAATGAAGCAGGGCTGTGCAGCCATTACAGCTGT
TAATCAAGAACTGTCAATGACATAAAAAACCAAGCTAACATTGTGTTAGA
TCAAAAAAACAAGATATAGATTATATGTGGTAGTCTGCATGTATGTAAAG
CTAAATATGTCCATATCTGCATATATGTCTTTAGGCAGGTAAAAGAATGA
AAGGAAATATACAAATGTCAGCAGTGGGTATGTCTCAAATGCTGGGCTTC
TTTTCATCTCCAGTTTTCACATGTTCCAGATTTTATGCAATGAGCATATA
TTACTCTGATAATGGGGTGGAGAGACACAGTAAGCATAAAACCAAACCCC
AAAC<< 350

Alpha-L-Iduronidase Promoter Sequence (SEQ ID NO: 1247)

Gene regionIDUA; Chromosome Accession NC_{—}000004.11; and Chromosome Coords, c980435-980784ref|NT_{—}037622.5|:970435-970784 Homo sapiens chromosome 4 genomic contig, GRCh37 reference primary assembly

1>> CTCAGGAGGCTGGGGTGAGAAAATCGCTGAAGCCCCGGAGATGGAG
GTTGCAGTGAGCTGAGATCGCGCCACTGCACCTCAGCCTGGGCGACAAAG
CAAGACTCTGTCTCAAAAACACACAAAAACAGAGAAAAACAAGACAGTAA
TGGCTCAACTCACATAGCACCAACGGGCGAAGCGTTCTTCTGAGCGCTTT
CCGAGTCATCGGTCCTCAGAGCAGCCCCTGAGGCCCGCAAGGAAGCGGGG
CTCCAAGCCCTGCCGTGCTCCCGGCTCCCCGAGGCTCCCCGAGGCCACCC
AACCCCTCCCACCCGGCCATCGCCCCCTCACCAAGGCCCCGCCCCGCGGC
GGCG<< 350

1. A method of producing a polypeptide with a modified glycosylation pattern at an N-linked glycosylation site, the method comprising:
(a) culturing a cell comprising a polypeptide to be modified in the presence of at least one RNA effector molecule that inhibits expression of a gene product involved in protein glycosylation such that at least one polypeptide N-linked glycosylation site is modified to have a terminal mannose, and wherein the cell is cultured under conditions permitting glycosylation and for a sufficient time to allow expression of the polypeptide to be modified; and
(b) isolating the polypeptide,
wherein the polypeptide produced by step (a) comprises a terminal mannose in at least one N-linked glycosylation site, thereby producing a polypeptide with a modified glycosylation pattern.

2. The method of claim 1, further comprising culturing the cell with an RNA effector molecule that inhibits expression of the mannose 6 phosphate receptor.

3. The method of claim 1, wherein at least two N-linked glycosylation sites are modified.

4. The method of claim 1, wherein at least three N-linked glycosylation sites are modified.

5. The method of claim 1, wherein at least four N-linked glycosylation sites are modified.

6. The method of claim 1, wherein the modified N-linked glycosylation site comprises an oligomannosyl structure.

7. The method of claim 6, wherein the modified N-linked glycosylation site consists of an oligomannosyl structure selected from the group consisting of: Man₂GlcNAc₂, Man₃GlcNAc₂, Man₄GlcNAc₂, Man₅ GlcNAc₂, Man₆GlcNAc₂, Man₇GlcNAc₂, Man₈GlcNAc₂, and Man₉GlcNAc₂.

8. The method of claim 1, wherein the polypeptide comprises 2, 3, 4, 5, 6, 7, 8, or 9 terminal mannoses in the at least one N-linked glycosylation site.

9. The method of claim 1, wherein the gene product that is inhibited is selected from the group consisting of: Mgat1, Mgat4, SLC35A1, SLC35A2, and GNE.

10. The method of claim 1, wherein the polypeptide binds a mannose receptor present on macrophages.

11. The method of claim 1, wherein the polypeptide is secreted from the cell.

12. The method of claim 1, wherein the at least one RNA effector molecule is an siRNA.

13. The method of claim 1, wherein the at least one RNA effector molecule comprises
(a) a sense strand comprising a sequence selected from the group consisting of: SEQ ID NO. 1-33, SEQ ID NO. 67-94, SEQ ID NO. 123-154, SEQ ID NO. 187-221, and SEQ ID NO. 257-282; and
(b) a complementary anti-sense strand comprising a sequence selected from the group consisting of SEQ ID NO. 34-66, SEQ ID NO. 95-122, SEQ ID NO. 155-186, SEQ ID NO. 222-256 and SEQ ID NO. 283-308.

14. The method of claim 1, wherein step (a) is performed by adding the RNA effector molecule to a culture medium used to produce the polypeptide.

15. The method of claim 14, wherein the RNA effector molecule is added in combination with a reagent that facilitates RNA effector molecule uptake into the cell.

16. The method of claim 1, wherein the polypeptide is used in treatment of a lysosomal storage disease.

17. The method of claim 16, wherein the polypeptide is selected from the group consisting of: glucocerebrosidase, idursulfase, alglucosidase alfa, galsulfase, agalsidase beta, and laronidase.

18. The method of claim 17, wherein the polypeptide comprises at least one mutation.

19. The method of claim 1, wherein the polypeptide is glucocerebrosidase.

20. The method of claim 19, wherein the glucocerebrosidase comprises an arginine to histidine mutation at amino acid 495.

21. The method of claim 1, wherein two or more RNA effector molecules are cultured with the cell.

22. An isolated polypeptide comprising a modified mannosylation pattern produced by the method of claim 1, wherein the polypeptide comprises a terminal mannose at at least one N-linked glycosylation site.

23. The polypeptide of claim 22, wherein the polypeptide lacks a mannose phosphate group.

24. The polypeptide of claim 22, wherein the polypeptide has a reduced affinity for the mannose 6 phosphate receptor.

25. The polypeptide of claim 22, wherein at least two N-linked glycosylation sites are modified.

26. The polypeptide of claim 22, wherein at least three N-linked glycosylation sites are modified.

27. The polypeptide of claim 22, wherein at least four N-linked glycosylation sites are modified.

28. The polypeptide of claim 22, wherein the modified N-linked glycosylation site comprises an oligomannosyl structure.

29. The polypeptide of claim 22, wherein the modified N-linked glycosylation site consists of an oligomannosyl structure selected from the group consisting of: Man₂GlcNAc₂, Man₃GlcNAc₂, Man₄GlcNAc₂, Man₅ GlcNAc₂, Man₆GlcNAc₂, Man₇GlcNAc₂, Man₈GlcNAc₂, and Man₉GlcNAc₂.

30. The polypeptide of claim 22, wherein the polypeptide comprises 2, 3, 4, 5, 6, 7, 8, or 9 terminal mannoses in the at least one N-linked glycosylation chain.

31. The polypeptide of claim 22, wherein the polypeptide binds a mannose receptor present on macrophages.

32. The polypeptide of claim 22, wherein the polypeptide is secreted from the cell.

33. The polypeptide of claim 22, wherein the polypeptide is used in treatment of lysosomal storage disease.

34. The polypeptide of claim 33, wherein the polypeptide is selected from the group consisting of: glucocerebrosidase, idursulfase, alglucosidase alfa, galsulfase, agalsidase beta, and laronidase.

35. The polypeptide of claim 22, wherein the polypeptide comprises at least one mutation.

36. The polypeptide of claim 22, wherein the polypeptide is glucocerebrosidase.

37. The polypeptide of claim 36, wherein the glucocerebrosidase comprises an arginine to histidine mutation at amino acid 495.

38. An isolated mammalian host cell, in which the mRNA expression of a target gene selected from the group consisting of: Mgat1, Mgat4, SLC35A1, SLC35A2, and GNE is inhibited by RNA interference, wherein when a gene encoding a polypeptide is introduced into the host cell and expressed, the host cell produces a polypeptide comprising the encoded polypeptide molecule which contains a terminal mannose in at least one glycosylation chain, said polypeptide having increased affinity for the mannose receptor when compared with the polypeptide produced in the presence of Mgat1, Mgat4, SLC35A1, SLC35A2, or GNE expression, thereby producing a polypeptide with increased macrophage internalization.

39. The host cell of claim 38, wherein the cell is a CHO cell.

40. The host cell of claim 38, wherein the polypeptide is used to treat a lysosomal storage disease.

41. The host cell of claim 38, wherein the polypeptide is selected from the group consisting of: glucocerebrosidase, idursulfase, alglucosidase alfa, galsulfase, agalsidase beta, and laronidase.

42. The host cell of claim 41, wherein the polypeptide comprises at least one mutation.

43. The host cell of claim 38, wherein the polypeptide is glucocerebrosidase.

44. The host cell of claim 43, wherein the glucocerebrosidase comprises an arginine to histidine mutation at amino acid 495.

45. The host cell of claim 38, wherein the polypeptide is introduced with an expression vector.

46. The host cell of claim 38, wherein the cell is cultured in suspension.

47. The host cell of claim 38, wherein the cell is cultured in a bioreactor.

48. The host cell of claim 46, wherein the cell is cultured in a volume selected from the group consisting of 0.1 L, 0.5 L, 1 L, 5 L, 40 L, 500 L, 5000 L, and 50,000 L.

49. The host cell of claim 38, wherein the polypeptide is secreted from the cell.

50. The host cell of claim 38, wherein at least two N-linked glycosylation sites of the polypeptide are modified.

51. The host cell of claim 38, wherein at least three N-linked glycosylation sites of the polypeptide are modified.

52. The host cell of claim 38, wherein at least four N-linked glycosylation sites of the polypeptide are modified.

53. The host cell of claim 38, wherein the modified N-linked glycosylation site of the polypeptide comprises an oligomannosyl structure.

54. The host cell of claim 38, wherein the modified N-linked glycosylation site of the peptide comprises a glycosylation chain selected from the group consisting of: Man₂GlcNAc₂, Man₃GlcNAc₂, Man₄GlcNAc₂, Man₅ GlcNAc₂, Man₆GlcNAc₂, Man₇GlcNAc₂, Man₈GlcNAc₂, and Man₉GlcNAc₂.

55. The host cell of claim 38, wherein the polypeptide comprises 2, 3, 4, 5, 6, 7, 8, or 9 terminal mannoses at the at least one N-linked glycosylation site.

56. The host cell of claim 38, wherein the polypeptide binds a mannose receptor present on macrophages.

57. The host cell of claim 38, wherein the mRNA expression of the target gene is transiently inhibited.

58. The host cell of claim 57, wherein the mRNA expression is transiently inhibited by contacting the cell with at least one RNA effector molecule.

59. The host cell of claim 38, further comprising adding a reagent that facilitates RNA effector molecule uptake into the cell.

60. The host cell of claim 38, wherein the at least one RNA effector molecule comprises an siRNA.

61. The host cell of claim 38, wherein the at least one RNA effector molecule comprises
(a) a sense strand comprising a sequence selected from the group consisting of: SEQ ID NO. 1-33, SEQ ID NO. 67-94, SEQ ID NO. 123-154, SEQ ID NO. 187-221, and SEQ ID NO. 257-282; and
(b) a complementary anti-sense strand comprising a sequence selected from the group consisting of SEQ ID NO. 34-66, SEQ ID NO. 95-122, SEQ ID NO. 155-186, SEQ ID NO. 222-256 and SEQ ID NO. 283-308.

62. The host cell of claim 58, wherein two or more RNA effector molecules are cultured with the cell.

63. A composition comprising at least one RNA effector molecule comprising a nucleic acid sequence complementary to at least one target gene of a host cell, wherein the RNA effector molecule is capable of modulating mannosylation patterns at an N-linked glycosylation site of a polypeptide produced in the host cell, and wherein the target gene is selected from the group consisting of: Mgat1, Mgat4, SLC35A1, SLC35A2, and GNE.

64. The composition of claim 63, wherein the at least one RNA effector molecule comprises a duplex region.

65. The composition of claim 63, wherein the at least one RNA effector molecule is 15-30 nucleotides in length.

66. The composition of claim 63, wherein the at least one RNA effector molecule is 17-28 nucleotides in length.

67. The composition of claim 63, wherein the at least one RNA effector molecule comprises a modified nucleotide.

68. The composition of claim 63, wherein the at least one RNA effector molecule comprises
(a) a sense strand comprising a sequence selected from the group consisting of: SEQ ID NO. 1-33, SEQ ID NO. 67-94, SEQ ID NO. 123-154, SEQ ID NO. 187-221, and SEQ ID NO. 257-282; and
(b) a complementary anti-sense strand comprising a sequence selected from the group consisting of SEQ ID NO. 34-66, SEQ ID NO. 95-122, SEQ ID NO. 155-186, SEQ ID NO. 222-256 and SEQ ID NO. 283-308.

69. The composition of claim 63, further comprising an RNA effector molecule that inhibits expression of the mannose 6 phosphate receptor.

70. An isolated polypeptide that comprises a terminal mannose in at least one N-linked glycosylation site, wherein the glycosylation pattern of the isolated polypeptide has not been modified enzymatically to contain the terminal mannose.

71. The isolated polypeptide of claim 70, wherein the polypeptide is glucocerebrosidase.

72. A composition comprising a dsRNA for inhibiting expression of a target gene selected from the group consisting of: Mgat1, Mgat4, SLC35A1, SLC35A2, and GNE, the dsRNA comprising
(a) a sense strand comprising a sequence selected from the group consisting of: SEQ ID NO. 1-33, SEQ ID NO. 67-94, SEQ ID NO. 123-154, SEQ ID NO. 187-221, and SEQ ID NO. 257-282; and
(b) a complementary anti-sense strand comprising a sequence selected from the group consisting of SEQ ID NO. 34-66, SEQ ID NO. 95-122, SEQ ID NO. 155-186, SEQ ID NO. 222-256 and SEQ ID NO. 283-308.


SEQUENCE LISTING (1266 sequences)

SEQ ID NO: 1 - RNA, 19 nt, Artificial Sequence
  Description of Artificial Sequence Synthetic oligonucleotide
agccaugguagagucagua

SEQ ID NO: 2 - RNA, 19 nt, Artificial Sequence
  Description of Artificial Sequence Synthetic oligonucleotide
caucaugauuguucaugga

SEQ ID NO: 3 - RNA, 19 nt, Artificial Sequence
  Description of Artificial Sequence Synthetic oligonucleotide
gcccuuccuaugacaaacu

SEQ ID NO: 4 - RNA, 19 nt, Artificial Sequence
  Description of Artificial Sequence Synthetic oligonucleotide
cuuccuaugacaaacugcu

SEQ ID NO: 5 - RNA, 19 nt, Artificial Sequence
  Description of Artificial Sequence Synthetic oligonucleotide
auaugagcaucauucggau

SEQ ID NO: 6 - RNA, 19 nt, Artificial Sequence
  Description of Artificial Sequence Synthetic oligonucleotide
aagauuacauuguugcacu

SEQ ID NO: 7 - RNA, 19 nt, Artificial Sequence
  Description of Artificial Sequence Synthetic oligonucleotide
uggaugcacuuaucucauu

SEQ ID NO: 8 - RNA, 19 nt, Artificial Sequence
  Description of Artificial Sequence Synthetic oligonucleotide
uucgugcagucaagcaugu

SEQ ID NO: 9 - RNA, 19 nt, Artificial Sequence
  Description of Artificial Sequence Synthetic oligonucleotide
agucaagcaugucccauuu

SEQ ID NO: 10 - RNA, 19 nt, Artificial Sequence
  Description of Artificial Sequence Synthetic oligonucleotide
ucccauuugaccaguuuau

SEQ ID NO: 11 - RNA, 19 nt, Artificial Sequence
  Description of Artificial Sequence Synthetic oligonucleotide
cccauuugaccaguuuaua

SEQ ID NO: 12 - RNA, 19 nt, Artificial Sequence
  Description of Artificial Sequence Synthetic oligonucleotide
cccaugcugguuguaugau

SEQ ID NO: 13 - RNA, 19 nt, Artificial Sequence
  Description of Artificial Sequence Synthetic oligonucleotide
aacacgccagauaggaaga

SEQ ID NO: 14 - RNA, 19 nt, Artificial Sequence
  Description of Artificial Sequence Synthetic oligonucleotide
augcugacacccaagauaa

SEQ ID NO: 15 - RNA, 19 nt, Artificial Sequence
  Description of Artificial Sequence Synthetic oligonucleotide
ugcugacacccaagauaaa

SEQ ID NO: 16 - RNA, 19 nt, Artificial Sequence
  Description of Artificial Sequence Synthetic oligonucleotide
uauuacaagcgcuccaccu

SEQ ID NO: 17 - RNA, 19 nt, Artificial Sequence
  Description of Artificial Sequence Synthetic oligonucleotide
ccaccuucaguucgguaaa

SEQ ID NO: 18 - RNA, 19 nt, Artificial Sequence
  Description of Artificial Sequence Synthetic oligonucleotide
uucaguucgguaaacagua

SEQ ID NO: 19 - RNA, 19 nt, Artificial Sequence
  Description of Artificial Sequence Synthetic oligonucleotide
aagggugaaauaguuaaga

SEQ ID NO: 20 - RNA, 19 nt, Artificial Sequence
  Description of Artificial Sequence Synthetic oligonucleotide
agggugaaauaguuaagaa

SEQ ID NO: 21 - RNA, 19 nt, Artificial Sequence
  Description of Artificial Sequence Synthetic oligonucleotide
auuaguuuaauccugcaga

SEQ ID NO: 22 - RNA, 19 nt, Artificial Sequence
  Description of Artificial Sequence Synthetic oligonucleotide
auucugggaguaggcauuu

SEQ ID NO: 23 - RNA, 19 nt, Artificial Sequence
  Description of Artificial Sequence Synthetic oligonucleotide
agaaaggaaguuuggccaa

SEQ ID NO: 24 - RNA, 19 nt, Artificial Sequence
  Description of Artificial Sequence Synthetic oligonucleotide
cuuugugacacucauuaca

SEQ ID NO: 25 - RNA, 19 nt, Artificial Sequence
  Description of Artificial Sequence Synthetic oligonucleotide
gugacacucauuacaggca

SEQ ID NO: 26 - RNA, 19 nt, Artificial Sequence
  Description of Artificial Sequence Synthetic oligonucleotide
acgugaaggcccagaauau

SEQ ID NO: 27 - RNA, 19 nt, Artificial Sequence
  Description of Artificial Sequence Synthetic oligonucleotide
ccagaauauccuacgaaca

SEQ ID NO: 28 - RNA, 19 nt, Artificial Sequence
  Description of Artificial Sequence Synthetic oligonucleotide
gaauauccuacgaacagcu

SEQ ID NO: 29 - RNA, 19 nt, Artificial Sequence
  Description of Artificial Sequence Synthetic oligonucleotide
uccacauugucaaggacgu

SEQ ID NO: 30 - RNA, 19 nt, Artificial Sequence
  Description of Artificial Sequence Synthetic oligonucleotide
uucagacuugguugacccu

SEQ ID NO: 31 - RNA, 19 nt, Artificial Sequence
  Description of Artificial Sequence Synthetic oligonucleotide
uuagaucaguacuucuuca

SEQ ID NO: 32 - RNA, 19 nt, Artificial Sequence
  Description of Artificial Sequence Synthetic oligonucleotide
cuagauuaaagguggaucu

SEQ ID NO: 33 - RNA, 19 nt, Artificial Sequence
  Description of Artificial Sequence Synthetic oligonucleotide
aaugggucuuuccucuuaa

SEQ ID NO: 34 - RNA, 19 nt, Artificial Sequence
  Description of Artificial Sequence Synthetic oligonucleotide
uacugacucuaccauggcu

SEQ ID NO: 35 - RNA, 19 nt, Artificial Sequence
  Description of Artificial Sequence Synthetic oligonucleotide
uccaugaacaaucaugaug

SEQ ID NO: 36 - RNA, 19 nt, Artificial Sequence
  Description of Artificial Sequence Synthetic oligonucleotide
aguuugucauaggaagggc

SEQ ID NO: 37 - RNA, 19 nt, Artificial Sequence
  Description of Artificial Sequence Synthetic oligonucleotide
agcaguuugucauaggaag

SEQ ID NO: 38 - RNA, 19 nt, Artificial Sequence
  Description of Artificial Sequence Synthetic oligonucleotide
auccgaaugaugcucauau

SEQ ID NO: 39 - RNA, 19 nt, Artificial Sequence
  Description of Artificial Sequence Synthetic oligonucleotide
agugcaacaauguaaucuu

SEQ ID NO: 40 - RNA, 19 nt, Artificial Sequence
  Description of Artificial Sequence Synthetic oligonucleotide
aaugagauaagugcaucca

SEQ ID NO: 41 - RNA, 19 nt, Artificial Sequence
  Description of Artificial Sequence Synthetic oligonucleotide
acaugcuugacugcacgaa

SEQ ID NO: 42 - RNA, 19 nt, Artificial Sequence
  Description of Artificial Sequence Synthetic oligonucleotide
aaaugggacaugcuugacu

SEQ ID NO: 43 - RNA, 19 nt, Artificial Sequence
  Description of Artificial Sequence Synthetic oligonucleotide
auaaacuggucaaauggga

SEQ ID NO: 44 - RNA, 19 nt, Artificial Sequence
  Description of Artificial Sequence Synthetic oligonucleotide
uauaaacuggucaaauggg

SEQ ID NO: 45 - RNA, 19 nt, Artificial Sequence
  Description of Artificial Sequence Synthetic oligonucleotide
aucauacaaccagcauggg

SEQ ID NO: 46 - RNA, 19 nt, Artificial Sequence
  Description of Artificial Sequence Synthetic oligonucleotide
ucuuccuaucuggcguguu

SEQ ID NO: 47 - RNA, 19 nt, Artificial Sequence
  Description of Artificial Sequence Synthetic oligonucleotide
uuaucuugggugucagcau

SEQ ID NO: 48 - RNA, 19 nt, Artificial Sequence
  Description of Artificial Sequence Synthetic oligonucleotide
uuuaucuugggugucagca

SEQ ID NO: 49 - RNA, 19 nt, Artificial Sequence
  Description of Artificial Sequence Synthetic oligonucleotide
agguggagcgcuuguaaua

SEQ ID NO: 50 - RNA, 19 nt, Artificial Sequence
  Description of Artificial Sequence Synthetic oligonucleotide
uuuaccgaacugaaggugg

SEQ ID NO: 51 - RNA, 19 nt, Artificial Sequence
  Description of Artificial Sequence Synthetic oligonucleotide
uacuguuuaccgaacugaa

SEQ ID NO: 52 - RNA, 19 nt, Artificial Sequence
  Description of Artificial Sequence Synthetic oligonucleotide
ucuuaacuauuucacccuu

SEQ ID NO: 53 - RNA, 19 nt, Artificial Sequence
  Description of Artificial Sequence Synthetic oligonucleotide
uucuuaacuauuucacccu

SEQ ID NO: 54 - RNA, 19 nt, Artificial Sequence
  Description of Artificial Sequence Synthetic oligonucleotide
ucugcaggauuaaacuaau

SEQ ID NO: 55 - RNA, 19 nt, Artificial Sequence
  Description of Artificial Sequence Synthetic oligonucleotide
aaaugccuacucccagaau

SEQ ID NO: 56 - RNA, 19 nt, Artificial Sequence
  Description of Artificial Sequence Synthetic oligonucleotide
uuggccaaacuuccuuucu

SEQ ID NO: 57 - RNA, 19 nt, Artificial Sequence
  Description of Artificial Sequence Synthetic oligonucleotide
uguaaugagugucacaaag

SEQ ID NO: 58 - RNA, 19 nt, Artificial Sequence
  Description of Artificial Sequence Synthetic oligonucleotide
ugccuguaaugagugucac

SEQ ID NO: 59 - RNA, 19 nt, Artificial Sequence
  Description of Artificial Sequence Synthetic oligonucleotide
auauucugggccuucacgu

SEQ ID NO: 60 - RNA, 19 nt, Artificial Sequence
  Description of Artificial Sequence Synthetic oligonucleotide
uguucguaggauauucugg

SEQ ID NO: 61 - RNA, 19 nt, Artificial Sequence
  Description of Artificial Sequence Synthetic oligonucleotide
agcuguucguaggauauuc

SEQ ID NO: 62 - RNA, 19 nt, Artificial Sequence
  Description of Artificial Sequence Synthetic oligonucleotide
acguccuugacaaugugga

SEQ ID NO: 63 - RNA, 19 nt, Artificial Sequence
  Description of Artificial Sequence Synthetic oligonucleotide
agggucaaccaagucugaa

SEQ ID NO: 64 - RNA, 19 nt, Artificial Sequence
  Description of Artificial Sequence Synthetic oligonucleotide
ugaagaaguacugaucuaa

SEQ ID NO: 65 - RNA, 19 nt, Artificial Sequence
  Description of Artificial Sequence Synthetic oligonucleotide
agauccaccuuuaaucuag

SEQ ID NO: 66 - RNA, 19 nt, Artificial Sequence
  Description of Artificial Sequence Synthetic oligonucleotide
uuaagaggaaagacccauu

SEQ ID NO: 67 - RNA, 19 nt, Artificial Sequence
  Description of Artificial Sequence Synthetic oligonucleotide
gguuacuacaagaucgcca

SEQ ID NO: 68 - RNA, 19 nt, Artificial Sequence
  Description of Artificial Sequence Synthetic oligonucleotide
gccagaucuucaacaaguu

SEQ ID NO: 69 - RNA, 19 nt, Artificial Sequence
  Description of Artificial Sequence Synthetic oligonucleotide
gcaccagacuucuuugagu

SEQ ID NO: 70 - RNA, 19 nt, Artificial Sequence
  Description of Artificial Sequence Synthetic oligonucleotide
caccagacuucuuugagua

SEQ ID NO: 71 - RNA, 19 nt, Artificial Sequence
  Description of Artificial Sequence Synthetic oligonucleotide
ccagacuucuuugaguacu

SEQ ID NO: 72 - RNA, 19 nt, Artificial Sequence
  Description of Artificial Sequence Synthetic oligonucleotide
gugugugucugcuuggaau

SEQ ID NO: 73 - RNA, 19 nt, Artificial Sequence
  Description of Artificial Sequence Synthetic oligonucleotide
gucugcuuggaaugacaau

SEQ ID NO: 74 - RNA, 19 nt, Artificial Sequence
  Description of Artificial Sequence Synthetic oligonucleotide
gcucuaucgaacagacuuu

SEQ ID NO: 75 - RNA, 19 nt, Artificial Sequence
  Description of Artificial Sequence Synthetic oligonucleotide
gccuguauucguccagaaa

SEQ ID NO: 76 - RNA, 19 nt, Artificial Sequence
  Description of Artificial Sequence Synthetic oligonucleotide
cuguauucguccagaaauu

SEQ ID NO: 77 - RNA, 19 nt, Artificial Sequence
  Description of Artificial Sequence Synthetic oligonucleotide
ccagaaauuucaagaacga

SEQ ID NO: 78 - RNA, 19 nt, Artificial Sequence
  Description of Artificial Sequence Synthetic oligonucleotide
cagaaauuucaagaacgau

SEQ ID NO: 79 - RNA, 19 nt, Artificial Sequence
  Description of Artificial Sequence Synthetic oligonucleotide
gaaauuucaagaacgauga

SEQ ID NO: 80 - RNA, 19 nt, Artificial Sequence
  Description of Artificial Sequence Synthetic oligonucleotide
ggcaguucuuugaucagca

SEQ ID NO: 81 - RNA, 19 nt, Artificial Sequence
  Description of Artificial Sequence Synthetic oligonucleotide
gaucagcaucuuaaguuca

SEQ ID NO: 82 - RNA, 19 nt, Artificial Sequence
  Description of Artificial Sequence Synthetic oligonucleotide
gucuuucacccaguuggau

SEQ ID NO: 83 - RNA, 19 nt, Artificial Sequence
  Description of Artificial Sequence Synthetic oligonucleotide
cuuucacccaguuggauuu

SEQ ID NO: 84 - RNA, 19 nt, Artificial Sequence
  Description of Artificial Sequence Synthetic oligonucleotide
cacccaguuggauuuguca

SEQ ID NO: 85 - RNA, 19 nt, Artificial Sequence
  Description of Artificial Sequence Synthetic oligonucleotide
caguuggauuugucauacu

SEQ ID NO: 86 - RNA, 19 nt, Artificial Sequence
  Description of Artificial Sequence Synthetic oligonucleotide
cuuaugaccgggauuuccu

SEQ ID NO: 87 - RNA, 19 nt, Artificial Sequence
  Description of Artificial Sequence Synthetic oligonucleotide
ggaccaaugaucagaagga

SEQ ID NO: 88 - RNA, 19 nt, Artificial Sequence
  Description of Artificial Sequence Synthetic oligonucleotide
gguacaguacacuagcaga

SEQ ID NO: 89 - RNA, 19 nt, Artificial Sequence
  Description of Artificial Sequence Synthetic oligonucleotide
caguacacuagcagagaca

SEQ ID NO: 90 - RNA, 19 nt, Artificial Sequence
  Description of Artificial Sequence Synthetic oligonucleotide
gcguugucacuuuccaguu

SEQ ID NO: 91 - RNA, 19 nt, Artificial Sequence
  Description of Artificial Sequence Synthetic oligonucleotide
gcuggaucugcuugucaua

SEQ ID NO: 92 - RNA, 19 nt, Artificial Sequence
  Description of Artificial Sequence Synthetic oligonucleotide
ggaucugcuugucauauca

SEQ ID NO: 93 - RNA, 19 nt, Artificial Sequence
  Description of Artificial Sequence Synthetic oligonucleotide
gaucugcuugucauaucau

SEQ ID NO: 94 - RNA, 19 nt, Artificial Sequence
  Description of Artificial Sequence Synthetic oligonucleotide
gucauaucaugagcugaga

SEQ ID NO: 95 - RNA, 19 nt, Artificial Sequence
  Description of Artificial Sequence Synthetic oligonucleotide
uggcgaucuuguaguaacc

SEQ ID NO: 96 - RNA, 19 nt, Artificial Sequence
  Description of Artificial Sequence Synthetic oligonucleotide
aacuuguugaagaucuggc

SEQ ID NO: 97 - RNA, 19 nt, Artificial Sequence
  Description of Artificial Sequence Synthetic oligonucleotide
acucaaagaagucuggugc

SEQ ID NO: 98 - RNA, 19 nt, Artificial Sequence
  Description of Artificial Sequence Synthetic oligonucleotide
uacucaaagaagucuggug

SEQ ID NO: 99 - RNA, 19 nt, Artificial Sequence
  Description of Artificial Sequence Synthetic oligonucleotide
aguacucaaagaagucugg

SEQ ID NO: 100 - RNA, 19 nt, Artificial Sequence
  Description of Artificial Sequence Synthetic oligonucleotide
auuccaagcagacacacac

SEQ ID NO: 101 - RNA, 19 nt, Artificial Sequence
  Description of Artificial Sequence Synthetic oligonucleotide
auugucauuccaagcagac

SEQ ID NO: 102 - RNA, 19 nt, Artificial Sequence
  Description of Artificial Sequence Synthetic oligonucleotide
aaagucuguucgauagagc

SEQ ID NO: 103 - RNA, 19 nt, Artificial Sequence
  Description of Artificial Sequence Synthetic oligonucleotide
uuucuggacgaauacaggc

SEQ ID NO: 104 - RNA, 19 nt, Artificial Sequence
  Description of Artificial Sequence Synthetic oligonucleotide
aauuucuggacgaauacag

SEQ ID NO: 105 - RNA, 19 nt, Artificial Sequence
  Description of Artificial Sequence Synthetic oligonucleotide
ucguucuugaaauuucugg

SEQ ID NO: 106 - RNA, 19 nt, Artificial Sequence
  Description of Artificial Sequence Synthetic oligonucleotide
aucguucuugaaauuucug

SEQ ID NO: 107 - RNA, 19 nt, Artificial Sequence
  Description of Artificial Sequence Synthetic oligonucleotide
ucaucguucuugaaauuuc

SEQ ID NO: 108 - RNA, 19 nt, Artificial Sequence
  Description of Artificial Sequence Synthetic oligonucleotide
ugcugaucaaagaacugcc

SEQ ID NO: 109 - RNA, 19 nt, Artificial Sequence
  Description of Artificial Sequence Synthetic oligonucleotide
ugaacuuaagaugcugauc

SEQ ID NO: 110 - RNA, 19 nt, Artificial Sequence
  Description of Artificial Sequence Synthetic oligonucleotide
auccaacugggugaaagac

SEQ ID NO: 111 - RNA, 19 nt, Artificial Sequence
  Description of Artificial Sequence Synthetic oligonucleotide
aaauccaacugggugaaag

SEQ ID NO: 112 - RNA, 19 nt, Artificial Sequence
  Description of Artificial Sequence Synthetic oligonucleotide
ugacaaauccaacugggug

SEQ ID NO: 113 - RNA, 19 nt, Artificial Sequence
  Description of Artificial Sequence Synthetic oligonucleotide
aguaugacaaauccaacug

SEQ ID NO: 114 - RNA, 19 nt, Artificial Sequence
  Description of Artificial Sequence Synthetic oligonucleotide
aggaaaucccggucauaag

SEQ ID NO: 115 - RNA, 19 nt, Artificial Sequence
  Description of Artificial Sequence Synthetic oligonucleotide
uccuucugaucauuggucc

SEQ ID NO: 116 - RNA, 19 nt, Artificial Sequence
  Description of Artificial Sequence Synthetic oligonucleotide
ucugcuaguguacuguacc

SEQ ID NO: 117 - RNA, 19 nt, Artificial Sequence
  Description of Artificial Sequence Synthetic oligonucleotide
ugucucugcuaguguacug

SEQ ID NO: 118 - RNA, 19 nt, Artificial Sequence
  Description of Artificial Sequence Synthetic oligonucleotide
aacuggaaagugacaacgc

SEQ ID NO: 119 - RNA, 19 nt, Artificial Sequence
  Description of Artificial Sequence Synthetic oligonucleotide
uaugacaagcagauccagc

SEQ ID NO: 120 - RNA, 19 nt, Artificial Sequence
  Description of Artificial Sequence Synthetic oligonucleotide
ugauaugacaagcagaucc

SEQ ID NO: 121 - RNA, 19 nt, Artificial Sequence
  Description of Artificial Sequence Synthetic oligonucleotide
augauaugacaagcagauc

SEQ ID NO: 122 - RNA, 19 nt, Artificial Sequence
  Description of Artificial Sequence Synthetic oligonucleotide
ucucagcucaugauaugac

SEQ ID NO: 123 - RNA, 19 nt, Artificial Sequence
  Description of Artificial Sequence Synthetic oligonucleotide
agcgcucuaaggagcuaaa

SEQ ID NO: 124 - RNA, 19 nt, Artificial Sequence
  Description of Artificial Sequence Synthetic oligonucleotide
gcucuaaggagcuaaaccu

SEQ ID NO: 125 - RNA, 19 nt, Artificial Sequence
  Description of Artificial Sequence Synthetic oligonucleotide
cguaccugacugacacauu

SEQ ID NO: 126 - RNA, 19 nt, Artificial Sequence
  Description of Artificial Sequence Synthetic oligonucleotide
acacauugcacucgcucau

SEQ ID NO: 127 - RNA, 19 nt, Artificial Sequence
  Description of Artificial Sequence Synthetic oligonucleotide
acauugcacucgcucaucu

SEQ ID NO: 128 - RNA, 19 nt, Artificial Sequence
  Description of Artificial Sequence Synthetic oligonucleotide
agaaccuggauuacugcuu

SEQ ID NO: 129 - RNA, 19 nt, Artificial Sequence
  Description of Artificial Sequence Synthetic oligonucleotide
acauuguagccaagcccaa

SEQ ID NO: 130 - RNA, 19 nt, Artificial Sequence
  Description of Artificial Sequence Synthetic oligonucleotide
aagcccaacuacuugagca

SEQ ID NO: 131 - RNA, 19 nt, Artificial Sequence
  Description of Artificial Sequence Synthetic oligonucleotide
uggaauucauccuuauguu

SEQ ID NO: 132 - RNA, 19 nt, Artificial Sequence
  Description of Artificial Sequence Synthetic oligonucleotide
cauccuuauguucuaccga

SEQ ID NO: 133 - RNA, 19 nt, Artificial Sequence
  Description of Artificial Sequence Synthetic oligonucleotide
uccuuauguucuaccgaga

SEQ ID NO: 134 - RNA, 19 nt, Artificial Sequence
  Description of Artificial Sequence Synthetic oligonucleotide
cuaccgagacaagccuauu

SEQ ID NO: 135 - RNA, 19 nt, Artificial Sequence
  Description of Artificial Sequence Synthetic oligonucleotide
accgagacaagccuauuga

SEQ ID NO: 136 - RNA, 19 nt, Artificial Sequence
  Description of Artificial Sequence Synthetic oligonucleotide
cgagacaagccuauugacu

SEQ ID NO: 137 - RNA, 19 nt, Artificial Sequence
  Description of Artificial Sequence Synthetic oligonucleotide
ugugggugaaagucugcaa

SEQ ID NO: 138 - RNA, 19 nt, Artificial Sequence
  Description of Artificial Sequence Synthetic oligonucleotide
aacccugagaaagaugcga

SEQ ID NO: 139 - RNA, 19 nt, Artificial Sequence
  Description of Artificial Sequence Synthetic oligonucleotide
acccugagaaagaugcgaa

SEQ ID NO: 140 - RNA, 19 nt, Artificial Sequence
  Description of Artificial Sequence Synthetic oligonucleotide
cccugagaaagaugcgaaa

SEQ ID NO: 141 - RNA, 19 nt, Artificial Sequence
  Description of Artificial Sequence Synthetic oligonucleotide
auacacuacuuuaugugcu

SEQ ID NO: 142 - RNA, 19 nt, Artificial Sequence
  Description of Artificial Sequence Synthetic oligonucleotide
acacuacuuuaugugcugu

SEQ ID NO: 143 - RNA, 19 nt, Artificial Sequence
  Description of Artificial Sequence Synthetic oligonucleotide
uuucacguuaaguucgcau

SEQ ID NO: 144 - RNA, 19 nt, Artificial Sequence
  Description of Artificial Sequence Synthetic oligonucleotide
uucacguuaaguucgcaua

SEQ ID NO: 145 - RNA, 19 nt, Artificial Sequence
  Description of Artificial Sequence Synthetic oligonucleotide
ucacguuaaguucgcauau

SEQ ID NO: 146 - RNA, 19 nt, Artificial Sequence
  Description of Artificial Sequence Synthetic oligonucleotide
cacguuaaguucgcauaua

SEQ ID NO: 147 - RNA, 19 nt, Artificial Sequence
  Description of Artificial Sequence Synthetic oligonucleotide
guuaaguucgcauauacuu

SEQ ID NO: 148 - RNA, 19 nt, Artificial Sequence
  Description of Artificial Sequence Synthetic oligonucleotide
uaaguucgcauauacuucu

SEQ ID NO: 149 - RNA, 19 nt, Artificial Sequence
  Description of Artificial Sequence Synthetic oligonucleotide
aaguucgcauauacuucua

SEQ ID NO: 150 - RNA, 19 nt, Artificial Sequence
  Description of Artificial Sequence Synthetic oligonucleotide
guucgcauauacuucuaua

SEQ ID NO: 151 - RNA, 19 nt, Artificial Sequence
  Description of Artificial Sequence Synthetic oligonucleotide
uauacuucuauaagagcgu

SEQ ID NO: 152 - RNA, 19 nt, Artificial Sequence
  Description of Artificial Sequence Synthetic oligonucleotide
uucuauaagagcgugacuu

SEQ ID NO: 153 - RNA, 19 nt, Artificial Sequence
  Description of Artificial Sequence Synthetic oligonucleotide
agagcgugacuuguaauaa

SEQ ID NO: 154 - RNA, 19 nt, Artificial Sequence
  Description of Artificial Sequence Synthetic oligonucleotide
gagcgugacuuguaauaaa

SEQ ID NO: 155 - RNA, 19 nt, Artificial Sequence
  Description of Artificial Sequence Synthetic oligonucleotide
uuuagcuccuuagagcgcu

SEQ ID NO: 156 - RNA, 19 nt, Artificial Sequence
  Description of Artificial Sequence Synthetic oligonucleotide
agguuuagcuccuuagagc

SEQ ID NO: 157 - RNA, 19 nt, Artificial Sequence
  Description of Artificial Sequence Synthetic oligonucleotide
aaugugucagucagguacg

SEQ ID NO: 158 - RNA, 19 nt, Artificial Sequence
  Description of Artificial Sequence Synthetic oligonucleotide
augagcgagugcaaugugu

SEQ ID NO: 159 - RNA, 19 nt, Artificial Sequence
  Description of Artificial Sequence Synthetic oligonucleotide
agaugagcgagugcaaugu

SEQ ID NO: 160 - RNA, 19 nt, Artificial Sequence
  Description of Artificial Sequence Synthetic oligonucleotide
aagcaguaauccagguucu

SEQ ID NO: 161 - RNA, 19 nt, Artificial Sequence
  Description of Artificial Sequence Synthetic oligonucleotide
uugggcuuggcuacaaugu

SEQ ID NO: 162 - RNA, 19 nt, Artificial Sequence
  Description of Artificial Sequence Synthetic oligonucleotide
ugcucaaguaguugggcuu

SEQ ID NO: 163 - RNA, 19 nt, Artificial Sequence
  Description of Artificial Sequence Synthetic oligonucleotide
aacauaaggaugaauucca

SEQ ID NO: 164 - RNA, 19 nt, Artificial Sequence
  Description of Artificial Sequence Synthetic oligonucleotide
ucgguagaacauaaggaug

SEQ ID NO: 165 - RNA, 19 nt, Artificial Sequence
  Description of Artificial Sequence Synthetic oligonucleotide
ucucgguagaacauaagga

SEQ ID NO: 166 - RNA, 19 nt, Artificial Sequence
  Description of Artificial Sequence Synthetic oligonucleotide
aauaggcuugucucgguag

SEQ ID NO: 167 - RNA, 19 nt, Artificial Sequence
  Description of Artificial Sequence Synthetic oligonucleotide
ucaauaggcuugucucggu

SEQ ID NO: 168 - RNA, 19 nt, Artificial Sequence
  Description of Artificial Sequence Synthetic oligonucleotide
agucaauaggcuugucucg

SEQ ID NO: 169 - RNA, 19 nt, Artificial Sequence
  Description of Artificial Sequence Synthetic oligonucleotide
uugcagacuuucacccaca

SEQ ID NO: 170 - RNA, 19 nt, Artificial Sequence
  Description of Artificial Sequence Synthetic oligonucleotide
ucgcaucuuucucaggguu

SEQ ID NO: 171 - RNA, 19 nt, Artificial Sequence
  Description of Artificial Sequence Synthetic oligonucleotide
uucgcaucuuucucagggu

SEQ ID NO: 172 - RNA, 19 nt, Artificial Sequence
  Description of Artificial Sequence Synthetic oligonucleotide
uuucgcaucuuucucaggg

SEQ ID NO: 173 - RNA, 19 nt, Artificial Sequence
  Description of Artificial Sequence Synthetic oligonucleotide
agcacauaaaguaguguau

SEQ ID NO: 174 - RNA, 19 nt, Artificial Sequence
  Description of Artificial Sequence Synthetic oligonucleotide
acagcacauaaaguagugu

SEQ ID NO: 175 - RNA, 19 nt, Artificial Sequence
  Description of Artificial Sequence Synthetic oligonucleotide
augcgaacuuaacgugaaa

SEQ ID NO: 176 - RNA, 19 nt, Artificial Sequence
  Description of Artificial Sequence Synthetic oligonucleotide
uaugcgaacuuaacgugaa

SEQ ID NO: 177 - RNA, 19 nt, Artificial Sequence
  Description of Artificial Sequence Synthetic oligonucleotide
auaugcgaacuuaacguga

SEQ ID NO: 178 - RNA, 19 nt, Artificial Sequence
  Description of Artificial Sequence Synthetic oligonucleotide
uauaugcgaacuuaacgug

SEQ ID NO: 179 - RNA, 19 nt, Artificial Sequence
  Description of Artificial Sequence Synthetic oligonucleotide
aaguauaugcgaacuuaac

SEQ ID NO: 180 - RNA, 19 nt, Artificial Sequence
  Description of Artificial Sequence Synthetic oligonucleotide
agaaguauaugcgaacuua

SEQ ID NO: 181 - RNA, 19 nt, Artificial Sequence
  Description of Artificial Sequence Synthetic oligonucleotide
uagaaguauaugcgaacuu

SEQ ID NO: 182 - RNA, 19 nt, Artificial Sequence
  Description of Artificial Sequence Synthetic oligonucleotide
uauagaaguauaugcgaac

SEQ ID NO: 183 - RNA, 19 nt, Artificial Sequence
  Description of Artificial Sequence Synthetic oligonucleotide
acgcucuuauagaaguaua

SEQ ID NO: 184 - RNA, 19 nt, Artificial Sequence
  Description of Artificial Sequence Synthetic oligonucleotide
aagucacgcucuuauagaa

SEQ ID NO: 185 - RNA, 19 nt, Artificial Sequence
  Description of Artificial Sequence Synthetic oligonucleotide
uuauuacaagucacgcucu

SEQ ID NO: 186 - RNA, 19 nt, Artificial Sequence
  Description of Artificial Sequence Synthetic oligonucleotide
uuuauuacaagucacgcuc

SEQ ID NO: 187 - RNA, 19 nt, Artificial Sequence
  Description of Artificial Sequence Synthetic oligonucleotide
gcagcuuauaccguagcuu

SEQ ID NO: 188 - RNA, 19 nt, Artificial Sequence
  Description of Artificial Sequence Synthetic oligonucleotide
cagcuuauaccguagcuuu

SEQ ID NO: 189 - RNA, 19 nt, Artificial Sequence
  Description of Artificial Sequence Synthetic oligonucleotide
cguagcuuuaagauacaca

SEQ ID NO: 190 - RNA, 19 nt, Artificial Sequence
  Description of Artificial Sequence Synthetic oligonucleotide
guagcuuuaagauacacaa

SEQ ID NO: 191 - RNA, 19 nt, Artificial Sequence
  Description of Artificial Sequence Synthetic oligonucleotide
gucacagaaguuauaaagu

SEQ ID NO: 192 - RNA, 19 nt, Artificial Sequence
  Description of Artificial Sequence Synthetic oligonucleotide
guuacugauaaguguugga

SEQ ID NO: 193 - RNA, 19 nt, Artificial Sequence
  Description of Artificial Sequence Synthetic oligonucleotide
cuggaaguuuggguagauu

SEQ ID NO: 194 - RNA, 19 nt, Artificial Sequence
  Description of Artificial Sequence Synthetic oligonucleotide
gaaguuuggguagauuuaa

SEQ ID NO: 195 - RNA, 19 nt, Artificial Sequence
  Description of Artificial Sequence Synthetic oligonucleotide
ggguagauuuaaggcaucu

SEQ ID NO: 196 - RNA, 19 nt, Artificial Sequence
  Description of Artificial Sequence Synthetic oligonucleotide
gguagauuuaaggcaucuu

SEQ ID NO: 197 - RNA, 19 nt, Artificial Sequence
  Description of Artificial Sequence Synthetic oligonucleotide
ggcuuuccuagcucuuagu

SEQ ID NO: 198 - RNA, 19 nt, Artificial Sequence
  Description of Artificial Sequence Synthetic oligonucleotide
cuuuccuagcucuuaguau

SEQ ID NO: 199 - RNA, 19 nt, Artificial Sequence
  Description of Artificial Sequence Synthetic oligonucleotide
caguauaccagguuaccua

SEQ ID NO: 200 - RNA, 19 nt, Artificial Sequence
  Description of Artificial Sequence Synthetic oligonucleotide
cucagcaaauuacaugggu

SEQ ID NO: 201 - RNA, 19 nt, Artificial Sequence
  Description of Artificial Sequence Synthetic oligonucleotide
gcuuuggugcaauagcuau

SEQ ID NO: 202 - RNA, 19 nt, Artificial Sequence
  Description of Artificial Sequence Synthetic oligonucleotide
caauagcuauugcuguauu

SEQ ID NO: 203 - RNA, 19 nt, Artificial Sequence
  Description of Artificial Sequence Synthetic oligonucleotide
gcuguauugugcucuggau

SEQ ID NO: 204 - RNA, 19 nt, Artificial Sequence
  Description of Artificial Sequence Synthetic oligonucleotide
ggauuugcaggaguuuauu

SEQ ID NO: 205 - RNA, 19 nt, Artificial Sequence
  Description of Artificial Sequence Synthetic oligonucleotide
gggugagaaacauucacuu

SEQ ID NO: 206 - RNA, 19 nt, Artificial Sequence
  Description of Artificial Sequence Synthetic oligonucleotide
gauacuccuauaacgacua

SEQ ID NO: 207 - RNA, 19 nt, Artificial Sequence
  Description of Artificial Sequence Synthetic oligonucleotide
cuccuauaacgacuaaacu

SEQ ID NO: 208 - RNA, 19 nt, Artificial Sequence
  Description of Artificial Sequence Synthetic oligonucleotide
cgacuaaacugucaauaau

SEQ ID NO: 209 - RNA, 19 nt, Artificial Sequence
  Description of Artificial Sequence Synthetic oligonucleotide
gacuaaacugucaauaaua

SEQ ID NO: 210 - RNA, 19 nt, Artificial Sequence
  Description of Artificial Sequence Synthetic oligonucleotide
ccagaugguagcuuaaaca

SEQ ID NO: 211 - RNA, 19 nt, Artificial Sequence
  Description of Artificial Sequence Synthetic oligonucleotide
gaugguagcuuaaacaaua

SEQ ID NO: 212 - RNA, 19 nt, Artificial Sequence
  Description of Artificial Sequence Synthetic oligonucleotide
gguagcuuaaacaauauca

SEQ ID NO: 213 - RNA, 19 nt, Artificial Sequence
  Description of Artificial Sequence Synthetic oligonucleotide
guagcuuaaacaauaucaa

SEQ ID NO: 214 - RNA, 19 nt, Artificial Sequence
  Description of Artificial Sequence Synthetic oligonucleotide
gugaaacuacaauauucaa

SEQ ID NO: 215 - RNA, 19 nt, Artificial Sequence
  Description of Artificial Sequence Synthetic oligonucleotide
gguaucugaagguucaagu

SEQ ID NO: 216 - RNA, 19 nt, Artificial Sequence
  Description of Artificial Sequence Synthetic oligonucleotide
cuguucucucguucaggua

SEQ ID NO: 217 - RNA, 19 nt, Artificial Sequence
  Description of Artificial Sequence Synthetic oligonucleotide
gugaagaaugaauaagaga

SEQ ID NO: 218 - RNA, 19 nt, Artificial Sequence
  Description of Artificial Sequence Synthetic oligonucleotide
cugacuucugucuggguaa

SEQ ID NO: 219 - RNA, 19 nt, Artificial Sequence
  Description of Artificial Sequence Synthetic oligonucleotide
gcuguuagcugauauacuu

SEQ ID NO: 220 - RNA, 19 nt, Artificial Sequence
  Description of Artificial Sequence Synthetic oligonucleotide
ggcucucaauuugugaacu

SEQ ID NO: 221 - RNA, 19 nt, Artificial Sequence
  Description of Artificial Sequence Synthetic oligonucleotide
gcucucaauuugugaacuu

SEQ ID NO: 222 - RNA, 19 nt, Artificial Sequence
  Description of Artificial Sequence Synthetic oligonucleotide
aagcuacgguauaagcugc

SEQ ID NO: 223 - RNA, 19 nt, Artificial Sequence
  Description of Artificial Sequence Synthetic oligonucleotide
aaagcuacgguauaagcug

SEQ ID NO: 224 - RNA, 19 nt, Artificial Sequence
  Description of Artificial Sequence Synthetic oligonucleotide
uguguaucuuaaagcuacg

SEQ ID NO: 225 - RNA, 19 nt, Artificial Sequence
  Description of Artificial Sequence Synthetic oligonucleotide
uuguguaucuuaaagcuac

SEQ ID NO: 226 - RNA, 19 nt, Artificial Sequence
  Description of Artificial Sequence Synthetic oligonucleotide
acuuuauaacuucugugac

SEQ ID NO: 227 - RNA, 19 nt, Artificial Sequence
  Description of Artificial Sequence Synthetic oligonucleotide
uccaacacuuaucaguaac

SEQ ID NO: 228 - RNA, 19 nt, Artificial Sequence
  Description of Artificial Sequence Synthetic oligonucleotide
aaucuacccaaacuuccag

SEQ ID NO: 229 - RNA, 19 nt, Artificial Sequence
  Description of Artificial Sequence Synthetic oligonucleotide
uuaaaucuacccaaacuuc

SEQ ID NO: 230 - RNA, 19 nt, Artificial Sequence
  Description of Artificial Sequence Synthetic oligonucleotide
agaugccuuaaaucuaccc

SEQ ID NO: 231 - RNA, 19 nt, Artificial Sequence
  Description of Artificial Sequence Synthetic oligonucleotide
aagaugccuuaaaucuacc

SEQ ID NO: 232 - RNA, 19 nt, Artificial Sequence
  Description of Artificial Sequence Synthetic oligonucleotide
acuaagagcuaggaaagcc

SEQ ID NO: 233 - RNA, 19 nt, Artificial Sequence
  Description of Artificial Sequence Synthetic oligonucleotide
auacuaagagcuaggaaag

SEQ ID NO: 234 - RNA, 19 nt, Artificial Sequence
  Description of Artificial Sequence Synthetic oligonucleotide
uagguaaccugguauacug

SEQ ID NO: 235 - RNA, 19 nt, Artificial Sequence
  Description of Artificial Sequence Synthetic oligonucleotide
acccauguaauuugcugag

SEQ ID NO: 236 - RNA, 19 nt, Artificial Sequence
  Description of Artificial Sequence Synthetic oligonucleotide
auagcuauugcaccaaagc

SEQ ID NO: 237 - RNA, 19 nt, Artificial Sequence
  Description of Artificial Sequence Synthetic oligonucleotide
aauacagcaauagcuauug

SEQ ID NO: 238 - RNA, 19 nt, Artificial Sequence
  Description of Artificial Sequence Synthetic oligonucleotide
auccagagcacaauacagc

SEQ ID NO: 239 - RNA, 19 nt, Artificial Sequence
  Description of Artificial Sequence Synthetic oligonucleotide
aauaaacuccugcaaaucc

SEQ ID NO: 240 - RNA, 19 nt, Artificial Sequence
  Description of Artificial Sequence Synthetic oligonucleotide
aagugaauguuucucaccc

SEQ ID NO: 241 - RNA, 19 nt, Artificial Sequence
  Description of Artificial Sequence Synthetic oligonucleotide
uagucguuauaggaguauc

SEQ ID NO: 242 - RNA, 19 nt, Artificial Sequence
  Description of Artificial Sequence Synthetic oligonucleotide
aguuuagucguuauaggag

SEQ ID NO: 243 - RNA, 19 nt, Artificial Sequence
  Description of Artificial Sequence Synthetic oligonucleotide
auuauugacaguuuagucg

SEQ ID NO: 244 - RNA, 19 nt, Artificial Sequence
  Description of Artificial Sequence Synthetic oligonucleotide
uauuauugacaguuuaguc

SEQ ID NO: 245 - RNA, 19 nt, Artificial Sequence
  Description of Artificial Sequence Synthetic oligonucleotide
uguuuaagcuaccaucugg

SEQ ID NO: 246 - RNA, 19 nt, Artificial Sequence
  Description of Artificial Sequence Synthetic oligonucleotide
uauuguuuaagcuaccauc

SEQ ID NO: 247 - RNA, 19 nt, Artificial Sequence
  Description of Artificial Sequence Synthetic oligonucleotide
ugauauuguuuaagcuacc

SEQ ID NO: 248 - RNA, 19 nt, Artificial Sequence
  Description of Artificial Sequence Synthetic oligonucleotide
uugauauuguuuaagcuac

SEQ ID NO: 249 - RNA, 19 nt, Artificial Sequence
  Description of Artificial Sequence Synthetic oligonucleotide
uugaauauuguaguuucac

SEQ ID NO: 250 - RNA, 19 nt, Artificial Sequence
  Description of Artificial Sequence Synthetic oligonucleotide
acuugaaccuucagauacc

SEQ ID NO: 251 - RNA, 19 nt, Artificial Sequence
  Description of Artificial Sequence Synthetic oligonucleotide
uaccugaacgagagaacag

SEQ ID NO: 252 - RNA, 19 nt, Artificial Sequence
  Description of Artificial Sequence Synthetic oligonucleotide
ucucuuauucauucuucac

SEQ ID NO: 253 - RNA, 19 nt, Artificial Sequence
  Description of Artificial Sequence Synthetic oligonucleotide
uuacccagacagaagucag

SEQ ID NO: 254 - RNA, 19 nt, Artificial Sequence
  Description of Artificial Sequence Synthetic oligonucleotide
aaguauaucagcuaacagc

SEQ ID NO: 255 - RNA, 19 nt, Artificial Sequence
  Description of Artificial Sequence Synthetic oligonucleotide
aguucacaaauugagagcc

SEQ ID NO: 256 - RNA, 19 nt, Artificial Sequence
  Description of Artificial Sequence Synthetic oligonucleotide
aaguucacaaauugagagc

SEQ ID NO: 257 - RNA, 19 nt, Artificial Sequence
  Description of Artificial Sequence Synthetic oligonucleotide
ucauccuuagcauccgaua

SEQ ID NO: 258 - RNA, 19 nt, Artificial Sequence
  Description of Artificial Sequence Synthetic oligonucleotide
cauccuuagcauccgauau

SEQ ID NO: 259 - RNA, 19 nt, Artificial Sequence
  Description of Artificial Sequence Synthetic oligonucleotide
agcauccgauaugcucgua

SEQ ID NO: 260 - RNA, 19 nt, Artificial Sequence
  Description of Artificial Sequence Synthetic oligonucleotide
cagcucaagauccugacua

SEQ ID NO: 261 - RNA, 19 nt, Artificial Sequence
  Description of Artificial Sequence Synthetic oligonucleotide
uggcuguuguagucaagua

SEQ ID NO: 262 - RNA, 19 nt, Artificial Sequence
  Description of Artificial Sequence Synthetic oligonucleotide
ucaccuggacccauuauuu

SEQ ID NO: 263 - RNA, 19 nt, Artificial Sequence
  Description of Artificial Sequence Synthetic oligonucleotide
agugcagucaaagccauaa

SEQ ID NO: 264 - RNA, 19 nt, Artificial Sequence
  Description of Artificial Sequence Synthetic oligonucleotide
ccacacuucuagagggaua

SEQ ID NO: 265 - RNA, 19 nt, Artificial Sequence
  Description of Artificial Sequence Synthetic oligonucleotide
cacacuucuagagggauau

SEQ ID NO: 266 - RNA, 19 nt, Artificial Sequence
  Description of Artificial Sequence Synthetic oligonucleotide
aggcuaaccucuuugggaa

SEQ ID NO: 267 - RNA, 19 nt, Artificial Sequence
  Description of Artificial Sequence Synthetic oligonucleotide
uucuucaguaacgacuaau

SEQ ID NO: 268 - RNA, 19 nt, Artificial Sequence
  Description of Artificial Sequence Synthetic oligonucleotide
gaagaucggccuguuguaa

SEQ ID NO: 269 - RNA, 19 nt, Artificial Sequence
  Description of Artificial Sequence Synthetic oligonucleotide
caauaagcaccauuuacuu

SEQ ID NO: 270 - RNA, 19 nt, Artificial Sequence
  Description of Artificial Sequence Synthetic oligonucleotide
uaugucgggcauuugugau

SEQ ID NO: 271 - RNA, 19 nt, Artificial Sequence
  Description of Artificial Sequence Synthetic oligonucleotide
augucgggcauuugugaua

SEQ ID NO: 272 - RNA, 19 nt, Artificial Sequence
  Description of Artificial Sequence Synthetic oligonucleotide
ugucgggcauuugugauau

SEQ ID NO: 273 - RNA, 19 nt, Artificial Sequence
  Description of Artificial Sequence Synthetic oligonucleotide
gcauuugugauaucagguu

SEQ ID NO: 274 - RNA, 19 nt, Artificial Sequence
  Description of Artificial Sequence Synthetic oligonucleotide
ucauggcggugucagaauu

SEQ ID NO: 275 - RNA, 19 nt, Artificial Sequence
  Description of Artificial Sequence Synthetic oligonucleotide
uggcggugucagaauugau

SEQ ID NO: 276 - RNA, 19 nt, Artificial Sequence
  Description of Artificial Sequence Synthetic oligonucleotide
aaagcuuacuaacuccuua

SEQ ID NO: 277 - RNA, 19 nt, Artificial Sequence
  Description of Artificial Sequence Synthetic oligonucleotide
uacuaacuccuuaacugua

SEQ ID NO: 278 - RNA, 19 nt, Artificial Sequence
  Description of Artificial Sequence Synthetic oligonucleotide
acuaacuccuuaacuguau

SEQ ID NO: 279 - RNA, 19 nt, Artificial Sequence
  Description of Artificial Sequence Synthetic oligonucleotide
aaugaacauaugucaggau

SEQ ID NO: 280 - RNA, 19 nt, Artificial Sequence
  Description of Artificial Sequence Synthetic oligonucleotide
augaacauaugucaggaua

SEQ ID NO: 281 - RNA, 19 nt, Artificial Sequence
  Description of Artificial Sequence Synthetic oligonucleotide
ucaggauacccaaugccaa

SEQ ID NO: 282 - RNA, 19 nt, Artificial Sequence
  Description of Artificial Sequence Synthetic oligonucleotide
gauacccaaugccaaauaa

SEQ ID NO: 283 - RNA, 19 nt, Artificial Sequence
  Description of Artificial Sequence Synthetic oligonucleotide
uaucggaugcuaaggauga

SEQ ID NO: 284 - RNA, 19 nt, Artificial Sequence
  Description of Artificial Sequence Synthetic oligonucleotide
auaucggaugcuaaggaug

SEQ ID NO: 285 - RNA, 19 nt, Artificial Sequence
  Description of Artificial Sequence Synthetic oligonucleotide
uacgagcauaucggaugcu

SEQ ID NO: 286 - RNA, 19 nt, Artificial Sequence
  Description of Artificial Sequence Synthetic oligonucleotide
uagucaggaucuugagcug

SEQ ID NO: 287 - RNA, 19 nt, Artificial Sequence
  Description of Artificial Sequence Synthetic oligonucleotide
uacuugacuacaacagcca

SEQ ID NO: 288 - RNA, 19 nt, Artificial Sequence
  Description of Artificial Sequence Synthetic oligonucleotide
aaauaauggguccagguga

SEQ ID NO: 289 - RNA, 19 nt, Artificial Sequence
  Description of Artificial Sequence Synthetic oligonucleotide
uuauggcuuugacugcacu

SEQ ID NO: 290 - RNA, 19 nt, Artificial Sequence
  Description of Artificial Sequence Synthetic oligonucleotide
uaucccucuagaagugugg

SEQ ID NO: 291 - RNA, 19 nt, Artificial Sequence
  Description of Artificial Sequence Synthetic oligonucleotide
auaucccucuagaagugug

SEQ ID NO: 292 - RNA, 19 nt, Artificial Sequence
  Description of Artificial Sequence Synthetic oligonucleotide
uucccaaagagguuagccu

SEQ ID NO: 293 - RNA, 19 nt, Artificial Sequence
  Description of Artificial Sequence Synthetic oligonucleotide
auuagucguuacugaagaa

SEQ ID NO: 294 - RNA, 19 nt, Artificial Sequence
  Description of Artificial Sequence Synthetic oligonucleotide
uuacaacaggccgaucuuc

SEQ ID NO: 295 - RNA, 19 nt, Artificial Sequence
  Description of Artificial Sequence Synthetic oligonucleotide
aaguaaauggugcuuauug

SEQ ID NO: 296 - RNA, 19 nt, Artificial Sequence
  Description of Artificial Sequence Synthetic oligonucleotide
aucacaaaugcccgacaua

SEQ ID NO: 297 - RNA, 19 nt, Artificial Sequence
  Description of Artificial Sequence Synthetic oligonucleotide
uaucacaaaugcccgacau

SEQ ID NO: 298 - RNA, 19 nt, Artificial Sequence
  Description of Artificial Sequence Synthetic oligonucleotide
auaucacaaaugcccgaca

SEQ ID NO: 299 - RNA, 19 nt, Artificial Sequence
  Description of Artificial Sequence Synthetic oligonucleotide
aaccugauaucacaaaugc

SEQ ID NO: 300 - RNA, 19 nt, Artificial Sequence
  Description of Artificial Sequence Synthetic oligonucleotide
aauucugacaccgccauga

SEQ ID NO: 301 - RNA, 19 nt, Artificial Sequence
  Description of Artificial Sequence Synthetic oligonucleotide
aucaauucugacaccgcca

SEQ ID NO: 302 - RNA, 19 nt, Artificial Sequence
  Description of Artificial Sequence Synthetic oligonucleotide
uaaggaguuaguaagcuuu

SEQ ID NO: 303 - RNA, 19 nt, Artificial Sequence
  Description of Artificial Sequence Synthetic oligonucleotide
uacaguuaaggaguuagua

SEQ ID NO: 304 - RNA, 19 nt, Artificial Sequence
  Description of Artificial Sequence Synthetic oligonucleotide
auacaguuaaggaguuagu

SEQ ID NO: 305 - RNA, 19 nt, Artificial Sequence
  Description of Artificial Sequence Synthetic oligonucleotide
auccugacauauguucauu

SEQ ID NO: 306 - RNA, 19 nt, Artificial Sequence
  Description of Artificial Sequence Synthetic oligonucleotide
uauccugacauauguucau

SEQ ID NO: 307 - RNA, 19 nt, Artificial Sequence
  Description of Artificial Sequence Synthetic oligonucleotide
uuggcauuggguauccuga

SEQ ID NO: 308 - RNA, 19 nt, Artificial Sequence
  Description of Artificial Sequence Synthetic oligonucleotide
uuauuuggcauuggguauc

SEQ ID NO: 309 - DNA, 5323 bp, Homo sapiens
gtggctgggcgagcgaggagtggggacaaggtcgagcgacgagtcgtctacccgcgcgcccgagcgcggaaaccgaggggcaggctcgcgctctgcctgcttcgtggcgcttggttcgtccctcgcccgaggagcgcggtggcggcgtgggagggagcctctgacggcgtctggaactctattttaagaacctctcaaaacgaaacaagcaaatcatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctgagtggaatcaagttcttgtctttaggatgaccgtttcttaacaatcaaatctggtattgaactgcaggtgactttggcagagaaatgttttcacttttggtctcctcttccagagtcacctttccccactcctatttttgtagatgctattctttctgatgtcttcttactaggggtcattttagctcaaaccctgtaagttacagtcacaattttctgtgccaaagcagctacaataatagagaggaagccttcttagaactctgcttactaatgtattaataccactgagaccttcaggccttgcctgggatatcacttcatcctgaagtttgcattaataatccttccaggccgggcacagtggctcacgcctgtaatcccagcactttgggaggccgaggcgggcggatcacgaggtcaggagatcgagaccgccctggctaacatggtgaaacatggtgaaaccccgtctctactaaaaatacaaaaaattagctgggtgtggtggcgggtcccagctactcgggaggctgaggcaggagaatggcatgaaccagggaggcggagctggcagtgaactgagaccgcaccactgcactccagcctgggtgacagagcaagactccatctcaaaaataataataataaataataataataataataataataataataatccttccagctgggcgcagtggctcacgcctgtaatcccaacattttgggaggccgagatgggcggatcacctgaggtcaggagttcaagaccagcctggccaacatggtgaaaccccatctctactaaaatacaaaaattagccgggcgtggtggcatgtgcctgtagtcccagctactcgggaggctgaggcaggagaattgcttgaacccgggaggcggaggttgcagtgagccgagatcatgccactgcactccaccctgggtgacagagcgagactccgtctacacacacacacacacacacacacacatccttcctcctctaaccccaaactaagatcacagaaggtgatccagtcagagaacagagggaaatcttaccaggaagggcttaagtacacttttttttaaaacagctttattgtttttaaagcctacaatttgataagccttgacatatgtatacctgtgaaagcatcaccacaatcaagacactggacatatctatcactcccccatctcagatatcccccctaatcctggataccatttgttgaaagatgttattactctagctgaacttacaagagactttagaccagggatctaaattacagtggccttagtgaccttgtccttatcttcttaggacagctgagaagccactgggacttagagcctttaaaaggagattaactgtcccaaaaggatctttgctactgaccagcagacacttcttccttcagtagcctttcatactgtgttgagtaacaccctagggtgtccattaaagttttgagttttacctagagcccagagccatgaatcaggattctgtctacatgattcgtgttttcattggtgtcaaaatacaaaagccaaagttctggctatgaattgttaacttggaagaaatactaactgccaccacttattaagtgcctactgtgtgccaggctctgaactaggtgcttcatatacattatcctaaattatctcaacatatgaggtaggtgttttaatttttattttatagaacttggtgtgtttgactgttaagctatggggctagagagagggtttgatcccaggtccctctgtgcttttgctgctgagccacacaacctctcatttcaaaaacactttcaaaatgctaacatattctaattcactctaggccaccaaaaactttaatactaatatctgatttgtaaatgacttaatgtatccttgaccctatcagctgaatttaatgaaatattcctctctgctgtgaaattttaccagtatagtatttggtctagtgacaggtgttctatttttttgagacgggtctcactctgtcacccaggctggagtgcagtggctcaatgcaacctctgccccccaagttcaagtgattctcctgcctcagcctcttgagtagctgggattacaggcacatgcaccacgcccggctgatttttttttgtatttttagtagacagggtttcaccatgttggccaggctggtcttgaactcctgacctcaggtgatccgcccgcctctgcctcccaaagtgctgggattacaggtgtaagccatcacacctggccctagtgacaggtttttatgggtacttttagatgatctaagaaatcatgtgcatatatctttcagatttctattttgggaaaatgaaggtttctacaacatattgtttcagtgttcaaataaactgaaggactcaacattacatttgaactatatccttcctagtgggttagtgtgaaaaagagtttggctgattcctaaaactctgccagccctgcagtaatctccagggcctggttattgttcagacattccatggtgattcctgggaaggaagcttggctgctcagtttctgagtctggggtgagataatgttctgggaagggacatctgttctttggtgtaatctctcatggtgaaatctgctctgtacatcagacaattgcattgctaccaagtttcataccaaatatttgaaaggatggtattgaatctaaaaccaaatattagtttttattaaactcatgggaaggctaatatattccaacgtaaattattacatatggttaagtaattgcatgttaatttattttaatgtaaatatttttgttactgttctgagccaaattctaaagaaaaaataaatacatttccttgttgaaaaaaaaaaaaaaaaaa

SEQ ID NO: 310 - RNA, 19 nt, Artificial Sequence
  Description of Artificial Sequence Synthetic oligonucleotide
acuuguaaccgugcagauu

SEQ ID NO: 311 - RNA, 19 nt, Artificial Sequence
  Description of Artificial Sequence Synthetic oligonucleotide
uauggaaauacauaucgaa

SEQ ID NO: 312 - RNA, 19 nt, Artificial Sequence
  Description of Artificial Sequence Synthetic oligonucleotide
ggaccauugaugacucuau

SEQ ID NO: 313 - RNA, 19 nt, Artificial Sequence
  Description of Artificial Sequence Synthetic oligonucleotide
ucgcauguggcuaggugau

SEQ ID NO: 314 - RNA, 19 nt, Artificial Sequence
  Description of Artificial Sequence Synthetic oligonucleotide
cccaacuuucgugcaguua

SEQ ID NO: 315 - RNA, 19 nt, Artificial Sequence
  Description of Artificial Sequence Synthetic oligonucleotide
uuggaacaccugugaucaa

SEQ ID NO: 316 - RNA, 19 nt, Artificial Sequence
  Description of Artificial Sequence Synthetic oligonucleotide
guuucucaaaucuaucgau

SEQ ID NO: 317 - RNA, 19 nt, Artificial Sequence
  Description of Artificial Sequence Synthetic oligonucleotide
ucucaaaucuaucgaucuu

SEQ ID NO: 318 - RNA, 19 nt, Artificial Sequence
  Description of Artificial Sequence Synthetic oligonucleotide
ucuaagugccuuggccguu

SEQ ID NO: 319 - RNA, 19 nt, Artificial Sequence
  Description of Artificial Sequence Synthetic oligonucleotide
cgaaccuccgaguugcaau

SEQ ID NO: 320 - RNA, 19 nt, Artificial Sequence
  Description of Artificial Sequence Synthetic oligonucleotide
auacucaguucaauccuaa

SEQ ID NO: 321 - RNA, 19 nt, Artificial Sequence
  Description of Artificial Sequence Synthetic oligonucleotide
cccuguguggguagacaau

SEQ ID NO: 322 - RNA, 19 nt, Artificial Sequence
  Description of Artificial Sequence Synthetic oligonucleotide
aucucauccaagcugcgaa

SEQ ID NO: 323 - RNA, 19 nt, Artificial Sequence
  Description of Artificial Sequence Synthetic oligonucleotide
ucucauccaagcugcgaaa

SEQ ID NO: 324 - RNA, 19 nt, Artificial Sequence
  Description of Artificial Sequence Synthetic oligonucleotide
cgaaacuuggcaaugcgaa

SEQ ID NO: 325 - RNA, 19 nt, Artificial Sequence
  Description of Artificial Sequence Synthetic oligonucleotide
acuacacaacacgcaggau

SEQ ID NO: 326 - RNA, 19 nt, Artificial Sequence
  Description of Artificial Sequence Synthetic oligonucleotide
acacaacacgcaggaucua

SEQ ID NO: 327 - RNA, 19 nt, Artificial Sequence
  Description of Artificial Sequence Synthetic oligonucleotide
gucuuuaggaugaccguuu

SEQ ID NO: 328 - RNA, 19 nt, Artificial Sequence
  Description of Artificial Sequence Synthetic oligonucleotide
uuaggaugaccguuucuua

SEQ ID NO: 329 - RNA, 19 nt, Artificial Sequence
  Description of Artificial Sequence Synthetic oligonucleotide
uaggaugaccguuucuuaa

SEQ ID NO: 330 - RNA, 19 nt, Artificial Sequence
  Description of Artificial Sequence Synthetic oligonucleotide
accguuucuuaacaaucaa

SEQ ID NO: 331 - RNA, 19 nt, Artificial Sequence
  Description of Artificial Sequence Synthetic oligonucleotide
acccuaggguguccauuaa

SEQ ID NO: 332 - RNA, 19 nt, Artificial Sequence
  Description of Artificial Sequence Synthetic oligonucleotide
acuaacugccaccacuuau

SEQ ID NO: 333 - RNA, 19 nt, Artificial Sequence
  Description of Artificial Sequence Synthetic oligonucleotide
uaucucaacauaugaggua

SEQ ID NO: 334 - RNA, 19 nt, Artificial Sequence
  Description of Artificial Sequence Synthetic oligonucleotide
ccuaguggguuagugugaa

SEQ ID NO: 335 - RNA, 19 nt, Artificial Sequence
  Description of Artificial Sequence Synthetic oligonucleotide
acucaugggaaggcuaaua

SEQ ID NO: 336 - RNA, 19 nt, Artificial Sequence
  Description of Artificial Sequence Synthetic oligonucleotide
ucaugggaaggcuaauaua

SEQ ID NO: 337 - RNA, 19 nt, Artificial Sequence
  Description of Artificial Sequence Synthetic oligonucleotide
aaucugcacgguuacaagu

SEQ ID NO: 338 - RNA, 19 nt, Artificial Sequence
  Description of Artificial Sequence Synthetic oligonucleotide
uucgauauguauuuccaua

SEQ ID NO: 339 - RNA, 19 nt, Artificial Sequence
  Description of Artificial Sequence Synthetic oligonucleotide
auagagucaucaauggucc

SEQ ID NO: 340 - RNA, 19 nt, Artificial Sequence
  Description of Artificial Sequence Synthetic oligonucleotide
aucaccuagccacaugcga

SEQ ID NO: 341 - RNA, 19 nt, Artificial Sequence
  Description of Artificial Sequence Synthetic oligonucleotide
uaacugcacgaaaguuggg

SEQ ID NO: 342 - RNA, 19 nt, Artificial Sequence
  Description of Artificial Sequence Synthetic oligonucleotide
uugaucacagguguuccaa

SEQ ID NO: 343 - RNA, 19 nt, Artificial Sequence
  Description of Artificial Sequence Synthetic oligonucleotide
aucgauagauuugagaaac

SEQ ID NO: 344 - RNA, 19 nt, Artificial Sequence
  Description of Artificial Sequence Synthetic oligonucleotide
aagaucgauagauuugaga

SEQ ID NO: 345 - RNA, 19 nt, Artificial Sequence
  Description of Artificial Sequence Synthetic oligonucleotide
aacggccaaggcacuuaga

SEQ ID NO: 346 - RNA, 19 nt, Artificial Sequence
  Description of Artificial Sequence Synthetic oligonucleotide
auugcaacucggagguucg

SEQ ID NO: 347 - RNA, 19 nt, Artificial Sequence
  Description of Artificial Sequence Synthetic oligonucleotide
uuaggauugaacugaguau

SEQ ID NO: 348 - RNA, 19 nt, Artificial Sequence
  Description of Artificial Sequence Synthetic oligonucleotide
auugucuacccacacaggg

SEQ ID NO: 349 - RNA, 19 nt, Artificial Sequence
  Description of Artificial Sequence Synthetic oligonucleotide
uucgcagcuuggaugagau

SEQ ID NO: 350 - RNA, 19 nt, Artificial Sequence
  Description of Artificial Sequence Synthetic oligonucleotide
uuucgcagcuuggaugaga

SEQ ID NO: 351 - RNA, 19 nt, Artificial Sequence
  Description of Artificial Sequence Synthetic oligonucleotide
uucgcauugccaaguuucg

SEQ ID NO: 352 - RNA, 19 nt, Artificial Sequence
  Description of Artificial Sequence Synthetic oligonucleotide
auccugcguguuguguagu

SEQ ID NO: 353 - RNA, 19 nt, Artificial Sequence
  Description of Artificial Sequence Synthetic oligonucleotide
uagauccugcguguugugu

SEQ ID NO: 354 - RNA, 19 nt, Artificial Sequence
  Description of Artificial Sequence Synthetic oligonucleotide
aaacggucauccuaaagac

SEQ ID NO: 355 - RNA, 19 nt, Artificial Sequence
  Description of Artificial Sequence Synthetic oligonucleotide
uaagaaacggucauccuaa

SEQ ID NO: 356 - RNA, 19 nt, Artificial Sequence
  Description of Artificial Sequence Synthetic oligonucleotide
uuaagaaacggucauccua

SEQ ID NO: 357 - RNA, 19 nt, Artificial Sequence
  Description of Artificial Sequence Synthetic oligonucleotide
uugauuguuaagaaacggu

SEQ ID NO: 358 - RNA, 19 nt, Artificial Sequence
  Description of Artificial Sequence Synthetic oligonucleotide
uuaauggacacccuagggu

SEQ ID NO: 359 - RNA, 19 nt, Artificial Sequence
  Description of Artificial Sequence Synthetic oligonucleotide
auaagugguggcaguuagu

SEQ ID NO: 360 - RNA, 19 nt, Artificial Sequence
  Description of Artificial Sequence Synthetic oligonucleotide
uaccucauauguugagaua

SEQ ID NO: 361 - RNA, 19 nt, Artificial Sequence
  Description of Artificial Sequence Synthetic oligonucleotide
uucacacuaacccacuagg

SEQ ID NO: 362 - RNA, 19 nt, Artificial Sequence
  Description of Artificial Sequence Synthetic oligonucleotide
uauuagccuucccaugagu

SEQ ID NO: 363 - RNA, 19 nt, Artificial Sequence
  Description of Artificial Sequence Synthetic oligonucleotide
uauauuagccuucccauga

SEQ ID NO: 364 - DNA, 2658 bp, Homo sapiens
gatcacatggcagcagcccttcttggggaagtcagctacccagcagcctgtagtcctcggctacccaccctcaccgcctggggtcccatggtgcatggctgcctcctaatcccatagtccagaggaggcatccctaggactgcgggcaagggagccgggcaagcccagggcagccttgaaccgtcccctggcctgccctccccggtgggggccaggatgctgaagaagcagtctgcagggcttgtgctgtggggcgctatcctctttgtggcctggaatgccctgctgctcctcttcttctggacgcgcccagcacctggcaggccaccctcagtcagcgctctcgatggcgaccccgccagcctcacccgggaagtgattcgcctggcccaagacgccgaggtggagctggagcggcagcgtgggctgctgcagcagatcggggatgccctgtcgagccagcgggggagggtgcccaccgcggcccctcccgcccagccgcgtgtgcctgtgacccccgcgccggcggtgattcccatcctggtcatcgcctgtgaccgcagcactgttcggcgctgcctggacaagctgctgcattatcggccctcggctgagctcttccccatcatcgttagccaggactgcgggcacgaggagacggcccaggccatcgcctcctacggcagcgcggtcacgcacatccggcagcccgacctgagcagcattgcggtgccgccggaccaccgcaagttccagggctactacaagatcgcgcgccactaccgctgggcgctgggccaggtcttccggcagtttcgcttccccgcggccgtggtggtggaggatgacctggaggtggccccggacttcttcgagtactttcgggccacctatccgctgctgaaggccgacccctccctgtggtgcgtctcggcctggaatgacaacggcaaggagcagatggtggacgccagcaggcctgagctgctctaccgcaccgactttttccctggcctgggctggctgctgttggccgagctctgggctgagctggagcccaagtggccaaaggccttctgggacgactggatgcggcggccggagcagcggcaggggcgggcctgcatacgccctgagatctcaagaacgatgacctttggccgcaagggtgtgagccacgggcagttctttgaccagcacctcaagtttatcaagctgaaccagcagtttgtgcacttcacccagctggacctgtcttacctgcagcgggaggcctatgaccgagatttcctcgcccgcgtctacggtgctccccagctgcaggtggagaaagtgaggaccaatgaccggaaggagctgggggaggtgcgggtgcagtatacgggcagggacagcttcaaggctttcgccaaggctctgggtgtcatggatgaccttaagtcgggggttccgagagctggctaccggggtattgtcaccttccagttccggggccgccgtgtccacctggcgcccccactgacgtgggagggctatgatcctagctggaattagcacctgcctgtccttcctgggcccctccttgccacatcatgagctgaggtgggaccacagtccccaggctgcatcggcctgcctgtgtttccctcttaggtgcatttatctttttgatttttccgagtggcatttaagtgcacaaatgataacaagaggattattctcccgttctcaagggagtcagatcaggggaactattctagggtatgttgcggggtattaagcaggaaaccactgtgtggtggggggcactgggcttgttggggccagaaatgtccacgtcctgagctttctcctggagcatgtgcagagagtttggcaacgttcgctctcttgaccagaccccttctccctgacctggctcttccagccagggcacgagccctccttctatacctgctccccttcccccagtggggactgagttatgggagaaggggacatatttgtggccaaaatgatactaaccaaaggggcttccttgtcagggcctggtggagttggtgggtcatcggggctcactgcctcctgcccttctctcctgtctgacccccacttagcccttctctccttgcagcctagcagtttatagttctgagatggaaagttgaagggggcaagcaagacctctcctcagcccatgcccagctgtcaggagagaggtgcagggaggaaggccttgtgctgggacaacctctctcttgccttacctcagagagggactatgccctgacccctcctttctgaaaatcagtgccctccctgttgctctaggaggctcctgctggcttggtagaagacagaattcgatctgcctgtccctttttcccctggggtttgacacacaggctcctctcagcatgaggtggagcagtgaccaggtggagcagtgaccaggacgcctctggcccagtgctgcccagcctccccgcccgctcccaggcgccccatgtcctcacaggccaggacgccatggcaggatggagaggacttggtggatttttgtttcttgcctgacctcagtttcatgaaagaaagtggaagctacagaattattttctaaaataaaggctgaattgtctgaaaaataaaaaaaaaaaa

SEQ ID NO: 365 - RNA, 19 nt, Artificial Sequence
  Description of Artificial Sequence Synthetic oligonucleotide
gcugccuccuaaucccaua

SEQ ID NO: 366 - RNA, 19 nt, Artificial Sequence
  Description of Artificial Sequence Synthetic oligonucleotide
ugccuccuaaucccauagu

SEQ ID NO: 367 - RNA, 19 nt, Artificial Sequence
  Description of Artificial Sequence Synthetic oligonucleotide
uugugcuguggggcgcuau

SEQ ID NO: 368 - RNA, 19 nt, Artificial Sequence
  Description of Artificial Sequence Synthetic oligonucleotide
cucaagaacgaugaccuuu

SEQ ID NO: 369 - RNA, 19 nt, Artificial Sequence
  Description of Artificial Sequence Synthetic oligonucleotide
cgaugaccuuuggccgcaa

SEQ ID NO: 370 - RNA, 19 nt, Artificial Sequence
  Description of Artificial Sequence Synthetic oligonucleotide
uugccacaucaugagcuga

SEQ ID NO: 371 - RNA, 19 nt, Artificial Sequence
  Description of Artificial Sequence Synthetic oligonucleotide
gauuauucucccguucuca

SEQ ID NO: 372 - RNA, 19 nt, Artificial Sequence
  Description of Artificial Sequence Synthetic oligonucleotide
ggggaacuauucuagggua

SEQ ID NO: 373 - RNA, 19 nt, Artificial Sequence
  Description of Artificial Sequence Synthetic oligonucleotide
guauguugcgggguauuaa

SEQ ID NO: 374 - RNA, 19 nt, Artificial Sequence
  Description of Artificial Sequence Synthetic oligonucleotide
ugcgggguauuaagcagga

SEQ ID NO: 375 - RNA, 19 nt, Artificial Sequence
  Description of Artificial Sequence Synthetic oligonucleotide
gcgggguauuaagcaggaa

SEQ ID NO: 376 - RNA, 19 nt, Artificial Sequence
  Description of Artificial Sequence Synthetic oligonucleotide
gcagagaguuuggcaacgu

SEQ ID NO: 377 - RNA, 19 nt, Artificial Sequence
  Description of Artificial Sequence Synthetic oligonucleotide
cagagaguuuggcaacguu

SEQ ID NO: 378 - RNA, 19 nt, Artificial Sequence
  Description of Artificial Sequence Synthetic oligonucleotide
gaguuuggcaacguucgcu

SEQ ID NO: 379 - RNA, 19 nt, Artificial Sequence
  Description of Artificial Sequence Synthetic oligonucleotide
uuggcaacguucgcucucu

SEQ ID NO: 380 - RNA, 19 nt, Artificial Sequence
  Description of Artificial Sequence Synthetic oligonucleotide
uggcaacguucgcucucuu

SEQ ID NO: 381 - RNA, 19 nt, Artificial Sequence
  Description of Artificial Sequence Synthetic oligonucleotide
cccaguggggacugaguua

SEQ ID NO: 382 - RNA, 19 nt, Artificial Sequence
  Description of Artificial Sequence Synthetic oligonucleotide
ccaguggggacugaguuau

SEQ ID NO: 383 - RNA, 19 nt, Artificial Sequence
  Description of Artificial Sequence Synthetic oligonucleotide
uguggccaaaaugauacua

SEQ ID NO: 384 - RNA, 19 nt, Artificial Sequence
  Description of Artificial Sequence Synthetic oligonucleotide
uaccucagagagggacuau

SEQ ID NO: 385 - RNA, 19 nt, Artificial Sequence
  Description of Artificial Sequence Synthetic oligonucleotide
gacagaauucgaucugccu

SEQ ID NO: 386 - RNA, 19 nt, Artificial Sequence
  Description of Artificial Sequence Synthetic oligonucleotide
uaugggauuaggaggcagc

SEQ ID NO: 387 - RNA, 19 nt, Artificial Sequence
  Description of Artificial Sequence Synthetic oligonucleotide
acuaugggauuaggaggca

SEQ ID NO: 388 - RNA, 19 nt, Artificial Sequence
  Description of Artificial Sequence Synthetic oligonucleotide
auagcgccccacagcacaa

SEQ ID NO: 389 - RNA, 19 nt, Artificial Sequence
  Description of Artificial Sequence Synthetic oligonucleotide
aaaggucaucguucuugag

SEQ ID NO: 390 - RNA, 19 nt, Artificial Sequence
  Description of Artificial Sequence Synthetic oligonucleotide
uugcggccaaaggucaucg

SEQ ID NO: 391 - RNA, 19 nt, Artificial Sequence
  Description of Artificial Sequence Synthetic oligonucleotide
ucagcucaugauguggcaa

SEQ ID NO: 392 - RNA, 19 nt, Artificial Sequence
  Description of Artificial Sequence Synthetic oligonucleotide
ugagaacgggagaauaauc

SEQ ID NO: 393 - RNA, 19 nt, Artificial Sequence
  Description of Artificial Sequence Synthetic oligonucleotide
uacccuagaauaguucccc

SEQ ID NO: 394 - RNA, 19 nt, Artificial Sequence
  Description of Artificial Sequence Synthetic oligonucleotide
uuaauaccccgcaacauac

SEQ ID NO: 395 - RNA, 19 nt, Artificial Sequence
  Description of Artificial Sequence Synthetic oligonucleotide
uccugcuuaauaccccgca

SEQ ID NO: 396 - RNA, 19 nt, Artificial Sequence
  Description of Artificial Sequence Synthetic oligonucleotide
uuccugcuuaauaccccgc

SEQ ID NO: 397 - RNA, 19 nt, Artificial Sequence
  Description of Artificial Sequence Synthetic oligonucleotide
acguugccaaacucucugc

SEQ ID NO: 398 - RNA, 19 nt, Artificial Sequence
  Description of Artificial Sequence Synthetic oligonucleotide
aacguugccaaacucucug

SEQ ID NO: 399 - RNA, 19 nt, Artificial Sequence
  Description of Artificial Sequence Synthetic oligonucleotide
agcgaacguugccaaacuc

SEQ ID NO: 400 - RNA, 19 nt, Artificial Sequence
  Description of Artificial Sequence Synthetic oligonucleotide
agagagcgaacguugccaa

SEQ ID NO: 401 - RNA, 19 nt, Artificial Sequence
  Description of Artificial Sequence Synthetic oligonucleotide
aagagagcgaacguugcca

SEQ ID NO: 402 - RNA, 19 nt, Artificial Sequence
  Description of Artificial Sequence Synthetic oligonucleotide
uaacucaguccccacuggg

SEQ ID NO: 403 - RNA, 19 nt, Artificial Sequence
  Description of Artificial Sequence Synthetic oligonucleotide
auaacucaguccccacugg

SEQ ID NO: 404 - RNA, 19 nt, Artificial Sequence
  Description of Artificial Sequence Synthetic oligonucleotide
uaguaucauuuuggccaca

SEQ ID NO: 405 - RNA, 19 nt, Artificial Sequence
  Description of Artificial Sequence Synthetic oligonucleotide
auagucccucucugaggua

SEQ ID NO: 406 - RNA, 19 nt, Artificial Sequence
  Description of Artificial Sequence Synthetic oligonucleotide
aggcagaucgaauucuguc

SEQ ID NO: 407 - DNA, 8382 bp, Homo sapiens
cgcagcggcggcggcagcggcggcggcggctacagcagctccgccggcagccgcgggagcacggcgacgccagcggcgggaagggaaaaggccgaggcatcagcgtgtgaagaccgcaaagacgatcccgagtacagttgtgaacagcattgctgctaggctcctcctgcagatcatctgaaatgaacctctcttattgatttttattggcctagagccaggagtactgcattcagttgactttcagggtaaaaagaaaacagtcctggttgttgtcatcataaacatatggaccagtgtgatggtgaaatgagatgaggctccgcaatggaactgtagccactgctttagcatttatcacttccttccttactttgtcttggtatactacatggcaaaatgggaaagaaaaactgattgcttatcaacgagaattccttgctttgaaagaacgtcttcgaatagctgaacacagaatctcacagcgctcttctgaattaaatacgattgtgcaacagttcaagcgtgtaggagcagaaacaaatggaagtaaggatgcgttgaataagttttcagataataccctaaagctgttaaaggagttaacaagcaaaaaatctcttcaagtgccaagtatttattatcatttgcctcatttattgaaaaatgaaggaagtcttcaacctgctgtacagattggcaacggaagaacaggagtttcaatagtcatgggcattcccacagtgaagagagaagttaaatcttacctcatagaaactcttcattcccttattgataacctgtatcctgaagagaagttggactgtgttatagtagtcttcataggagagacagatattgattatgtacatggtgttgtagccaacctggagaaagaattttctaaagaaatcagttctggcttggtggaagtcatatcaccccctgaaagctattatcctgacttgacaaacctaaaggagacatttggagactccaaagaaagagtaagatggagaacaaagcaaaacctagattactgttttctaatgatgtatgctcaagaaaagggcatatattacattcagcttgaagatgatattattgtcaaacaaaattattttaataccataaaaaattttgcacttcaactttcttctgaggaatggatgattctagagttttcccagctgggcttcattggtaaaatgtttcaagcgccggatcttactctgattgtagaattcatattcatgttttacaaggagaaacccattgattggctcctggaccatattctctgggtgaaagtctgcaaccctgaaaaagatgcaaaacattgtgatagacagaaagcaaatctgcgaattcgcttcagaccttcccttttccaacatgttggtctgcactcatcactatcaggaaaaatccaaaaactcacggataaagattatatgaaaccattacttcttaaaatccatgtaaacccacctgcggaggtatctacttccttgaaggtctaccaagggcatacgctggagaaaacttacatgggagaggatttcttctgggctatcacaccgatagctggagactacatcttgtttaaatttgataaaccagtcaatgtagaaagttatttgttccatagcggcaaccaagaacatcctggagatattctgctaaacacaactgtggaagttttgccttttaagagtgaaggtttggaaataagcaaagaaaccaaagacaaacgattagaagatggctatttcagaataggaaaatttgagaatggtgttgcagaaggaatggtggatccaagtctcaatcccatttcagcctttcgactttcagttattcagaattctgctgtttgggccattcttaatgagattcatattaaaaaagccaccaactgatcatctgagaaaccaacacattttttcctgtgaatttgttaattaaagatagttaagcatgtatcttttttttatttctacttgaacactacctcttgtgaagtctactgtagataagacgattgtcatttccacttggaaagtgaatctcccataataattgtatttgtttgaaactaagctgtcctcagattttaacttgactcaaacatttttcaattatgacagcctgttaatatgacttgtactattttggtattatactaatacataagagttgtacatattgttacattctttaaatttgagaaaaactaatgttacatacattttatgaagggggtacttttgaggttcacttattttactattatagaccctcttttatagattatcagggattatatatataaatatataaatatacataaaaatgttatggaattaatttattagaaacacttaagaagtacatatttttgtgcagtagatttttgaatcgatactttttttgaaaaccagtttccttgcttttttaagttctttctatatttttctttggaaatgcaaacattacaaaccaatgccatttttcaaatgcactgccatttaagattgattatagatggatttcttaattgaagtacttttataatcacagtgactgaacaaaatattttcaaagacatttgtcattccttaaagccaagattttaaagactaatgtccttcctgagggttactttactatactgtgtatggtgtatagccacagaaagtcagtctgataaattttcaatgtgtaagtgtgatgcattcaacccagattattgtagacttaacttattatatttcctctgatatgtaaaatgacaaactcttctttcctttttaatctttgtctttaatccatgaattaggtgcattatctgctgttgattcttttatatctgcactcattttatgacgtcagacaaaatgttttctaggtgttgctattgtatgagtcataattggtataattttaaagaaaattgtaatattgttttttggtgaaatatattattagtagtatattttccaaaatgattttattgtagtatgaactgataagtgagtcccgtgtccaagagactgtatttattactcttaaacacgctattccaaatgatattactactataaacaaagaatattaccggtctcagcaatcagtgatctttataaaagtacttttaaaaaatgatgaattatcaccaggcgctgtggctcacgcctgtaatcccagcactttgggaggccgaggcgggcggatcacgaggtcaggagatcaagaccatcctggctaacacggtgaaaccctgtctctactaaaaatacaaaaaattagccaggcgtggtggcaggtgcctgtaatcccagctacttgggaggctgaggcaggagaatggcgtgaacccaggaggcagagctggcagtgagctgagatcgcaccactgcactccagcctgggcgacacagctagactccgtctcaaaaaaaaaaaaagatgaagtatcaacatcagttcccagccagtctgcttggacacactggggtgccacaagtgggttgcaggttggttgcagacgggcagagatggattctgctgagctttcagagtgaccactggctggggcctagcgctggggcaaggttccagctggctgcctctagcctggaggagcttcatctagttacccaatgtgctgaaccaacagcatgaaatagcatcatttcctgtgctgtgttacaaagtgcaaacgtttggaaacactgatacagtacaattatttccatcactccatgttgttataatttttagttacttttcttgattgaaaagtcaatcacattgccaaaaatgatttttatgtcagttaaaggccagttgaaatttttttattaggatgttttaaatttacttttcactttcttgaatttttttcttctcaaatttccttggcccagtgcccatgagcaaatttcattcaaaaacaggaataaccttttgtcataaaattttggcaccagcttggtatactaaattactgcaaagataactgagtaccagaagtaaaaatctctgcaggtgggcttaggagtactcttgggtccctggttcctcagttgccaactctggtttcatgaagaaagaccatgctctgaaaatctagggaggagaaagttattgcagcacctggtgtgatttgttacaacttctttaaagacagcctgcctggctagctttttcctgttttcatgcagtttgccctcccagcattggtccacctgttgttcccaagaaactcattcttgttcccaagactccttttagccaaatctttcctatcattccaggacttttttgtgcttctctccccatgaatctacccgtgcctagggtgtgtgccaggcacgtggcaggtggccagtcagcatctggatgtgagaccaattattccagcctgccctcctctttctctttttatcattttgtattatttttgagacagggtctcgctttgttgcccaggctggagtgcagtggtgctctcatagctcactgcagcctcgatctctggcctcaagcaatcctcctgcctcagcctcccaaagtgctgggattataggtgtgtgccaccatgtccagccccctttctctctcttaactccctctccctgctgtcgttgccatactcttgcatacacaatcggttgtctagtttttgattgtttgtgtgtatggtgtgtgctgtctctctgaccagatttcaggttcctgaggcgagcctgcagctcatactgctcatctgtcctctcctatggtgggtgctcagggcctctcactgttagttactccctcctttctgcccagttctgcactcaactagtagaagcagccatcctttccccaagcaggaaattgtagtggtcgcccttaagagcagtgtgagggcagaagattaagggaggggaagagtccctggaactggaagaaggtaaatactttgccttgagagggcgccgaatcattttaccaaaatagtaaatggaaaaagtgtcaaaggttgggactagttttaaaaacacatatagccagtagacacatgggggctgtttaaataaaaacatttttttacccggttctgccatcacacacactagtgacattccaagtactcagtggccatgtggagctagtggccaccacacttgacaccacggatgcagaacttttccatcactgcagaaagctctgttgaacagtactgtgtccagagtgctaattatgaacagcttagaaaccacaattaatacattctcatgaggttaaaagcagacatgggaatagaacataatgcaaatgatgatgaatattttgggtgagaagagaaagctgaggacccatttctaggattctaagatgtaagatttcttaagttctttatcttagtctcatgcattctccacatcacgcgctgtaccatactgtgtagtcagaacagacagtgtgattgaaaagctttggaaaaagttaacacaaaggattatttagcacataggctgtagatacgtatgtgtgtatttgttcaacaattggagatggttgaatacccttgaacaaagtgtgtatcttctcaaatcagtggttgcactagtcaataattagaaggtgttgttatttttaaaactataagcaaaattatgaaggcctttaaaaaatctatcataataatgaaaaagaggttgtctcccaacagtgctgtccctcaaagaaaagactggttatgtggaaacagcacgtttggagagattattctagtgaataacagtgtatatttgtggtaggcaaatttctaagaatgacccctactgacacccccaccaatgaccttgtataatatcctcccactttgagcatgggtggaacctgtttgatccatgattatatcatgttatgtagcaaaaggaagactgtctgggggtggctaatcaaatcttgtgactcctttaaaagcagagagttttctcgggctggtggcagaagtcaagagacaaagcagaaggacctcaggcaccattgctgttttaaggtagagggggctgcatgagaaggaatgcacatggctttgaggagctgagagaggccttggctaacagcaaaggagctgggaactgcagtcctacagccacgggaactgaattcagcccataagctcattcataggcttggaaaggcattcatctccagtctccagataagacccagcctggccaacactttgaatccagccttgtgagaccctaagccgagaactcacttaagcctgcttggacttctgacctacagaactatgagataataaatcagtggtgttttaagctgctaaatttgtggcaatttgttatgtaggaataggaaatgaatacagtattttaaaagaggtaaagaggaaatcttcatacttgattccctgtagtatgctggatatttcttggtaaatgcatgaataaagtctttagtagcaagtcatgtacctggaccttgttggggatgttctcgccagagatctcaattagtcttcattggacgtttgttggcaaagttgcagcatcttactgagtagcgtttggtaaaggaactgctagaatgagactataggaataggactgaaacagtgtgtaagggatcttttttatttctagaagaggtgttactttatactgatttagactgctctgagcctgtcagaagtaaattcaagaatgattaacagtggtattttagatacattaagagcaaaaataacctttgccttgtagagatcagataatctgtgtatcgagggatttaattttttttaaccaaaacttagtttctctttttatcatctagcacagatcaaatcctgtatgggccagccaaatgtttgtagtttgagaatgttttgcaaatacctctctgcttccaagccattaatagctaatgaagttttattttggtttgtccattaatttttctgcttatttttactgtattgccatgtccttgatacgagacaagtagaactaaaagagagaaaatgagaggttaagactgtcagaagatgagaaatgtgagtcataaccataccttagtgttttaagggaactttatccagtccaatccctttatagaaaaggaacttttccaagttatgttgcagtatttatagtgtgtaaaattttgtttgactttggtagttgtgcattttaaattatgaatcttatatgctacgataactagtatgcttactttctttaatgcatgtgatcatacaatgctaaaaaggtattttttaaagacccaaccaaactgaggattttgttatacatatctgtatcaacacctatatataaactcttcttcttacctgaaacactccttcacccggtttgggcactaactagatcattttcaaaggaacaaacaggaaggagcaagctggcactccagggtgcatctgagctgagaatttagaagacctcattcaagaaagtaaactgagcattgatgggaaagcactttgcgcgaagtaacattcccactgttaatgttgcagtcaaaattgtggaaaaaaaaaagtggtgctaaaattgttctgaaatgaagattgtttgttgtggggaatattttaatactcttggactttagggaggctattttatgtgtcacttgctcctagttgtttcattgtattagtaattagcaaactgtcctggaaccaagggtttggtacttttgcctgattagcattccattatttatttgaaatgctcagtgtttgctcaaatttggtgaaaatatcttttgaagacatattctctgagccttaaaaatggctgctatcgacagtactgtaagatgcactgccagtagatttgatacttttcatttcagtgtaggcagtaatgttttctaagaaagatttagaaatcagcaaagtgtaacaattgtgtttcatttatggaatggaaaaactgaagtgtcccattcagaacactttggaaaggttgtctgcattaggggatgtttctggaagcatcccttagagaagctgcctgaggaatctccttgccaggttgtaccagatttttttttttcttaaatgtattgtatatattttccttaaaatgtcttcattggctgaatgattcctatccgtagtaatattttaggtacttgttggatttttaaaggaagattatttattttcagatttgctgctataattgaaaacctagtaactggtcctgttgattgtgccttcatcactgtttctctgtgccacgaccgttcatgtgtatttgaaataaagaagtttaaaaagccatgttgaattcaagattccttctaataaaaataggtgaaacttaa

SEQ ID NO: 408 - RNA, 19 nt, Artificial Sequence
  Description of Artificial Sequence Synthetic oligonucleotide
acuuugucuugguauacua

SEQ ID NO: 409 - RNA, 19 nt, Artificial Sequence
  Description of Artificial Sequence Synthetic oligonucleotide
aaguaaggaugcguugaau

SEQ ID NO: 410 - RNA, 19 nt, Artificial Sequence
  Description of Artificial Sequence Synthetic oligonucleotide
aguaaggaugcguugaaua

SEQ ID NO: 411 - RNA, 19 nt, Artificial Sequence
  Description of Artificial Sequence Synthetic oligonucleotide
guacagauuggcaacggaa

SEQ ID NO: 412 - RNA, 19 nt, Artificial Sequence
  Description of Artificial Sequence Synthetic oligonucleotide
ccuuauugauaaccuguau

SEQ ID NO: 413 - RNA, 19 nt, Artificial Sequence
  Description of Artificial Sequence Synthetic oligonucleotide
aaagauaguuaagcaugua

SEQ ID NO: 414 - RNA, 19 nt, Artificial Sequence
  Description of Artificial Sequence Synthetic oligonucleotide
ggaaagugaaucucccaua

SEQ ID NO: 415 - RNA, 19 nt, Artificial Sequence
  Description of Artificial Sequence Synthetic oligonucleotide
agugaaucucccauaauaa

SEQ ID NO: 416 - RNA, 19 nt, Artificial Sequence
  Description of Artificial Sequence Synthetic oligonucleotide
ccagcuugguauacuaaau

SEQ ID NO: 417 - RNA, 19 nt, Artificial Sequence
  Description of Artificial Sequence Synthetic oligonucleotide
cuggaugugagaccaauua

SEQ ID NO: 418 - RNA, 19 nt, Artificial Sequence
  Description of Artificial Sequence Synthetic oligonucleotide
cuugcauacacaaucgguu

SEQ ID NO: 419 - RNA, 19 nt, Artificial Sequence
  Description of Artificial Sequence Synthetic oligonucleotide
auuguaguggucgcccuua

SEQ ID NO: 420 - RNA, 19 nt, Artificial Sequence
  Description of Artificial Sequence Synthetic oligonucleotide
caaagguugggacuaguuu

SEQ ID NO: 421 - RNA, 19 nt, Artificial Sequence
  Description of Artificial Sequence Synthetic oligonucleotide
uuaucuuagucucaugcau

SEQ ID NO: 422 - RNA, 19 nt, Artificial Sequence
  Description of Artificial Sequence Synthetic oligonucleotide
gagaugguugaauacccuu

SEQ ID NO: 423 - RNA, 19 nt, Artificial Sequence
  Description of Artificial Sequence Synthetic oligonucleotide
gugguugcacuagucaaua

SEQ ID NO: 424 - RNA, 19 nt, Artificial Sequence
  Description of Artificial Sequence Synthetic oligonucleotide
uaucauguuauguagcaaa

SEQ ID NO: 425 - RNA, 19 nt, Artificial Sequence
  Description of Artificial Sequence Synthetic oligonucleotide
ccuguaguaugcuggauau

SEQ ID NO: 426 - RNA, 19 nt, Artificial Sequence
  Description of Artificial Sequence Synthetic oligonucleotide
caucuuacugaguagcguu

SEQ ID NO: 427 - RNA, 19 nt, Artificial Sequence
  Description of Artificial Sequence Synthetic oligonucleotide
uacugaguagcguuuggua

SEQ ID NO: 428 - RNA, 19 nt, Artificial Sequence
  Description of Artificial Sequence Synthetic oligonucleotide
acugaguagcguuugguaa

SEQ ID NO: 429 - RNA, 19 nt, Artificial Sequence
  Description of Artificial Sequence Synthetic oligonucleotide
aucuguguaucgagggauu

SEQ ID NO: 430 - RNA, 19 nt, Artificial Sequence
  Description of Artificial Sequence Synthetic oligonucleotide
uaugcuacgauaacuagua

SEQ ID NO: 431 - RNA, 19 nt, Artificial Sequence
  Description of Artificial Sequence Synthetic oligonucleotide
acgauaacuaguaugcuua

SEQ ID NO: 432 - RNA, 19 nt, Artificial Sequence
  Description of Artificial Sequence Synthetic oligonucleotide
ggcacuaacuagaucauuu

SEQ ID NO: 433 - RNA, 19 nt, Artificial Sequence
  Description of Artificial Sequence Synthetic oligonucleotide
agcacuuugcgcgaaguaa

SEQ ID NO: 434 - RNA, 19 nt, Artificial Sequence
  Description of Artificial Sequence Synthetic oligonucleotide
gucccauucagaacacuuu

SEQ ID NO: 435 - RNA, 19 nt, Artificial Sequence
  Description of Artificial Sequence Synthetic oligonucleotide
auuccuauccguaguaaua

SEQ ID NO: 436 - RNA, 19 nt, Artificial Sequence
  Description of Artificial Sequence Synthetic oligonucleotide
uaguauaccaagacaaagu

SEQ ID NO: 437 - RNA, 19 nt, Artificial Sequence
  Description of Artificial Sequence Synthetic oligonucleotide
auucaacgcauccuuacuu

SEQ ID NO: 438 - RNA, 19 nt, Artificial Sequence
  Description of Artificial Sequence Synthetic oligonucleotide
uauucaacgcauccuuacu

SEQ ID NO: 439 - RNA, 19 nt, Artificial Sequence
  Description of Artificial Sequence Synthetic oligonucleotide
uuccguugccaaucuguac

SEQ ID NO: 440 - RNA, 19 nt, Artificial Sequence
  Description of Artificial Sequence Synthetic oligonucleotide
auacagguuaucaauaagg

SEQ ID NO: 441 - RNA, 19 nt, Artificial Sequence
  Description of Artificial Sequence Synthetic oligonucleotide
uacaugcuuaacuaucuuu

SEQ ID NO: 442 - RNA, 19 nt, Artificial Sequence
  Description of Artificial Sequence Synthetic oligonucleotide
uaugggagauucacuuucc

SEQ ID NO: 443 - RNA, 19 nt, Artificial Sequence
  Description of Artificial Sequence Synthetic oligonucleotide
uuauuaugggagauucacu

SEQ ID NO: 444 - RNA, 19 nt, Artificial Sequence
  Description of Artificial Sequence Synthetic oligonucleotide
auuuaguauaccaagcugg

SEQ ID NO: 445 - RNA, 19 nt, Artificial Sequence
  Description of Artificial Sequence Synthetic oligonucleotide
uaauuggucucacauccag

SEQ ID NO: 446 - RNA, 19 nt, Artificial Sequence
  Description of Artificial Sequence Synthetic oligonucleotide
aaccgauuguguaugcaag

SEQ ID NO: 447 - RNA, 19 nt, Artificial Sequence
  Description of Artificial Sequence Synthetic oligonucleotide
uaagggcgaccacuacaau

SEQ ID NO: 448 - RNA, 19 nt, Artificial Sequence
  Description of Artificial Sequence Synthetic oligonucleotide
aaacuagucccaaccuuug

SEQ ID NO: 449 - RNA, 19 nt, Artificial Sequence
  Description of Artificial Sequence Synthetic oligonucleotide
augcaugagacuaagauaa

SEQ ID NO: 450 - RNA, 19 nt, Artificial Sequence
  Description of Artificial Sequence Synthetic oligonucleotide
aaggguauucaaccaucuc

SEQ ID NO: 451 - RNA, 19 nt, Artificial Sequence
  Description of Artificial Sequence Synthetic oligonucleotide
uauugacuagugcaaccac

SEQ ID NO: 452 - RNA, 19 nt, Artificial Sequence
  Description of Artificial Sequence Synthetic oligonucleotide
uuugcuacauaacaugaua

SEQ ID NO: 453 - RNA, 19 nt, Artificial Sequence
  Description of Artificial Sequence Synthetic oligonucleotide
auauccagcauacuacagg

SEQ ID NO: 454 - RNA, 19 nt, Artificial Sequence
  Description of Artificial Sequence Synthetic oligonucleotide
aacgcuacucaguaagaug

SEQ ID NO: 455 - RNA, 19 nt, Artificial Sequence
  Description of Artificial Sequence Synthetic oligonucleotide
uaccaaacgcuacucagua

SEQ ID NO: 456 - RNA, 19 nt, Artificial Sequence
  Description of Artificial Sequence Synthetic oligonucleotide
uuaccaaacgcuacucagu

SEQ ID NO: 457 - RNA, 19 nt, Artificial Sequence
  Description of Artificial Sequence Synthetic oligonucleotide
aaucccucgauacacagau

SEQ ID NO: 458 - RNA, 19 nt, Artificial Sequence
  Description of Artificial Sequence Synthetic oligonucleotide
uacuaguuaucguagcaua

SEQ ID NO: 459 - RNA, 19 nt, Artificial Sequence
  Description of Artificial Sequence Synthetic oligonucleotide
uaagcauacuaguuaucgu

SEQ ID NO: 460 - RNA, 19 nt, Artificial Sequence
  Description of Artificial Sequence Synthetic oligonucleotide
aaaugaucuaguuagugcc

SEQ ID NO: 461 - RNA, 19 nt, Artificial Sequence
  Description of Artificial Sequence Synthetic oligonucleotide
uuacuucgcgcaaagugcu

SEQ ID NO: 462 - RNA, 19 nt, Artificial Sequence
  Description of Artificial Sequence Synthetic oligonucleotide
aaaguguucugaaugggac

SEQ ID NO: 463 - RNA, 19 nt, Artificial Sequence
  Description of Artificial Sequence Synthetic oligonucleotide
uauuacuacggauaggaau

SEQ ID NO: 464 - DNA, 2482 bp, Homo sapiens
cccagatccaggccgggccgcggctctcgccgcccagcccagcccagcccggcccggcccggccctgccgcggaggcgaggccgccagtgtcccgcgcccctgatatctgcagtgagcctgatacctgcctctgcccttctgagcctgttcctcttccctgagtacagggcacaaagcttgcgccctgaggggcggccggcgcgctccctggcccggtccccgcccggccccgggccccccgcccctccccgacccggggccggggcccctgccgccgccgccgccgccttccgacccctgcgccccggccccggtcccccgggccatgcagcctcggccccgcgggcgcccgccgcgcacccgaggagatgaggctccgcaatggcaccttcctgacgctgctgctcttctgcctgtgcgccttcctctcgctgtcctggtacgcggcactcagcggccagaaaggcgacgttgtggacgtttaccagcgggagttcctggcgctgcgcgatcggttgcacgcagctgagcaggagagcctcaagcgctccaaggagctcaacctggtgctggacgagatcaagagggccgtgtcagaaaggcaggcgctgcgagacggagacggcaatcgcacctggggccgcctaacagaggacccccgattgaagccgtggaacggctcacaccggcacgtgctgcacctgcccaccgtcttccatcacctgccacacctgctggccaaggagagcagtctgcagcccgcggtgcgcgtgggccagggccgcaccggagtgtcggtggtgatgggcatcccgagcgtgcggcgcgaggtgcactcgtacctgactgacactctgcactcgctcatctccgagctgagcccgcaggagaaggaggactcggtcatcgtggtgctgatcgccgagactgactcacagtacacttcggcagtgacagagaacatcaaggccttgttccccacggagatccattctgggctcctggaggtcatctcaccctccccccacttctaccctgacttctcccgcctccgagagtcctttggggaccccaaggagagagtcaggtggaggaccaaacagaacctcgattactgcttcctcatgatgtacgcgcagtccaaaggcatctactacgtgcagctggaggatgacatcgtggccaagcccaactacctgagcaccatgaagaactttgcactgcagcagccttcagaggactggatgatcctggagttctcccagctgggcttcattggtaagatgttcaagtcgctggacctgagcctgattgtagagttcattctcatgttctaccgggacaagcccatcgactggctcctggaccatattctgtgggtgaaagtctgcaaccccgagaaggatgcgaagcactgtgaccggcagaaagccaacctgcggatccgcttcaaaccgtccctcttccagcacgtgggcactcactcctcgctggctggcaagatccagaaactgaaggacaaagactttggaaagcaggcgctgcggaaggagcatgtgaacccgccagcagaggtgagcacgagcctgaagacataccagcacttcaccctggagaaagcctacctgcgcgaggacttcttctgggccttcacccctgccgcgggggacttcatccgcttccgcttcttccaacctctaagactggagcggttcttcttccgcagtgggaacatcgagcacccggaggacaagctcttcaacacgtctgtggaggtgctgcccttcgacaaccctcagtcagacaaggaggccctgcaggagggccgcaccgccaccctccggtaccctcggagccccgacggctacctccagatcggctccttctacaagggagtggcagagggagaggtggacccagccttcggccctctggaagcactgcgcctctcgatccagacggactcccctgtgtgggtgattctgagcgagatcttcctgaaaaaggccgactaagctgcgggcttctgagggtaccctgtggccagccctgaagcccacatttctgggggtgtcgtcactgccgtccccggagggccagatacggccccgcccaaagggttctgcctggcgtcgggcttgggccggcctggggtccgccgctggcccggaggccctaggagctggtgctgcccccgcccgccgggccgcggaggaggcaggcggcccccacactgtgcctgaggcccggaaccgttcgcacccggcctgccccagtcaggccgttttagaagagcttttacttgggcgcccgccgtctctggcgcgaacactggaatgcatatactactttatgtgctgtgttttttattcttggatacatttgattttttcacgtaagtccacatatacttctataagagcgtgacttgtaataaagggttaatgaagtgtgtgcctcaaaaaaaaaaaaaaaaaaaaa

SEQ ID NO: 465 - RNA, 19 nt, Artificial Sequence
  Description of Artificial Sequence Synthetic oligonucleotide
aagagggccgugucagaaa

SEQ ID NO: 466 - RNA, 19 nt, Artificial Sequence
  Description of Artificial Sequence Synthetic oligonucleotide
cucguaccugacugacacu

SEQ ID NO: 467 - RNA, 19 nt, Artificial Sequence
  Description of Artificial Sequence Synthetic oligonucleotide
cguaccugacugacacucu

SEQ ID NO: 468 - RNA, 19 nt, Artificial Sequence
  Description of Artificial Sequence Synthetic oligonucleotide
ccgagacugacucacagua

SEQ ID NO: 469 - RNA, 19 nt, Artificial Sequence
  Description of Artificial Sequence Synthetic oligonucleotide
uuccccacggagauccauu

SEQ ID NO: 470 - RNA, 19 nt, Artificial Sequence
  Description of Artificial Sequence Synthetic oligonucleotide
ccaaacagaaccucgauua

SEQ ID NO: 471 - RNA, 19 nt, Artificial Sequence
  Description of Artificial Sequence Synthetic oligonucleotide
aaacagaaccucgauuacu

SEQ ID NO: 472 - RNA, 19 nt, Artificial Sequence
  Description of Artificial Sequence Synthetic oligonucleotide
cagaaccucgauuacugcu

SEQ ID NO: 473 - RNA, 19 nt, Artificial Sequence
  Description of Artificial Sequence Synthetic oligonucleotide
agaaccucgauuacugcuu

SEQ ID NO: 474 - RNA, 19 nt, Artificial Sequence
  Description of Artificial Sequence Synthetic oligonucleotide
uccucaugauguacgcgca

SEQ ID NO: 475 - RNA, 19 nt, Artificial Sequence
  Description of Artificial Sequence Synthetic oligonucleotide
gccugauuguagaguucau

SEQ ID NO: 476 - RNA, 19 nt, Artificial Sequence
  Description of Artificial Sequence Synthetic oligonucleotide
aaagucugcaaccccgaga

SEQ ID NO: 477 - RNA, 19 nt, Artificial Sequence
  Description of Artificial Sequence Synthetic oligonucleotide
gagaaggaugcgaagcacu

SEQ ID NO: 478 - RNA, 19 nt, Artificial Sequence
  Description of Artificial Sequence Synthetic oligonucleotide
gacaacccucagucagaca

SEQ ID NO: 479 - RNA, 19 nt, Artificial Sequence
  Description of Artificial Sequence Synthetic oligonucleotide
ugggugauucugagcgaga

SEQ ID NO: 480 - RNA, 19 nt, Artificial Sequence
  Description of Artificial Sequence Synthetic oligonucleotide
aggccguuuuagaagagcu

SEQ ID NO: 481 - RNA, 19 nt, Artificial Sequence
  Description of Artificial Sequence Synthetic oligonucleotide
gccguuuuagaagagcuuu

SEQ ID NO: 482 - RNA, 19 nt, Artificial Sequence
  Description of Artificial Sequence Synthetic oligonucleotide
uucacguaaguccacauau

SEQ ID NO: 483 - RNA, 19 nt, Artificial Sequence
  Description of Artificial Sequence Synthetic oligonucleotide
ucacguaaguccacauaua

SEQ ID NO: 484 - RNA, 19 nt, Artificial Sequence
  Description of Artificial Sequence Synthetic oligonucleotide
cguaaguccacauauacuu

SEQ ID NO: 485 - RNA, 19 nt, Artificial Sequence
  Description of Artificial Sequence Synthetic oligonucleotide
uauacuucuauaagagcgu

SEQ ID NO: 486 - RNA, 19 nt, Artificial Sequence
  Description of Artificial Sequence Synthetic oligonucleotide
uauaagagcgugacuugua

SEQ ID NO: 487 - RNA, 19 nt, Artificial Sequence
  Description of Artificial Sequence Synthetic oligonucleotide
uaagagcgugacuuguaau

SEQ ID NO: 488 - RNA, 19 nt, Artificial Sequence
  Description of Artificial Sequence Synthetic oligonucleotide
agagcgugacuuguaauaa

SEQ ID NO: 489 - RNA, 19 nt, Artificial Sequence
  Description of Artificial Sequence Synthetic oligonucleotide
guuaaugaagugugugccu

SEQ ID NO: 490 - RNA, 19 nt, Artificial Sequence
  Description of Artificial Sequence Synthetic oligonucleotide
uuucugacacggcccucuu

SEQ ID NO: 491 - RNA, 19 nt, Artificial Sequence
  Description of Artificial Sequence Synthetic oligonucleotide
agugucagucagguacgag

SEQ ID NO: 492 - RNA, 19 nt, Artificial Sequence
  Description of Artificial Sequence Synthetic oligonucleotide
agagugucagucagguacg

SEQ ID NO: 493 - RNA, 19 nt, Artificial Sequence
  Description of Artificial Sequence Synthetic oligonucleotide
uacugugagucagucucgg

SEQ ID NO: 494 - RNA, 19 nt, Artificial Sequence
  Description of Artificial Sequence Synthetic oligonucleotide
aauggaucuccguggggaa

SEQ ID NO: 495 - RNA, 19 nt, Artificial Sequence
  Description of Artificial Sequence Synthetic oligonucleotide
uaaucgagguucuguuugg

SEQ ID NO: 496 - RNA, 19 nt, Artificial Sequence
  Description of Artificial Sequence Synthetic oligonucleotide
aguaaucgagguucuguuu

SEQ ID NO: 497 - RNA, 19 nt, Artificial Sequence
  Description of Artificial Sequence Synthetic oligonucleotide
agcaguaaucgagguucug

SEQ ID NO: 498 - RNA, 19 nt, Artificial Sequence
  Description of Artificial Sequence Synthetic oligonucleotide
aagcaguaaucgagguucu

SEQ ID NO: 499 - RNA, 19 nt, Artificial Sequence
  Description of Artificial Sequence Synthetic oligonucleotide
ugcgcguacaucaugagga

SEQ ID NO: 500 - RNA, 19 nt, Artificial Sequence
  Description of Artificial Sequence Synthetic oligonucleotide
augaacucuacaaucaggc

SEQ ID NO: 501 - RNA, 19 nt, Artificial Sequence
  Description of Artificial Sequence Synthetic oligonucleotide
ucucgggguugcagacuuu

SEQ ID NO: 502 - RNA, 19 nt, Artificial Sequence
  Description of Artificial Sequence Synthetic oligonucleotide
agugcuucgcauccuucuc

SEQ ID NO: 503 - RNA, 19 nt, Artificial Sequence
  Description of Artificial Sequence Synthetic oligonucleotide
ugucugacugaggguuguc

SEQ ID NO: 504 - RNA, 19 nt, Artificial Sequence
  Description of Artificial Sequence Synthetic oligonucleotide
ucucgcucagaaucaccca

SEQ ID NO: 505 - RNA, 19 nt, Artificial Sequence
  Description of Artificial Sequence Synthetic oligonucleotide
agcucuucuaaaacggccu

SEQ ID NO: 506 - RNA, 19 nt, Artificial Sequence
  Description of Artificial Sequence Synthetic oligonucleotide
aaagcucuucuaaaacggc

SEQ ID NO: 507 - RNA, 19 nt, Artificial Sequence
  Description of Artificial Sequence Synthetic oligonucleotide
auauguggacuuacgugaa

SEQ ID NO: 508 - RNA, 19 nt, Artificial Sequence
  Description of Artificial Sequence Synthetic oligonucleotide
uauauguggacuuacguga

SEQ ID NO: 509 - RNA, 19 nt, Artificial Sequence
  Description of Artificial Sequence Synthetic oligonucleotide
aaguauauguggacuuacg

SEQ ID NO: 510 - RNA, 19 nt, Artificial Sequence
  Description of Artificial Sequence Synthetic oligonucleotide
acgcucuuauagaaguaua

SEQ ID NO: 511 - RNA, 19 nt, Artificial Sequence
  Description of Artificial Sequence Synthetic oligonucleotide
uacaagucacgcucuuaua

SEQ ID NO: 512 - RNA, 19 nt, Artificial Sequence
  Description of Artificial Sequence Synthetic oligonucleotide
auuacaagucacgcucuua

SEQ ID NO: 513 - RNA, 19 nt, Artificial Sequence
  Description of Artificial Sequence Synthetic oligonucleotide
uuauuacaagucacgcucu

SEQ ID NO: 514 - RNA, 19 nt, Artificial Sequence
  Description of Artificial Sequence Synthetic oligonucleotide
aggcacacacuucauuaac

SEQ ID NO: 515 - DNA, 5384 bp, Mus musculus
acttcgtggcagctaaaccagaggccagacggcagctcggggagtccgaccacacctcaggaaacagctgtgccacaggatggaaacacacgcgcatctccacagggagcagagctacgcaggacctcatgaactctattttaagaaactctcaagtaaaaagaagcaagtcatggagaagaacgggaacaaccgaaagctccgggtttgcgttgccacctgcaaccgagctgactactccaaactggccccgatcatgttcggcatcaagacagagcccgcgttctttgagttggacgtggtggtgctcggctcccacctgattgacgactatggaaacacataccgcatgattgagcaagatgactttgacattaacaccaggctccacacgattgttagaggggaagatgaagcggccatggtagagtcggtaggcctagcgctcgtgaagctaccggacgtcctcaatcgcctgaagcccgacatcatgattgttcacggagaccgatttgacgcccttgctctggctacgtctgctgccttgatgaacatccgcatccttcacattgaaggaggcgaggtcagcgggaccattgatgactctatcagacacgccataacaaaactggctcactaccatgtgtgctgcactagaagtgcagagcagcacctgatctctatgtgcgaggaccacgaccgcatcctgttggcaggctgcccttcctatgacaaactgctctccgccaagaacaaagactatatgagcatcattcggatgtggctaggcgatgatgtaaaatgtaaggattacatcgttgccctgcagcatcccgtgaccactgacattaagcattccataaagatgtttgagctaacactggatgccctgatctcgtttaacaagaggaccctagttctgtttccaaatatcgatgcaggcagcaaggagatggttcgagtgatgcggaagaagggcatcgagcatcaccccaatttccgtgcagtcaagcacgtcccgtttgaccagttcatacagctggtcgcccacgctggctgcatgattgggaatagcagctgcggcgtgcgagaggttggcgctttcggaacacccgtgatcaacctgggcacaaggcagataggaagagaaaccggggagaatgttcttcatgtcagggatgctgacacccaagataaaatattgcaagcactacacctccagttcggcaaacagtacccttgctcaaagatatatggggatgggaatgctgttccaaggattttaaagtttctcaaatccattgaccttcaagagccactacagaagaaattctgcttcccccctgtaaaggagaacatctctcaagacattgaccacatcctggaaactctgagtgccttggctgttgatcttggcgggacaaacctgagggtggcaatagttagcatgaagggtgaaatcgttaagaagtacactcagttcaaccctaaaacctatgaagaaaggattagtttaatcctgcagatgtgtgtggaagctgccgcggaagctgtgaaactcaattgcagaattctgggagtaggcatctccacaggtggccgcgtgaatccccaggaaggagttgtgctgcattcaaccaagctgatccaggaatggaactccgtggacctcaggacacccctctccgacaccctgcatctccccgtgtgggtggacaatgacggcaactgtgccgccatggcagagaggaagttcggccaaggaaaaggacaggagaacttcgtgacgctcatcacggggacagggatcggtggggggatcatccaccagcacgaactgatccacggcagctccttctgcgcggcggagctcggccatctcgtggtgtccctggacggtcctgactgctcctgtggaagccatgggtgcatcgaagcgtacgcctctggaatggccttgcagagggaagcaaagaaactccatgatgaggacctgctcttggtggaagggatgtcagtaccaaaagacgaagctgtgggtgccctccatctcatccaggctgccaagctgggcaacgtgaaggcccagagcatcttacgaacagctggaactgctttgggacttggggttgtgaacatcctccacactatgaatccttccctggtgatcctgtctggagtcctggccagtcactacatccacatcgtgaaggacgtcatccgccagcaagccttgtcctccgtgcaggatgtggacgtggtggtctcagacttggtggacccggccctgcttggcgcagccagcatggttctggactacacaacgcgcaggatccactaggtctcccgggaacggacacggacagagactcgggagctgcttagagtggaaccatgctcttctagatcagtgtttctgcgaaggcaaatttggggggagggctgctgagacagctcagtggttaagagcctgccctgctcctgccagtccccagcacccatgtcaggcagctcagctgcctggaagccaagctccaggggacccaatgcctctctgccgggggcagctgcactcagatgtacatacccctctccacacacatacaaataaagcttatttttcaaaaggcaaacttgatatctggtcccagaaataccagaatctctctataaaagggattatcagaatggctcacaggctgtggttcagctagtccaacagtggctgtcttgcaagggacagcccaagagtcctgttcagtgcctagggctggatgtctcagctggtcttcagtacacgtcagactcgtgcagcaggctttaataaccgtgaatggaaagcaagggccaccaggcaatagcaattgcttccttcttccttgtactttacagaagctgtcaccgaaaggtgtggccccgattaaaggtggattttcccacctcaaaagatgtggatcaaaagtggaccttcctacttcaaaggatttaattaaaaatctctcccaggtgtacccagcctcttaggctttagttaattccagatgtaatcaagttgacaaccaagaatagtatcacacctttcaaatccactttccccatgagctttcgccaaggctgctctgatagggtcatctcactgcaaaccctgctgtgggttccagggacacttcctgcaccaaaaggagcaatagcacctgacccagaagacttaggactcctcatagacctcgggatgctagctagcatcatgggaaggcttagtttactagtccttcctcagaccacagagagagcctgtcacacaacagaagacaagctcaccagaaatgcccactcactttgatttttttagcaacataatacatttcacaacttttcaagaaggcatactcctgttaagtagccaccaccagccaagggattggacagatgtgtcaccgcaccccgggacaggacagatgtgtcaccgcacccctgtgtctgctgagagatgctttacttgaggagtttgtgagatactttagactggggtccaagttatgacggccttagtggctgtccttatcttcccagagattgcaggcaggcaggccggcctcagagcttctggaaagaaagcctttcactgctctctgtgtcccagaatgtcagcatacacatttacgtgtactttgtgacacctaaagcccagagccatggatctggatttggactacattattgtctttttgatgtgtttgttttcgagacagaatctcgcttgtctctgtaatctcgcttggttcatcttaaacttacagcaatcctcctgcccccgcttccagagtactgggattataaacaatcaatcaccacacccagcttaacatctgctttttcttggatgacaaaatataaaagtcctcagttctgcctaccgactgtaccctggagtaagcacccacacgtgccagtgtatcagttaccttctgctgctgtgatggaagtgaccaaggcaacttacagaagggagagtttcattagcttacagttctagaggattcataaaggcagggaaggcacaggaacaggaagccagggcagggagcttgagaaaccacatcttcaacgacaagccaacagcagcagaagtgaatgcaaatagggcaggccacggactcacccagactgcaccagcaaacagcaccactgaggagcacgtaccgacatacctgagcctacagagacattcccattcaaactgcatccactcctgactcccataaactcatggacgtaccgccatgcaaagcagtcagtcagtccaccattcaagtccccggagcctaacagtctccatactaagcactacgtttcttctgagacgtaaggggacctctcaacgacgacgtcctggtaaacccacaatggcagagtatacattttgcttccaaagagagcagtggaagcatgggaataggggcatgaagaatggggcacggaaggaattagggacacagtgggaaagaggcagggagaactcagcaggacaaactccaaccctgcagcttcatacctatcggggccatgagtgtcaaagggcttagaggttcagccctccagctttgctgtctgtcgcctacatacttgccggtcttgggctggttctatgcagccctttaaccagtggttctcagcctccctaatgctataatcctttactatagttccacgtatttattaatacaactcatgttgtggtgacccacaaccagaaaattactttgctgctattttgctaactgtacttctactgtaatgaatcataatgtacatatgtgatatgcaggatatgatatatgcccatgaaaaggatcttccgaccccacaaaggggcaacgacccacaggatgagaactgctgccgtcaatcatcacctgggtctggttattatccaggcctggagcaaaggatgcccacgaagcggtcatcactcaccagtgggtcaccctgtaatcactgcaaccaaacaactgcactgttaccacgctgtgagccatgtatttgaaaggataataccaaactgaaaaccaaatcctggttttaatcaactctgtggaaagtgtaatatattctgacaaaatatatatggtgaaactgtctcaatttgaataaatatttttattgcaattctgaaccaattttaaaagaaaagatacacttccttgtccaagtcttacagtgtgcttaccatcacctctgccaaaatcataaaatcatttcttttcccaaaagagctctgtatttgtggccctgctttatcattatctaatttatgttatcaagtgccactgctttctgaaccaagggaaaaggagctattataaaggaccattattaaacta

SEQ ID NO: 516 - RNA, 19 nt, Artificial Sequence
  Description of Artificial Sequence Synthetic oligonucleotide
gauggaaacacacgcgcau

SEQ ID NO: 517 - RNA, 19 nt, Artificial Sequence
  Description of Artificial Sequence Synthetic oligonucleotide
cgaucauguucggcaucaa

SEQ ID NO: 518 - RNA, 19 nt, Artificial Sequence
  Description of Artificial Sequence Synthetic oligonucleotide
cccaccugauugacgacua

SEQ ID NO: 519 - RNA, 19 nt, Artificial Sequence
  Description of Artificial Sequence Synthetic oligonucleotide
ccaccugauugacgacuau

SEQ ID NO: 520 - RNA, 19 nt, Artificial Sequence
  Description of Artificial Sequence Synthetic oligonucleotide
gaaacacauaccgcaugau

SEQ ID NO: 521 - RNA, 19 nt, Artificial Sequence
  Description of Artificial Sequence Synthetic oligonucleotide
acguccucaaucgccugaa

SEQ ID NO: 522 - RNA, 19 nt, Artificial Sequence
  Description of Artificial Sequence Synthetic oligonucleotide
auaugagcaucauucggau

SEQ ID NO: 523 - RNA, 19 nt, Artificial Sequence
  Description of Artificial Sequence Synthetic oligonucleotide
uggcuaggcgaugauguaa

SEQ ID NO: 524 - RNA, 19 nt, Artificial Sequence
  Description of Artificial Sequence Synthetic oligonucleotide
acauuaagcauuccauaaa

SEQ ID NO: 525 - RNA, 19 nt, Artificial Sequence
  Description of Artificial Sequence Synthetic oligonucleotide
uggaugcccugaucucguu

SEQ ID NO: 526 - RNA, 19 nt, Artificial Sequence
  Description of Artificial Sequence Synthetic oligonucleotide
cccugaucucguuuaacaa

SEQ ID NO: 527 - RNA, 19 nt, Artificial Sequence
  Description of Artificial Sequence Synthetic oligonucleotide
agucaagcacgucccguuu

SEQ ID NO: 528 - RNA, 19 nt, Artificial Sequence
  Description of Artificial Sequence Synthetic oligonucleotide
ucggaacacccgugaucaa

SEQ ID NO: 529 - RNA, 19 nt, Artificial Sequence
  Description of Artificial Sequence Synthetic oligonucleotide
uacaccuccaguucggcaa

SEQ ID NO: 530 - RNA, 19 nt, Artificial Sequence
  Description of Artificial Sequence Synthetic oligonucleotide
acucaguucaacccuaaaa

SEQ ID NO: 531 - RNA, 19 nt, Artificial Sequence
  Description of Artificial Sequence Synthetic oligonucleotide
ugaacauccuccacacuau

SEQ ID NO: 532 - RNA, 19 nt, Artificial Sequence
  Description of Artificial Sequence Synthetic oligonucleotide
gggaaggcuuaguuuacua

SEQ ID NO: 533 - RNA, 19 nt, Artificial Sequence
  Description of Artificial Sequence Synthetic oligonucleotide
gcuuaguuuacuaguccuu

SEQ ID NO: 534 - RNA, 19 nt, Artificial Sequence
  Description of Artificial Sequence Synthetic oligonucleotide
uccaaguuaugacggccuu

SEQ ID NO: 535 - RNA, 19 nt, Artificial Sequence
  Description of Artificial Sequence Synthetic oligonucleotide
ccaaguuaugacggccuua

SEQ ID NO: 536 - RNA, 19 nt, Artificial Sequence
  Description of Artificial Sequence Synthetic oligonucleotide
ugucucuguaaucucgcuu

SEQ ID NO: 537 - RNA, 19 nt, Artificial Sequence
  Description of Artificial Sequence Synthetic oligonucleotide
aggagcacguaccgacaua

SEQ ID NO: 538 - RNA, 19 nt, Artificial Sequence
  Description of Artificial Sequence Synthetic oligonucleotide
uuacuauaguuccacguau

SEQ ID NO: 539 - RNA, 19 nt, Artificial Sequence
  Description of Artificial Sequence Synthetic oligonucleotide
gaucuuccgaccccacaaa

SEQ ID NO: 540 - RNA, 19 nt, Artificial Sequence
  Description of Artificial Sequence Synthetic oligonucleotide
gagaacugcugccgucaau

SEQ ID NO: 541 - RNA, 19 nt, Artificial Sequence
  Description of Artificial Sequence Synthetic oligonucleotide
cuauuauaaaggaccauua

SEQ ID NO: 542 - RNA, 19 nt, Artificial Sequence
  Description of Artificial Sequence Synthetic oligonucleotide
augcgcguguguuuccauc

SEQ ID NO: 543 - RNA, 19 nt, Artificial Sequence
  Description of Artificial Sequence Synthetic oligonucleotide
uugaugccgaacaugaucg

SEQ ID NO: 544 - RNA, 19 nt, Artificial Sequence
  Description of Artificial Sequence Synthetic oligonucleotide
uagucgucaaucagguggg

SEQ ID NO: 545 - RNA, 19 nt, Artificial Sequence
  Description of Artificial Sequence Synthetic oligonucleotide
auagucgucaaucaggugg

SEQ ID NO: 546 - RNA, 19 nt, Artificial Sequence
  Description of Artificial Sequence Synthetic oligonucleotide
aucaugcgguauguguuuc

SEQ ID NO: 547 - RNA, 19 nt, Artificial Sequence
  Description of Artificial Sequence Synthetic oligonucleotide
uucaggcgauugaggacgu

SEQ ID NO: 548 - RNA, 19 nt, Artificial Sequence
  Description of Artificial Sequence Synthetic oligonucleotide
auccgaaugaugcucauau

SEQ ID NO: 549 - RNA, 19 nt, Artificial Sequence
  Description of Artificial Sequence Synthetic oligonucleotide
uuacaucaucgccuagcca

SEQ ID NO: 550 - RNA, 19 nt, Artificial Sequence
  Description of Artificial Sequence Synthetic oligonucleotide
uuuauggaaugcuuaaugu

SEQ ID NO: 551 - RNA, 19 nt, Artificial Sequence
  Description of Artificial Sequence Synthetic oligonucleotide
aacgagaucagggcaucca

SEQ ID NO: 552 - RNA, 19 nt, Artificial Sequence
  Description of Artificial Sequence Synthetic oligonucleotide
uuguuaaacgagaucaggg

SEQ ID NO: 553 - RNA, 19 nt, Artificial Sequence
  Description of Artificial Sequence Synthetic oligonucleotide
aaacgggacgugcuugacu

SEQ ID NO: 554 - RNA, 19 nt, Artificial Sequence
  Description of Artificial Sequence Synthetic oligonucleotide
uugaucacggguguuccga

SEQ ID NO: 555 - RNA, 19 nt, Artificial Sequence
  Description of Artificial Sequence Synthetic oligonucleotide
uugccgaacuggaggugua

SEQ ID NO: 556 - RNA, 19 nt, Artificial Sequence
  Description of Artificial Sequence Synthetic oligonucleotide
uuuuaggguugaacugagu

SEQ ID NO: 557 - RNA, 19 nt, Artificial Sequence
  Description of Artificial Sequence Synthetic oligonucleotide
auaguguggaggauguuca

SEQ ID NO: 558 - RNA, 19 nt, Artificial Sequence
  Description of Artificial Sequence Synthetic oligonucleotide
uaguaaacuaagccuuccc

SEQ ID NO: 559 - RNA, 19 nt, Artificial Sequence
  Description of Artificial Sequence Synthetic oligonucleotide
aaggacuaguaaacuaagc

SEQ ID NO: 560 - RNA, 19 nt, Artificial Sequence
  Description of Artificial Sequence Synthetic oligonucleotide
aaggccgucauaacuugga

SEQ ID NO: 561 - RNA, 19 nt, Artificial Sequence
  Description of Artificial Sequence Synthetic oligonucleotide
uaaggccgucauaacuugg

SEQ ID NO: 562 - RNA, 19 nt, Artificial Sequence
  Description of Artificial Sequence Synthetic oligonucleotide
aagcgagauuacagagaca

SEQ ID NO: 563 - RNA, 19 nt, Artificial Sequence
  Description of Artificial Sequence Synthetic oligonucleotide
uaugucgguacgugcuccu

SEQ ID NO: 564 - RNA, 19 nt, Artificial Sequence
  Description of Artificial Sequence Synthetic oligonucleotide
auacguggaacuauaguaa

SEQ ID NO: 565 - RNA, 19 nt, Artificial Sequence
  Description of Artificial Sequence Synthetic oligonucleotide
uuuguggggucggaagauc

SEQ ID NO: 566 - RNA, 19 nt, Artificial Sequence
  Description of Artificial Sequence Synthetic oligonucleotide
auugacggcagcaguucuc

SEQ ID NO: 567 - RNA, 19 nt, Artificial Sequence
  Description of Artificial Sequence Synthetic oligonucleotide
uaaugguccuuuauaauag

SEQ ID NO: 568 - DNA, 3040 bp, Mus musculus
ggtgggaggggagaaggagcgcgagggggagggccgggccgcagggggtcgagggggtggggcctgcgggcggcagggacctggccccggcctggatctggagccgggagggcggaggctcccagcccggaccgatgggcggggcccaggttcccacagagatcagcctcccgggccatgttggtgttaaagcctgtttcttctcctgagccccggcagaacacaagtggaccagtggatgacttctcttgtctgagcaagagatggcctaatccctgcaatctctctaccactggcctctgagctgtaaaagccagctggatgaagggatgtaacacgcgcctgggagggacactgaagtcaggagtggggggggaggggcccggacacaccccttccccccccagactcctccccatcagggtgcacgtggccatggactttggacctggataagtggagagaagctgtactttgggggatctgattctcagttttggaaatgaagtattaggttgggggagggggcggggttgatgatcttcagggaagcgacagagagaagaaaagaaaggtacatggcttcttcctactctcagcctagaagaataaacccttagtgtggggacctgagccaggcaagccaaaggcagccttgagccctccctcccctgccctcccctgcgggggccaggatgctgaagaagcagactgcagggcttgtgctttggggtgctatcatctttgtgggctggaatgccctgctgcttctcttcttctggacacgcccagcacctggcaggctgccctcagacagcgcccttggtgatgaccctgccagcctcacccgtgaggtcatccacctggccgaggacgctgaggcggagttggagcggcagaggggactgttgcagcaaatcaaggagcattatgctttgtggaggcagaggtggagagttcccactgtggcccctccagcctggccccgtgtgcctgtgaccccctcaccagtgcagatccccatcctggtcattgcctgtgaccgcagcactgtccggcgctgcttggataagttgttgcactatcggccctcagctgagcgtttccccattattgtcagtcaggactgtgggcatgaagagacagcacaggtcattgcttcctatggcactgcagtcacacacatccggcagccggacctgagtaacattgccgtgcagccagaccaccgcaagttccagggttactacaagattgccaggcactaccgctgggcactaggccagatcttcaacaagttcaagttcccggccgctgtggtagtggaggatgatctggaagtggcaccagacttctttgagtacttccaggccacctacccactgctgagaacagacccctccctttggtgtgtgtctgcttggaatgacaatggcaaggagcagatggtagactcaagcaaacctgagctgctctatcgaacagacttttttcctggccttggatggctgctgttggctgatctgtgggcagagctggagcccaagtggcccaaggccttttgggacgactggatgcgccgacctgagcagcggaaaggacgggcttgtattcgtccagaaatttcaagaactatgacctttggtcgcaagggtgtgagccacgggcagttctttgaccagcatcttaagttcatcaagctgaaccagcagttcgtccccttcacccagttggacctgtcgtacctgcagcaggaggcctatgaccgggacttcctcgcccaggtctatggtgccccccagctacaggtggagaaagtaaggaccaatgatcagaaggagctgggggaggtgcgggtacagtacactagcagagacagcttcaaggcctttgctaaggccctgggtgtcatggatgacctcaagtctggtgtccccagggctggctaccgtggcattgtcactttccagtttcggggccggcgtgtgcacctggcacccccacagacatggactggctatgatcctagctggaattagcagcacctgcctttccctcctgggtctccttgccgcgtgctgagccgaggcaggcctgcagggcctgggctacaccatcctgccagtgtctccctctcagtgtctccctcttggatctgtagatcttccctttgtgtcctggccagcccccaaatggcattctagtgcacaaatcataggatgagagttatactcctgttgtcaagggagtattgtgtggtatgttcggggcatattgaacaagaaaccactgtgtggtatggggagggttgggcctgttggggccaggtttgtaatgtctccaagggcatctgcagagagcttggcagctccagctctcctcagcaggcctctccaccctgatctggcccctgttagcccatgagccctctttatgtacccatcctccctttctgaaccttccctcagggtggggctggattatggaggaaggaatggatctttggtcagatgagactaacagagggactcactgtcagagttagcgtggattgctgctgccggggcactacctcctggcttctctccctgctgacgtggctcttgtcctggagcagagcagtgcgttgttgggagatggaaagtctatgcgtgggtaccctgtcttcttgcctgtggcccccctcctttctgaaagttagttctctccgcattctttaggaggattcaggggcggcctagtggaagacagtgggctccatgcctctttcccctggggtttgcttcaggggctcctccccgagggcttgctcctggtggcagcagctgagatcaggacatctgtggccctgagccggctctggtcctcggcctttcccagccttcccacatcttcccaggccaagatgcagggggcaggctgaagagaaatttgtgtgtccttgtttgttgcctgatcttagtttcatggaagaaaatggaatctacagaattattttcaaaaataaagtaaaggctgaattgctgaaaaaaaaaaaaaa

SEQ ID NO: 569 - RNA, 19 nt, Artificial Sequence
  Description of Artificial Sequence Synthetic oligonucleotide
aucaaggagcauuaugcuu

SEQ ID NO: 570 - RNA, 19 nt, Artificial Sequence
  Description of Artificial Sequence Synthetic oligonucleotide
ucaaggagcauuaugcuuu

SEQ ID NO: 571 - RNA, 19 nt, Artificial Sequence
  Description of Artificial Sequence Synthetic oligonucleotide
ggcgcugcuuggauaaguu

SEQ ID NO: 572 - RNA, 19 nt, Artificial Sequence
  Description of Artificial Sequence Synthetic oligonucleotide
accugaguaacauugccgu

SEQ ID NO: 573 - RNA, 19 nt, Artificial Sequence
  Description of Artificial Sequence Synthetic oligonucleotide
gagcagaugguagacucaa

SEQ ID NO: 574 - RNA, 19 nt, Artificial Sequence
  Description of Artificial Sequence Synthetic oligonucleotide
ugagcugcucuaucgaaca

SEQ ID NO: 575 - RNA, 19 nt, Artificial Sequence
  Description of Artificial Sequence Synthetic oligonucleotide
gcucuaucgaacagacuuu

SEQ ID NO: 576 - RNA, 19 nt, Artificial Sequence
  Description of Artificial Sequence Synthetic oligonucleotide
aggacgggcuuguauucgu

SEQ ID NO: 577 - RNA, 19 nt, Artificial Sequence
  Description of Artificial Sequence Synthetic oligonucleotide
gggcuuguauucguccaga

SEQ ID NO: 578 - RNA, 19 nt, Artificial Sequence
  Description of Artificial Sequence Synthetic oligonucleotide
ggcuuguauucguccagaa

SEQ ID NO: 579 - RNA, 19 nt, Artificial Sequence
  Description of Artificial Sequence Synthetic oligonucleotide
acuaugaccuuuggucgca

SEQ ID NO: 580 - RNA, 19 nt, Artificial Sequence
  Description of Artificial Sequence Synthetic oligonucleotide
guaaggaccaaugaucaga

SEQ ID NO: 581 - RNA, 19 nt, Artificial Sequence
  Description of Artificial Sequence Synthetic oligonucleotide
gugcggguacaguacacua

SEQ ID NO: 582 - RNA, 19 nt, Artificial Sequence
  Description of Artificial Sequence Synthetic oligonucleotide
uucuagugcacaaaucaua

SEQ ID NO: 583 - RNA, 19 nt, Artificial Sequence
  Description of Artificial Sequence Synthetic oligonucleotide
aaucauaggaugagaguua

SEQ ID NO: 584 - RNA, 19 nt, Artificial Sequence
  Description of Artificial Sequence Synthetic oligonucleotide
ugagaguuauacuccuguu

SEQ ID NO: 585 - RNA, 19 nt, Artificial Sequence
  Description of Artificial Sequence Synthetic oligonucleotide
ccuguugucaagggaguau

SEQ ID NO: 586 - RNA, 19 nt, Artificial Sequence
  Description of Artificial Sequence Synthetic oligonucleotide
cuguugucaagggaguauu

SEQ ID NO: 587 - RNA, 19 nt, Artificial Sequence
  Description of Artificial Sequence Synthetic oligonucleotide
gugguauguucggggcaua

SEQ ID NO: 588 - RNA, 19 nt, Artificial Sequence
  Description of Artificial Sequence Synthetic oligonucleotide
gcccaugagcccucuuuau

SEQ ID NO: 589 - RNA, 19 nt, Artificial Sequence
  Description of Artificial Sequence Synthetic oligonucleotide
cugucagaguuagcgugga

SEQ ID NO: 590 - RNA, 19 nt, Artificial Sequence
  Description of Artificial Sequence Synthetic oligonucleotide
ugucagaguuagcguggau

SEQ ID NO: 591 - RNA, 19 nt, Artificial Sequence
  Description of Artificial Sequence Synthetic oligonucleotide
gucagaguuagcguggauu

SEQ ID NO: 592 - RNA, 19 nt, Artificial Sequence
  Description of Artificial Sequence Synthetic oligonucleotide
agaguuagcguggauugcu

SEQ ID NO: 593 - RNA, 19 nt, Artificial Sequence
  Description of Artificial Sequence Synthetic oligonucleotide
agcagagcagugcguuguu

SEQ ID NO: 594 - RNA, 19 nt, Artificial Sequence
  Description of Artificial Sequence Synthetic oligonucleotide
agauggaaagucuaugcgu

SEQ ID NO: 595 - RNA, 19 nt, Artificial Sequence
  Description of Artificial Sequence Synthetic oligonucleotide
ggaaagucuaugcgugggu

SEQ ID NO: 596 - RNA, 19 nt, Artificial Sequence
  Description of Artificial Sequence Synthetic oligonucleotide
aagcauaaugcuccuugau

SEQ ID NO: 597 - RNA, 19 nt, Artificial Sequence
  Description of Artificial Sequence Synthetic oligonucleotide
aaagcauaaugcuccuuga

SEQ ID NO: 598 - RNA, 19 nt, Artificial Sequence
  Description of Artificial Sequence Synthetic oligonucleotide
aacuuauccaagcagcgcc

SEQ ID NO: 599 - RNA, 19 nt, Artificial Sequence
  Description of Artificial Sequence Synthetic oligonucleotide
acggcaauguuacucaggu

SEQ ID NO: 600 - RNA, 19 nt, Artificial Sequence
  Description of Artificial Sequence Synthetic oligonucleotide
uugagucuaccaucugcuc

SEQ ID NO: 601 - RNA, 19 nt, Artificial Sequence
  Description of Artificial Sequence Synthetic oligonucleotide
uguucgauagagcagcuca

SEQ ID NO: 602 - RNA, 19 nt, Artificial Sequence
  Description of Artificial Sequence Synthetic oligonucleotide
aaagucuguucgauagagc

SEQ ID NO: 603 - RNA, 19 nt, Artificial Sequence
  Description of Artificial Sequence Synthetic oligonucleotide
acgaauacaagcccguccu

SEQ ID NO: 604 - RNA, 19 nt, Artificial Sequence
  Description of Artificial Sequence Synthetic oligonucleotide
ucuggacgaauacaagccc

SEQ ID NO: 605 - RNA, 19 nt, Artificial Sequence
  Description of Artificial Sequence Synthetic oligonucleotide
uucuggacgaauacaagcc

SEQ ID NO: 606 - RNA, 19 nt, Artificial Sequence
  Description of Artificial Sequence Synthetic oligonucleotide
ugcgaccaaaggucauagu

SEQ ID NO: 607 - RNA, 19 nt, Artificial Sequence
  Description of Artificial Sequence Synthetic oligonucleotide
ucugaucauugguccuuac

SEQ ID NO: 608 - RNA, 19 nt, Artificial Sequence
  Description of Artificial Sequence Synthetic oligonucleotide
uaguguacuguacccgcac

SEQ ID NO: 609 - RNA, 19 nt, Artificial Sequence
  Description of Artificial Sequence Synthetic oligonucleotide
uaugauuugugcacuagaa

SEQ ID NO: 610 - RNA, 19 nt, Artificial Sequence
  Description of Artificial Sequence Synthetic oligonucleotide
uaacucucauccuaugauu

SEQ ID NO: 611 - RNA, 19 nt, Artificial Sequence
  Description of Artificial Sequence Synthetic oligonucleotide
aacaggaguauaacucuca

SEQ ID NO: 612 - RNA, 19 nt, Artificial Sequence
  Description of Artificial Sequence Synthetic oligonucleotide
auacucccuugacaacagg

SEQ ID NO: 613 - RNA, 19 nt, Artificial Sequence
  Description of Artificial Sequence Synthetic oligonucleotide
aauacucccuugacaacag

SEQ ID NO: 614 - RNA, 19 nt, Artificial Sequence
  Description of Artificial Sequence Synthetic oligonucleotide
uaugccccgaacauaccac

SEQ ID NO: 615 - RNA, 19 nt, Artificial Sequence
  Description of Artificial Sequence Synthetic oligonucleotide
auaaagagggcucaugggc

SEQ ID NO: 616 - RNA, 19 nt, Artificial Sequence
  Description of Artificial Sequence Synthetic oligonucleotide
uccacgcuaacucugacag

SEQ ID NO: 617 - RNA, 19 nt, Artificial Sequence
  Description of Artificial Sequence Synthetic oligonucleotide
auccacgcuaacucugaca

SEQ ID NO: 618 - RNA, 19 nt, Artificial Sequence
  Description of Artificial Sequence Synthetic oligonucleotide
aauccacgcuaacucugac

SEQ ID NO: 619 - RNA, 19 nt, Artificial Sequence
  Description of Artificial Sequence Synthetic oligonucleotide
agcaauccacgcuaacucu

SEQ ID NO: 620 - RNA, 19 nt, Artificial Sequence
  Description of Artificial Sequence Synthetic oligonucleotide
aacaacgcacugcucugcu

SEQ ID NO: 621 - RNA, 19 nt, Artificial Sequence
  Description of Artificial Sequence Synthetic oligonucleotide
acgcauagacuuuccaucu

SEQ ID NO: 622 - RNA, 19 nt, Artificial Sequence
  Description of Artificial Sequence Synthetic oligonucleotide
acccacgcauagacuuucc

SEQ ID NO: 623 - DNA, 7054 bp, Mus musculus
ttttttttttttttttttttttttggcccgggaccaaggcgttctggattctgttgatttgaggctggagagagaccggtcttgcttgtcatcgccaacttgctaaccagtgtgatggtgaaatgagatgaggctccgaaatggaactgtggccactgcgctggtatttgtcacgtccttccttaccctatcctggtataccacgtggcaaaatgggaaagaaaaactaattgcttatcaacgagaattccttgctctaaaagagcgtcttcgagtggccgagcataggatatctcagcgctcctcggagctaaacaccattgtccagcagttccgcagagctggagcagagactaatggaagtaagacagctctgagtacaatctcagataataccataaagcttctaaaagagttgacaagcaaaaaatcacttcgagtgccaagtatttattatcatttgcctcatctattgcaaaatgaaagaagccttcagcccgccgtacagattggcagtggaagaacgggagtttcaatagttatgggaattcctactgtgaagagagaagttaaatcttacctcgtagaaacccttcactcccttattgataatctgtatcctgaagagaagctggactgtgttatagtcgtcttcataggagagacagatcttgattatgttcacagcgttgttgccaacctggagaaagaattttctagagaaattagttctggcctgctggaaataatctctcctcctgaaagctattaccccgacttgacaaacctgaaggagacgttcggagactccaaggaaagagtgagatggagaaccaagcaaaacctggattactgttttctgatgatgtatgctcaggagaagggcatctactacattcagcttgaagacgatattattgtcaaacaaaactattttaataccataaagaattttgcacttcaactttcttcggaagaatggatgattctagagttttcccagcttggcttcattggaaaaatgttccaggcgccggacctggcgctggtcgtggagttcatcctcatgttctataaggagaagcccattgactggctgctggaccacattctctgggtgaaggtctgcaaccccgaaaaagatgctaaacactgcgacagacagaaggcaaacctacgaatccgcttccgaccctccctcttccagcacgtgggcctacactcgtctctgtcggggaagattcagaaactcacggataaagattacatgaagccattgcttctcaaggtccacgtgaacccgcctgcagaggtctccacctccctgaaggtgtaccaagggcacaccctggagaagacctacatgggggaagacttcttttgggccatcacccccacggctggagactacatcttgtttaaatttgacaaaccggtcaacgtggagagttatttgttccacagcggcaatcaagagcacccaggagacatcctgctgaacacgaccgtggatgttctccctcttaagagcgacagtttggaaatcagcaaagaaaccaaagacaaacgattagaagatggctatttcagaataggaaaatttgagtatggtgttgcagagggaattgtggatcctggtctaaaccctatttcagcctttcgactttccgttattcagaactcagctgtttgggccattcttaatgagattcatattaaaaaagtcaccagttgatctgcttagaaaccaacacgtcctttcttatgactcttgattaaagataattagcgcgtcctcttctgtttggacttgaacactacctctcttgaagccactgtagatgagatgattgttgcctccacgcggacagggactcttcccatggataactgcattcatttgaagctaagctgtcctccaatttgaacttgacacaaacgttttacaatcctgacagcctgttaacatgactgagactattttggtattatactaatacacaagagttgtacatattgttacattccttaaaatttgagaaaactgatgttaaatacattttgtgaagggggtacttttgaacttcacttattttactattatagaccctcttttatagacgatcagggatattacatatatatatatacatatatatatatatatatatataaatatataaatacacatgaagatgttatgaagttaatttattagaagcacttaagaagtacatatttttgtgcagtaaagttttgaatcaacacctttttttgaagaagcggttccccggctttttttaagttctttctatatttttatttggagatgccgacattatagatcaacgccattttttccagtgcactgccattccagattggttctagatgggtttcttaactcgaagtacttttataatcacagcaattctgaacaaaatattttcagaggcatttgtcattcctaaaaatcaagattttaaaagaccaatgttctctcgagggttatattactgtactgtgtatggtgtgtagccacagaaaaccagtgtgactgtcattgtccaggtgggaagcccctgccgtgtctccacagactccttttcccgttcatagttcctttcatgtgggtgatgagactcccttcccttttgctctgtctttaatcagtagttagacaaactgtctcttttctccctggttggtccttttctagatctgtggtaggttttggagttcagatactgttttctgagtaatgctgttgggtgtgtcctgttgggtatgggactcggagaaagacgaccgtagcacttctgtttccatagcatgatttgctccgctgtgtcatgaacgtggaggtaaatgctgtgtccacagagtgtttgtgcatcctcactttgcagtcccaagtagcgctagttggcagaagtgctttactcttatgattaggaaggccgagcaacagttgccagtcttctctggctctccagaaactgcgtgtacagcaggtgtggtggaggcctagggctgtgggaggtcccggctggtagcctccgtcgggggctttcttagttgcccagtgtatgggacacacagaactgagtggaagccattttgtatgttctgcctgggaaaatgtagataaacactactgtgcatgtcaaagcccagctgaagcttttcttggtgttaagtttcccctctcatttccttcagctttttaataattagtactgcatttggcacttttcctatgccctgtgcccacaaataaatcccactatcttcccacactcaagtgtgcagcagcaggataacagactgtgaaaccatgcagatgggcacaggggccccgttaggtctctgctttctcagtggttgtgagttcagaaggccaagccttgaaactccactggaccgtgtgaatgtctcctgcgtgggtctttgagcatatccacttacaaggctctactcatcagtcttgcctctaaggactcttttagccaagtcctttccttcattcaaggaattccccctttccccatgagctagttcctggtgcctggaacacgcctgagccttggcaggctcggttggcatctggattaactagccaggttgctcacatctctctcctagcgatgtctgcctactatcctgccgtgcacgggcacacacacactccgtgtctagtcttcattgtgtgtggtgtgtgttctccaatacactccaggctctggaggccagctctcctgtcttccatcacatcaggcgctctcctgctccctcagtgccagttgcttcctcctcttgtgcccagttcagcactagactattggaaacagatgtttccaatagattttgtgggagccctggaggttgatgaggatgaagacgggggaggtgaagacagagtgcttagaactgtaaagtgttcatccacattatcccttggaggccaccaaccgactttcacccacatagtgaataggaaaagtgtcaaatgcagtactaattatatctgtgaaataggtcatagttggtatcggtgggtatttcacatagcccagactctagagcagtgctggcctctacagaagccacaaaacacatctgttgaaatgaaaagttgagttggcagcctttggagtgctcagtggccacatggccagcagccaccatattggatcctaggaatatgggacatcgacatcacggtgggaggttctgctggatgtgtaaacatgagcattctagaaaggagtagtaatgtatcctgctgctgtggttaaaaatagacctatggattcatcgtagtgcaggtgaaggtgtgtgttttggtgggacagtcaagattacccggctacatgttagtgtcatccttcttcccagcatgcattgcttcgggatcctcaagaggtacgtttgaaaagcatgagaagtgaatgcggatggatggtttcgcttgtaggttagacgtgtgttatgtccatcagagtgtgtttgaacaaaggttcttgaaatcattggttcttgaaatcattggtaaaaagattaggtgtgcttatctcaaaagctatcaggaaagttgggagactagacttctttttaagtctttcataattacgagaactacagttctcaacagtgctgtccctcaaggaaaagttggcttatgtgcaaagtgttgttgttggcaaaggttgttctagttaataagtgtgtatctgtcacatgcaggattctaagaacgacccctcatgaccttgtataatatcctccccttctgagtgtatcgagaactgtgaccacatctgtgggtgattggcccaatcttgagagccatgtcaggctggcagaagtgagagacatcagcagcagaactctaaacagcatcgctggtctaagtggaaggggctcccctaaatgactgcaggtcacctgccttaaggagccagaggctctgttacccaccaagtgggacctgcagaccttttgccataggacaatttgacccagacacttacccaggcttggacatggaaatccttgagcagccagcctggtaaacaccttcactttagctttgtaagtgggaacctacctagacttctggtgtgcagcagtggataataaacgggcagtgctgtatgtggctagatttgtggcatttttaaaaagcaacagaatctgtcagtcctgagaagtctccatatatgattgcctgcggtagactagtttttaagacaaggtctcaccttgcaccccaggccgattctcccggttctgcctctcaagtgctgatgtgtatcaccatgcccggctgactgatggtttttgacataagaataaactcattcccagcaagccctattcctgggtctctgctaggatgtttggtgccaggacccgtgtctcctctacaagtcagacttgtattagtttttgttctgtaaaactaccagggtgaggctgtaggcatctttttctcctagatgctcctaaattatactgacttaaaggcctgccagaagtacaggaaagaatgatgaacggtattttagaccagactgatggagaagaaaaattcccacctttgtgttgacttgctggtgtgtctaacctgacagtgcagcgtgaggctgtcatctacagagtccatgctcaagaaccacatggtgaagttacaaaaacaaggacactgttaaaagctctgagtaagtttacaatcttgtattgtgccattcattgctgtcccgggccacaggctgaacacacctgcaagagcttcggctgtgacaggtgtaattttttacaaaaatcattcagctgagtagctcacggctcaaaccctgtatgttcccaccaaggttttgtgtttacgtggttcctaaaatacctgcacttccaagctattaactgctaatgaggtttctgatcttggttcactcagccatgcttcttattaaccatgctgccacagccttgaaggaaaggaacccagaggttaaacactgtcctcatcctcacgtggtaagggatcaggcctcagttcaagacttctagaaaagggcattccgagctatgtagcaactcttacagcttgtaaaacttcaaactttgatagttgaggacttaaatgatattaacatatatgctacgctaattaatatgctaaatttaatgcatgtgaacatacaatgctacaaagatgaccacagccttttacaaagagaaaaagaataaaacaaacaaagacacctcaaggatttgctgcccgtgcccctgtactctatcactagctaaatggacctgtttctgaaatgctccctttccagtttctgcattacctaaaactgcaagtgcgagagcagacaggcagcaggtgacctgaatgtgtgtagtgtgaaccctggtccttagcagggctcctgctgctctgaacacaggcttgtggggacagctaaatacctgtgatactaaggaggccgcttcctgccgctgagttagcaactggtcccagaatagcaagagtggggtcctttagcttgcatagcattctgttctttattggaactgctccagctggtaagcgtacctgaagggcatgctcgtgagccttacaaagagctgctgggacagtactggctgagtccactaagccactgttttccgcagtggagtcaagggcagctcagttagagcacttcctccacagtctgtctttgagttggggagacggtcttggagcagcccaaggaacccctttgctaggttgcaccaggttcttttctctgcacatatttggctcacaatgtcttcatgggttgaacaataactgcagttatatttgaggtacttgttgaatttttaaaggcagattatttattttcaagtttgctgctataattgaaacctaataatgtcctgttgactgtgcctttcataattgtctctgtgctacaactgtccatgtgtatttcaaataaagaagtttgaaaagctatgttgaatcaagattccttctaataaaaatggcttaaaccaa

SEQ ID NO: 624 - RNA, 19 nt, Artificial Sequence
  Description of Artificial Sequence Synthetic oligonucleotide
cgggaguuucaauaguuau

SEQ ID NO: 625 - RNA, 19 nt, Artificial Sequence
  Description of Artificial Sequence Synthetic oligonucleotide
gcuugaagacgauauuauu

SEQ ID NO: 626 - RNA, 19 nt, Artificial Sequence
  Description of Artificial Sequence Synthetic oligonucleotide
cagaaggcaaaccuacgaa

SEQ ID NO: 627 - RNA, 19 nt, Artificial Sequence
  Description of Artificial Sequence Synthetic oligonucleotide
cuuucgacuuuccguuauu

SEQ ID NO: 628 - RNA, 19 nt, Artificial Sequence
  Description of Artificial Sequence Synthetic oligonucleotide
cauuauagaucaacgccau

SEQ ID NO: 629 - RNA, 19 nt, Artificial Sequence
  Description of Artificial Sequence Synthetic oligonucleotide
cgaggguuauauuacugua

SEQ ID NO: 630 - RNA, 19 nt, Artificial Sequence
  Description of Artificial Sequence Synthetic oligonucleotide
gucccaaguagcgcuaguu

SEQ ID NO: 631 - RNA, 19 nt, Artificial Sequence
  Description of Artificial Sequence Synthetic oligonucleotide
cacaaauaaaucccacuau

SEQ ID NO: 632 - RNA, 19 nt, Artificial Sequence
  Description of Artificial Sequence Synthetic oligonucleotide
gugucaaaugcaguacuaa

SEQ ID NO: 633 - RNA, 19 nt, Artificial Sequence
  Description of Artificial Sequence Synthetic oligonucleotide
gucaaaugcaguacuaauu

SEQ ID NO: 634 - RNA, 19 nt, Artificial Sequence
  Description of Artificial Sequence Synthetic oligonucleotide
ggugggacagucaagauua

SEQ ID NO: 635 - RNA, 19 nt, Artificial Sequence
  Description of Artificial Sequence Synthetic oligonucleotide
caagauuacccggcuacau

SEQ ID NO: 636 - RNA, 19 nt, Artificial Sequence
  Description of Artificial Sequence Synthetic oligonucleotide
cucaagagguacguuugaa

SEQ ID NO: 637 - RNA, 19 nt, Artificial Sequence
  Description of Artificial Sequence Synthetic oligonucleotide
cauugguucuugaaaucau

SEQ ID NO: 638 - RNA, 19 nt, Artificial Sequence
  Description of Artificial Sequence Synthetic oligonucleotide
ggcaaagguuguucuaguu

SEQ ID NO: 639 - RNA, 19 nt, Artificial Sequence
  Description of Artificial Sequence Synthetic oligonucleotide
ccugcgguagacuaguuuu

SEQ ID NO: 640 - RNA, 19 nt, Artificial Sequence
  Description of Artificial Sequence Synthetic oligonucleotide
cuagaugcuccuaaauuau

SEQ ID NO: 641 - RNA, 19 nt, Artificial Sequence
  Description of Artificial Sequence Synthetic oligonucleotide
caagcuauuaacugcuaau

SEQ ID NO: 642 - RNA, 19 nt, Artificial Sequence
  Description of Artificial Sequence Synthetic oligonucleotide
gggcauuccgagcuaugua

SEQ ID NO: 643 - RNA, 19 nt, Artificial Sequence
  Description of Artificial Sequence Synthetic oligonucleotide
cauauaugcuacgcuaauu

SEQ ID NO: 644 - RNA, 19 nt, Artificial Sequence
  Description of Artificial Sequence Synthetic oligonucleotide
gcugguaagcguaccugaa

SEQ ID NO: 645 - RNA, 19 nt, Artificial Sequence
  Description of Artificial Sequence Synthetic oligonucleotide
auaacuauugaaacucccg

SEQ ID NO: 646 - RNA, 19 nt, Artificial Sequence
  Description of Artificial Sequence Synthetic oligonucleotide
aauaauaucgucuucaagc

SEQ ID NO: 647 - RNA, 19 nt, Artificial Sequence
  Description of Artificial Sequence Synthetic oligonucleotide
uucguagguuugccuucug

SEQ ID NO: 648 - RNA, 19 nt, Artificial Sequence
  Description of Artificial Sequence Synthetic oligonucleotide
aauaacggaaagucgaaag

SEQ ID NO: 649 - RNA, 19 nt, Artificial Sequence
  Description of Artificial Sequence Synthetic oligonucleotide
auggcguugaucuauaaug

SEQ ID NO: 650 - RNA, 19 nt, Artificial Sequence
  Description of Artificial Sequence Synthetic oligonucleotide
uacaguaauauaacccucg

SEQ ID NO: 651 - RNA, 19 nt, Artificial Sequence
  Description of Artificial Sequence Synthetic oligonucleotide
aacuagcgcuacuugggac

SEQ ID NO: 652 - RNA, 19 nt, Artificial Sequence
  Description of Artificial Sequence Synthetic oligonucleotide
auagugggauuuauuugug

SEQ ID NO: 653 - RNA, 19 nt, Artificial Sequence
  Description of Artificial Sequence Synthetic oligonucleotide
uuaguacugcauuugacac

SEQ ID NO: 654 - RNA, 19 nt, Artificial Sequence
  Description of Artificial Sequence Synthetic oligonucleotide
aauuaguacugcauuugac

SEQ ID NO: 655 - RNA, 19 nt, Artificial Sequence
  Description of Artificial Sequence Synthetic oligonucleotide
uaaucuugacugucccacc

SEQ ID NO: 656 - RNA, 19 nt, Artificial Sequence
  Description of Artificial Sequence Synthetic oligonucleotide
auguagccggguaaucuug

SEQ ID NO: 657 - RNA, 19 nt, Artificial Sequence
  Description of Artificial Sequence Synthetic oligonucleotide
uucaaacguaccucuugag

SEQ ID NO: 658 - RNA, 19 nt, Artificial Sequence
  Description of Artificial Sequence Synthetic oligonucleotide
augauuucaagaaccaaug

SEQ ID NO: 659 - RNA, 19 nt, Artificial Sequence
  Description of Artificial Sequence Synthetic oligonucleotide
aacuagaacaaccuuugcc

SEQ ID NO: 660 - RNA, 19 nt, Artificial Sequence
  Description of Artificial Sequence Synthetic oligonucleotide
aaaacuagucuaccgcagg

SEQ ID NO: 661 - RNA, 19 nt, Artificial Sequence
  Description of Artificial Sequence Synthetic oligonucleotide
auaauuuaggagcaucuag

SEQ ID NO: 662 - RNA, 19 nt, Artificial Sequence
  Description of Artificial Sequence Synthetic oligonucleotide
auuagcaguuaauagcuug

SEQ ID NO: 663 - RNA, 19 nt, Artificial Sequence
  Description of Artificial Sequence Synthetic oligonucleotide
uacauagcucggaaugccc

SEQ ID NO: 664 - RNA, 19 nt, Artificial Sequence
  Description of Artificial Sequence Synthetic oligonucleotide
aauuagcguagcauauaug

SEQ ID NO: 665 - RNA, 19 nt, Artificial Sequence
  Description of Artificial Sequence Synthetic oligonucleotide
uucagguacgcuuaccagc

SEQ ID NO: 666 - DNA, 2429 bp, Mus musculus
ccgtcgactcggcggcggtcccggatccagcccggccgcggctcttgccgccttgccccaaccctgctcggccccggcccagcctcagatcgaggccgccagcaccgcgcaccgcggaggagcagctcgcacgcgccctccacccccgtccccattcccgccctgcctgtagcctcctctcccgggcccgcagctcctccgcgctccggggccggggcccccgccgcctccgggcccccgtgccccggccccggtcccccgggccatgcggcctcggccccgccggcgcccgccgcgcatcgggggagatgaggctccgcaatggcaccttcctgacgctgctgctcttctgcttgtgcgccttcctctcgctctcctggtacgcagcgctcagcggccagaaaggtgacgtggtggacatttaccagcgcgagttcctggctctgcgagaccgtttgcacgcggctgagcaagagagcctgaagcgctccaaggagctaaacctggtgctggaagaaatcaagagggcagtatccgagaggcaagcgctgcgggacggagaaggcaatcgcacttggggccgcctaacagaggatccgcgactgaagccgtggaacgtctcgcacaggcacgtacttcatctgcccaccgtcttccaccatctgccgcacctgctggctaaggagagcagtctgcagcccgcagtgcgagtgggccagggccgcaccggagtatccgtggtgatgggcattccgagcgtacggcgcgaggtgcactcgtacttgactgacacattgcactcgctcatctcggagctgagcccgcaggagaaggaagactcagtcatcgtggtgctgatcgccgagactgacccacagtacacttcggcagtgacagagaacatcaaggccttgttccccacagagatccattctgggctcctggaagtcatctccccttcccctcacttctaccctgacttctcccgccttcgagagtcctttggggaccccaaggagagagtcaggtggaggaccaaacagaacctcgattactgcttcctcatgatgtatgcacagtccaaaggcatctactatgtgcagctggaggatgacattgtagccaagcccaactacttgagcactatgaagaactttgccctccagcagccctccgaggactggatgatcctggagttctcgcagttgggcttcattgggaagatgttcaagtcactggatctgagcctgattgtggagttcatcctcatgttctaccgggacaagcccatagactggctcctggaccacatcctgtgggtgaaagtctgcaaccctgagaaggatgcgaaacattgtgatcggcagaaggccaaccttcggatccgcttcaagccgtcccttttccagcatgtgggcactcactcatcactggcgggcaaaatccagaaactgaaggataaagactttggaaagcatgctctccggaaggagcacgtgaacccaccggcagaggtgagcacaagcctcaagacgtaccagcatttcaccctggagaaggcctacttgcgggaggatttcttctgggccttcacacctgccgcaggagactttatccggttccgcttcttccagccactgcgccttgagcggttcttcttccgaagcgggaacatcgagcacccggaagataagctcttcaacacttctgtggaggtgctgccctttgataacccccagtcagagaaggaggcccttcaggaaggccgctcagccactctccggtaccctaggagcccagatggatacctccagattggctccttctacaagggtgtagctgaaggagaagtggatcctgcctttggccccctggaagcactacgtctctccattcagactgactccccggtgtgggtcattttgagtgagatctttctgaaaaaggccgactagaaggcttccaagggtgctctgtggccggctctggagcccacggttccgagggtgtcgctgctactgctgctgctgccccagaggaccaggcatatccaccccacctggagggttctgcctggcaggcggctcgggctggcctggggttcaccgctggcccggaggccccagaagctggtgctgctcctgcccgccgggccgcgggagaggcaggcggccccccacactgtgcctgagggccggcctgctgccgcccaaaaagaactgaaccgaaccgtttgcccctggccggtggtcttgagccgggccgttagaagagctttttcttgggtgcccgccgtgtgcggcgcaaacactggaatgcatacactactttatgtgctgtgttttttattcttggatacatttgattttttcacgtaagttcgcatatacttctataagagcgtgacttgtaataaagggttaatgaag

SEQ ID NO: 667 - RNA, 19 nt, Artificial Sequence
  Description of Artificial Sequence Synthetic oligonucleotide
uuuaccagcgcgaguuccu

SEQ ID NO: 668 - RNA, 19 nt, Artificial Sequence
  Description of Artificial Sequence Synthetic oligonucleotide
ggagaaggcaaucgcacuu

SEQ ID NO: 669 - RNA, 19 nt, Artificial Sequence
  Description of Artificial Sequence Synthetic oligonucleotide
uauccguggugaugggcau

SEQ ID NO: 670 - RNA, 19 nt, Artificial Sequence
  Description of Artificial Sequence Synthetic oligonucleotide
auccguggugaugggcauu

SEQ ID NO: 671 - RNA, 19 nt, Artificial Sequence
  Description of Artificial Sequence Synthetic oligonucleotide
gaggugcacucguacuuga

SEQ ID NO: 672 - RNA, 19 nt, Artificial Sequence
  Description of Artificial Sequence Synthetic oligonucleotide
ucguacuugacugacacau

SEQ ID NO: 673 - RNA, 19 nt, Artificial Sequence
  Description of Artificial Sequence Synthetic oligonucleotide
guacacuucggcagugaca

SEQ ID NO: 674 - RNA, 19 nt, Artificial Sequence
  Description of Artificial Sequence Synthetic oligonucleotide
accaaacagaaccucgauu

SEQ ID NO: 675 - RNA, 19 nt, Artificial Sequence
  Description of Artificial Sequence Synthetic oligonucleotide
agaaccucgauuacugcuu

SEQ ID NO: 676 - RNA, 19 nt, Artificial Sequence
  Description of Artificial Sequence Synthetic oligonucleotide
aguccaaaggcaucuacua

SEQ ID NO: 677 - RNA, 19 nt, Artificial Sequence
  Description of Artificial Sequence Synthetic oligonucleotide
auccuggaguucucgcagu

SEQ ID NO: 678 - RNA, 19 nt, Artificial Sequence
  Description of Artificial Sequence Synthetic oligonucleotide
aguucucgcaguugggcuu

SEQ ID NO: 679 - RNA, 19 nt, Artificial Sequence
  Description of Artificial Sequence Synthetic oligonucleotide
gagaaggaugcgaaacauu

SEQ ID NO: 680 - RNA, 19 nt, Artificial Sequence
  Description of Artificial Sequence Synthetic oligonucleotide
gaaggaugcgaaacauugu

SEQ ID NO: 681 - RNA, 19 nt, Artificial Sequence
  Description of Artificial Sequence Synthetic oligonucleotide
aaacauugugaucggcaga

SEQ ID NO: 682 - RNA, 19 nt, Artificial Sequence
  Description of Artificial Sequence Synthetic oligonucleotide
aacauugugaucggcagaa

SEQ ID NO: 683 - RNA, 19 nt, Artificial Sequence
  Description of Artificial Sequence Synthetic oligonucleotide
caucacuggcgggcaaaau

SEQ ID NO: 684 - RNA, 19 nt, Artificial Sequence
  Description of Artificial Sequence Synthetic oligonucleotide
gcacaagccucaagacgua

SEQ ID NO: 685 - RNA, 19 nt, Artificial Sequence
  Description of Artificial Sequence Synthetic oligonucleotide
caagccucaagacguacca

SEQ ID NO: 686 - RNA, 19 nt, Artificial Sequence
  Description of Artificial Sequence Synthetic oligonucleotide
uacuugcgggaggauuucu

SEQ ID NO: 687 - RNA, 19 nt, Artificial Sequence
  Description of Artificial Sequence Synthetic oligonucleotide
caggagacuuuauccgguu

SEQ ID NO: 688 - RNA, 19 nt, Artificial Sequence
  Description of Artificial Sequence Synthetic oligonucleotide
aucgagcacccggaagaua

SEQ ID NO: 689 - RNA, 19 nt, Artificial Sequence
  Description of Artificial Sequence Synthetic oligonucleotide
ucgagcacccggaagauaa

SEQ ID NO: 690 - RNA, 19 nt, Artificial Sequence
  Description of Artificial Sequence Synthetic oligonucleotide
acccggaagauaagcucuu

SEQ ID NO: 691 - RNA, 19 nt, Artificial Sequence
  Description of Artificial Sequence Synthetic oligonucleotide
cuucuacaaggguguagcu

SEQ ID NO: 692 - RNA, 19 nt, Artificial Sequence
  Description of Artificial Sequence Synthetic oligonucleotide
cuggaagcacuacgucucu

SEQ ID NO: 693 - RNA, 19 nt, Artificial Sequence
  Description of Artificial Sequence Synthetic oligonucleotide
agcacuacgucucuccauu

SEQ ID NO: 694 - RNA, 19 nt, Artificial Sequence
  Description of Artificial Sequence Synthetic oligonucleotide
ccggugugggucauuuuga

SEQ ID NO: 695 - RNA, 19 nt, Artificial Sequence
  Description of Artificial Sequence Synthetic oligonucleotide
gggucauuuugagugagau

SEQ ID NO: 696 - RNA, 19 nt, Artificial Sequence
  Description of Artificial Sequence Synthetic oligonucleotide
aggaacucgcgcugguaaa

SEQ ID NO: 697 - RNA, 19 nt, Artificial Sequence
  Description of Artificial Sequence Synthetic oligonucleotide
aagugcgauugccuucucc

SEQ ID NO: 698 - RNA, 19 nt, Artificial Sequence
  Description of Artificial Sequence Synthetic oligonucleotide
augcccaucaccacggaua

SEQ ID NO: 699 - RNA, 19 nt, Artificial Sequence
  Description of Artificial Sequence Synthetic oligonucleotide
aaugcccaucaccacggau

SEQ ID NO: 700 - RNA, 19 nt, Artificial Sequence
  Description of Artificial Sequence Synthetic oligonucleotide
ucaaguacgagugcaccuc

SEQ ID NO: 701 - RNA, 19 nt, Artificial Sequence
  Description of Artificial Sequence Synthetic oligonucleotide
augugucagucaaguacga

SEQ ID NO: 702 - RNA, 19 nt, Artificial Sequence
  Description of Artificial Sequence Synthetic oligonucleotide
ugucacugccgaaguguac

SEQ ID NO: 703 - RNA, 19 nt, Artificial Sequence
  Description of Artificial Sequence Synthetic oligonucleotide
aaucgagguucuguuuggu

SEQ ID NO: 704 - RNA, 19 nt, Artificial Sequence
  Description of Artificial Sequence Synthetic oligonucleotide
aagcaguaaucgagguucu

SEQ ID NO: 705 - RNA, 19 nt, Artificial Sequence
  Description of Artificial Sequence Synthetic oligonucleotide
uaguagaugccuuuggacu

SEQ ID NO: 706 - RNA, 19 nt, Artificial Sequence
  Description of Artificial Sequence Synthetic oligonucleotide
acugcgagaacuccaggau

SEQ ID NO: 707 - RNA, 19 nt, Artificial Sequence
  Description of Artificial Sequence Synthetic oligonucleotide
aagcccaacugcgagaacu

SEQ ID NO: 708 - RNA, 19 nt, Artificial Sequence
  Description of Artificial Sequence Synthetic oligonucleotide
aauguuucgcauccuucuc

SEQ ID NO: 709 - RNA, 19 nt, Artificial Sequence
  Description of Artificial Sequence Synthetic oligonucleotide
acaauguuucgcauccuuc

SEQ ID NO: 710 - RNA, 19 nt, Artificial Sequence
  Description of Artificial Sequence Synthetic oligonucleotide
ucugccgaucacaauguuu

SEQ ID NO: 711 - RNA, 19 nt, Artificial Sequence
  Description of Artificial Sequence Synthetic oligonucleotide
uucugccgaucacaauguu

SEQ ID NO: 712 - RNA, 19 nt, Artificial Sequence
  Description of Artificial Sequence Synthetic oligonucleotide
auuuugcccgccagugaug

SEQ ID NO: 713 - RNA, 19 nt, Artificial Sequence
  Description of Artificial Sequence Synthetic oligonucleotide
uacgucuugaggcuugugc

SEQ ID NO: 714 - RNA, 19 nt, Artificial Sequence
  Description of Artificial Sequence Synthetic oligonucleotide
ugguacgucuugaggcuug

SEQ ID NO: 715 - RNA, 19 nt, Artificial Sequence
  Description of Artificial Sequence Synthetic oligonucleotide
agaaauccucccgcaagua

SEQ ID NO: 716 - RNA, 19 nt, Artificial Sequence
  Description of Artificial Sequence Synthetic oligonucleotide
aaccggauaaagucuccug

SEQ ID NO: 717 - RNA, 19 nt, Artificial Sequence
  Description of Artificial Sequence Synthetic oligonucleotide
uaucuuccgggugcucgau

SEQ ID NO: 718 - RNA, 19 nt, Artificial Sequence
  Description of Artificial Sequence Synthetic oligonucleotide
uuaucuuccgggugcucga

SEQ ID NO: 719 - RNA, 19 nt, Artificial Sequence
  Description of Artificial Sequence Synthetic oligonucleotide
aagagcuuaucuuccgggu

SEQ ID NO: 720 - RNA, 19 nt, Artificial Sequence
  Description of Artificial Sequence Synthetic oligonucleotide
agcuacacccuuguagaag

SEQ ID NO: 721 - RNA, 19 nt, Artificial Sequence
  Description of Artificial Sequence Synthetic oligonucleotide
agagacguagugcuuccag

SEQ ID NO: 722 - RNA, 19 nt, Artificial Sequence
  Description of Artificial Sequence Synthetic oligonucleotide
aauggagagacguagugcu

SEQ ID NO: 723 - RNA, 19 nt, Artificial Sequence
  Description of Artificial Sequence Synthetic oligonucleotide
ucaaaaugacccacaccgg

SEQ ID NO: 724 - RNA, 19 nt, Artificial Sequence
  Description of Artificial Sequence Synthetic oligonucleotide
aucucacucaaaaugaccc

SEQ ID NO: 725 - DNA, 2843 bp, Rattus norvegicus
tagcggaagccgcggggtgcgcttgccggggcctgccccgtggacccgctggacccgagcctgcagagtgccgaggtggcgccggagggaactaacccggcggaggccacgagtcagtcggtgactcacgagcatcgtgccaggctctgacaccatcgctgcagttgtgggcctcgcgctgtgtgcccgggttctgcaccgcagggcgcctctgctgtcccggtgcgttgctccggcgtgggatcccggagctgtcaggctcagcgcgagggtgaatggggaccggggcgtgcaaaggaactctattttaaaaaactctcaagtaaaaaaaagcaagtcatggagaagaacgggaataaccggaagcttcgggtttgcgttgccacctgcaaccgagccgattactccaaactggcccccatcatgttcggcattaagacggagcctgcgttcttcgagctcgacgtggtggtgctgggctctcacctgatcgacgactacggaaacacataccgcatgattgagcaggacgactttgacatcaacaccaggctacacacgattgttagaggggaagacgaagcagccatggtagagtcagtgggcctagcgctagtgaagctaccggatgtcctcaaccgcctgaagcctgacatcatgattgttcacggagaccgatttgacgccctcgctctggctacatctgctgccctgatgaacatccgcatccttcacattgaaggaggagaggtcagcgggactattgatgactctatcagacacgccataacaaaactggctcactaccacgtgtgctgcaccaggagtgcagagcaacacctgatctccatgtgtgaggaccacgaccgcatccttttggctggctgcccttcctatgacaaactgctctcagccaagaataaagactatatgagcatcattcggatgtggctaggtgatgatgtaaaatgtaaagattacattgttgccctgcaacacccggtgaccaccgacattaagcattccataaagatgttcgaactgacactggatgctcttatctcatttaacaagaggaccctagttctgtttccaaatatcgatgcaggcagcaaggagatggttcgagtgatgcggaagaagggcatcgagcatcaccccaatttccgcgcagtcaagcacgtcccgtttgaccagttcattcagctggtcgcccacgctggctgcatgattgggaatagcagctgtggagtgcgtgaggttggcgcctttggaacccctgtgatcaacctgggcacgcggcagataggaagagaaacgggggagaatgttcttcatgtccgggatgctgacacccaagacaaaatattacaagcactacacctccagttcggtaaacagtacccttgctcaaagatatatggggatggaaatgctgttccaaggattttaaagtttctcaaatccatcgaccttcaagagccactacagaagaaattctgcttccctcccgtgaaggagaacatctctcaggatattgaccatatcctcgaaactctgagtgccttggctgttgatctcggggggacgaatctgagagtggcgatagttagcatgaagggtgaaatagttaagaagtacacccagttcaatcctaaaacctatgaggaaaggattagtctaatcctgcagatgtgtgtggaagcggcagcagaagccgtgaagctcaattgcagaattctgggagtaggcatctccacaggtggccgtgtgaatccccaggaaggagttgtgctgcactcgaccaagctgatacaggagtggaactctgtggacctcaggacaccactctccgacaccctgcatctccccgtgtgggtggacaacgacggcaactgcgctgccatggcggagaggaagtttggccaaggaaaaggacaggagaactttgtgacgctcatcacagggacagggatcggtgggggaatcatccaccagcacgagctgatccacggcagctccttctgtgcggcagagcttggccacctcgtggtgtctctggatggtcctgactgctcctgtggaagccatgggtgcattgaagcctacgcctctggaatggccttgcagagggaagcaaagaagctccacgacgaggacctgctcttggtggaagggatgtcagtgccaaaagacgaagctgtgggcgccctccatctcatccaagccgccaagctgggcaacgtgaaggcccagagcatcttacggacagctggaactgctttgggactcggagttgtgaatatcctccacactatgaatccttccctggtgatcctgtctggagtcctggctagtcactacatccacattgtgagggacgtcatccgccagcaagccctgtcctccgtgcaggatgtggatgtagtggtttcagacttggttgacccggccctgcttggtgcggccagcatggttctggactacacgacccgcaggatccactaggcctcctgggaatagacctggactgagacccaagagctactgagtggaaccacgctctcttagatcagtatttcttcaaaggccagtgtgggaggctgcggagccagctcagtggtcaagagcctaaactgctcttgccaaagacccaagttcagttcccagcacccatgtcaggcactcaactgcctgaaagccaagctccaggggatccagtgcctcagctgcctcaggcatctgcactcaaatgtacatagtcctctccagaatacacgtaaattaaaataaatcttattttttaaaaggcaaaaaaaaaaaaaaaaaaaaaaaaaaaaaaa

SEQ ID NO: 726 - RNA, 19 nt, Artificial Sequence
  Description of Artificial Sequence Synthetic oligonucleotide
agaacgggaauaaccggaa

SEQ ID NO: 727 - RNA, 19 nt, Artificial Sequence
  Description of Artificial Sequence Synthetic oligonucleotide
gaagcuucggguuugcguu

SEQ ID NO: 728 - RNA, 19 nt, Artificial Sequence
  Description of Artificial Sequence Synthetic oligonucleotide
uuugcguugccaccugcaa

SEQ ID NO: 729 - RNA, 19 nt, Artificial Sequence
  Description of Artificial Sequence Synthetic oligonucleotide
cucaccugaucgacgacua

SEQ ID NO: 730 - RNA, 19 nt, Artificial Sequence
  Description of Artificial Sequence Synthetic oligonucleotide
acaccaggcuacacacgau

SEQ ID NO: 731 - RNA, 19 nt, Artificial Sequence
  Description of Artificial Sequence Synthetic oligonucleotide
caggcuacacacgauuguu

SEQ ID NO: 732 - RNA, 19 nt, Artificial Sequence
  Description of Artificial Sequence Synthetic oligonucleotide
cacgauuguuagaggggaa

SEQ ID NO: 733 - RNA, 19 nt, Artificial Sequence
  Description of Artificial Sequence Synthetic oligonucleotide
ucggauguggcuaggugau

SEQ ID NO: 734 - RNA, 19 nt, Artificial Sequence
  Description of Artificial Sequence Synthetic oligonucleotide
accaccgacauuaagcauu

SEQ ID NO: 735 - RNA, 19 nt, Artificial Sequence
  Description of Artificial Sequence Synthetic oligonucleotide
cgacauuaagcauuccaua

SEQ ID NO: 736 - RNA, 19 nt, Artificial Sequence
  Description of Artificial Sequence Synthetic oligonucleotide
ccaauuuccgcgcagucaa

SEQ ID NO: 737 - RNA, 19 nt, Artificial Sequence
  Description of Artificial Sequence Synthetic oligonucleotide
agucaagcacgucccguuu

SEQ ID NO: 738 - RNA, 19 nt, Artificial Sequence
  Description of Artificial Sequence Synthetic oligonucleotide
uacaccuccaguucgguaa

SEQ ID NO: 739 - RNA, 19 nt, Artificial Sequence
  Description of Artificial Sequence Synthetic oligonucleotide
uucgguaaacaguacccuu

SEQ ID NO: 740 - RNA, 19 nt, Artificial Sequence
  Description of Artificial Sequence Synthetic oligonucleotide
uccaaggauuuuaaaguuu

SEQ ID NO: 741 - RNA, 19 nt, Artificial Sequence
  Description of Artificial Sequence Synthetic oligonucleotide
cgaaucugagaguggcgau

SEQ ID NO: 742 - RNA, 19 nt, Artificial Sequence
  Description of Artificial Sequence Synthetic oligonucleotide
gaaucugagaguggcgaua

SEQ ID NO: 743 - RNA, 19 nt, Artificial Sequence
  Description of Artificial Sequence Synthetic oligonucleotide
cugagaguggcgauaguua

SEQ ID NO: 744 - RNA, 19 nt, Artificial Sequence
  Description of Artificial Sequence Synthetic oligonucleotide
uggcgauaguuagcaugaa

SEQ ID NO: 745 - RNA, 19 nt, Artificial Sequence
  Description of Artificial Sequence Synthetic oligonucleotide
uuagcaugaagggugaaau

SEQ ID NO: 746 - RNA, 19 nt, Artificial Sequence
  Description of Artificial Sequence Synthetic oligonucleotide
aggaaaggauuagucuaau

SEQ ID NO: 747 - RNA, 19 nt, Artificial Sequence
  Description of Artificial Sequence Synthetic oligonucleotide
agaacuuugugacgcucau

SEQ ID NO: 748 - RNA, 19 nt, Artificial Sequence
  Description of Artificial Sequence Synthetic oligonucleotide
aguggaaccacgcucucuu

SEQ ID NO: 749 - RNA, 19 nt, Artificial Sequence
  Description of Artificial Sequence Synthetic oligonucleotide
cucuccagaauacacguaa

SEQ ID NO: 750 - RNA, 19 nt, Artificial Sequence
  Description of Artificial Sequence Synthetic oligonucleotide
ucuccagaauacacguaaa

SEQ ID NO: 751 - RNA, 19 nt, Artificial Sequence
  Description of Artificial Sequence Synthetic oligonucleotide
uccagaauacacguaaauu

SEQ ID NO: 752 - RNA, 19 nt, Artificial Sequence
  Description of Artificial Sequence Synthetic oligonucleotide
uuccgguuauucccguucu

SEQ ID NO: 753 - RNA, 19 nt, Artificial Sequence
  Description of Artificial Sequence Synthetic oligonucleotide
aacgcaaacccgaagcuuc

SEQ ID NO: 754 - RNA, 19 nt, Artificial Sequence
  Description of Artificial Sequence Synthetic oligonucleotide
uugcagguggcaacgcaaa

SEQ ID NO: 755 - RNA, 19 nt, Artificial Sequence
  Description of Artificial Sequence Synthetic oligonucleotide
uagucgucgaucaggugag

SEQ ID NO: 756 - RNA, 19 nt, Artificial Sequence
  Description of Artificial Sequence Synthetic oligonucleotide
aucguguguagccuggugu

SEQ ID NO: 757 - RNA, 19 nt, Artificial Sequence
  Description of Artificial Sequence Synthetic oligonucleotide
aacaaucguguguagccug

SEQ ID NO: 758 - RNA, 19 nt, Artificial Sequence
  Description of Artificial Sequence Synthetic oligonucleotide
uuccccucuaacaaucgug

SEQ ID NO: 759 - RNA, 19 nt, Artificial Sequence
  Description of Artificial Sequence Synthetic oligonucleotide
aucaccuagccacauccga

SEQ ID NO: 760 - RNA, 19 nt, Artificial Sequence
  Description of Artificial Sequence Synthetic oligonucleotide
aaugcuuaaugucgguggu

SEQ ID NO: 761 - RNA, 19 nt, Artificial Sequence
  Description of Artificial Sequence Synthetic oligonucleotide
uauggaaugcuuaaugucg

SEQ ID NO: 762 - RNA, 19 nt, Artificial Sequence
  Description of Artificial Sequence Synthetic oligonucleotide
uugacugcgcggaaauugg

SEQ ID NO: 763 - RNA, 19 nt, Artificial Sequence
  Description of Artificial Sequence Synthetic oligonucleotide
aaacgggacgugcuugacu

SEQ ID NO: 764 - RNA, 19 nt, Artificial Sequence
  Description of Artificial Sequence Synthetic oligonucleotide
uuaccgaacuggaggugua

SEQ ID NO: 765 - RNA, 19 nt, Artificial Sequence
  Description of Artificial Sequence Synthetic oligonucleotide
aaggguacuguuuaccgaa

SEQ ID NO: 766 - RNA, 19 nt, Artificial Sequence
  Description of Artificial Sequence Synthetic oligonucleotide
aaacuuuaaaauccuugga

SEQ ID NO: 767 - RNA, 19 nt, Artificial Sequence
  Description of Artificial Sequence Synthetic oligonucleotide
aucgccacucucagauucg

SEQ ID NO: 768 - RNA, 19 nt, Artificial Sequence
  Description of Artificial Sequence Synthetic oligonucleotide
uaucgccacucucagauuc

SEQ ID NO: 769 - RNA, 19 nt, Artificial Sequence
  Description of Artificial Sequence Synthetic oligonucleotide
uaacuaucgccacucucag

SEQ ID NO: 770 - RNA, 19 nt, Artificial Sequence
  Description of Artificial Sequence Synthetic oligonucleotide
uucaugcuaacuaucgcca

SEQ ID NO: 771 - RNA, 19 nt, Artificial Sequence
  Description of Artificial Sequence Synthetic oligonucleotide
auuucacccuucaugcuaa

SEQ ID NO: 772 - RNA, 19 nt, Artificial Sequence
  Description of Artificial Sequence Synthetic oligonucleotide
auuagacuaauccuuuccu

SEQ ID NO: 773 - RNA, 19 nt, Artificial Sequence
  Description of Artificial Sequence Synthetic oligonucleotide
augagcgucacaaaguucu

SEQ ID NO: 774 - RNA, 19 nt, Artificial Sequence
  Description of Artificial Sequence Synthetic oligonucleotide
aagagagcgugguuccacu

SEQ ID NO: 775 - RNA, 19 nt, Artificial Sequence
  Description of Artificial Sequence Synthetic oligonucleotide
uuacguguauucuggagag

SEQ ID NO: 776 - RNA, 19 nt, Artificial Sequence
  Description of Artificial Sequence Synthetic oligonucleotide
uuuacguguauucuggaga

SEQ ID NO: 777 - RNA, 19 nt, Artificial Sequence
  Description of Artificial Sequence Synthetic oligonucleotide
aauuuacguguauucugga

SEQ ID NO: 778 - DNA, 2546 bp, Rattus norvegicus
ctccagggaagcaacagagagaagaaaagaaaggtacatggctacttcccactctcagcctagaagaataaacccttagattgcgggggaactgagccaggcaagccacaggcagccttgagcgcccccttgcctgccctcccctgcgggggccaggatgctgaagaagcagtctgcagggcttgtgctttggggtgctatcatctttgtgggctggaatgccctgctgctcctcttcttctggacacgcccagcacctggcaggctgccctcagacagtgcccttggtgatgaccctgccagcctcacccgtgaggtcatccacctggccgaggacgccgaggcggagttggagcggcagcggggactactgcagcagatcaaggagcattattctctgtggaggcagaggtggagagttcccaccgtggcccctccagcctggccccgtgtgcctgggaccccctcaccagctgtgatccccattctggtcattgcctgtgaccgcagcactgtccggcgctgcctggataagttgttgcactatcggccctctgctgagcatttccccattattgtcagtcaagactgtgggcatgaagagacagcacaggtcattgcttcttatggcaccgctgtcacacacatccggcagccagacctgagtaacattgccgtgcagccagaccaccgtaagttccagggttactacaagattgccaggcactaccgctgggcgctaggccagatcttcaacaagttcaagttcccggctgctgtggtagtggaggatgatctggaagtggcgccggacttcttcgagtacttccaggccacctacccgctgctgaaagcagacccctccctttggtgtgtgtcggcttggaatgataatggtaaggagcagatggtggactccagcaaacctgagctgctctatcgaacagacttttttcctggccttggatggctgctgttggctgatctctgggcagaactagagcccaagtggcccaaggccttctgggacgactggatgcgcagacctgagcagcggaaggggcgggcttgtattcgtccagaaatttcaagaactatgacatttggtcgcaagggtgtgagccatgggcagttctttgaccagcatcttaaattcatcaagctgaaccagcagttcgtgcccttcacccagttggacctgtcgtacctgcagcgggaggcctatgaccgggacttccttgcccaggtctatggtgccccccagctgcaggtggagaaagtgaggaccaatgatcggaaggaactgggggaggtgcgggtacagtacactagcagagacagctttaaggcctttgctaaggccctgggtgtcatggacgacctcaagtctggtgtccccagagctggctaccgtggcattgtcactttccagttccggggtcggcgtgtccacctggcacccccagagacatggaatggctatgatcctagctggaattagtggcacctgcctttccctcctgggtctccttgccgtatgatgagccgaggcagcctgcaggccctgggctataccattctgccagtgtttcctcttggatctatagatcttccctttttgtcctggccttcccccaagtggcattctagtgcacaaatcataagatgagggttatactcctcttgtcaagggagtactgtgtggtatgttcggggcatattgaacaagaaaccactgtgtggtgtggggagggttggacctgttggaccaggcttgtagtgtcctgagttctctccaagggcatctgcggagagcttggccactccagctctcctgaccaggcctctccaccctgatctggctcctgttagtacatgagcccactttatgtaccttccccactttctccaccttccccagggtggggccggattatggaagaaggaatagatttgtggccaactgagactaaccaaagggattcactgtcagagttagattgcatggccgggttgccggggtcactgcctcctggcttctcttcctgccgactttgccagtgctcttcttgtcctgcagtggagcagtgtgttgtgagatggaaagtaggagctaaagcaagacctctcccgtggtggagcagttgtcaggaacaatgacaggcggaaagcctacacttgggcacccctcttcttgcccatggccccccacttctgaaagttagttccctttgcattctttagggggattcagtggcagcctagtgggggacagtgggctccacgcctctttcccctggggttggtacagcctcctcccacagggcttgttcctggtggccatctcagcaaagtcaccagcagctgagaccaggacacctgtggcccttcctaagcattcccacatcttcccaggccgaggggcaggtggcaggctagagagaaagaaatttgtgtgttttgtttgttgcctgatcttagtttcatggaagaaaatggaatctacagaattattttcaaaaataaagtaaaggctgaattgtctg

SEQ ID NO: 779 - RNA, 19 nt, Artificial Sequence
  Description of Artificial Sequence Synthetic oligonucleotide
ccaggguuacuacaagauu

SEQ ID NO: 780 - RNA, 19 nt, Artificial Sequence
  Description of Artificial Sequence Synthetic oligonucleotide
ugcucuaucgaacagacuu

SEQ ID NO: 781 - RNA, 19 nt, Artificial Sequence
  Description of Artificial Sequence Synthetic oligonucleotide
gcucuaucgaacagacuuu

SEQ ID NO: 782 - RNA, 19 nt, Artificial Sequence
  Description of Artificial Sequence Synthetic oligonucleotide
ggcuuguauucguccagaa

SEQ ID NO: 783 - RNA, 19 nt, Artificial Sequence
  Description of Artificial Sequence Synthetic oligonucleotide
ugaggaccaaugaucggaa

SEQ ID NO: 784 - RNA, 19 nt, Artificial Sequence
  Description of Artificial Sequence Synthetic oligonucleotide
gugcggguacaguacacua

SEQ ID NO: 785 - RNA, 19 nt, Artificial Sequence
  Description of Artificial Sequence Synthetic oligonucleotide
gagacagcuuuaaggccuu

SEQ ID NO: 786 - RNA, 19 nt, Artificial Sequence
  Description of Artificial Sequence Synthetic oligonucleotide
uggaauggcuaugauccua

SEQ ID NO: 787 - RNA, 19 nt, Artificial Sequence
  Description of Artificial Sequence Synthetic oligonucleotide
ucuuggaucuauagaucuu

SEQ ID NO: 788 - RNA, 19 nt, Artificial Sequence
  Description of Artificial Sequence Synthetic oligonucleotide
guggcauucuagugcacaa

SEQ ID NO: 789 - RNA, 19 nt, Artificial Sequence
  Description of Artificial Sequence Synthetic oligonucleotide
uggcauucuagugcacaaa

SEQ ID NO: 790 - RNA, 19 nt, Artificial Sequence
  Description of Artificial Sequence Synthetic oligonucleotide
ggcauucuagugcacaaau

SEQ ID NO: 791 - RNA, 19 nt, Artificial Sequence
  Description of Artificial Sequence Synthetic oligonucleotide
ucuagugcacaaaucauaa

SEQ ID NO: 792 - RNA, 19 nt, Artificial Sequence
  Description of Artificial Sequence Synthetic oligonucleotide
agugcacaaaucauaagau

SEQ ID NO: 793 - RNA, 19 nt, Artificial Sequence
  Description of Artificial Sequence Synthetic oligonucleotide
ugugguauguucggggcau

SEQ ID NO: 794 - RNA, 19 nt, Artificial Sequence
  Description of Artificial Sequence Synthetic oligonucleotide
gugguauguucggggcaua

SEQ ID NO: 795 - RNA, 19 nt, Artificial Sequence
  Description of Artificial Sequence Synthetic oligonucleotide
uuaguacaugagcccacuu

SEQ ID NO: 796 - RNA, 19 nt, Artificial Sequence
  Description of Artificial Sequence Synthetic oligonucleotide
uuguggccaacugagacua

SEQ ID NO: 797 - RNA, 19 nt, Artificial Sequence
  Description of Artificial Sequence Synthetic oligonucleotide
gggauucacugucagaguu

SEQ ID NO: 798 - RNA, 19 nt, Artificial Sequence
  Description of Artificial Sequence Synthetic oligonucleotide
uagauugcauggccggguu

SEQ ID NO: 799 - RNA, 19 nt, Artificial Sequence
  Description of Artificial Sequence Synthetic oligonucleotide
ccacuucugaaaguuaguu

SEQ ID NO: 800 - RNA, 19 nt, Artificial Sequence
  Description of Artificial Sequence Synthetic oligonucleotide
aaguuaguucccuuugcau

SEQ ID NO: 801 - RNA, 19 nt, Artificial Sequence
  Description of Artificial Sequence Synthetic oligonucleotide
uuccuaagcauucccacau

SEQ ID NO: 802 - RNA, 19 nt, Artificial Sequence
  Description of Artificial Sequence Synthetic oligonucleotide
cuaagcauucccacaucuu

SEQ ID NO: 803 - RNA, 19 nt, Artificial Sequence
  Description of Artificial Sequence Synthetic oligonucleotide
aaucuuguaguaacccugg

SEQ ID NO: 804 - RNA, 19 nt, Artificial Sequence
  Description of Artificial Sequence Synthetic oligonucleotide
aagucuguucgauagagca

SEQ ID NO: 805 - RNA, 19 nt, Artificial Sequence
  Description of Artificial Sequence Synthetic oligonucleotide
aaagucuguucgauagagc

SEQ ID NO: 806 - RNA, 19 nt, Artificial Sequence
  Description of Artificial Sequence Synthetic oligonucleotide
uucuggacgaauacaagcc

SEQ ID NO: 807 - RNA, 19 nt, Artificial Sequence
  Description of Artificial Sequence Synthetic oligonucleotide
uuccgaucauugguccuca

SEQ ID NO: 808 - RNA, 19 nt, Artificial Sequence
  Description of Artificial Sequence Synthetic oligonucleotide
uaguguacuguacccgcac

SEQ ID NO: 809 - RNA, 19 nt, Artificial Sequence
  Description of Artificial Sequence Synthetic oligonucleotide
aaggccuuaaagcugucuc

SEQ ID NO: 810 - RNA, 19 nt, Artificial Sequence
  Description of Artificial Sequence Synthetic oligonucleotide
uaggaucauagccauucca

SEQ ID NO: 811 - RNA, 19 nt, Artificial Sequence
  Description of Artificial Sequence Synthetic oligonucleotide
aagaucuauagauccaaga

SEQ ID NO: 812 - RNA, 19 nt, Artificial Sequence
  Description of Artificial Sequence Synthetic oligonucleotide
uugugcacuagaaugccac

SEQ ID NO: 813 - RNA, 19 nt, Artificial Sequence
  Description of Artificial Sequence Synthetic oligonucleotide
uuugugcacuagaaugcca

SEQ ID NO: 814 - RNA, 19 nt, Artificial Sequence
  Description of Artificial Sequence Synthetic oligonucleotide
auuugugcacuagaaugcc

SEQ ID NO: 815 - RNA, 19 nt, Artificial Sequence
  Description of Artificial Sequence Synthetic oligonucleotide
uuaugauuugugcacuaga

SEQ ID NO: 816 - RNA, 19 nt, Artificial Sequence
  Description of Artificial Sequence Synthetic oligonucleotide
aucuuaugauuugugcacu

SEQ ID NO: 817 - RNA, 19 nt, Artificial Sequence
  Description of Artificial Sequence Synthetic oligonucleotide
augccccgaacauaccaca

SEQ ID NO: 818 - RNA, 19 nt, Artificial Sequence
  Description of Artificial Sequence Synthetic oligonucleotide
uaugccccgaacauaccac

SEQ ID NO: 819 - RNA, 19 nt, Artificial Sequence
  Description of Artificial Sequence Synthetic oligonucleotide
aagugggcucauguacuaa

SEQ ID NO: 820 - RNA, 19 nt, Artificial Sequence
  Description of Artificial Sequence Synthetic oligonucleotide
uagucucaguuggccacaa

SEQ ID NO: 821 - RNA, 19 nt, Artificial Sequence
  Description of Artificial Sequence Synthetic oligonucleotide
aacucugacagugaauccc

SEQ ID NO: 822 - RNA, 19 nt, Artificial Sequence
  Description of Artificial Sequence Synthetic oligonucleotide
aacccggccaugcaaucua

SEQ ID NO: 823 - RNA, 19 nt, Artificial Sequence
  Description of Artificial Sequence Synthetic oligonucleotide
aacuaacuuucagaagugg

SEQ ID NO: 824 - RNA, 19 nt, Artificial Sequence
  Description of Artificial Sequence Synthetic oligonucleotide
augcaaagggaacuaacuu

SEQ ID NO: 825 - RNA, 19 nt, Artificial Sequence
  Description of Artificial Sequence Synthetic oligonucleotide
augugggaaugcuuaggaa

SEQ ID NO: 826 - RNA, 19 nt, Artificial Sequence
  Description of Artificial Sequence Synthetic oligonucleotide
aagaugugggaaugcuuag

SEQ ID NO: 827 - DNA, 7181 bp, Rattus norvegicus
gctccagctgcgtcgccggcggccgcggtagaagcgccctggcccgcggcagaaagaagaaaaagaaacagcagcccttagccctcagcctctgaaaacctcgaagacaatctggaatagcaatacccacgcttgctttgctgcttgacaccttctgcccagcatctgaagtgaacctcaatcatcaattgctttgtttttggcccaggtccaaggcgttctgccttctgttgacttgaggctggagagagactggtcttgcttgctgttgccaacttgtgaaccagtgtgatggtgaaatgagatgaggctccgaaatggaactgtggccactgcgctggtgtttgtcacgtccttccttaccctgtcctggtataccacatggcaaaatgggaaagaaaaattaattgcttaccaacgagaattcctcgctttaaaagaacgtcttcgagtggccgaacataggatatctcagcgctcctctgagctaaacaccattgtccaacagttccgccgagctggagcggagactaatggaaataataccataaagcttctaaaagagttgacaagcaaaaaatcacttcaagtgccaagtatttattatcatttgcctcatctattgcaaaatgaaagaagccttcagcccgccgtacagattggcagtggaagaacgggagtttcaatagtcatggggattcctactgtgaagagagaagttaaatcttacctcatagagacccttcactcccttattgataatctgtatcctgaagagaagctggactgtgttatagttgtcttcataggagagacagatcttgattatgttcacagcgttgttgccaacctggagaaagaattttctagagaaattagttccggcttgctagaagtaatctctcctcctgaaagctattaccccgacttaacaaacctgaaggagacgttcggagactccaaggaaagagtaagatggagaacgaagcaaaacctggactattgttttctgatgatgtatgctcaggagaagggcatttactacattcagcttgaagatgatattattgtcaagcaaaactattttaataccataaaaaattttgcacttcaactttcttctgaagaatggatgattctagagttttcccagctgggcttcattgggaagatgttccaggcgccggacctagctctggttgtggagttcattctcatgttctataaggagaagcccattgactggctcttggaccacattctctgggtgaaggtctgcaaccccgaaaaagatgccaaacactgcgacagacagaaggcaaacctacgaatccgcttccgaccctccctcttccagcacgtgggtctacactcatctctgtcggggaaaattcagaaacttacggataaagattacatgaaaccattgcttctcaagatccacgtgaacccgcctgcagaggtctccacttccctgaaggtgtaccaagggcacacactggagaagacctacatgggggaagacttcttttgggccatcacccccacagctggagactacatcttgtttaaatttgataaaccggtcaatgtggagagttatttgttccacagcggcaatcaagagcacccaggggacatcctgctgaacacgaccgtggaggttctgcctcttaagagcgacagtttggagatcagcaaagaaaccaaagacaaacgattagaagatggctatttcagaatagggaaatttgagtacggagttgcagagggaattgtggatcctggactgaaccctatctcagcctttcgactgtcggttattcagaactctgctgtctgggccattctcaatgagattcatattaagaaagtcaccagttgatctgcttagaaaccaacacgtcctttcttgtgactcttgattaaagatactgagcacgccgtcctcttctgctctgacttgaacactacctctcgtgaagcctactgtagataagatgattaccatctccacttgggcagggacccttcccaccgacaactgcattcatttgaaacgaggctgtcctccaaatttgaacttgacacaaacattttacaattctgacagcctggtaacacgcctgagactattttggtattatactaatacacaagagttgtacatattgttacattccttaaaatttgagaaaactgatgttaaatacattttgtgaagggggtacttttgaagttcacttattttactattatagaccctcttttatagacgatcagggatgttatatatatataaatataaatatataaatacacatgaagatgttatgaagttaatttattagaagtacttaagaagtacatatttttgtgcagtaaagttttgaatcaacacctttttttgaagaagcggttcctggcttttttaagttccttctatatttttatttgtagatgcagacattctagatcaacgccatttttttccagtgcactgccattccagattggttctagatgggtttcttaacttgaagtacttttataatcacagcaattctgaacaaaatattttcagaggcatttgtcattcctaaaaaaacaatcaagattttaaaagaccagagccctctggagggttatattactgtactgtgtgtggtgtgtagccacagaaacccgtgtgattgtcagtgtcgggtgggaggccctgcctcgtctccatacactccttcaccccctcatagttccctccatgtgggtgacgagactcctttcccttttgctctgtctttaatcagtagttagacacactgtcttcttttctccctggtccttttatagacctgtggtgggttttgacgttcagataatgtttcctgggtaatgctgttggatgagtcatattgggtataattttacaaccaacattgttatgtggggctctggtgaaagacaaccgtagcgcttctaggtttccacagcatgtttgctctgctatgtcctgaatgcaagggtgaacgcgtgtgcagtcccaagaagggctagttggcaaacgtgctttactcttacacttaggaaggccgggcaagttgccagtcttctctggctctccaggagctgcgcatgcccagcaggtgtgggggcctaggtgtggaagttcccagctggtggcctccaccctggaactttctcagttgcccagtgtatggaacacagtacaaagcagcagccattttgtatgttctgactggggaaacgtagataaacactactgtgcatgtcaaagcccagctgaagcttttcttggtgttttgtaaactttgtaaacatggtgttttattcaagtttttaatttcttctctgatttcctttgccctgtgcccacaagcacatccttagaaaatttaagcactatcttcccacactcaagtgtgcagcagggtaacagactgaaaccatgcggatggatgcaggggcccttttaggtctctgctttctcagtggctgcaagttcagagctttgaaaatccagtgggtcacttgactgtctcctgcgtgggtctttgagctgtcctcccgccaggctcctctcatttgtcttgcctgtagggactcttagccaagtcctttccttcattccaggaatcccttttccctttgtgcgcttactcccagtgcctagaacatgcctgagcctcggcaggttcttttagcatctggatgagtggattaactagccaggttgctcacatctctctccgagcactatttgcctgctatcttgtcatgcatgggcacacacactccgtgtctagtcttcattctgtgtggtgtgtgttctccaacacactccgagctctggaggccagctctcctgtcttccacgtcaggtgctcatttgatccatcagtgttagttgctctcacttttgtgcccagttcagcactagactatcagaaacagtttggttggcccaagcaagatttttgtgggagccctggagatttgtgaaggtaaagactagaggaggtgaagccagagtacttagaactgcaaggtgtccaccttgtgggcaaccaacggaccttcacccacagtgaatgggaaaagtgccaaatacagtaccaattatttctgtgaagtagatggtggttggtaccagtgtatttcagatactccagactctagagtggtgctggcctctacagaagccacacatctgttgaaatgaaatgttgagttctgtgcccatgttggcagcatttggagtggtcagcggccacatggccagcagccaccatattggatcccagaaatatgggacactgacatcatggttctgctggtcataaatataagcattctagaaaggagtgatgcatcctgctgctgtgattaaaagtagacctatggattcatggtagtacaggtgaaggtgtgtattttgatgggacggccaaaattgcctggctacatgttagcattatgattcttcccagcatgcattgtttcagtgtccccaagaggtacatttgaaaagcatgagaagtgaatacaggtggatgatttagtttgtagcctagatgtgtgttatgtccgtcagagtgtgtttgaacaaatgtcattggttacatgcaaaagattatttcaaaagctgtcaggaaaagttgagagattagagttctttttagtctttcagaattacaagaactatagttctcaacagtgctgtctctcaaggaagtttgcttatgtggaaagaggtgttgttagcaaaggtttttccagttaataagtgtgtatctgtcatatgcaggattctaagaatgacccctcccatgactttgtataatatcctcctccccttctgagtatagcgacaactgtgaccatgtcatgtcttgttacttaacaaaagggagattacctgtcagtggctgatccaatgttgagagtcatgtcaggctggtacaagtgagagacagcagcagtagaactctgaacagcattgctgggcctgccttaaggagccagaggctttgttacccactgagtgggagctgcagaccttttgccacaggaccatttgacccagacacttaaccggtttggacatggaaatgcttgggcgagcagtcagctgggtaaaccttccctgcagctttgtaagcggggccctacctaggcttctgatgtgcagcagtgggtaagaaattggcagtgctgtacgtggctaggtttgtgttttgtaaaaagtaacaatctcagtcctgagaaggcttcatatgtgattgcctgcagtgactggtttttaagacaaggtctcatcttgcaccccaggctggttctcccagttctgcccctcaagtgtcgatgtgcatcaccatgcccagttgactgatggcttttgacataagaataaacttgttcctagcaagccctattcctggctctctgctaggatgcttggtgccaggacccgtctccactactagccagacttgcgttattttttgttgtgtaaaactggcaggatgaagctgaaggtgtgggctgtaggcatcttctcctagatactcctaaattgtactgacttaaaggcctgccggaagtagggggaagaatgaggaacagtattttagatcagatcaacaagaagaaaaattcccaccgtcatgtcgatttgctggtgtgtccaaccttctgacagtgcagtgggacactgttctctacagagtccaggctcaagaacctcacggtccagttacaaaaacaacgaaactgttaaaaagctttgagtaagtttacaattttgtattgtgccattcattgctgtcctgggccacaggctgaacatacctgcaagagcttcagctacgacagctgtaatttttaaaagatcacttagctgagtagctcacgggtcaaaccctgtatgttccaaggttttgtatttacatgtttcccaaaataccatgcttattgttgctattattcaaccatgctgctgtagccttgagggaaataaacagaactgaaggaaaggaacccagaggttaaacactgtcgtcatcttcgcatggtgagggatcgagtcccagttcaagacttcagggaagggcattcccaagccacgtagcaacatttacggcttgtaaaacttaaactgtgatagttgtgaggacttaaatgattaatattaacatagatgctatgctaattaatatgctaaatttaatgcatgtgaacatatgatgctacaaagatgaccacagtctttacaaagagcaaaagaataaaaccaaccaacccacccaagggttttgttactcgtgcccctgtactctatcactagctaaacagacctttgttgtttctgaaatgctcccttttccagtttctgcactacctaaggatgcaagtgtgagagcagacaggcagcaggtggccctgtgtgagcaggggccggagaaggactggaagtgtccgtgcaggcacacaggcggccactgcgagtgcagcgggaacccctggactgcagagggattcttgctgctctcaacacatgtgttgtgggaacagctaggtacttgtgatactaaggaggctgcttcttgaagctgctctgctgctgagttagcaattatcccaaaatacagagtggggtcctttagcttgcgtagcattccaccctttattggaaatgctccagcgctgtgtaagaggacctgaagcgcattctcttgagccttacaaagagctgctgggacagtactgtccagtccactaagctcacacctctttcccccagtggagtcaaagacgctcagttggtgcacgtcctccacaaggctgtcttcacactgggggacgttctggagtcttgctaggttgtaccaggtcctcttttcctctgctcacaatgtctttatgggttgaacattaactgcagtaatatttgaggtacctgttgaatttttaaaggcagattatttatttttaagtttgctgctataatcgaaacctaataatgtcctgttgactgtgcctttcataactgtctctgtgctacaactgtccatgtgtatttgaaataaagatgtttgaaaagctatgttgaatcaagattccttctaataaaaatggcttaaaccaaaaaaaaaaaaaaaaaa

SEQ ID NO: 828 - RNA, 19 nt, Artificial Sequence
  Description of Artificial Sequence Synthetic oligonucleotide
auucuagaucaacgccauu

SEQ ID NO: 829 - RNA, 19 nt, Artificial Sequence
  Description of Artificial Sequence Synthetic oligonucleotide
uucuagaucaacgccauuu

SEQ ID NO: 830 - RNA, 19 nt, Artificial Sequence
  Description of Artificial Sequence Synthetic oligonucleotide
cugccauuccagauugguu

SEQ ID NO: 831 - RNA, 19 nt, Artificial Sequence
  Description of Artificial Sequence Synthetic oligonucleotide
cacagaaacccgugugauu

SEQ ID NO: 832 - RNA, 19 nt, Artificial Sequence
  Description of Artificial Sequence Synthetic oligonucleotide
ggaugagucauauugggua

SEQ ID NO: 833 - RNA, 19 nt, Artificial Sequence
  Description of Artificial Sequence Synthetic oligonucleotide
gagucauauuggguauaau

SEQ ID NO: 834 - RNA, 19 nt, Artificial Sequence
  Description of Artificial Sequence Synthetic oligonucleotide
uggaugaguggauuaacua

SEQ ID NO: 835 - RNA, 19 nt, Artificial Sequence
  Description of Artificial Sequence Synthetic oligonucleotide
uagccuagauguguguuau

SEQ ID NO: 836 - RNA, 19 nt, Artificial Sequence
  Description of Artificial Sequence Synthetic oligonucleotide
uagccagacuugcguuauu

SEQ ID NO: 837 - RNA, 19 nt, Artificial Sequence
  Description of Artificial Sequence Synthetic oligonucleotide
agccagacuugcguuauuu

SEQ ID NO: 838 - RNA, 19 nt, Artificial Sequence
  Description of Artificial Sequence Synthetic oligonucleotide
ucuccuagauacuccuaaa

SEQ ID NO: 839 - RNA, 19 nt, Artificial Sequence
  Description of Artificial Sequence Synthetic oligonucleotide
cuccuagauacuccuaaau

SEQ ID NO: 840 - RNA, 19 nt, Artificial Sequence
  Description of Artificial Sequence Synthetic oligonucleotide
guagcaacauuuacggcuu

SEQ ID NO: 841 - RNA, 19 nt, Artificial Sequence
  Description of Artificial Sequence Synthetic oligonucleotide
gcaacauuuacggcuugua

SEQ ID NO: 842 - RNA, 19 nt, Artificial Sequence
  Description of Artificial Sequence Synthetic oligonucleotide
cauaugaugcuacaaagau

SEQ ID NO: 843 - RNA, 19 nt, Artificial Sequence
  Description of Artificial Sequence Synthetic oligonucleotide
acagcuagguacuugugau

SEQ ID NO: 844 - RNA, 19 nt, Artificial Sequence
  Description of Artificial Sequence Synthetic oligonucleotide
cagcuagguacuugugaua

SEQ ID NO: 845 - RNA, 19 nt, Artificial Sequence
  Description of Artificial Sequence Synthetic oligonucleotide
ccuuuagcuugcguagcau

SEQ ID NO: 846 - RNA, 19 nt, Artificial Sequence
  Description of Artificial Sequence Synthetic oligonucleotide
guagcauuccacccuuuau

SEQ ID NO: 847 - RNA, 19 nt, Artificial Sequence
  Description of Artificial Sequence Synthetic oligonucleotide
ugagguaccuguugaauuu

SEQ ID NO: 848 - RNA, 19 nt, Artificial Sequence
  Description of Artificial Sequence Synthetic oligonucleotide
ugcuauaaucgaaaccuaa

SEQ ID NO: 849 - RNA, 19 nt, Artificial Sequence
  Description of Artificial Sequence Synthetic oligonucleotide
gcuauaaucgaaaccuaau

SEQ ID NO: 850 - RNA, 19 nt, Artificial Sequence
  Description of Artificial Sequence Synthetic oligonucleotide
aauggcguugaucuagaau

SEQ ID NO: 851 - RNA, 19 nt, Artificial Sequence
  Description of Artificial Sequence Synthetic oligonucleotide
aaauggcguugaucuagaa

SEQ ID NO: 852 - RNA, 19 nt, Artificial Sequence
  Description of Artificial Sequence Synthetic oligonucleotide
aaccaaucuggaauggcag

SEQ ID NO: 853 - RNA, 19 nt, Artificial Sequence
  Description of Artificial Sequence Synthetic oligonucleotide
aaucacacggguuucugug

SEQ ID NO: 854 - RNA, 19 nt, Artificial Sequence
  Description of Artificial Sequence Synthetic oligonucleotide
uacccaauaugacucaucc

SEQ ID NO: 855 - RNA, 19 nt, Artificial Sequence
  Description of Artificial Sequence Synthetic oligonucleotide
auuauacccaauaugacuc

SEQ ID NO: 856 - RNA, 19 nt, Artificial Sequence
  Description of Artificial Sequence Synthetic oligonucleotide
uaguuaauccacucaucca

SEQ ID NO: 857 - RNA, 19 nt, Artificial Sequence
  Description of Artificial Sequence Synthetic oligonucleotide
auaacacacaucuaggcua

SEQ ID NO: 858 - RNA, 19 nt, Artificial Sequence
  Description of Artificial Sequence Synthetic oligonucleotide
aauaacgcaagucuggcua

SEQ ID NO: 859 - RNA, 19 nt, Artificial Sequence
  Description of Artificial Sequence Synthetic oligonucleotide
aaauaacgcaagucuggcu

SEQ ID NO: 860 - RNA, 19 nt, Artificial Sequence
  Description of Artificial Sequence Synthetic oligonucleotide
uuuaggaguaucuaggaga

SEQ ID NO: 861 - RNA, 19 nt, Artificial Sequence
  Description of Artificial Sequence Synthetic oligonucleotide
auuuaggaguaucuaggag

SEQ ID NO: 862 - RNA, 19 nt, Artificial Sequence
  Description of Artificial Sequence Synthetic oligonucleotide
aagccguaaauguugcuac

SEQ ID NO: 863 - RNA, 19 nt, Artificial Sequence
  Description of Artificial Sequence Synthetic oligonucleotide
uacaagccguaaauguugc

SEQ ID NO: 864 - RNA, 19 nt, Artificial Sequence
  Description of Artificial Sequence Synthetic oligonucleotide
aucuuuguagcaucauaug

SEQ ID NO: 865 - RNA, 19 nt, Artificial Sequence
  Description of Artificial Sequence Synthetic oligonucleotide
aucacaaguaccuagcugu

SEQ ID NO: 866 - RNA, 19 nt, Artificial Sequence
  Description of Artificial Sequence Synthetic oligonucleotide
uaucacaaguaccuagcug

SEQ ID NO: 867 - RNA, 19 nt, Artificial Sequence
  Description of Artificial Sequence Synthetic oligonucleotide
augcuacgcaagcuaaagg

SEQ ID NO: 868 - RNA, 19 nt, Artificial Sequence
  Description of Artificial Sequence Synthetic oligonucleotide
auaaaggguggaaugcuac

SEQ ID NO: 869 - RNA, 19 nt, Artificial Sequence
  Description of Artificial Sequence Synthetic oligonucleotide
aaauucaacagguaccuca

SEQ ID NO: 870 - RNA, 19 nt, Artificial Sequence
  Description of Artificial Sequence Synthetic oligonucleotide
uuagguuucgauuauagca

SEQ ID NO: 871 - RNA, 19 nt, Artificial Sequence
  Description of Artificial Sequence Synthetic oligonucleotide
auuagguuucgauuauagc

SEQ ID NO: 872 - DNA, 2395 bp, Rattus norvegicus
gctcttgccgccttgccccaaccctgctctgcccgggcccagcctcagatcgaggccgccagcgccgtgcaccgcggaggagcagcccgcacgcatcccccacctccaccccggttcccgccctgcccgtagcggtctctcccgggcccgcagttcctccggggccggggcccccgccgcctccgggcccccgtgcccgggccccggtcccccgggccatgcggcctcggccccgccggcgcccgccgcgcactgggggagatgaggctccgcaatggcaccttcctgacgctgctgctcttctgcttgtgcgccttcctctctctctcctggtacgcagcgctcagcggccagaaaggtgacgtggtggacatttaccagcgcgagttcctggcactgagagaccgtttgcacgcggctgagcaagagagcctgaagcgctccaaggagctaaacctggtgctggaagaaatcaagagggcagtgtccgagaggcaggcgctgcgggacggagaaggcaatcgcacttggggccgcctaacagaggatccgcgactgaagccgtggaacgtctcgcacaggcacgtacttcatctgcccaccgtcttccaccatctgccgcacctgctggctaaggaaagcagtctgcagcccgcagtgcgagtgggccagggccgcaccggagtgtccgtggtgatgggcattccgagcgtgcggcgcgaggtgcactcgtacttgactgacacattgcactcgctcatctcggagctgagcccgcaggagaaggaagactcggtcatcgtggtgctgatcgccgagactgatacacagtacacgtcggcagtgacagagaacatcaaagccttgttccccacagagatccattctgggctcctggaagtcatctccccttcccctcacttctatcctgacttctcccgccttcgagagtcctttggggatcccaaggagagagtcaggtggaggaccaaacagaacctcgattactgcttcctcatgatgtatgcacagtccaaaggcatctactacgtgcagctggaggatgacattgtagccaagcccaactacttgagcactatgaagaactttgccctccagcagccctctgaggactggatgatcctggagttctcgcagctgggcttcattgggaagatgttcaagtcactggatctgagcctgattgtggaattcatccttatgttctaccgagacaagcccatcgactggctcctggaccacatcctgtgggtgaaagtctgcaaccctgagaaggatgcgaaacattgtgatcggcagaaggccaaccttcggatccgcttcaagccgtccctcttccagcacgtgggcactcactcatccctggcgggcaaaatccagaaactgaaggataaagactttggaaagcatgctctacggaaggagcacgtgaacccaccggcggaggtgagcacaagcctgaagacgtaccagcatttcaccctggagaaggcctacttgcgagaggatttcttctgggccttcacacctgctgccggagacttcatccggttccgcttcttccagccactgcgcctcgagcggttctttttccgaagcgggaacattgagcacccagaagataagctcttcaacacttctgtggaggtgctgcccttcgataacccccagtcagagaaggaggcccttcaggagggccgctcagccactctccggtaccctcggagccctgatggctacctccagattggctccttctacaagggtgtagctgaaggagaagtggatcctgcctttggccccctggaagcactgcgtctctccattcagactgactccccggtgtgggtcattttgagtgagatctttctgaaaaaggccgactaagtagaaggcttccaagggtgctctgtggccggctctggagcccacggttctgagggtgtcgctgctactgctgctgctgccccagaggaccaggcatatccaccccacctggagggttctgcctggcaggcggctcgggctggcctggggttcaccgctggcccggaggccccagaagctggtgctgctcctgcccgccgggccgcgggagaggcaggcggcccccacactgtgcctgagggccggcctgctgccgcccaaaaagaactgaaccgaaccgtttgccccggccagtggtcatgagccaggccattagaagagctttttcttgggtgcccgctgtgtgcggcgcggaacactggaatgcatacactactttatgtgctgtgttttttattcttggatacatttgattttttcacgtaagttcgcatatacttctataagagcgtgacttgtaataaagggttaatgaagtatctctctc

SEQ ID NO: 873 - RNA, 19 nt, Artificial Sequence
  Description of Artificial Sequence Synthetic oligonucleotide
cacugagagaccguuugca

SEQ ID NO: 874 - RNA, 19 nt, Artificial Sequence
  Description of Artificial Sequence Synthetic oligonucleotide
acuugacugacacauugca

SEQ ID NO: 875 - RNA, 19 nt, Artificial Sequence
  Description of Artificial Sequence Synthetic oligonucleotide
gccgagacugauacacagu

SEQ ID NO: 876 - RNA, 19 nt, Artificial Sequence
  Description of Artificial Sequence Synthetic oligonucleotide
ccccacagagauccauucu

SEQ ID NO: 877 - RNA, 19 nt, Artificial Sequence
  Description of Artificial Sequence Synthetic oligonucleotide
accaaacagaaccucgauu

SEQ ID NO: 878 - RNA, 19 nt, Artificial Sequence
  Description of Artificial Sequence Synthetic oligonucleotide
aaacagaaccucgauuacu

SEQ ID NO: 879 - RNA, 19 nt, Artificial Sequence
  Description of Artificial Sequence Synthetic oligonucleotide
uccucaugauguaugcaca

SEQ ID NO: 880 - RNA, 19 nt, Artificial Sequence
  Description of Artificial Sequence Synthetic oligonucleotide
gaauucauccuuauguucu

SEQ ID NO: 881 - RNA, 19 nt, Artificial Sequence
  Description of Artificial Sequence Synthetic oligonucleotide
cccugagaaggaugcgaaa

SEQ ID NO: 882 - RNA, 19 nt, Artificial Sequence
  Description of Artificial Sequence Synthetic oligonucleotide
uuuccgaagcgggaacauu

SEQ ID NO: 883 - RNA, 19 nt, Artificial Sequence
  Description of Artificial Sequence Synthetic oligonucleotide
uccgaagcgggaacauuga

SEQ ID NO: 884 - RNA, 19 nt, Artificial Sequence
  Description of Artificial Sequence Synthetic oligonucleotide
acccagaagauaagcucuu

SEQ ID NO: 885 - RNA, 19 nt, Artificial Sequence
  Description of Artificial Sequence Synthetic oligonucleotide
cuucuacaaggguguagcu

SEQ ID NO: 886 - RNA, 19 nt, Artificial Sequence
  Description of Artificial Sequence Synthetic oligonucleotide
gaacugaaccgaaccguuu

SEQ ID NO: 887 - RNA, 19 nt, Artificial Sequence
  Description of Artificial Sequence Synthetic oligonucleotide
gcggaacacuggaaugcau

SEQ ID NO: 888 - RNA, 19 nt, Artificial Sequence
  Description of Artificial Sequence Synthetic oligonucleotide
cacuggaaugcauacacua

SEQ ID NO: 889 - RNA, 19 nt, Artificial Sequence
  Description of Artificial Sequence Synthetic oligonucleotide
uuuucacguaaguucgcau

SEQ ID NO: 890 - RNA, 19 nt, Artificial Sequence
  Description of Artificial Sequence Synthetic oligonucleotide
acguaaguucgcauauacu

SEQ ID NO: 891 - RNA, 19 nt, Artificial Sequence
  Description of Artificial Sequence Synthetic oligonucleotide
uucgcauauacuucuauaa

SEQ ID NO: 892 - RNA, 19 nt, Artificial Sequence
  Description of Artificial Sequence Synthetic oligonucleotide
auaagagcgugacuuguaa

SEQ ID NO: 893 - RNA, 19 nt, Artificial Sequence
  Description of Artificial Sequence Synthetic oligonucleotide
uguaauaaaggguuaauga

SEQ ID NO: 894 - RNA, 19 nt, Artificial Sequence
  Description of Artificial Sequence Synthetic oligonucleotide
guaauaaaggguuaaugaa

SEQ ID NO: 895 - RNA, 19 nt, Artificial Sequence
  Description of Artificial Sequence Synthetic oligonucleotide
ugcaaacggucucucagug

SEQ ID NO: 896 - RNA, 19 nt, Artificial Sequence
  Description of Artificial Sequence Synthetic oligonucleotide
ugcaaugugucagucaagu

SEQ ID NO: 897 - RNA, 19 nt, Artificial Sequence
  Description of Artificial Sequence Synthetic oligonucleotide
acuguguaucagucucggc

SEQ ID NO: 898 - RNA, 19 nt, Artificial Sequence
  Description of Artificial Sequence Synthetic oligonucleotide
agaauggaucucugugggg

SEQ ID NO: 899 - RNA, 19 nt, Artificial Sequence
  Description of Artificial Sequence Synthetic oligonucleotide
aaucgagguucuguuuggu

SEQ ID NO: 900 - RNA, 19 nt, Artificial Sequence
  Description of Artificial Sequence Synthetic oligonucleotide
aguaaucgagguucuguuu

SEQ ID NO: 901 - RNA, 19 nt, Artificial Sequence
  Description of Artificial Sequence Synthetic oligonucleotide
ugugcauacaucaugagga

SEQ ID NO: 902 - RNA, 19 nt, Artificial Sequence
  Description of Artificial Sequence Synthetic oligonucleotide
agaacauaaggaugaauuc

SEQ ID NO: 903 - RNA, 19 nt, Artificial Sequence
  Description of Artificial Sequence Synthetic oligonucleotide
uuucgcauccuucucaggg

SEQ ID NO: 904 - RNA, 19 nt, Artificial Sequence
  Description of Artificial Sequence Synthetic oligonucleotide
aauguucccgcuucggaaa

SEQ ID NO: 905 - RNA, 19 nt, Artificial Sequence
  Description of Artificial Sequence Synthetic oligonucleotide
ucaauguucccgcuucgga

SEQ ID NO: 906 - RNA, 19 nt, Artificial Sequence
  Description of Artificial Sequence Synthetic oligonucleotide
aagagcuuaucuucugggu

SEQ ID NO: 907 - RNA, 19 nt, Artificial Sequence
  Description of Artificial Sequence Synthetic oligonucleotide
agcuacacccuuguagaag

SEQ ID NO: 908 - RNA, 19 nt, Artificial Sequence
  Description of Artificial Sequence Synthetic oligonucleotide
aaacgguucgguucaguuc

SEQ ID NO: 909 - RNA, 19 nt, Artificial Sequence
  Description of Artificial Sequence Synthetic oligonucleotide
augcauuccaguguuccgc

SEQ ID NO: 910 - RNA, 19 nt, Artificial Sequence
  Description of Artificial Sequence Synthetic oligonucleotide
uaguguaugcauuccagug

SEQ ID NO: 911 - RNA, 19 nt, Artificial Sequence
  Description of Artificial Sequence Synthetic oligonucleotide
augcgaacuuacgugaaaa

SEQ ID NO: 912 - RNA, 19 nt, Artificial Sequence
  Description of Artificial Sequence Synthetic oligonucleotide
aguauaugcgaacuuacgu

SEQ ID NO: 913 - RNA, 19 nt, Artificial Sequence
  Description of Artificial Sequence Synthetic oligonucleotide
uuauagaaguauaugcgaa

SEQ ID NO: 914 - RNA, 19 nt, Artificial Sequence
  Description of Artificial Sequence Synthetic oligonucleotide
uuacaagucacgcucuuau

SEQ ID NO: 915 - RNA, 19 nt, Artificial Sequence
  Description of Artificial Sequence Synthetic oligonucleotide
ucauuaacccuuuauuaca

SEQ ID NO: 916 - RNA, 19 nt, Artificial Sequence
  Description of Artificial Sequence Synthetic oligonucleotide
uucauuaacccuuuauuac

SEQ ID NO: 917 - DNA, 1909 bp, Rattus norvegicus
ttccagaaaatgtcagtctattcttcaaactatactgcttgacagtgatgactctggtggctgccgcttataccatagctttaagatatacaaggacaacagcggaaggactctacttttcaaccacagccgtgtgcatcacagaagttataaagttactgataagtgtcggccttctagctaaagaaacaggcagtttgggtagatttaaagcctctttgagtgaaaacgtcttggggagccctaaggagctgctgaagttaagtgtgccgtcactggtgtatgctgtgcagaacaacatggctttcctagctctcagtaacctggatgcagcagtgtaccaggtgacctatcaactgaagattccctgcactgctttatgtactgttttaatgttaaatcgatcactcagcaaactacagtggatttcggtcttcatgctgtgtggtggggtcacacttgtacagtggaaaccagcccaagctacaaaagtcgtggtagcgcagaacccgttgttaggctttggagctatagccattgctgtgctgtgctcgggatttgcaggagtttattttgaaaaagttttaaagagttcagacacttccctttgggtgagaaacattcagatgtatctgtcagggatcgctgtgacattagctggtacctacttgtcggatggcgctgaaattaaagaaaaaggatttttctatggctacacgtattatgtctggtttgttatcttccttgctagtgtgggaggcctctacacgtcagtggtggtgaagtacacagacaacatcatgaaaggcttctctgcggccgcagccattgttctgtctaccgttgcctcagtcatactgtttggattgcagataacactttcatttacactgggagctcttcttgtatgtgtttccatttatctctatgggttacccagacaagataccacatccattcaacaagaaacaacttcaaaagaaagaatcattggtgtgtgatttgaatctcaagagattcctataaggactaaactgttgataataaattagagccttaagtcaaccccagatggtagtttaaacattgtcaacaaattaactgtatgacataagaatcaagaagaaaactctgaatgaaatgctaaaacagatttaatttgggtgtgtttggtgtcaagttatattatttcagaatgaaggactttgtgtgtgtgtgtgtgtgtgtgtgtgagagagagagagagagagagagagagatagagagatagagagatcttgtacatagagcatggaggtgacaatggtgtgtgtgtgtgtgtgtgtttgtgtgtgtgtgtgtgtgtgtgtgtgtgtgtgtgtgtgtgtgtgtgtgtgatatccacagagcatggagacaacaatgtaggtcagagcaccaggtgaaactacaatattcaagcaacaacgtttaagacagtgtctgagggttcaagtgccaaagccgtttctgtgcacactgttctgttgttcaggtactgggagaggaaggtgagccttcttctccagtttatggatagtactttgtccccatagcagtaaaagatttggcttctcttatggaagtgaggaaggacagttcggaacactactcagtgtgtaacttataaaacaggtctacttcatagctcagacactgacttctaggtgacgactgagaacttcagtgatgtatttgtgccatttactgactggttcctatttctttgctgtcagctgatatacttttcagaaaattttataagccgcttttatactttcttttttataaagtatggttacctgttgggctctcaatttgtgacttttagtgattttaaaatatttctataatgttaatgaggaaatccagcaataaacttatttatatc

SEQ ID NO: 918 - RNA, 19 nt, Artificial Sequence
  Description of Artificial Sequence Synthetic oligonucleotide
gccgcuuauaccauagcuu

SEQ ID NO: 919 - RNA, 19 nt, Artificial Sequence
  Description of Artificial Sequence Synthetic oligonucleotide
ccgcuuauaccauagcuuu

SEQ ID NO: 920 - RNA, 19 nt, Artificial Sequence
  Description of Artificial Sequence Synthetic oligonucleotide
auaccauagcuuuaagaua

SEQ ID NO: 921 - RNA, 19 nt, Artificial Sequence
  Description of Artificial Sequence Synthetic oligonucleotide
uaccauagcuuuaagauau

SEQ ID NO: 922 - RNA, 19 nt, Artificial Sequence
  Description of Artificial Sequence Synthetic oligonucleotide
accauagcuuuaagauaua

SEQ ID NO: 923 - RNA, 19 nt, Artificial Sequence
  Description of Artificial Sequence Synthetic oligonucleotide
gcggaaggacucuacuuuu

SEQ ID NO: 924 - RNA, 19 nt, Artificial Sequence
  Description of Artificial Sequence Synthetic oligonucleotide
acugauaagugucggccuu

SEQ ID NO: 925 - RNA, 19 nt, Artificial Sequence
  Description of Artificial Sequence Synthetic oligonucleotide
gugucggccuucuagcuaa

SEQ ID NO: 926 - RNA, 19 nt, Artificial Sequence
  Description of Artificial Sequence Synthetic oligonucleotide
aggcaguuuggguagauuu

SEQ ID NO: 927 - RNA, 19 nt, Artificial Sequence
  Description of Artificial Sequence Synthetic oligonucleotide
gcaguuuggguagauuuaa

SEQ ID NO: 928 - RNA, 19 nt, Artificial Sequence
  Description of Artificial Sequence Synthetic oligonucleotide
ugguggggucacacuugua

SEQ ID NO: 929 - RNA, 19 nt, Artificial Sequence
  Description of Artificial Sequence Synthetic oligonucleotide
uugucggauggcgcugaaa

SEQ ID NO: 930 - RNA, 19 nt, Artificial Sequence
  Description of Artificial Sequence Synthetic oligonucleotide
ucggauggcgcugaaauua

SEQ ID NO: 931 - RNA, 19 nt, Artificial Sequence
  Description of Artificial Sequence Synthetic oligonucleotide
uuuucuauggcuacacgua

SEQ ID NO: 932 - RNA, 19 nt, Artificial Sequence
  Description of Artificial Sequence Synthetic oligonucleotide
ucuauggcuacacguauua

SEQ ID NO: 933 - RNA, 19 nt, Artificial Sequence
  Description of Artificial Sequence Synthetic oligonucleotide
acacguauuaugucugguu

SEQ ID NO: 934 - RNA, 19 nt, Artificial Sequence
  Description of Artificial Sequence Synthetic oligonucleotide
uggauugcagauaacacuu

SEQ ID NO: 935 - RNA, 19 nt, Artificial Sequence
  Description of Artificial Sequence Synthetic oligonucleotide
ggacuaaacuguugauaau

SEQ ID NO: 936 - RNA, 19 nt, Artificial Sequence
  Description of Artificial Sequence Synthetic oligonucleotide
uauucaagcaacaacguuu

SEQ ID NO: 937 - RNA, 19 nt, Artificial Sequence
  Description of Artificial Sequence Synthetic oligonucleotide
uucaagugccaaagccguu

SEQ ID NO: 938 - RNA, 19 nt, Artificial Sequence
  Description of Artificial Sequence Synthetic oligonucleotide
ucuaggugacgacugagaa

SEQ ID NO: 939 - RNA, 19 nt, Artificial Sequence
  Description of Artificial Sequence Synthetic oligonucleotide
uuugcugucagcugauaua

SEQ ID NO: 940 - RNA, 19 nt, Artificial Sequence
  Description of Artificial Sequence Synthetic oligonucleotide
gccgcuuuuauacuuucuu

SEQ ID NO: 941 - RNA, 19 nt, Artificial Sequence
  Description of Artificial Sequence Synthetic oligonucleotide
ccgcuuuuauacuuucuuu

SEQ ID NO: 942 - RNA, 19 nt, Artificial Sequence
  Description of Artificial Sequence Synthetic oligonucleotide
uaaaguaugguuaccuguu

SEQ ID NO: 943 - RNA, 19 nt, Artificial Sequence
  Description of Artificial Sequence Synthetic oligonucleotide
aagcuaugguauaagcggc

SEQ ID NO: 944 - RNA, 19 nt, Artificial Sequence
  Description of Artificial Sequence Synthetic oligonucleotide
aaagcuaugguauaagcgg

SEQ ID NO: 945 - RNA, 19 nt, Artificial Sequence
  Description of Artificial Sequence Synthetic oligonucleotide
uaucuuaaagcuaugguau

SEQ ID NO: 946 - RNA, 19 nt, Artificial Sequence
  Description of Artificial Sequence Synthetic oligonucleotide
auaucuuaaagcuauggua

SEQ ID NO: 947 - RNA, 19 nt, Artificial Sequence
  Description of Artificial Sequence Synthetic oligonucleotide
uauaucuuaaagcuauggu

SEQ ID NO: 948 - RNA, 19 nt, Artificial Sequence
  Description of Artificial Sequence Synthetic oligonucleotide
aaaaguagaguccuuccgc

SEQ ID NO: 949 - RNA, 19 nt, Artificial Sequence
  Description of Artificial Sequence Synthetic oligonucleotide
aaggccgacacuuaucagu

SEQ ID NO: 950 - RNA, 19 nt, Artificial Sequence
  Description of Artificial Sequence Synthetic oligonucleotide
uuagcuagaaggccgacac

SEQ ID NO: 951 - RNA, 19 nt, Artificial Sequence
  Description of Artificial Sequence Synthetic oligonucleotide
aaaucuacccaaacugccu

SEQ ID NO: 952 - RNA, 19 nt, Artificial Sequence
  Description of Artificial Sequence Synthetic oligonucleotide
uuaaaucuacccaaacugc

SEQ ID NO: 953 - RNA, 19 nt, Artificial Sequence
  Description of Artificial Sequence Synthetic oligonucleotide
uacaagugugaccccacca

SEQ ID NO: 954 - RNA, 19 nt, Artificial Sequence
  Description of Artificial Sequence Synthetic oligonucleotide
uuucagcgccauccgacaa

SEQ ID NO: 955 - RNA, 19 nt, Artificial Sequence
  Description of Artificial Sequence Synthetic oligonucleotide
uaauuucagcgccauccga

SEQ ID NO: 956 - RNA, 19 nt, Artificial Sequence
  Description of Artificial Sequence Synthetic oligonucleotide
uacguguagccauagaaaa

SEQ ID NO: 957 - RNA, 19 nt, Artificial Sequence
  Description of Artificial Sequence Synthetic oligonucleotide
uaauacguguagccauaga

SEQ ID NO: 958 - RNA, 19 nt, Artificial Sequence
  Description of Artificial Sequence Synthetic oligonucleotide
aaccagacauaauacgugu

SEQ ID NO: 959 - RNA, 19 nt, Artificial Sequence
  Description of Artificial Sequence Synthetic oligonucleotide
aaguguuaucugcaaucca

SEQ ID NO: 960 - RNA, 19 nt, Artificial Sequence
  Description of Artificial Sequence Synthetic oligonucleotide
auuaucaacaguuuagucc

SEQ ID NO: 961 - RNA, 19 nt, Artificial Sequence
  Description of Artificial Sequence Synthetic oligonucleotide
aaacguuguugcuugaaua

SEQ ID NO: 962 - RNA, 19 nt, Artificial Sequence
  Description of Artificial Sequence Synthetic oligonucleotide
aacggcuuuggcacuugaa

SEQ ID NO: 963 - RNA, 19 nt, Artificial Sequence
  Description of Artificial Sequence Synthetic oligonucleotide
uucucagucgucaccuaga

SEQ ID NO: 964 - RNA, 19 nt, Artificial Sequence
  Description of Artificial Sequence Synthetic oligonucleotide
uauaucagcugacagcaaa

SEQ ID NO: 965 - RNA, 19 nt, Artificial Sequence
  Description of Artificial Sequence Synthetic oligonucleotide
aagaaaguauaaaagcggc

SEQ ID NO: 966 - RNA, 19 nt, Artificial Sequence
  Description of Artificial Sequence Synthetic oligonucleotide
aaagaaaguauaaaagcgg

SEQ ID NO: 967 - RNA, 19 nt, Artificial Sequence
  Description of Artificial Sequence Synthetic oligonucleotide
aacagguaaccauacuuua

SEQ ID NO: 968 - DNA, 1918 bp, Homo sapiens
acaaggggcggtccccggtgtcctgcgcgggggcgcggagggggcgggcgtcagttccgcggggggctgtcggggaaccatggctgccccgagagacaatgtcactttattattcaagttatactgcttggcagtgatgaccctgatggctgcagtctataccatagctttaagatacacaaggacatcagacaaagaactctacttttcaaccacagccgtgtgtatcacagaagttataaagttattgctaagtgtgggaattttagctaaagaaactggtagtctgggtagattcaaagcatctttaagagaaaatgtcttggggagccccaaggaactgttgaagttaagtgtgccatcgttagtgtatgctgttcagaacaacatggctttcctagctcttagcaatctggatgcagcagtgtaccaggtgacctaccagttgaagattccgtgtactgctttatgcactgttttaatgttaaaccggacactcagcaaattacagtgggtttcagtttttatgctgtgtgctggagttacgcttgtacagtggaaaccagcccaagctacaaaagtggtggtggaacaaaatccattattagggtttggcgctatagctattgctgtattgtgctcaggatttgcaggagtatattttgaaaaagttttaaagagttcagatacttctctttgggtgagaaacattcaaatgtatctatcagggattattgtgacattagctggcgtctacttgtcagatggagctgaaattaaagaaaaaggatttttctatggttacacatattatgtctggtttgtcatctttcttgcaagtgttggtggcctctacacttctgttgtggttaagtacacagacaacatcatgaaaggcttttctgcagcagcggccattgtcctttccaccattgcttcagtaatgctgtttggattacagataacactcacctttgccctgggtactcttcttgtatgtgtttccatatatctctatggattacccagacaagacactacatccatccaacaaggagaaacagcttcaaaggagagagttattggtgtgtgattttagcctcacgtgagactccttttaagactaaaccatttgcattaaactagagccttaagtcaatctcagaaggtagcataaacaaataaaaattaactgtatggcatgatcagtgcggttatgtggaaacaacaacaaacaaacgaagctatctgagtgaactgctaatacagaaacttaatgtagacctgtttggggtctactattgttttagaatgaaggaattgtattattgtgtgtatatataatttgtaaataaaaagtatggagatgatacggtgttaaaaaaaatcatggtaaggctacaatactcaagtaacaaggtttgggacaatgtctaagggttaaagtgccaaagccatttctgtactaactgttctcttgttccggtaccggggagaaggatgacccctccttattctccaattcatgtacagtattttgtcctagcagcataaagacctagctcttttcttacaagaggcagaaacaagacaggctagttcataaacaaactgtgtaacttctcaaaatgaatctatttcataactcggacaatttctgggtggtgactgagtacccctttagtgagtacccctttagtgctatatttgtgccattcattatctggttcatatttcttttctgttagatgatacacatttcttcaaaaaaatttctaatgtcacttttgtacttttttaaataaagtatgtttaactgttgggctctcaataatttgtgaaatttcagtgttttctataatgttaatggggaaattcagcaataaactttatttgtaagaaagaaaaaaaaaaaaaa

SEQ ID NO: 969 - RNA, 19 nt, Artificial Sequence
  Description of Artificial Sequence Synthetic oligonucleotide
cagucuauaccauagcuuu

SEQ ID NO: 970 - RNA, 19 nt, Artificial Sequence
  Description of Artificial Sequence Synthetic oligonucleotide
cagacaaagaacucuacuu

SEQ ID NO: 971 - RNA, 19 nt, Artificial Sequence
  Description of Artificial Sequence Synthetic oligonucleotide
agccguguguaucacagaa

SEQ ID NO: 972 - RNA, 19 nt, Artificial Sequence
  Description of Artificial Sequence Synthetic oligonucleotide
uagucuggguagauucaaa

SEQ ID NO: 973 - RNA, 19 nt, Artificial Sequence
  Description of Artificial Sequence Synthetic oligonucleotide
guuaagugugccaucguua

SEQ ID NO: 974 - RNA, 19 nt, Artificial Sequence
  Description of Artificial Sequence Synthetic oligonucleotide
ugcuggaguuacgcuugua

SEQ ID NO: 975 - RNA, 19 nt, Artificial Sequence
  Description of Artificial Sequence Synthetic oligonucleotide
uacgcuuguacaguggaaa

SEQ ID NO: 976 - RNA, 19 nt, Artificial Sequence
  Description of Artificial Sequence Synthetic oligonucleotide
ggguuuggcgcuauagcua

SEQ ID NO: 977 - RNA, 19 nt, Artificial Sequence
  Description of Artificial Sequence Synthetic oligonucleotide
gguuuggcgcuauagcuau

SEQ ID NO: 978 - RNA, 19 nt, Artificial Sequence
  Description of Artificial Sequence Synthetic oligonucleotide
guaucuaucagggauuauu

SEQ ID NO: 979 - RNA, 19 nt, Artificial Sequence
  Description of Artificial Sequence Synthetic oligonucleotide
uucuaugguuacacauauu

SEQ ID NO: 980 - RNA, 19 nt, Artificial Sequence
  Description of Artificial Sequence Synthetic oligonucleotide
cacauauuaugucugguuu

SEQ ID NO: 981 - RNA, 19 nt, Artificial Sequence
  Description of Artificial Sequence Synthetic oligonucleotide
ugcauuaaacuagagccuu

SEQ ID NO: 982 - RNA, 19 nt, Artificial Sequence
  Description of Artificial Sequence Synthetic oligonucleotide
aacuagagccuuaagucaa

SEQ ID NO: 983 - RNA, 19 nt, Artificial Sequence
  Description of Artificial Sequence Synthetic oligonucleotide
agaagguagcauaaacaaa

SEQ ID NO: 984 - RNA, 19 nt, Artificial Sequence
  Description of Artificial Sequence Synthetic oligonucleotide
gagaugauacgguguuaaa

SEQ ID NO: 985 - RNA, 19 nt, Artificial Sequence
  Description of Artificial Sequence Synthetic oligonucleotide
aaucaugguaaggcuacaa

SEQ ID NO: 986 - RNA, 19 nt, Artificial Sequence
  Description of Artificial Sequence Synthetic oligonucleotide
gggacaaugucuaaggguu

SEQ ID NO: 987 - RNA, 19 nt, Artificial Sequence
  Description of Artificial Sequence Synthetic oligonucleotide
ggacaaugucuaaggguua

SEQ ID NO: 988 - RNA, 19 nt, Artificial Sequence
  Description of Artificial Sequence Synthetic oligonucleotide
aagacaggcuaguucauaa

SEQ ID NO: 989 - RNA, 19 nt, Artificial Sequence
  Description of Artificial Sequence Synthetic oligonucleotide
agacaggcuaguucauaaa

SEQ ID NO: 990 - RNA, 19 nt, Artificial Sequence
  Description of Artificial Sequence Synthetic oligonucleotide
auuucauaacucggacaau

SEQ ID NO: 991 - RNA, 19 nt, Artificial Sequence
  Description of Artificial Sequence Synthetic oligonucleotide
uuucauaacucggacaauu

SEQ ID NO: 992 - RNA, 19 nt, Artificial Sequence
  Description of Artificial Sequence Synthetic oligonucleotide
uucauaacucggacaauuu

SEQ ID NO: 993 - RNA, 19 nt, Artificial Sequence
  Description of Artificial Sequence Synthetic oligonucleotide
ugccauucauuaucugguu

SEQ ID NO: 994 - RNA, 19 nt, Artificial Sequence
  Description of Artificial Sequence Synthetic oligonucleotide
acuguugggcucucaauaa

SEQ ID NO: 995 - RNA, 19 nt, Artificial Sequence
  Description of Artificial Sequence Synthetic oligonucleotide
aaagcuaugguauagacug

SEQ ID NO: 996 - RNA, 19 nt, Artificial Sequence
  Description of Artificial Sequence Synthetic oligonucleotide
aaguagaguucuuugucug

SEQ ID NO: 997 - RNA, 19 nt, Artificial Sequence
  Description of Artificial Sequence Synthetic oligonucleotide
uucugugauacacacggcu

SEQ ID NO: 998 - RNA, 19 nt, Artificial Sequence
  Description of Artificial Sequence Synthetic oligonucleotide
uuugaaucuacccagacua

SEQ ID NO: 999 - RNA, 19 nt, Artificial Sequence
  Description of Artificial Sequence Synthetic oligonucleotide
uaacgauggcacacuuaac

SEQ ID NO: 1000 - RNA, 19 nt, Artificial Sequence
  Description of Artificial Sequence Synthetic oligonucleotide
uacaagcguaacuccagca

SEQ ID NO: 1001 - RNA, 19 nt, Artificial Sequence
  Description of Artificial Sequence Synthetic oligonucleotide
uuuccacuguacaagcgua

SEQ ID NO: 1002 - RNA, 19 nt, Artificial Sequence
  Description of Artificial Sequence Synthetic oligonucleotide
uagcuauagcgccaaaccc

SEQ ID NO: 1003 - RNA, 19 nt, Artificial Sequence
  Description of Artificial Sequence Synthetic oligonucleotide
auagcuauagcgccaaacc

SEQ ID NO: 1004 - RNA, 19 nt, Artificial Sequence
  Description of Artificial Sequence Synthetic oligonucleotide
aauaaucccugauagauac

SEQ ID NO: 1005 - RNA, 19 nt, Artificial Sequence
  Description of Artificial Sequence Synthetic oligonucleotide
aauauguguaaccauagaa

SEQ ID NO: 1006 - RNA, 19 nt, Artificial Sequence
  Description of Artificial Sequence Synthetic oligonucleotide
aaaccagacauaauaugug

SEQ ID NO: 1007 - RNA, 19 nt, Artificial Sequence
  Description of Artificial Sequence Synthetic oligonucleotide
aaggcucuaguuuaaugca

SEQ ID NO: 1008 - RNA, 19 nt, Artificial Sequence
  Description of Artificial Sequence Synthetic oligonucleotide
uugacuuaaggcucuaguu

SEQ ID NO: 1009 - RNA, 19 nt, Artificial Sequence
  Description of Artificial Sequence Synthetic oligonucleotide
uuuguuuaugcuaccuucu

SEQ ID NO: 1010 - RNA, 19 nt, Artificial Sequence
  Description of Artificial Sequence Synthetic oligonucleotide
uuuaacaccguaucaucuc

SEQ ID NO: 1011 - RNA, 19 nt, Artificial Sequence
  Description of Artificial Sequence Synthetic oligonucleotide
uuguagccuuaccaugauu

SEQ ID NO: 1012 - RNA, 19 nt, Artificial Sequence
  Description of Artificial Sequence Synthetic oligonucleotide
aacccuuagacauuguccc

SEQ ID NO: 1013 - RNA, 19 nt, Artificial Sequence
  Description of Artificial Sequence Synthetic oligonucleotide
uaacccuuagacauugucc

SEQ ID NO: 1014 - RNA, 19 nt, Artificial Sequence
  Description of Artificial Sequence Synthetic oligonucleotide
uuaugaacuagccugucuu

SEQ ID NO: 1015 - RNA, 19 nt, Artificial Sequence
  Description of Artificial Sequence Synthetic oligonucleotide
uuuaugaacuagccugucu

SEQ ID NO: 1016 - RNA, 19 nt, Artificial Sequence
  Description of Artificial Sequence Synthetic oligonucleotide
auuguccgaguuaugaaau

SEQ ID NO: 1017 - RNA, 19 nt, Artificial Sequence
  Description of Artificial Sequence Synthetic oligonucleotide
aauuguccgaguuaugaaa

SEQ ID NO: 1018 - RNA, 19 nt, Artificial Sequence
  Description of Artificial Sequence Synthetic oligonucleotide
aaauuguccgaguuaugaa

SEQ ID NO: 1019 - RNA, 19 nt, Artificial Sequence
  Description of Artificial Sequence Synthetic oligonucleotide
aaccagauaaugaauggca

SEQ ID NO: 1020 - RNA, 19 nt, Artificial Sequence
  Description of Artificial Sequence Synthetic oligonucleotide
uuauugagagcccaacagu

SEQ ID NO: 1021 - DNA, 2039 bp, Mus musculus
ggaagctgcggttttccgagcccccgcaggaccgagtctcccagacttgtcgctccgcacgccgcgcggcttccgccgtagcccccgcttcctcctcgcgcgtgtggtgcggcgggctctctaggccggtgcgtctctatggccgcaggggcgtcagttccgcagactctctcggcaccatggctccggcgagagaaaatgtcagtttattcttcaagctgtactgcttgacggtgatgactctggtggctgccgcttacaccgtagctttaagatacacaaggacaacagctgaagaactctacttctcaaccactgccgtgtgtatcacagaagtgataaagttactgataagtgttggcctgttagctaaggaaactggcagtttgggtagatttaaagcctcattaagtgaaaatgtcttggggagccccaaggaactggcgaagttgagtgtgccatcactagtgtatgctgtgcagaacaacatggccttcctggctctcagtaatctggatgcagcagtgtaccaggtgacctatcaactgaagatcccctgcactgctttatgtactgttttaatgttaaatcgaacactcagcaaattacagtggatttccgtcttcatgctgtgtggtggggtcacactcgtacagtggaaaccagcccaagctacaaaagtcgtggtagcgcagaatccattgttaggctttggtgctatagctattgctgtattgtgctctggatttgcaggagtttattttgaaaaagtcttaaagagttccgacacttccctttgggtgagaaacattcagatgtatctgtcagggatcgttgtgacgttagctggtacctacttgtcagatggagctgaaattcaagaaaaaggattcttctatggctacacgtattatgtctggtttgttatcttccttgctagtgtgggaggcctctacacgtcagtggtggtgaagtatacagacaacatcatgaaaggcttctctgctgccgcagccattgttctttctaccattgcttcagtcctactgtttggattacagataacactttcatttgcactgggagctcttcttgtgtgtgtttccatatatctctatgggttacccagacaagatactacatccattcaacaagaagcaacttcaaaagagagaatcattggtgtgtgatttgaatctcaagagattcctataaggacttaaactgttgataataaattagagccttaagtcaaccccagatggtaggttaaataatgtcaacaaaataattgtatgacataagaatcaagaagaaaactctgaatgaaatgctaaaacagatttaatttgggtgtgtttggtgtcaagttatattatttcaaaatgaaggactttatatatatgagagagagagagagagagagagagagaaagagagagagagatcttgtacacagagcatggaggtgccatggtatttttgtgtgtgtgtgtgtgtgtgtgtgtgtgtgatgtacacagagcacggaggcaatgatgcaggaccagagagcatcaggtgaaactataatattcaagcaacgaggtttaagaccgtgtctgagggttacagtgccaaagccatttctgtacacactgttctcttgttcaggtacctggagaggaaggctagccttcttctccagtccatggatagtactttgtccccatagcagtgaggatctagcttctcttctcagagtgaggaaggagtaagacagttgaacacacctcagggtgaatctacttcgtagctcagacactgacttctgggtgaagactgagaactctagtgatgcatttgtgccatttactgtctggttcctatttctttgctgtcagctgatatacttttcagaaaattttataagctgcttttatactttcttttttataaagtatggttacctgttgggctctcaatttgtgactttcagtgattttaaaatatttctataatgttaatggggaaatccagcaataaacttatttctacca

SEQ ID NO: 1022 - RNA, 19 nt, Artificial Sequence
  Description of Artificial Sequence Synthetic oligonucleotide
ccgcuuacaccguagcuuu

SEQ ID NO: 1023 - RNA, 19 nt, Artificial Sequence
  Description of Artificial Sequence Synthetic oligonucleotide
cgcuuacaccguagcuuua

SEQ ID NO: 1024 - RNA, 19 nt, Artificial Sequence
  Description of Artificial Sequence Synthetic oligonucleotide
acuggcaguuuggguagau

SEQ ID NO: 1025 - RNA, 19 nt, Artificial Sequence
  Description of Artificial Sequence Synthetic oligonucleotide
ccaaggaacuggcgaaguu

SEQ ID NO: 1026 - RNA, 19 nt, Artificial Sequence
  Description of Artificial Sequence Synthetic oligonucleotide
ugugccaucacuaguguau

SEQ ID NO: 1027 - RNA, 19 nt, Artificial Sequence
  Description of Artificial Sequence Synthetic oligonucleotide
guaccaggugaccuaucaa

SEQ ID NO: 1028 - RNA, 19 nt, Artificial Sequence
  Description of Artificial Sequence Synthetic oligonucleotide
aguggauuuccgucuucau

SEQ ID NO: 1029 - RNA, 19 nt, Artificial Sequence
  Description of Artificial Sequence Synthetic oligonucleotide
agcuacaaaagucguggua

SEQ ID NO: 1030 - RNA, 19 nt, Artificial Sequence
  Description of Artificial Sequence Synthetic oligonucleotide
uuaaagaguuccgacacuu

SEQ ID NO: 1031 - RNA, 19 nt, Artificial Sequence
  Description of Artificial Sequence Synthetic oligonucleotide
cagggaucguugugacguu

SEQ ID NO: 1032 - RNA, 19 nt, Artificial Sequence
  Description of Artificial Sequence Synthetic oligonucleotide
uggauuacagauaacacuu

SEQ ID NO: 1033 - RNA, 19 nt, Artificial Sequence
  Description of Artificial Sequence Synthetic oligonucleotide
auuugaaucucaagagauu

SEQ ID NO: 1034 - RNA, 19 nt, Artificial Sequence
  Description of Artificial Sequence Synthetic oligonucleotide
aaccccagaugguagguua

SEQ ID NO: 1035 - RNA, 19 nt, Artificial Sequence
  Description of Artificial Sequence Synthetic oligonucleotide
accccagaugguagguuaa

SEQ ID NO: 1036 - RNA, 19 nt, Artificial Sequence
  Description of Artificial Sequence Synthetic oligonucleotide
ccccagaugguagguuaaa

SEQ ID NO: 1037 - RNA, 19 nt, Artificial Sequence
  Description of Artificial Sequence Synthetic oligonucleotide
cauggaggugccaugguau

SEQ ID NO: 1038 - RNA, 19 nt, Artificial Sequence
  Description of Artificial Sequence Synthetic oligonucleotide
auauucaagcaacgagguu

SEQ ID NO: 1039 - RNA, 19 nt, Artificial Sequence
  Description of Artificial Sequence Synthetic oligonucleotide
uauucaagcaacgagguuu

SEQ ID NO: 1040 - RNA, 19 nt, Artificial Sequence
  Description of Artificial Sequence Synthetic oligonucleotide
auucaagcaacgagguuua

SEQ ID NO: 1041 - RNA, 19 nt, Artificial Sequence
  Description of Artificial Sequence Synthetic oligonucleotide
cugucagcugauauacuuu

SEQ ID NO: 1042 - RNA, 19 nt, Artificial Sequence
  Description of Artificial Sequence Synthetic oligonucleotide
uaaaguaugguuaccuguu

SEQ ID NO: 1043 - RNA, 19 nt, Artificial Sequence
  Description of Artificial Sequence Synthetic oligonucleotide
aaagcuacgguguaagcgg

SEQ ID NO: 1044 - RNA, 19 nt, Artificial Sequence
  Description of Artificial Sequence Synthetic oligonucleotide
uaaagcuacgguguaagcg

SEQ ID NO: 1045 - RNA, 19 nt, Artificial Sequence
  Description of Artificial Sequence Synthetic oligonucleotide
aucuacccaaacugccagu

SEQ ID NO: 1046 - RNA, 19 nt, Artificial Sequence
  Description of Artificial Sequence Synthetic oligonucleotide
aacuucgccaguuccuugg

SEQ ID NO: 1047 - RNA, 19 nt, Artificial Sequence
  Description of Artificial Sequence Synthetic oligonucleotide
auacacuagugauggcaca

SEQ ID NO: 1048 - RNA, 19 nt, Artificial Sequence
  Description of Artificial Sequence Synthetic oligonucleotide
uugauaggucaccugguac

SEQ ID NO: 1049 - RNA, 19 nt, Artificial Sequence
  Description of Artificial Sequence Synthetic oligonucleotide
augaagacggaaauccacu

SEQ ID NO: 1050 - RNA, 19 nt, Artificial Sequence
  Description of Artificial Sequence Synthetic oligonucleotide
uaccacgacuuuuguagcu

SEQ ID NO: 1051 - RNA, 19 nt, Artificial Sequence
  Description of Artificial Sequence Synthetic oligonucleotide
aagugucggaacucuuuaa

SEQ ID NO: 1052 - RNA, 19 nt, Artificial Sequence
  Description of Artificial Sequence Synthetic oligonucleotide
aacgucacaacgaucccug

SEQ ID NO: 1053 - RNA, 19 nt, Artificial Sequence
  Description of Artificial Sequence Synthetic oligonucleotide
aaguguuaucuguaaucca

SEQ ID NO: 1054 - RNA, 19 nt, Artificial Sequence
  Description of Artificial Sequence Synthetic oligonucleotide
aaucucuugagauucaaau

SEQ ID NO: 1055 - RNA, 19 nt, Artificial Sequence
  Description of Artificial Sequence Synthetic oligonucleotide
uaaccuaccaucugggguu

SEQ ID NO: 1056 - RNA, 19 nt, Artificial Sequence
  Description of Artificial Sequence Synthetic oligonucleotide
uuaaccuaccaucuggggu

SEQ ID NO: 1057 - RNA, 19 nt, Artificial Sequence
  Description of Artificial Sequence Synthetic oligonucleotide
uuuaaccuaccaucugggg

SEQ ID NO: 1058 - RNA, 19 nt, Artificial Sequence
  Description of Artificial Sequence Synthetic oligonucleotide
auaccauggcaccuccaug

SEQ ID NO: 1059 - RNA, 19 nt, Artificial Sequence
  Description of Artificial Sequence Synthetic oligonucleotide
aaccucguugcuugaauau

SEQ ID NO: 1060 - RNA, 19 nt, Artificial Sequence
  Description of Artificial Sequence Synthetic oligonucleotide
aaaccucguugcuugaaua

SEQ ID NO: 1061 - RNA, 19 nt, Artificial Sequence
  Description of Artificial Sequence Synthetic oligonucleotide
uaaaccucguugcuugaau

SEQ ID NO: 1062 - RNA, 19 nt, Artificial Sequence
  Description of Artificial Sequence Synthetic oligonucleotide
aaaguauaucagcugacag

SEQ ID NO: 1063 - RNA, 19 nt, Artificial Sequence
  Description of Artificial Sequence Synthetic oligonucleotide
aacagguaaccauacuuua

SEQ ID NO: 1064 - DNA, 2037 bp, Rattus norvegicus
cgggcagatgccaacatggcagcggttggggttggcggatctaccgctgcggccggggcaggggctgtgtccgcgggcgcgttggaacctggatctgctacagcggctcaccggcgcctcaagtatatatccttagctgtgctggtggtccagaatgcctccctcatcctcagcatccgctatgctcgcacactgcctggggaccgcttctttgccaccactgctgtggtcatggctgaagtgctcaaaggtctcacctgtctcctgctgctcttcgcacaaaagagggggaatgtgaagcacctggtcctcttccttcacgaggctgtcctggtgcaatatgtcgacacactgaagctcgcggtgccctctctcatctataccttgcagaataacctccagtatgttgccatctccaacctgccagctgccactttccaggtgacgtatcagctgaagatcctgactacagcactgttctcggtgcttatgttgaatcgcagcctctcacgcctgcagtgggcctctctgctactgctcttcactggtgtcgccattgtccaggcacagcaagctggtgggagtggcccacggccactggatcagaacccgggggtgggcttagcagctgttgtggcctcctgtctctcctcaggctttgctggtgtctactttgagaagatcctcaaaggcagctcaggttctgtgtggctgcgcaacctccagctcggcctctttggcacagcgctgggcctggtggggctctggtgggctgagggcactgccgtggccagtcaaggcttcttctttgggtacacacctgctgtctggggtgtcgtactaaaccaagccttcggtgggctcctggtggctgttgttgtcaagtatgctgacaacatcctcaagggctttgccacctccctgtccattgtgctgtccactgttgcctccattcgcctctttggcttccacctggacccattatttgccctgggtgcagggctcgtcattggtgccgtctacctctacagccttccccgaggtgcagtcaaagccatagcctctgcctctgcctctgcctctgggccgtgcattcaccagcagcctcctgggcagccaccaccaccgcagctgtcttcccgaggagacctcatcacggagccctttctgccaaagtcagtgctggtgaagtgagagctggtggtggttggggaaacagggcagggggttgggttggagggggttgggcttctgcaggtcctgcaggttgtcgccagggcctgactcttgtggggttggaggttttttttctcccgtacttctaaagggatatggagctagggctgaatgtcacatgaacgcttcctgatagatggactcccctctcctggaggagctttttagagctgcttcctctgcctcgggctaacctctttgggaacaggggttggggtactgctatttcaggcctctccctcacgaccctctgctggagatgtcctgtctcacatgcctgggacagttcatcccagccatcctgctgactggacaaaagccccgcagctcttcagtaacgactaatgactactcgtggggttccatttcctattgtatgaggccttctctcctgcaccatcaccctggatcatgacaacagcttggtctctgatgtggctttgggccagtttccctggtacagagacccttgaagatcaatcagcctgttgttgctcaccaaggtgaagggctcgtagctgctggaattgaagacgctggcctgccttcgttctcccttcttgccctggcccagctgggactaaactcttatcagtattaggggtagggtgaggtagacatggaactaactccctgtccccaccaaccctgccccacgtagggctgacatgactgactaacctctgttaatgggcccacctcaactcctgctatctttacagtatttcttaggtgagtttctgcaaataaaatgtgttttgcatcttgtgaaaaaaaaaaaaaaaaaaaaaaaaaaaaaaa

SEQ ID NO: 1065 - RNA, 19 nt, Artificial Sequence
  Description of Artificial Sequence Synthetic oligonucleotide
accggcgccucaaguauau

SEQ ID NO: 1066 - RNA, 19 nt, Artificial Sequence
  Description of Artificial Sequence Synthetic oligonucleotide
ccggcgccucaaguauaua

SEQ ID NO: 1067 - RNA, 19 nt, Artificial Sequence
  Description of Artificial Sequence Synthetic oligonucleotide
cggcgccucaaguauauau

SEQ ID NO: 1068 - RNA, 19 nt, Artificial Sequence
  Description of Artificial Sequence Synthetic oligonucleotide
gccucaaguauauauccuu

SEQ ID NO: 1069 - RNA, 19 nt, Artificial Sequence
  Description of Artificial Sequence Synthetic oligonucleotide
ggugcccucucucaucuau

SEQ ID NO: 1070 - RNA, 19 nt, Artificial Sequence
  Description of Artificial Sequence Synthetic oligonucleotide
gcagaauaaccuccaguau

SEQ ID NO: 1071 - RNA, 19 nt, Artificial Sequence
  Description of Artificial Sequence Synthetic oligonucleotide
ugucuggggugucguacua

SEQ ID NO: 1072 - RNA, 19 nt, Artificial Sequence
  Description of Artificial Sequence Synthetic oligonucleotide
gucuggggugucguacuaa

SEQ ID NO: 1073 - RNA, 19 nt, Artificial Sequence
  Description of Artificial Sequence Synthetic oligonucleotide
ucuggggugucguacuaaa

SEQ ID NO: 1074 - RNA, 19 nt, Artificial Sequence
  Description of Artificial Sequence Synthetic oligonucleotide
gggugucguacuaaaccaa

SEQ ID NO: 1075 - RNA, 19 nt, Artificial Sequence
  Description of Artificial Sequence Synthetic oligonucleotide
uuccaccuggacccauuau

SEQ ID NO: 1076 - RNA, 19 nt, Artificial Sequence
  Description of Artificial Sequence Synthetic oligonucleotide
uccaccuggacccauuauu

SEQ ID NO: 1077 - RNA, 19 nt, Artificial Sequence
  Description of Artificial Sequence Synthetic oligonucleotide
ugaacgcuuccugauagau

SEQ ID NO: 1078 - RNA, 19 nt, Artificial Sequence
  Description of Artificial Sequence Synthetic oligonucleotide
cucuuugggaacagggguu

SEQ ID NO: 1079 - RNA, 19 nt, Artificial Sequence
  Description of Artificial Sequence Synthetic oligonucleotide
agcucuucaguaacgacua

SEQ ID NO: 1080 - RNA, 19 nt, Artificial Sequence
  Description of Artificial Sequence Synthetic oligonucleotide
aaugacuacucgugggguu

SEQ ID NO: 1081 - RNA, 19 nt, Artificial Sequence
  Description of Artificial Sequence Synthetic oligonucleotide
guuccauuuccuauuguau

SEQ ID NO: 1082 - RNA, 19 nt, Artificial Sequence
  Description of Artificial Sequence Synthetic oligonucleotide
ucacccuggaucaugacaa

SEQ ID NO: 1083 - RNA, 19 nt, Artificial Sequence
  Description of Artificial Sequence Synthetic oligonucleotide
gcugggacuaaacucuuau

SEQ ID NO: 1084 - RNA, 19 nt, Artificial Sequence
  Description of Artificial Sequence Synthetic oligonucleotide
gacuaaacucuuaucagua

SEQ ID NO: 1085 - RNA, 19 nt, Artificial Sequence
  Description of Artificial Sequence Synthetic oligonucleotide
acuaaacucuuaucaguau

SEQ ID NO: 1086 - RNA, 19 nt, Artificial Sequence
  Description of Artificial Sequence Synthetic oligonucleotide
gacugacuaaccucuguua

SEQ ID NO: 1087 - RNA, 19 nt, Artificial Sequence
  Description of Artificial Sequence Synthetic oligonucleotide
uccugcuaucuuuacagua

SEQ ID NO: 1088 - RNA, 19 nt, Artificial Sequence
  Description of Artificial Sequence Synthetic oligonucleotide
ccugcuaucuuuacaguau

SEQ ID NO: 1089 - RNA, 19 nt, Artificial Sequence
  Description of Artificial Sequence Synthetic oligonucleotide
guauuucuuaggugaguuu

SEQ ID NO: 1090 - RNA, 19 nt, Artificial Sequence
  Description of Artificial Sequence Synthetic oligonucleotide
auauacuugaggcgccggu

SEQ ID NO: 1091 - RNA, 19 nt, Artificial Sequence
  Description of Artificial Sequence Synthetic oligonucleotide
uauauacuugaggcgccgg

SEQ ID NO: 1092 - RNA, 19 nt, Artificial Sequence
  Description of Artificial Sequence Synthetic oligonucleotide
auauauacuugaggcgccg

SEQ ID NO: 1093 - RNA, 19 nt, Artificial Sequence
  Description of Artificial Sequence Synthetic oligonucleotide
aaggauauauacuugaggc

SEQ ID NO: 1094 - RNA, 19 nt, Artificial Sequence
  Description of Artificial Sequence Synthetic oligonucleotide
auagaugagagagggcacc

SEQ ID NO: 1095 - RNA, 19 nt, Artificial Sequence
  Description of Artificial Sequence Synthetic oligonucleotide
auacuggagguuauucugc

SEQ ID NO: 1096 - RNA, 19 nt, Artificial Sequence
  Description of Artificial Sequence Synthetic oligonucleotide
uaguacgacaccccagaca

SEQ ID NO: 1097 - RNA, 19 nt, Artificial Sequence
  Description of Artificial Sequence Synthetic oligonucleotide
uuaguacgacaccccagac

SEQ ID NO: 1098 - RNA, 19 nt, Artificial Sequence
  Description of Artificial Sequence Synthetic oligonucleotide
uuuaguacgacaccccaga

SEQ ID NO: 1099 - RNA, 19 nt, Artificial Sequence
  Description of Artificial Sequence Synthetic oligonucleotide
uugguuuaguacgacaccc

SEQ ID NO: 1100 - RNA, 19 nt, Artificial Sequence
  Description of Artificial Sequence Synthetic oligonucleotide
auaauggguccagguggaa

SEQ ID NO: 1101 - RNA, 19 nt, Artificial Sequence
  Description of Artificial Sequence Synthetic oligonucleotide
aauaauggguccaggugga

SEQ ID NO: 1102 - RNA, 19 nt, Artificial Sequence
  Description of Artificial Sequence Synthetic oligonucleotide
aucuaucaggaagcguuca

SEQ ID NO: 1103 - RNA, 19 nt, Artificial Sequence
  Description of Artificial Sequence Synthetic oligonucleotide
aaccccuguucccaaagag

SEQ ID NO: 1104 - RNA, 19 nt, Artificial Sequence
  Description of Artificial Sequence Synthetic oligonucleotide
uagucguuacugaagagcu

SEQ ID NO: 1105 - RNA, 19 nt, Artificial Sequence
  Description of Artificial Sequence Synthetic oligonucleotide
aaccccacgaguagucauu

SEQ ID NO: 1106 - RNA, 19 nt, Artificial Sequence
  Description of Artificial Sequence Synthetic oligonucleotide
auacaauaggaaauggaac

SEQ ID NO: 1107 - RNA, 19 nt, Artificial Sequence
  Description of Artificial Sequence Synthetic oligonucleotide
uugucaugauccaggguga

SEQ ID NO: 1108 - RNA, 19 nt, Artificial Sequence
  Description of Artificial Sequence Synthetic oligonucleotide
auaagaguuuagucccagc

SEQ ID NO: 1109 - RNA, 19 nt, Artificial Sequence
  Description of Artificial Sequence Synthetic oligonucleotide
uacugauaagaguuuaguc

SEQ ID NO: 1110 - RNA, 19 nt, Artificial Sequence
  Description of Artificial Sequence Synthetic oligonucleotide
auacugauaagaguuuagu

SEQ ID NO: 1111 - RNA, 19 nt, Artificial Sequence
  Description of Artificial Sequence Synthetic oligonucleotide
uaacagagguuagucaguc

SEQ ID NO: 1112 - RNA, 19 nt, Artificial Sequence
  Description of Artificial Sequence Synthetic oligonucleotide
uacuguaaagauagcagga

SEQ ID NO: 1113 - RNA, 19 nt, Artificial Sequence
  Description of Artificial Sequence Synthetic oligonucleotide
auacuguaaagauagcagg

SEQ ID NO: 1114 - RNA, 19 nt, Artificial Sequence
  Description of Artificial Sequence Synthetic oligonucleotide
aaacucaccuaagaaauac

SEQ ID NO: 1115 - DNA, 1763 bp, Homo sapiens
cgggccgggcagatgccaacatggcagcggttggggctggtggttccaccgcggcgcccgggccaggggcggtttccgcgggtgcattggagccggggaccgccagtgcggctcacaggcgcctgaagtacatatccctagctgtgctggtggtccagaatgcctccctcatcctcagcatccgctacgcccgcacgttgccaggggaccgcttctttgccaccactgctgtggtcatggcggaagtgctcaaaggtctcacctgcctgctgctgctcttcgcacagaagaggggtaacgtgaagcacctggttctcttcctccatgaggctgtcctggtgcagtatgtggacacgctcaagctcgcagtgccctctctcatctacaccttgcagaataacctccagtatgttgccatctctaacctaccagctgccactttccagccttccccgaggtgcagccaaagccatagcctctgcctctgcctccgcctccgggccctgcgttcaccagcagcctcccgggcagccaccaccaccgcagctgtcttcccaccgtggagacctcatcacggagccctttctgccaaagtcagtgctggtgaagtgagggctggcagcaatggggggacacaagggagggggactggggtggagggtgttgggcatctgcaggacccaagtcgccaccctccggggcctggctcctctgggtttgggagatggtcttttctcccaggtcactgagacttctggaggggtgtgggactagagctgggtgtcacgtgaacccttcctggtagggtgacccccttcccctggagggggttttagagctgccgcctctgctccctctaacctctttggaggcagggttgggggtattgtcattcaaggccttttttttgtctgctccctccccgaccctgtgccctcttctggaggtttctcgtctgggagagtccctcccagcagtccctccacctccataaggacacactggacaaaactcccgcagctcttcaggaatgaccgatgcctacctgtggggttcagttgcccatagtttgaggccttctctcctcccttaccaccgctctggatcatgttactagttccgtcttttgtgtggccttgggccagcttccttgataccttgaagatgggctccttgtgagtccccagggagaaagggacaagagctaagatttttgcatcagcccttctggcagaaggtgtggtaggggccatttgttttttttagtggacttgggatttgtggtgtaatcatatcattaatgatccagggtgtgggaaaaatggaggtccttgaagtggctgaatctcattgtatttaagacactgtcagttgccagatgtaggcttatttttggagatgtctaggagaggaaaaagctaccaatcatactcttgatatccgtctggctgtgtgaggcacccctacctcatgggggtgtcttgggattgatgaactgtggaacctgcctcctgcgctccccaaagcttattaaccccttaactgtatcggggcggggtgtgtgtgtgcatggaagatgcctgggctgtctttgctatatgtaaatagagccattggatctttatttttgattaatttgttctgattttttggtttgttttttaaggaactgtaatgaacaaatgtcaggatatccaatgccaaataaagatgttgtatttttttaaaaaaaaaaaaaaaaaaaaaaaaaaaaaaaaaaaaaa

SEQ ID NO: 1116 - RNA, 19 nt, Artificial Sequence
  Description of Artificial Sequence Synthetic oligonucleotide
gccugaaguacauaucccu

SEQ ID NO: 1117 - RNA, 19 nt, Artificial Sequence
  Description of Artificial Sequence Synthetic oligonucleotide
ccugaaguacauaucccua

SEQ ID NO: 1118 - RNA, 19 nt, Artificial Sequence
  Description of Artificial Sequence Synthetic oligonucleotide
gaaguacauaucccuagcu

SEQ ID NO: 1119 - RNA, 19 nt, Artificial Sequence
  Description of Artificial Sequence Synthetic oligonucleotide
aguacauaucccuagcugu

SEQ ID NO: 1120 - RNA, 19 nt, Artificial Sequence
  Description of Artificial Sequence Synthetic oligonucleotide
acauaucccuagcugugcu

SEQ ID NO: 1121 - RNA, 19 nt, Artificial Sequence
  Description of Artificial Sequence Synthetic oligonucleotide
ucauccucagcauccgcua

SEQ ID NO: 1122 - RNA, 19 nt, Artificial Sequence
  Description of Artificial Sequence Synthetic oligonucleotide
gugcucaaaggucucaccu

SEQ ID NO: 1123 - RNA, 19 nt, Artificial Sequence
  Description of Artificial Sequence Synthetic oligonucleotide
cugcucuucgcacagaaga

SEQ ID NO: 1124 - RNA, 19 nt, Artificial Sequence
  Description of Artificial Sequence Synthetic oligonucleotide
uucgcacagaagaggggua

SEQ ID NO: 1125 - RNA, 19 nt, Artificial Sequence
  Description of Artificial Sequence Synthetic oligonucleotide
cacagaagagggguaacgu

SEQ ID NO: 1126 - RNA, 19 nt, Artificial Sequence
  Description of Artificial Sequence Synthetic oligonucleotide
cagaagagggguaacguga

SEQ ID NO: 1127 - RNA, 19 nt, Artificial Sequence
  Description of Artificial Sequence Synthetic oligonucleotide
uaacgugaagcaccugguu

SEQ ID NO: 1128 - RNA, 19 nt, Artificial Sequence
  Description of Artificial Sequence Synthetic oligonucleotide
caguauguggacacgcuca

SEQ ID NO: 1129 - RNA, 19 nt, Artificial Sequence
  Description of Artificial Sequence Synthetic oligonucleotide
auguggacacgcucaagcu

SEQ ID NO: 1130 - RNA, 19 nt, Artificial Sequence
  Description of Artificial Sequence Synthetic oligonucleotide
ccucucucaucuacaccuu

SEQ ID NO: 1131 - RNA, 19 nt, Artificial Sequence
  Description of Artificial Sequence Synthetic oligonucleotide
cucaucuacaccuugcaga

SEQ ID NO: 1132 - RNA, 19 nt, Artificial Sequence
  Description of Artificial Sequence Synthetic oligonucleotide
acaccuugcagaauaaccu

SEQ ID NO: 1133 - RNA, 19 nt, Artificial Sequence
  Description of Artificial Sequence Synthetic oligonucleotide
ccuugcagaauaaccucca

SEQ ID NO: 1134 - RNA, 19 nt, Artificial Sequence
  Description of Artificial Sequence Synthetic oligonucleotide
gcagaauaaccuccaguau

SEQ ID NO: 1135 - RNA, 19 nt, Artificial Sequence
  Description of Artificial Sequence Synthetic oligonucleotide
agaauaaccuccaguaugu

SEQ ID NO: 1136 - RNA, 19 nt, Artificial Sequence
  Description of Artificial Sequence Synthetic oligonucleotide
gaauaaccuccaguauguu

SEQ ID NO: 1137 - RNA, 19 nt, Artificial Sequence
  Description of Artificial Sequence Synthetic oligonucleotide
accuccaguauguugccau

SEQ ID NO: 1138 - RNA, 19 nt, Artificial Sequence
  Description of Artificial Sequence Synthetic oligonucleotide
ugccaucucuaaccuacca

SEQ ID NO: 1139 - RNA, 19 nt, Artificial Sequence
  Description of Artificial Sequence Synthetic oligonucleotide
caucucuaaccuaccagcu

SEQ ID NO: 1140 - RNA, 19 nt, Artificial Sequence
  Description of Artificial Sequence Synthetic oligonucleotide
accuaccagcugccacuuu

SEQ ID NO: 1141 - RNA, 19 nt, Artificial Sequence
  Description of Artificial Sequence Synthetic oligonucleotide
uaccagcugccacuuucca

SEQ ID NO: 1142 - RNA, 19 nt, Artificial Sequence
  Description of Artificial Sequence Synthetic oligonucleotide
agggauauguacuucaggc

SEQ ID NO: 1143 - RNA, 19 nt, Artificial Sequence
  Description of Artificial Sequence Synthetic oligonucleotide
uagggauauguacuucagg

SEQ ID NO: 1144 - RNA, 19 nt, Artificial Sequence
  Description of Artificial Sequence Synthetic oligonucleotide
agcuagggauauguacuuc

SEQ ID NO: 1145 - RNA, 19 nt, Artificial Sequence
  Description of Artificial Sequence Synthetic oligonucleotide
acagcuagggauauguacu

SEQ ID NO: 1146 - RNA, 19 nt, Artificial Sequence
  Description of Artificial Sequence Synthetic oligonucleotide
agcacagcuagggauaugu

SEQ ID NO: 1147 - RNA, 19 nt, Artificial Sequence
  Description of Artificial Sequence Synthetic oligonucleotide
uagcggaugcugaggauga

SEQ ID NO: 1148 - RNA, 19 nt, Artificial Sequence
  Description of Artificial Sequence Synthetic oligonucleotide
aggugagaccuuugagcac

SEQ ID NO: 1149 - RNA, 19 nt, Artificial Sequence
  Description of Artificial Sequence Synthetic oligonucleotide
ucuucugugcgaagagcag

SEQ ID NO: 1150 - RNA, 19 nt, Artificial Sequence
  Description of Artificial Sequence Synthetic oligonucleotide
uaccccucuucugugcgaa

SEQ ID NO: 1151 - RNA, 19 nt, Artificial Sequence
  Description of Artificial Sequence Synthetic oligonucleotide
acguuaccccucuucugug

SEQ ID NO: 1152 - RNA, 19 nt, Artificial Sequence
  Description of Artificial Sequence Synthetic oligonucleotide
ucacguuaccccucuucug

SEQ ID NO: 1153 - RNA, 19 nt, Artificial Sequence
  Description of Artificial Sequence Synthetic oligonucleotide
aaccaggugcuucacguua

SEQ ID NO: 1154 - RNA, 19 nt, Artificial Sequence
  Description of Artificial Sequence Synthetic oligonucleotide
ugagcguguccacauacug

SEQ ID NO: 1155 - RNA, 19 nt, Artificial Sequence
  Description of Artificial Sequence Synthetic oligonucleotide
agcuugagcguguccacau

SEQ ID NO: 1156 - RNA, 19 nt, Artificial Sequence
  Description of Artificial Sequence Synthetic oligonucleotide
aagguguagaugagagagg

SEQ ID NO: 1157 - RNA, 19 nt, Artificial Sequence
  Description of Artificial Sequence Synthetic oligonucleotide
ucugcaagguguagaugag

SEQ ID NO: 1158 - RNA, 19 nt, Artificial Sequence
  Description of Artificial Sequence Synthetic oligonucleotide
agguuauucugcaaggugu

SEQ ID NO: 1159 - RNA, 19 nt, Artificial Sequence
  Description of Artificial Sequence Synthetic oligonucleotide
uggagguuauucugcaagg

SEQ ID NO: 1160 - RNA, 19 nt, Artificial Sequence
  Description of Artificial Sequence Synthetic oligonucleotide
auacuggagguuauucugc

SEQ ID NO: 1161 - RNA, 19 nt, Artificial Sequence
  Description of Artificial Sequence Synthetic oligonucleotide
acauacuggagguuauucu

SEQ ID NO: 1162 - RNA, 19 nt, Artificial Sequence
  Description of Artificial Sequence Synthetic oligonucleotide
aacauacuggagguuauuc

SEQ ID NO: 1163 - RNA, 19 nt, Artificial Sequence
  Description of Artificial Sequence Synthetic oligonucleotide
auggcaacauacuggaggu

SEQ ID NO: 1164 - RNA, 19 nt, Artificial Sequence
  Description of Artificial Sequence Synthetic oligonucleotide
ugguagguuagagauggca

SEQ ID NO: 1165 - RNA, 19 nt, Artificial Sequence
  Description of Artificial Sequence Synthetic oligonucleotide
agcugguagguuagagaug

SEQ ID NO: 1166 - RNA, 19 nt, Artificial Sequence
  Description of Artificial Sequence Synthetic oligonucleotide
aaaguggcagcugguaggu

SEQ ID NO: 1167 - RNA, 19 nt, Artificial Sequence
  Description of Artificial Sequence Synthetic oligonucleotide
uggaaaguggcagcuggua

SEQ ID NO: 1168 - DNA, 1569 bp, Mus musculus
ccagctaacgccccgccccccccagaggtccggaaacggacgtgatggatacggaagtagcctgctgttgccgaggggacgggacgggcagatgccaacatggcagcggttggggttggtggatctaccgctgcggccggggctggggctgtgtcctcgggcgcgttggaacctgggtccactacagcggctcaccggcgcctcaagtatatatccttagctgtgctggtggtccagaatgcctccctcatcctcagcatccgctacgctcgcacactgcctggggaccgcttctttgccaccactgctgtggtcatggctgaagtgctcaaaggtctcacctgtctcctgctgctcttcgcacaaaagaggggtaatgtgaagcacctggtcctcttcctccatgaggctgtcctggtccaatatgtggacacactcaagcttgcggtgccctctctcatctataccttgcagaataacctccagtatgttgccatcagcaacctgccagctgccactttccaggtgacatatcagctgaagatcctgactacagcgctcttctctgtgctcatgttgaatcgcagcctctcacgcctgcagtgggcctctctgctgctgctcttcactggtgtcgccattgtccaggcacagcaagctggtgggagtggcccacggccactggatcagaacccgggggcgggcttagcggcagttgtggcctcctgtctctcctcaggcttcgctggggtctactttgagaagatcctcaaaggcagctcaggttctgtgtggcttcgtaacctacagctcggcctctttggcacagcgctgggcctggtggggctctggtgggctgagggcaccgccgtggccagtcaaggcttcttctttgggtacacacctgctgtctggggtgtagtactaaaccaagcctttggtgggcttctggtggctgttgttgtcaagtatgctgacaacatcctcaagggctttgccacctccctgtctattgtgctgtccactgttgcctccattcgcctctttggcttccacctggacccattatttgccctgggcgctgggctcgtcattggtgctgtctacctctacagccttccccgaggtgcagtcaaagccatagcctcggcctcggcctctgggccctgcattcaccagcagcctcctgggcagccaccaccaccgcagctgtcttctcgaggagacctcaccacggagccctttctgccaaagttgctcaccaaggtgaaggggtcgtagctgctggacttgaagatgctggcctgtcttcgttctcccttcttgccctggcccaactgggactaaactcttatcagtattaggggtagggtgaggtagacacgggaactccctgtccttaccaacccctgccccacatagggctgacatgactaacctctgttaatgggcccacctctactcctgctatctttacagtatttcttaggtgagtttctgcaaataaaatgttttgcaccttgtgaagttgggttggaaagcctggaaaggatgaaaaaaaaaaaaaaa

SEQ ID NO: 1169 - RNA, 19 nt, Artificial Sequence
  Description of Artificial Sequence Synthetic oligonucleotide
gaaacggacgugauggaua

SEQ ID NO: 1170 - RNA, 19 nt, Artificial Sequence
  Description of Artificial Sequence Synthetic oligonucleotide
uuggaaccuggguccacua

SEQ ID NO: 1171 - RNA, 19 nt, Artificial Sequence
  Description of Artificial Sequence Synthetic oligonucleotide
gccucaaguauauauccuu

SEQ ID NO: 1172 - RNA, 19 nt, Artificial Sequence
  Description of Artificial Sequence Synthetic oligonucleotide
aguauauauccuuagcugu

SEQ ID NO: 1173 - RNA, 19 nt, Artificial Sequence
  Description of Artificial Sequence Synthetic oligonucleotide
gugcucaaaggucucaccu

SEQ ID NO: 1174 - RNA, 19 nt, Artificial Sequence
  Description of Artificial Sequence Synthetic oligonucleotide
cugcugcucuucgcacaaa

SEQ ID NO: 1175 - RNA, 19 nt, Artificial Sequence
  Description of Artificial Sequence Synthetic oligonucleotide
ugcugcucuucgcacaaaa

SEQ ID NO: 1176 - RNA, 19 nt, Artificial Sequence
  Description of Artificial Sequence Synthetic oligonucleotide
cugcucuucgcacaaaaga

SEQ ID NO: 1177 - RNA, 19 nt, Artificial Sequence
  Description of Artificial Sequence Synthetic oligonucleotide
cccucucucaucuauaccu

SEQ ID NO: 1178 - RNA, 19 nt, Artificial Sequence
  Description of Artificial Sequence Synthetic oligonucleotide
cucucaucuauaccuugca

SEQ ID NO: 1179 - RNA, 19 nt, Artificial Sequence
  Description of Artificial Sequence Synthetic oligonucleotide
uugcagaauaaccuccagu

SEQ ID NO: 1180 - RNA, 19 nt, Artificial Sequence
  Description of Artificial Sequence Synthetic oligonucleotide
agauccugacuacagcgcu

SEQ ID NO: 1181 - RNA, 19 nt, Artificial Sequence
  Description of Artificial Sequence Synthetic oligonucleotide
guucuguguggcuucguaa

SEQ ID NO: 1182 - RNA, 19 nt, Artificial Sequence
  Description of Artificial Sequence Synthetic oligonucleotide
cuguguggcuucguaaccu

SEQ ID NO: 1183 - RNA, 19 nt, Artificial Sequence
  Description of Artificial Sequence Synthetic oligonucleotide
uguggcuucguaaccuaca

SEQ ID NO: 1184 - RNA, 19 nt, Artificial Sequence
  Description of Artificial Sequence Synthetic oligonucleotide
gucugggguguaguacuaa

SEQ ID NO: 1185 - RNA, 19 nt, Artificial Sequence
  Description of Artificial Sequence Synthetic oligonucleotide
gggguguaguacuaaacca

SEQ ID NO: 1186 - RNA, 19 nt, Artificial Sequence
  Description of Artificial Sequence Synthetic oligonucleotide
ggguguaguacuaaaccaa

SEQ ID NO: 1187 - RNA, 19 nt, Artificial Sequence
  Description of Artificial Sequence Synthetic oligonucleotide
guaguacuaaaccaagccu

SEQ ID NO: 1188 - RNA, 19 nt, Artificial Sequence
  Description of Artificial Sequence Synthetic oligonucleotide
uaguacuaaaccaagccuu

SEQ ID NO: 1189 - RNA, 19 nt, Artificial Sequence
  Description of Artificial Sequence Synthetic oligonucleotide
uccaccuggacccauuauu

SEQ ID NO: 1190 - RNA, 19 nt, Artificial Sequence
  Description of Artificial Sequence Synthetic oligonucleotide
augcuggccugucuucguu

SEQ ID NO: 1191 - RNA, 19 nt, Artificial Sequence
  Description of Artificial Sequence Synthetic oligonucleotide
aacugggacuaaacucuua

SEQ ID NO: 1192 - RNA, 19 nt, Artificial Sequence
  Description of Artificial Sequence Synthetic oligonucleotide
ggacuaaacucuuaucagu

SEQ ID NO: 1193 - RNA, 19 nt, Artificial Sequence
  Description of Artificial Sequence Synthetic oligonucleotide
cuaaacucuuaucaguauu

SEQ ID NO: 1194 - RNA, 19 nt, Artificial Sequence
  Description of Artificial Sequence Synthetic oligonucleotide
cuuaucaguauuaggggua

SEQ ID NO: 1195 - RNA, 19 nt, Artificial Sequence
  Description of Artificial Sequence Synthetic oligonucleotide
gggcugacaugacuaaccu

SEQ ID NO: 1196 - RNA, 19 nt, Artificial Sequence
  Description of Artificial Sequence Synthetic oligonucleotide
uaaugggcccaccucuacu

SEQ ID NO: 1197 - RNA, 19 nt, Artificial Sequence
  Description of Artificial Sequence Synthetic oligonucleotide
uauccaucacguccguuuc

SEQ ID NO: 1198 - RNA, 19 nt, Artificial Sequence
  Description of Artificial Sequence Synthetic oligonucleotide
uaguggacccagguuccaa

SEQ ID NO: 1199 - RNA, 19 nt, Artificial Sequence
  Description of Artificial Sequence Synthetic oligonucleotide
aaggauauauacuugaggc

SEQ ID NO: 1200 - RNA, 19 nt, Artificial Sequence
  Description of Artificial Sequence Synthetic oligonucleotide
acagcuaaggauauauacu

SEQ ID NO: 1201 - RNA, 19 nt, Artificial Sequence
  Description of Artificial Sequence Synthetic oligonucleotide
aggugagaccuuugagcac

SEQ ID NO: 1202 - RNA, 19 nt, Artificial Sequence
  Description of Artificial Sequence Synthetic oligonucleotide
uuugugcgaagagcagcag

SEQ ID NO: 1203 - RNA, 19 nt, Artificial Sequence
  Description of Artificial Sequence Synthetic oligonucleotide
uuuugugcgaagagcagca

SEQ ID NO: 1204 - RNA, 19 nt, Artificial Sequence
  Description of Artificial Sequence Synthetic oligonucleotide
ucuuuugugcgaagagcag

SEQ ID NO: 1205 - RNA, 19 nt, Artificial Sequence
  Description of Artificial Sequence Synthetic oligonucleotide
agguauagaugagagaggg

SEQ ID NO: 1206 - RNA, 19 nt, Artificial Sequence
  Description of Artificial Sequence Synthetic oligonucleotide
ugcaagguauagaugagag

SEQ ID NO: 1207 - RNA, 19 nt, Artificial Sequence
  Description of Artificial Sequence Synthetic oligonucleotide
acuggagguuauucugcaa

SEQ ID NO: 1208 - RNA, 19 nt, Artificial Sequence
  Description of Artificial Sequence Synthetic oligonucleotide
agcgcuguagucaggaucu

SEQ ID NO: 1209 - RNA, 19 nt, Artificial Sequence
  Description of Artificial Sequence Synthetic oligonucleotide
uuacgaagccacacagaac

SEQ ID NO: 1210 - RNA, 19 nt, Artificial Sequence
  Description of Artificial Sequence Synthetic oligonucleotide
agguuacgaagccacacag

SEQ ID NO: 1211 - RNA, 19 nt, Artificial Sequence
  Description of Artificial Sequence Synthetic oligonucleotide
uguagguuacgaagccaca

SEQ ID NO: 1212 - RNA, 19 nt, Artificial Sequence
  Description of Artificial Sequence Synthetic oligonucleotide
uuaguacuacaccccagac

SEQ ID NO: 1213 - RNA, 19 nt, Artificial Sequence
  Description of Artificial Sequence Synthetic oligonucleotide
ugguuuaguacuacacccc

SEQ ID NO: 1214 - RNA, 19 nt, Artificial Sequence
  Description of Artificial Sequence Synthetic oligonucleotide
uugguuuaguacuacaccc

SEQ ID NO: 1215 - RNA, 19 nt, Artificial Sequence
  Description of Artificial Sequence Synthetic oligonucleotide
aggcuugguuuaguacuac

SEQ ID NO: 1216 - RNA, 19 nt, Artificial Sequence
  Description of Artificial Sequence Synthetic oligonucleotide
aaggcuugguuuaguacua

SEQ ID NO: 1217 - RNA, 19 nt, Artificial Sequence
  Description of Artificial Sequence Synthetic oligonucleotide
aauaauggguccaggugga

SEQ ID NO: 1218 - RNA, 19 nt, Artificial Sequence
  Description of Artificial Sequence Synthetic oligonucleotide
aacgaagacaggccagcau

SEQ ID NO: 1219 - RNA, 19 nt, Artificial Sequence
  Description of Artificial Sequence Synthetic oligonucleotide
uaagaguuuagucccaguu

SEQ ID NO: 1220 - RNA, 19 nt, Artificial Sequence
  Description of Artificial Sequence Synthetic oligonucleotide
acugauaagaguuuagucc

SEQ ID NO: 1221 - RNA, 19 nt, Artificial Sequence
  Description of Artificial Sequence Synthetic oligonucleotide
aauacugauaagaguuuag

SEQ ID NO: 1222 - RNA, 19 nt, Artificial Sequence
  Description of Artificial Sequence Synthetic oligonucleotide
uaccccuaauacugauaag

SEQ ID NO: 1223 - RNA, 19 nt, Artificial Sequence
  Description of Artificial Sequence Synthetic oligonucleotide
agguuagucaugucagccc

SEQ ID NO: 1224 - RNA, 19 nt, Artificial Sequence
  Description of Artificial Sequence Synthetic oligonucleotide
aguagaggugggcccauua

SEQ ID NO: 1225 - DNA, 1551 bp, Homo sapiens
atggctggcagcctcacaggtttgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcatcgccagtga

SEQ ID NO: 1226 - protein, 516 aa, Homo sapiens
MAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 1227 - DNA, 6122 bp, Artificial Sequence
  Description of Artificial Sequence Synthetic polynucleotide
ctagagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctttatccccgctgccatcatggttcgaccattgaactgcatcgtcgccgtgtcccaagatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgggacttttgctggctttagatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaattgtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattctacgggccagatttacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgaagcaaatgggcggtaggcgtgtacggtgggaggtctatataagcaggagctcgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagtcggagattgggctcgcaccgctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgcttgcaatcgcggccgcaggcgcgccggatccgctgatcagcctcgactgtgccttctagttgccagccatctgttgtttgcccctcccccgtgccttccttgaccctggaaggtgccactctcactgtcctttcctaataaaatgaggaaattgcatcgcattgtctgagtaggtgtcattctattctggggggtggggtggggcaggacagcaagggggaggattgggaagacaatagcaggcatgctggggt

SEQ ID NO: 1228 - DNA, 2324 bp, Homo sapiens
acatcgggaagccggaattacttgcagggctaacctagtgcctatagctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggtggtgccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagcccaaaaaaaaaaaaaaaaaaaaa

SEQ ID NO: 1229 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 1230 - DNA, 5876 bp, Homo sapiens
agaacccgccccggaggggagggacgcagggaagagtcgcacggacgcactcgcgctgcggccagcgcccgggcctgcgggcccgggcggcggctgtgttgcgcagtcttcatgggttcccgacgaggaggtctctgtggctgcggcggcggctgctaactgcgccacctgctgcagcctgtccccgccgctctgaagcggccgcgtcgaagccgaaatgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttgagttttgccaaccatggatggcaaatgtgatgtgctcccttccagctggtgagaggaggagttagagctggtcgttttgtgattacccataatattggaagcagcctgagggctagttaatccaaacatgcatcaacaatttggcctgagaatatgtaacagccaaaccttttcgtttagtctttattaaaatttataattggtaattggaccagttttttttttaatttccctctttttaaaacagttacggcttatttactgaataaatacaaagcaaacaaactcaagttatgtcatacctttggatacgaagaccatacataataaccaaacataacattatacacaaagaatactttcattatttgtggaatttagtgcatttcaaaaagtaatcatatatcaaactaggcaccacactaagttcctgattattttgtttataatttaataatatatcttatgagccctatatattcaaaatattatgttaacatgtaatccatgtttctttttcaaatctaaagttaaaaaaaaatagcagaagccagtgtcttaaagtctatcttttgtttctaagaccatgggatttcataatctcaagataaaatatgtatgaagtaattaatgtagaatttttacaccaaataataaataatgcttaataaactagagatatgagatgtgtaggaaatttggttaaacttttttcagatactttctggcccaaataataatttgttagcaaataatatgacccttgaactcaatggccatctattaaaagactgttgttcacactggaaaacatttaaagatgtgactatatccatgggtggattgaatcactcaaaatatattagtatccttctttagggatggttggttacagacatgtatttattcaggaggcagaaaatattccattttaattgcttattaaagaaaacattaaattctaaattattttgaggactgtgaagacttttcattagtgtaatattaggtcattgtcaatctcccagaatgtagttctatattctctaaatatgaaagtatccagaaaggccagtggtagtaaaaagcttagtgtatataatctcaaaagggatggaatatttacagctcatatttataacatgttgaatcttctcagttatcagtagtcatcagaagtgtcaatagctttctaaataaatattaaatatctactgtcctgtagtgaaggagtaatttttagtaattttctctttacaaagtctccagtgtttccaggtaaatatttgtgaaacaaaatacagcaaactacattgttacttcagtgtattgttgccaaaaatgacaagatattatattaaaatcagtaaattttagacagattttaaaaattaattagcctacaatagaggttatatggtaacacggtgatcttctaagcagttaagtgactgactgttctggcaacaacgacttctccgtgactgaagggccctgttcatttcctgatcctgaagctcgtctctcttttgagcctccgcttgctttggtcgatggtttccctcagctttttctttgctgttcttcatcctcgttgttgctgtcatcatgttcactgtggcttttacaatacagcctgtaaattccttatgacatagttcagtgcatttggctttatcgcctgctccacagttctttacctttacttggcttagagaaactgtatctttgttgcttcatataacctttccccaaccccactaagctggacataacttattagtggtcctcccgtcactttatttgtagaaatctctctttcacatgagcaggggttctttcatgtggtttagctgacagcagaactagtgattctagacattttgcatggccctcattcagtggctcacaaacatgagggagcatcagaactacttgaggggcttgttaaaacccagtgcgttagaagtcggatgcggtggctcacacctgtaatcccagcactttgggaggcccaggcaggcggatcacttgaggttaggagttcaagaccagcctggccaacatggtgaaaccccgtctctactaaaaatacaaaagttagccgggtgtggtggtgcatgcctgtaatcccagcttcttgggaggccaaggcacaagaatcgcttgaaccaggagacggaggtttcagtgaatgaagatcgtgccattgtattccagcctcggcaacacagcaggactgtgattttctttggagactcctagattttctgtggttttgaactgaatttgttggatgttggcaagtgcctcttatgagctgtttctttatcctgcatttgccccacaaagacttatctggaggtgagcaaagtatgtttggtagtgaggtcacaaaggcaatcagccccttcctccccactcccattgccatcttctcagtccttctccctttctttccaagtagtttacccacccctcctctttcctcccctgtccctaaaataatccacgtgtcttcctaaaatctctctttgatcctgtcctttgataacaccgtcagtgcctactactgggtctagacagacctctgttgagcagtcagagtcttccctgactccacaatgcccctttccttggctgaccagtatgactactggtccccacctttcccttgcctatccctacctccctcctactaggttgtcccatccctctcttcacccattcattcatgaccatttttcactaccaagctccccccctcccgaaggaggctgaggtttttgtgactctctagactctattgtgggatggaatgaacattgctaaagaatcttgtgttcgctttactttaaaaaggtatttttttcctaattataaaactgatgtgtcagttacggaaaaattagaaatgcagcacaaatacatgaatattttaccacgaaattgccatataatatcttgtcttttttgggggtgtgaattttttgcattgttctgatcatattctttatcatgtaatttatgttcttttttactaagtattatgtgtggttattatagattttcacaaagatatattgctggtaatatattttattgtgtagtcttataatttacttaaccttctttcaattgttagaaatttaggctatttccagattttcagtattgtaaataatgctgtgatgaccaattttgtgaataaaatgtttttatgtatttcagattattcccttaggatagtctctcagtgccaagttgtcaaaaacatctctattttgcttatcttcctgctctcttgctgccttagggggtagtaaactgaaacataaagtaaacatgcatacaaataaaaaacataaaacaaaaataagcaacctgatggtaataggtgaaagtggtaacctgttttaactttgaattcttgccgggcgcggtggctcacgcctgtaatcccagcactttgggaggctgaggcgggtggatcacgaggtcaggagttcaaaaccagcctggccaagatggtgaaatcccgtctctactaaaaatacaaaaattagccgggcgtggtggcgggcgcctgtaatcccagctacttgggaggctgaggcagagaattgcttgaacccaggaggcggaggttgcagtgagccaagatcgcgccactgcactccagcctgggtgacagagcgagactccgtctcaaataaaaaacaacaaaaaacaaaaaaaacttaaaattctttgcttgttagtgaccttgatcatggttctctttgtacgatagttgggcatctgtatttccacttgtgtgaatttgcctttaaattttggttatgggtttcaccttttaaaataatcaaacatatttatcttttcctgtgtgataggtttttttctgtatcttttcctgttaaacacacagacccctccccaatctggacattgaataaatattcattttcctttgcattgttaaaaaaaaaaaaaaaaaaaaaaaa

SEQ ID NO: 1231 - protein, 550 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 1232 - DNA, 3782 bp, Homo sapiens
acccgcctctgcgcgcccccgggcacgaccccggagtctccgcgggcggccagggcgcgcgtgcgcggaggtgagccgggccggggctgcggggcttccctgagcgcgggccgggtcggtggggcggtcggctgcccgcgcggcctctcagttgggaaagctgaggttgtcgccggggccgcgggtggaggtcggggatgaggcagcaggtaggacagtgacctcggtgacgcgaaggaccccggccacctctaggttctcctcgtccgcccgttgttcagcgagggaggctctgcgcgtgccgcagctgacggggaaactgaggcacggagcgggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgcatgtgtcacctggagctgggcactaaccattccaagccgccgcatcgcttgtttccacctcctgggccggggctctggcccccaacgtgtctaggagagctttctccctagatcgcactgtgggccggggccctggagggctgctctgtgttaataagattgtaaggtttgccctcctcacctgttgccggcatgcgggtagtattagccacccccctccatctgttcccagcaccggagaagggggtgctcaggtggaggtgtggggtatgcacctgagctcctgcttcgcgcctgctgctctgccccaacgcgaccgctgcccggctgcccagagggctggatgcctgccggtccccgagcaagcctgggaactcaggaaaattcacaggacttgggagattctaaatcttaagtgcaattatttttaataaaaggggcatttggaatcaaaaaaaa

SEQ ID NO: 1233 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 1234 - DNA, 6076 bp, Homo sapiens
aaaagtgaatacatgattttatttaactcattaataaggaaattggtaaggtgttaaaaccaattcaaaggacaatccaaagaacagatcaggaatactaaaataaatatgcaagcggaggtgaaactgttttccttggtagtggtggaggggaaggattgctactccgctggataaagttcatttgtgtatatataaataagaattattttccattgttatttatctataacttataaagttgtaaacaacttccacggaatcagactcaacctggaagggtatggtctctaggcaatgcaaaaattttcccctacacctgttaacaactataatatctccagacagagtagacagaaagtctggatggcaacgggaatctactggtcatacggctaacttcctaattcaataagcacgtgactaaaggattttttccttccactcagatatttcaggctaactagatactgtgtgcttcttagtgtcactgcttagtgggggagccagctctgagtggggtcatatccggacaagcgaatgagctatttattcaatgaccacgcaacactccaaatcctcccagggcaacttgaaagtaaccgcaccttccaaagggcaccgtgcaatcagactgtgtgtttggcctcctgtttgctagtggggaggaagcggcttcatgggtgtacactacgcataaatgaatgtgaaaggctatttagacctctgccttttcaccgtcctcccacctgccacaggctgggctcttgtgctagaaatgacttgctagctagacatcatggttcaggatctgagtcagaggtttaaccatttataagcttttttcttatgaaaaattggcactaattataatgtctaactgtcagagttgttgcaggctttacaggagacgcgggctgtgaagatgctttgtaaattgtgaagcgttattaaagaacacatcttttttttttaggaaaccacagtgcaaatttaattgccggggaagataacgggccttggtgccctccaagcgtcagctgagtttccaagaagccgggcagcgggcgcccgcgggttcgtctctggctcctcctccgccacagcagccgggggcccgggtcggaggcggcgggggccgagcgcccggcctcgcaagcccacggcccgctgggggtgccgtcccgcgccggggcggagcaggccccggcagcccagttcctcattctatcagcggtacaaggggctggtggcgccacaggcgctgggaccgcgggcggacaaggatgggtccgcgcggcgcggcgagcttgccccgaggccccggacctcggcggctgctcctccccgtcgtcctcccgctgctgctgctgctgttgttggcgccgccgggctcgggcgccggggccagccggccgccccacctggtcttcttgctggcagacgacctaggctggaacgacgtcggcttccacggctcccgcatccgcacgccgcacctggacgcgctggcggccggcggggtgctcctggacaactactacacgcagccgctgtgcacgccgtcgcggagccagctgctcactggccgctaccagatccgtacaggtttacagcaccaaataatctggccctgtcagcccagctgtgttcctctggatgaaaaactcctgccccagctcctaaaagaagcaggttatactacccatatggtcggaaaatggcacctgggaatgtaccggaaagaatgccttccaacccgccgaggatttgatacctactttggatatctcctgggtagtgaagattattattcccatgaacgctgtacattaattgacgctctgaatgtcacacgatgtgctcttgattttcgagatggcgaagaagttgcaacaggatataaaaatatgtattcaacaaacatattcaccaaaagggctatagccctcataactaaccatccaccagagaagcctctgtttctctaccttgctctccagtctgtgcatgagccccttcaggtccctgaggaatacttgaagccatatgactttatccaagacaagaacaggcatcactatgcaggaatggtgtcccttatggatgaagcagtaggaaatgtcactgcagctttaaaaagcagtgggctctggaacaacacggtgttcatcttttctacagataacggagggcagactttggcagggggtaataactggccccttcgaggaagaaaatggagcctgtgggaaggaggcgtccgaggggtgggctttgtggcaagccccttgctgaagcagaagggcgtgaagaaccgggagctcatccacatctctgactggctgccaacactcgtgaagctggccaggggacacaccaatggcacaaagcctctggatggcttcgacgtgtggaaaaccatcagtgaaggaagcccatcccccagaattgagctgctgcataatattgacccgaacttcgtggactcttcaccgtgtcccaggaacagcatggctccagcaaaggatgactcttctcttccagaatattcagcctttaacacatctgtccatgctgcaattagacatggaaattggaaactcctcacgggctacccaggctgtggttactggttccctccaccgtctcaatacaatgtttctgagataccctcatcagacccaccaaccaagaccctctggctctttgatattgatcgggaccctgaagaaagacatgacctgtccagagaatatcctcacatcgtcacaaagctcctgtcccgcctacagttctaccataaacactcagtccccgtgtacttccctgcacaggacccccgctgtgatcccaaggccactggggtgtggggcccttggatgtaggatttcagggaggctagaaaacctttcaattggaagttggacctcaggccttttctcacgactcttgtctcatttgttatcccaacctgggttcacttggcccttctcttgctcttaaaccacaccgaggtgtctaatttcaacccctaatgcatttaagaagctgataaaatctgcaacactcctgctgttggctggagcatgtgtctagaggtgggggtggctgggtttatccccctttcctaagccttgggacagctgggaacttaacttgaaataggaagttctcactgaatcctggaggctggaacagctggctcttttagactcacaagtcagacgttcgattcccctctgccaatagccagttttattggagtgaatcacatttcttacgcaaatgaagggagcagacagtgattaatggttctgttggccaaggcttctccctgtcggtgaaggatcatgttcaggcactccaagtgaaccacccctcttggttcaccccttactcacttatctcatcacagagcataaggcccattttgttgttcaggtcaacagcaaaatgcctgcaccatgactgtggcttttaaaataaagaaatgtgtttttatcgtaatttatttccccccagccattgctcactctgtctagacttcctgccacttccaattcttctgtggcttttcctgcctttccttttgacctcagtagtcctatccctgggaaggccactttgcttctctacctgagcacccctgatttctggaacgctgctgagccctgccttacttttgcccctagggctgaagctagaggcctccccgtaataggcggtggagttgctctgtgaggatgttcatggtagacactaagagggctgggtgggagatgcttggctctgtggcatctgttcagcgaggcttttcctatattgcatggagttagtcattgtgattgtagctttatttcataatatattaagacttgcactgctatttactagcagtgagaagaaacctcaggaaaggatatgaaaaagcaagtggccagtgtctgggatactgggccttggtaaagcagaggagggcacacccacagtcctcttattctctgttttactgcttgttttgaggttctggggtctggcaaagaggatgcagtttgacacctgcagccctttctcaatcccactaatgtcttactaatgtggaacagtccatattagctccagagagtgtcaaacccagagaaatgtgtgcaaaaatgatactcttttctgcattagccccaccattgtgttcaccaatgcttggaacactgcctgaaggcactcattttttaatttttattttatttttaattttttatatctttatgagacgatctcactctgtcaccaggttggagtacagtggtacaatcacaactcaccgtagcctcaaactcctgggctcaagtgattctcccacctcaggcacccaaatagctggaactacaggcatataccgccacacccagctaattttattttttgaaaagacaaggttccctatgttgcccagctggtcttaaactcctgggctccagcaattatcccagcttgggctccaaaagtgctgggattacaggcatgagtcaccatgcctggcctcattttttaaaacaaatgaataaatggacaaatgagtaaatgagaaagtctcacaccatgaaagatgctagtccaatgagctgaatacagaggtaatataaatgtcttccagctgttgcttttctgttctcaagctgcccctcctggggtaggagcataatctacatcactgggcagtcacaggacactctatagcaaggttgtagcgtcctctccagtggggggagaaaaggaactgtgcctaccaaaggtactctcttgtcagcaatttccatttctatactttatgggacactagaaactaaaagcaacaaataatctgatataagtccttgtatagtcatccttcaattcagtagcaatattttctggtcactactaacctgtattgtattaaaatgagactattggaaggaaatggtgctaaaactaataacatctcttaccaacctttacccaactcctgggttggcaaacagctgaccaaactgccatcacctcccacttggaagtgtatggccgacagcatgaaatagctgagcccagatgttccttctgcatcctccgaatcccagggctgggtgtaggtagccgttggaggccatcgctacagggcacctatctgttatcgctgctgtcctcccaacagctgtctccagttctagttccttggttttcaggcacagtgggggatgttctgcacccagtggacttcaaaagagttttgaagacttaattttttgtaaaacaagtacttgagattttggtttatccataatagaatgtatttcattagattctctgattctatataagaatgtgaaaagattgatatattgttgttagaaataatgttatttctttccaattttttttttttttttttttgagatggagtctcgctctgtcacccaggctggagtgcagtggtgtgatctcggctcactgcagcctctaactcccaggttcaagctattctcctgcctcagcctcccaagtagctggattacaggcatacaccaccacgcctggctatgttttgtatttttcgtagagatagggtttcaccatgttggccaggctggtctcaaactcctgacctcaagtgatccacccacttcagcttcccaaagcactgggattacaggtgtgagccactgtgcccggcaaatttttttacctttacagaaggttttgcttatttaattgtgagctcatttttctttgttacttttgtccccccagatttgggggacaaaataaaattaatcttttaaaatgtgtcagccatatgtatggggcttccatttggggtgaggagaaagttctggaactagatagtggtcatggttatacaacatcataaatgcaattactgccactgaattgtatgttttaaagtggttaaaatgttaagttttatgttttattacaatttttaaatgtgtcaaccaactttatagtacataaattatatctcagtaaagctgttaaataaataaatatagtaaaaattttagaactaaaaaaa

SEQ ID NO: 1235 - protein, 533 aa, Homo sapiens
MGPRGAASLPRGPGPRRLLLPVVLPLLLLLLLAPPGSGAGASRPPHLVFLLADDLGWNDVGFHGSRIRTPHLDALAAGGVLLDNYYTQPLCTPSRSQLLTGRYQIRTGLQHQIIWPCQPSCVPLDEKLLPQLLKEAGYTTHMVGKWHLGMYRKECLPTRRGFDTYFGYLLGSEDYYSHERCTLIDALNVTRCALDFRDGEEVATGYKNMYSTNIFTKRAIALITNHPPEKPLFLYLALQSVHEPLQVPEEYLKPYDFIQDKNRHHYAGMVSLMDEAVGNVTAALKSSGLWNNTVFIFSTDNGGQTLAGGNNWPLRGRKWSLWEGGVRGVGFVASPLLKQKGVKNRELIHISDWLPTLVKLARGHTNGTKPLDGFDVWKTISEGSPSPRIELLHNIDPNFVDSSPCPRNSMAPAKDDSSLPEYSAFNTSVHAAIRHGNWKLLTGYPGCGYWFPPPSQYNVSEIPSSDPPTKTLWLFDIDRDPEERHDLSREYPHIVTKLLSRLQFYHKHSVPVYFPAQDPRCDPKATGVWGPWM

SEQ ID NO: 1236 - DNA, 1418 bp, Homo sapiens
aaacaataacgtcattatttaataagtcatcggtgattggtccgcccctgaggttaatcttaaaagcccaggttacccgcggaaatttatgctgtccggtcaccgtgacaatgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaaaatgtttattttattgcc

SEQ ID NO: 1237 - protein, 429 aa, Homo sapiens
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 1238 - DNA, 2203 bp, Homo sapiens
gtcacatggggtgcgcgcccagactccgacccggaggcggaaccggcagtgcagcccgaagccccgcagtccccgagcacgcgtggccatgcgtcccctgcgcccccgcgccgcgctgctggcgctcctggcctcgctcctggccgcgcccccggtggccccggccgaggccccgcacctggtgcatgtggacgcggcccgcgcgctgtggcccctgcggcgcttctggaggagcacaggcttctgccccccgctgccacacagccaggctgaccagtacgtcctcagctgggaccagcagctcaacctcgcctatgtgggcgccgtccctcaccgcggcatcaagcaggtccggacccactggctgctggagcttgtcaccaccagggggtccactggacggggcctgagctacaacttcacccacctggacgggtacctggaccttctcagggagaaccagctcctcccagggtttgagctgatgggcagcgcctcgggccacttcactgactttgaggacaagcagcaggtgtttgagtggaaggacttggtctccagcctggccaggagatacatcggtaggtacggactggcgcatgtttccaagtggaacttcgagacgtggaatgagccagaccaccacgactttgacaacgtctccatgaccatgcaaggcttcctgaactactacgatgcctgctcggagggtctgcgcgccgccagccccgccctgcggctgggaggccccggcgactccttccacaccccaccgcgatccccgctgagctggggcctcctgcgccactgccacgacggtaccaacttcttcactggggaggcgggcgtgcggctggactacatctccctccacaggaagggtgcgcgcagctccatctccatcctggagcaggagaaggtcgtcgcgcagcagatccggcagctcttccccaagttcgcggacacccccatttacaacgacgaggcggacccgctggtgggctggtccctgccacagccgtggagggcggacgtgacctacgcggccatggtggtgaaggtcatcgcgcagcatcagaacctgctactggccaacaccacctccgccttcccctacgcgctcctgagcaacgacaatgccttcctgagctaccacccgcaccccttcgcgcagcgcacgctcaccgcgcgcttccaggtcaacaacacccgcccgccgcacgtgcagctgttgcgcaagccggtgctcacggccatggggctgctggcgctgctggatgaggagcagctctgggccgaagtgtcgcaggccgggaccgtcctggacagcaaccacacggtgggcgtcctggccagcgcccaccgcccccagggcccggccgacgcctggcgcgccgcggtgctgatctacgcgagcgacgacacccgcgcccaccccaaccgcagcgtcgcggtgaccctgcggctgcgcggggtgccccccggcccgggcctggtctacgtcacgcgctacctggacaacgggctctgcagccccgacggcgagtggcggcgcctgggccggcccgtcttccccacggcagagcagttccggcgcatgcgcgcggctgaggacccggtggccgcggcgccccgccccttacccgccggcggccgcctgaccctgcgccccgcgctgcggctgccgtcgcttttgctggtgcacgtgtgtgcgcgccccgagaagccgcccgggcaggtcacgcggctccgcgccctgcccctgacccaagggcagctggttctggtctggtcggatgaacacgtgggctccaagtgcctgtggacatacgagatccagttctctcaggacggtaaggcgtacaccccggtcagcaggaagccatcgaccttcaacctctttgtgttcagcccagacacaggtgctgtctctggctcctaccgagttcgagccctggactactgggcccgaccaggccccttctcggaccctgtgccgtacctggaggtccctgtgccaagagggcccccatccccgggcaatccatgagcctgtgctgagccccagtgggttgcacctccaccggcagtcagcgagctggggctgcactgtgcccatgctgccctcccatcaccccctttgcaatatatttttatattttattattttcttttatatcttggtaaaaaaaaaaaaaaaaaa

SEQ ID NO: 1239 - protein, 653 aa, Homo sapiens
MRPLRPRAALLALLASLLAAPPVAPAEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 1240 - DNA, 350 bp, Homo sapiens
aggagctgaggcctccagccaagagccttgtgagtgagtcagcttaaaagtggagcctgcagccccactcaagtcttcagatgaccacaggcctgcttgacaacaatctcacgagagaccctgagccagaaccactgggctaaaccactcccagtgtccttaccctcatagaatgtgtgaagttatgaatgtttattgttttaagctgctaagttttggggtaatttgttacgcagcaatagagagctaatactctcttttactgtcactttgttttcactcctacccttccttgacctccctctaataccttattttatagtttatattcttagggcaatgattgacac

SEQ ID NO: 1241 - DNA, 350 bp, Homo sapiens
gctcactgcagcctctaactcccaggttcaagctattctcctgcctcagcctcccaagtagctggattacaggcatacaccaccacgcctggctatgttttgtatttttcgtagagatagggtttcaccatgttggccaggctggtctcaaactcctgacctcaagtgatccacccacttcagcttcccaaagcactgggattacaggtgtgagccactgtgcccggcaaatttttttacctttacagaaggttttgcttatttaattgtgagctcatttttctttgttacttttgtccccccagatttgggggacaaaataaaattaatcttttaaaatgtgtcagcca

SEQ ID NO: 1242 - DNA, 350 bp, Homo sapiens
ctacgagctgcggggacccaggccggggcagcgggggccacgccccatctccgaccccacggggaccgggccgggactgcgccagcgggggcctcgccccgtctctgaccccagaggaaccggcagcgggcagcacgcgtgggcctctccccgcgggacgccggacgcgcagccagacgcgctccccaggccccctccgagagcgaggacgcgcccaggcccgctctgccggagccgccactggggggcgtagcgcggacgcgcacccttgcctcgggcgcctgcgcgggaggccgcgtcacgtgacccaccgcggccccgccccgcgacgagctcccgccggtcacgtg

SEQ ID NO: 1243 - DNA, 350 bp, Homo sapiens
ctgtcacccaggctggagtgctgtggcgccatcttcactcactgtaacctctgcctcctgagttcaagcaattctcctgcctcagccttccaagtagctgggattataggcgcctgccaccaggcccagctgatttttctatttttagtagagacggggtttcgccaggctgttctcgaactcctgaactcaagtgatccacctgcctcggcttcccaaagtgctgggattacaggtgtgagccaccacacccagctggtctggtccactttcttggccggatcattcatgacctttctcttgccaggttcctggatgcctatgctgagcacaagttacagttctgggca

SEQ ID NO: 1244 - DNA, 350 bp, Homo sapiens
gtcagtgtgagtggctttattctgggtggcagcaccccgtgtccggctgtaccaacaacgaggaggcacgggggcctctggaatgcatgagagtagaaaaaccagtcttgggagcgtgaggacaaatcattcctcttcatcctcctcagccatgcccagggtccgggtgcctggggcccgagcaggcgttgcccgctggatggagacaatgccgctgagcaaggcgtagcccaccatggctgccagtcctgccagcacagataggatctggttccggcgccggtatggctcctcctcagtctctgggcctgctggtgtctggcgttgcggtggtacctcagctgagggtc

SEQ ID NO: 1245 - DNA, 350 bp, Homo sapiens
aactactacttcctgtccacctttttctccattcactttaaaagctcaaggctaggtggctcatgcctgtaatcccagcactttgggaggctgaggcgggcagatcacctgaggtcgggactttgagacccgcctggacaacatggtgaaaccccatttctaataaaaatataaaaattagccaggtgtggtggcgcacgcctgtggtcccagctactctgggggctgaggcatgagaatcgcttgaacccgggaggtggaggttgcattgagctgagatcatgccacctcactccagcctgggcaacaaagattccatctcaaaaaaaaaaaaaaaagccaggcacagt

SEQ ID NO: 1246 - DNA, 350 bp, Homo sapiens
aaaaaaaaaaattaatgaagcagggctgtgcagccattacagctgttaatcaagaactgtcaatgacataaaaaaccaagctaacattgtgttagatcaaaaaaacaagatatagattatatgtggtagtctgcatgtatgtaaagctaaatatgtccatatctgcatatatgtctttaggcaggtaaaagaatgaaaggaaatatacaaatgtcagcagtgggtatgtctcaaatgctgggcttcttttcatctccagttttcacatgttccagattttatgcaatgagcatatattactctgataatggggtggagagacacagtaagcataaaaccaaaccccaaac

SEQ ID NO: 1247 - DNA, 350 bp, Homo sapiens
ctcaggaggctggggtgagaaaatcgctgaagccccggagatggaggttgcagtgagctgagatcgcgccactgcacctcagcctgggcgacaaagcaagactctgtctcaaaaacacacaaaaacagagaaaaacaagacagtaatggctcaactcacatagcaccaacgggcgaagcgttcttctgagcgctttccgagtcatcggtcctcagagcagcccctgaggcccgcaaggaagcggggctccaagccctgccgtgctcccggctccccgaggctccccgaggccacccaacccctcccacccggccatcgccccctcaccaaggccccgccccgcggcggcg

SEQ ID NO: 1248 - protein, 16 aa, Unknown
  Description of Unknown Penetratin cell permeation peptide
RQIKIWFQNRRMKWKK

SEQ ID NO: 1249 - protein, 14 aa, Human Immunodeficiency Virus
GRKKRRQRRRPPQC

SEQ ID NO: 1250 - protein, 27 aa, Unknown
  Description of Unknown Signal sequence-based cell permeation peptide
GALFLGWLGAAGSTMGAWSQPKKKRKV

SEQ ID NO: 1251 - protein, 18 aa, Unknown
  Description of Unknown PVEC cell permeation peptide
LLIILRRRIRKQAHAHSK

SEQ ID NO: 1252 - protein, 26 aa, Unknown
  Description of Unknown Transportan cell permeation peptide
GWTLNSAGYLLKINLKALAALAKKIL

SEQ ID NO: 1253 - protein, 18 aa, Unknown
  Description of Unknown Amphiphilic model cell permeation peptide
KLALKLALKALKAALKLA

SEQ ID NO: 1254 - protein, 9 aa, Unknown
  Description of Unknown Arg9 cell permeation peptide
RRRRRRRRR

SEQ ID NO: 1255 - protein, 10 aa, Unknown
  Description of Unknown Bacterial cell wall permeation peptide
KFFKFFKFFK

SEQ ID NO: 1256 - protein, 37 aa, Unknown
  Description of Unknown LL-37 cell permeation peptide
LLGDFFRKSKEKIGKEFKRIVQRIKDFLRNLVPRTES

SEQ ID NO: 1257 - protein, 31 aa, Unknown
  Description of Unknown Cecropin P1 cell permeation peptide
SWLSKTAKKLENSAKKRISEGIAIAIQGGPR

SEQ ID NO: 1258 - protein, 30 aa, Unknown
  Description of Unknown Alpha-defensin cell permeation peptide
ACYCRIPACIAGERRYGTCIYQGRLWAFCC

SEQ ID NO: 1259 - protein, 36 aa, Unknown
  Description of Unknown B-defensin cell permeation peptide
DHYNCVSSGGQCLYSACPIFTKIQGTCYRGKAKCCK

SEQ ID NO: 1260 - protein, 12 aa, Unknown
  Description of Unknown Bactenecin cell permeation peptide
RKCRIVVIRVCR

SEQ ID NO: 1261 - protein, 42 aa, Unknown
  Description of Unknown PR-39 cell permeation peptide
RRRPRPPYLPRPRPPPFFPPRLPPRIPPGFPPRFPPRFPGKR

SEQ ID NO: 1262 - protein, 13 aa, Unknown
  Description of Unknown Indolicidin cell permeation peptide
ILPWKWPWWPWRR

SEQ ID NO: 1263 - protein, 16 aa, Artificial Sequence
  Description of Artificial Sequence Synthetic peptide
AAVALLPAVLLALLAP

SEQ ID NO: 1264 - protein, 11 aa, Artificial Sequence
  Description of Artificial Sequence Synthetic peptide
AALLPVLLAAP

SEQ ID NO: 1265 - protein, 13 aa, Human Immunodeficiency Virus
GRKKRRQRRRPPQ

SEQ ID NO: 1266 - protein, 16 aa, Drosophila melanogaster
RQIKIWFQNRRMKWKK